(19) United States
(12) Patent Application Publication
(10) Pub. No.: US 2015/0105252 A1
(43) Pub. Date: Apr. 16, 2015

(54) SUBSTITUTED PYRAZOLES AS HERBICIDES

(71) Applicant: E I DU PONT DE NEMOURS AND COMPANY, Wilmington, DE (US)

(72) Inventor: THOMAS MARTIN STEVENSON, Newark, DE (US)

(21) Appl. No.: 14/515,029

(22) Filed: Oct. 15, 2014

Related U.S. Application Data
(60) Provisional application No. 61/891,071, filed on Oct. 15, 2013.

(51) Int. Cl. A01N 43/56; C07D 231/20; C07D 401/06; A01N 43/82; A01N 43/653; C07D 413/06; A01N 43/80; C07D 231/14; C07D 403/06

(52) U.S. Cl. CPC A01N 43/56; C07D 231/14; C07D 231/20; C07D 401/06; C07D 403/06; A01N 43/653; C07D 413/06; A01N 43/80; A01N 43/82
USPC 504/103; 548/374.1; 548/369.7; 546/275.4; 548/266.6; 548/365.4; 548/247; 548/143; 548/131; 504/280; 504/139; 504/282; 504/272; 504/271; 504/265

(57) ABSTRACT

Disclosed is a method for controlling the growth of undesired vegetation comprising contacting the vegetation or its environment with a herbicidally effective amount of a compound of Formula 1, including all stereoisomers, N-oxides, and salts thereof,

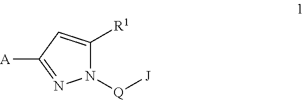

wherein A, R¹, Q and J are as defined in the disclosure.

Also disclosed is a method wherein the compound of Formula 1 (i.e. in a herbicidally effective amount) is comprised in a herbicidal composition further comprising at least one component selected from the group consisting of surfactants, solid diluents and liquid diluents. Also disclosed are compound of Formula 1 (including all stereoisomers), including N-oxides and salts thereof, their use as herbicides wherein A, R¹, Q and J are as defined in the disclosure.

### FIELD OF THE INVENTION

This invention relates to certain pyrazoles, their N-oxides, salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safe or have different sites of action.

### SUMMARY OF THE INVENTION

This invention is directed to a method for controlling the growth of undesired vegetation comprising contacting the vegetation or its environment with a herbicidally effective amount of a compound of Formula 1 (including all stereoisomers), including N-oxides and salts thereof, agricultural compositions containing them and their use as herbicides:

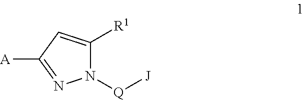

A is C(═O)N(R^{A})(R^{B}); or
A is a radical selected from the group consisting of

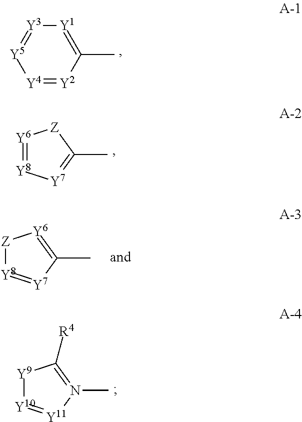

R^{A} is C₁-C₆ alkyl, C₂-C₆ alkenyl, C₂-C₆ alkynyl or C₁-C₄ alkoxy, each optionally substituted with halogen, cyano, C₁-C₄ alkoxy or C₁-C₄ alkylthio; or C₃-C₆ cycloalkyl, C₄-C₈ cycloalkylalkyl, C₃-C₇ oxiranylalkyl, C₃-C₇ oxetanylalkyl or C₃-C₇ thietanylalkyl, each optionally substituted with halogen, cyano, C₁-C₄ alkyl, C₁-C₄ alkoxy or C₁-C₄ alkylthio; or —N(R^{A1})(R^{A2}); or —SOₘ(R^{A3}); or phenyl optionally substituted with halogen, cyano, C₁-C₄ alkyl, C₁-C₃ haloalkyl, C₁-C₄ alkoxy or C₁-C₄ alkylthio;
R^{B} is H, C₁-C₄ alkyl, C₂-C₆ alkylcarbonyl or C₂-C₆ alkoxycarbonyl;
each Y¹, Y², Y³, Y⁴ and Y⁵ is independently N or CR², provided no more than 3 of Y¹, Y², Y³, Y⁴ and Y⁵ are N;
each Y⁶, Y⁷ and Y⁸ is independently N or CR³, provided no more than 2 of Y⁶, Y⁷ and Y⁸ are N;
each Y⁹, Y¹⁰ and Y¹¹ is independently N or CR⁴, provided no more than 2 of Y⁹, Y¹⁰ and Y¹¹ are N;
each Z is independently O or S;
R¹ is halogen, cyano, C₁-C₄ alkoxy, C₁-C₄ haloalkoxy, C₂-C₄ alkoxyalkyl, C₁-C₄ alkyl, C₁-C₄ haloalkyl, C₂-C₄ alkenyl, C₂-C₄ alkynyl, C₃-C₄ alkenyloxy, C₃-C₄ alkynyloxy, C₁-C₄ hydroxyalkyl, SOₙ(R^{S1}), C₂-C₄ alkylthioalkyl, C₂-C₄ alkylsulfonylalkyl, C₁-C₄ alkylamino or C₂-C₄ dialkylamino;
Q is —C(R⁵)(R⁶)— or —O—;
J is phenyl substituted with 1 R⁷ and optionally substituted with up to 2 R⁸; or
J is a 6-membered aromatic heterocyclic ring substituted with 1 R⁷ and optionally substituted with up to 2 R⁸ on carbon ring members; or
J is a 5-membered aromatic heterocyclic ring substituted with 1 R⁹ on carbon ring members and R¹⁰ on nitrogen ring members; and optionally substituted with 1 R¹¹ on carbon ring members;
each R² is independently H, halogen, cyano, nitro, SF₅, C₁-C₄ alkoxy, C₁-C₄ haloalkoxy, C₁-C₄ alkyl, C₁-C₄ haloalkyl, C₂-C₄ alkenyl, C₂-C₄ alkynyl, C₃-C₄ alkenyloxy, C₃-C₄ alkynyloxy or S(O)ₙR^{S2};
each R³ is independently H, halogen, cyano, C₁-C₄ alkyl, C₁-C₄ haloalkyl, C₁-C₄ alkoxy, C₁-C₄ haloalkoxy or S(O)ₙR^{S3};
each R⁴ is independently H, halogen, cyano, C₁-C₄ alkyl, C₁-C₄ haloalkyl, C₁-C₄ alkoxy, C₁-C₄ haloalkoxy or S(O)ₙR^{S4};
R⁵ is H, F, Cl, Br, cyano, C₁-C₄ alkyl, C₁-C₄ haloalkyl, C₂-C₆ alkylcarbonyl or C₂-C₆ alkoxycarbonyl;
R⁶ is H, F, C₁-C₄ alkyl, C₁-C₄ alkoxy or OH; or
R⁵ and R⁶ are taken together with the carbon to which they are attached to form C(═O);
R⁷ is halogen, cyano, SF₅, C₁-C₄ alkyl, C₁-C₄ haloalkyl, C₁-C₄ alkoxy, C₁-C₄ haloalkoxy or S(O)ₙR^{S5};
each R⁸ is independently halogen, cyano, C₁-C₄ alkyl, C₁-C₄ haloalkyl, C₁-C₄ alkoxy, C₁-C₄ haloalkoxy or S(O)ₙR^{S6}; or
R⁷ and R⁸ are taken together to form a 5-membered carbocyclic ring containing ring members selected from up to 2 O atoms and up to 2 S atoms, and optionally substituted on carbon atom ring members with up to 5 halogen atoms;
R⁹ is halogen, cyano, SF₅, C₁-C₄ alkyl, C₁-C₄ haloalkyl, C₁-C₄ alkoxy, C₁-C₄ haloalkoxy or S(O)ₙR^{S7};
R¹⁰ is C₁-C₄ alkyl or C₁-C₄ haloalkyl;
R¹¹ is halogen, cyano, C₁-C₄ alkyl, C₁-C₄ haloalkyl, C₁-C₄ alkoxy, C₁-C₄ haloalkoxy or S(O)ₙR^{S8};
each R^{S1}, R^{S2}, R^{S3}, R^{S4}, R^{S5}, R^{S6}, R^{S7} and R^{S8} is independently C₁-C₄ alkyl or C₁-C₄ haloalkyl; and
each m is independently 0, 1 or 2;
each n is independently 0, 1 or 2;
R^{A1} is H or C₁-C₄ alkyl;
R^{A2} is H or C₁-C₄ alkyl; and
R^{A3} is C₁-C₄ alkyl or C₁-C₄ haloalkyl;
provided when
   i) A is A-1; and each Y¹, Y², Y³, Y⁴ and Y⁵ is CH, then J is other than phenyl substituted with 1 R⁷ and optionally substituted with up to 2 R⁸;
   ii) R⁷ is CF₃, then R⁸ is other than CF₃;
   iii) R⁷ is CF₃, then R¹ is other than i-Pr; and
   iv) R^{A} is CH₃, then R^{B} is other than CH₃.

More particularly, this invention pertains to a method wherein the compound of Formula 1 (including all stereoisomers), in an N-oxide or a salt thereof. This invention also relates to a method wherein the compound of Formula 1 (i.e. in a herbicidally effective amount) is comprised in a herbicidal composition further comprising at least one component selected from the group consisting of surfactants, solid diluents and liquid diluents.

This invention also includes a method wherein the compound of Formula 1 is comprised in herbicidal mixture further comprising (b) at least one additional active ingredient selected from (b1) through (b16); and salts of compounds of (b1) through (b16).

This invention is directed to a compound of Formula 1 (including all stereoisomers), including N-oxides and salts thereof, agricultural compositions containing them and their use as herbicides:

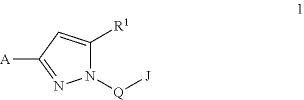

A is C(═O)N(R^{A})(R^{B}); or
A is a radical selected from the group consisting of

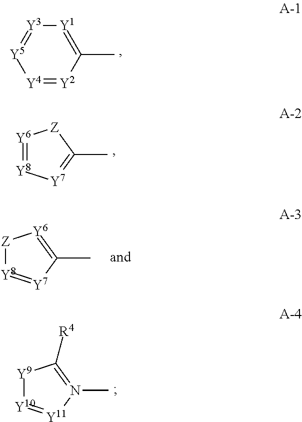

R^{A} is C₁-C₆ alkyl, C₂-C₆ alkenyl, C₂-C₆ alkynyl or C₁-C₄ alkoxy, each optionally substituted with halogen, cyano, C₁-C₄ alkoxy or C₁-C₄ alkylthio; or C₃-C₆ cycloalkyl, C₄-C₈ cycloalkylalkyl, C₃-C₇ oxiranylalkyl, C₃-C₇ oxetanylalkyl or C₃-C₇ thietanylalkyl, each optionally substituted with halogen, cyano, C₁-C₄ alkyl, C₁-C₄ alkoxy or C₁-C₄ alkylthio; or —N(R^{A1})(R^{A2}); or —SOₘ(R^{A3}); or phenyl optionally substituted with halogen, cyano, C₁-C₄ alkyl, C₁-C₃ haloalkyl, C₁-C₄ alkoxy or C₁-C₄ alkylthio;
R^{B} is H, C₁-C₄ alkyl, C₂-C₆ alkylcarbonyl or C₂-C₆ alkoxycarbonyl;
each Y¹, Y², Y³, Y⁴ and Y⁵ is independently N or CR², provided no more than 3 of Y¹, Y², Y³, Y⁴ and Y⁵ are N;
each Y⁶, Y⁷ and Y⁸ is independently N or CR³, provided no more than 2 of Y⁶, Y⁷ and Y⁸ are N;
each Y⁹, Y¹⁰ and Y¹¹ is independently N or CR⁴, provided no more than 2 of Y⁹, Y¹⁰ and Y¹¹ are N;
each Z is independently O or S;
R¹ is halogen, cyano, C₁-C₄ alkoxy, C₁-C₄ haloalkoxy, C₂-C₄ alkoxyalkyl, C₁-C₄ alkyl, C₁-C₄ haloalkyl, C₂-C₄ alkenyl, C₂-C₄ alkynyl, C₃-C₄ alkenyloxy, C₃-C₄ alkynyloxy, C₁-C₄ hydroxyalkyl, SOₙ(R^{S1}), C₂-C₄ alkylthioalkyl, C₂-C₄ alkylsulfonylalkyl, C₁-C₄ alkylamino or C₂-C₄ dialkylamino;
Q is —C(R⁵)(R⁶)— or —O—;
J is phenyl substituted with 1 R⁷ and optionally substituted with up to 2 R⁸; or
J is a 6-membered aromatic heterocyclic ring substituted with 1 R⁷ and optionally substituted with up to 2 R⁸ on carbon ring members; or
J is a 5-membered aromatic heterocyclic ring substituted with 1 R⁹ on carbon ring members and R¹⁰ on nitrogen ring members; and optionally substituted with 1 R¹¹ on carbon ring members;
each R² is independently H, halogen, cyano, nitro, SF₅, C₁-C₄ alkoxy, C₁-C₄ haloalkoxy, C₁-C₄ alkyl, C₁-C₄ haloalkyl, C₂-C₄ alkenyl, C₂-C₄ alkynyl, C₃-C₄ alkenyloxy, C₃-C₄ alkynyloxy or S(O)ₙR^{S2};
each R³ is independently H, halogen, cyano, C₁-C₄ alkyl, C₁-C₄ haloalkyl, C₁-C₄ alkoxy, C₁-C₄ haloalkoxy or S(O)ₙR^{S3};
each R⁴ is independently H, halogen, cyano, C₁-C₄ alkyl, C₁-C₄ haloalkyl, C₁-C₄ alkoxy, C₁-C₄ haloalkoxy or S(O)ₙR^{S4};
R⁵ is F, Cl, Br, cyano, C₁-C₄ alkyl, C₁-C₄ haloalkyl, C₂-C₆ alkylcarbonyl or C₂-C₆ alkoxycarbonyl;
R⁶ is H, F, C₁-C₄ alkyl, C₁-C₄ alkoxy or OH; or
R⁵ and R⁶ are taken together with the carbon to which they are attached to form C(═O);
R⁷ is halogen, cyano, SF₅, C₁-C₄ alkyl, C₁-C₄ haloalkyl, C₁-C₄ alkoxy, C₁-C₄ haloalkoxy or S(O)ₙR^{S5};
each R⁸ is independently halogen, cyano, C₁-C₄ alkyl, C₁-C₄ haloalkyl, C₁-C₄ alkoxy, C₁-C₄ haloalkoxy or S(O)ₙR^{S6}; or
R⁷ and R⁸ are taken together to form a 5-membered carbocyclic ring containing ring members selected from up to 2 O atoms and up to 2 S atoms, and optionally substituted on carbon atom ring members with up to 5 halogen atoms;
R⁹ is halogen, cyano, SF₅, C₁-C₄ alkyl, C₁-C₄ haloalkyl, C₁-C₄ alkoxy, C₁-C₄ haloalkoxy or S(O)ₙR^{S7};
R¹⁰ is C₁-C₄ alkyl or C₁-C₄ haloalkyl;
R¹¹ is halogen, cyano, C₁-C₄ alkyl, C₁-C₄ haloalkyl, C₁-C₄ alkoxy, C₁-C₄ haloalkoxy or S(O)ₙR^{S8};
each R^{S1}, R^{S2}, R^{S3}, R^{S4}, R^{S5}, R^{S6}, R^{S7} and R^{S8} is independently C₁-C₄ alkyl or C₁-C₄ haloalkyl; and
each m is independently 0, 1 or 2;
each n is independently 0, 1 or 2;
R^{A1} is H or C₁-C₄ alkyl;
R^{A2} is H or C₁-C₄ alkyl; and
R^{A3} is C₁-C₄ alkyl or C₁-C₄ haloalkyl;
provided when:
   i) A is A-1; and each Y¹, Y², Y³, Y⁴ and Y⁵ is CH, then J is other than phenyl substituted with 1 R⁷ and optionally substituted with up to 2 R⁸;
   ii) R⁷ is CF₃, then R⁸ is other than CF₃;
   iii) R⁷ is CF₃, then R¹ is other than i-Pr; and
   iv) R^{A} is CH₃, then R^{B} is other than CH₃;
and provided the compound of Formula 1 is other than a) [3-phenyl-5-(trichloromethyl)-1H-pyrazol-1-yl]-2-thienyl-methanone (949912-41-8), b) 2-furanyl[3-phenyl-5-trichloromethyl-1H-pyrazol-1-yl]-methanone (949912-42-9), c) [3-phenyl-5-(trifluoromethyl)-1H-pyrazol-1-yl]-2-thienyl-methanone (860262-65-3), d) 2-furanyl[3-phenyl-5-(trifluoromethyl)-1H-pyrazol-1-yl]-methanone (860262-66-4) and e) [3-phenyl-5-(trifluoromethyl)-1H-pyrazol-1-yl]-4-pyridinyl-methanone (860262-67-5).

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process, method, that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed. As referred to herein, the term “broadleaf” used either alone or in words such as “broadleaf weed” means dicot or dicotyledon, a term used to describe a group of angiosperms characterized by embryos having two cotyledons. As used herein, the term “alkylating reagent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals specified for R¹.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. The term “cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Alkenyl” includes straight-chain or branched alkenes such as ethenyl, 1-propenyl, 2-propenyl, and the different butenyl isomers. “Alkenyl” also includes polyenes such as 1,2-propadienyl 1,3-butadienyl. “Alkynyl” includes straight-chain or branched alkynes such as ethynyl, 1-propynyl, 2-propynyl and the different butynyl and pentynyl isomers. “Alkynyl” can also include moieties comprised of multiple triple bonds such as 1,3-butadiynyl. “Alkoxy” includes, for example, methoxy, ethoxy, n-propyloxy, isopropyloxy and the different butoxy isomers. “Alkoxyalkyl” denotes alkoxy substitution on alkyl. Examples of “alkoxyalkyl” include CH₃OCH₂, CH₃OCH₂CH₂, CH₃CH₂OCH₂ and CH₃CH₂OCH₂CH₂. “Alkenyloxy” includes straight-chain or branched alkenyloxy moieties. Examples of “alkenyloxy” include H₂C═CHCH₂O, (CH₃)CH═CHCH₂O and CH₂═CHCH₂CH₂O. “Alkynyloxy” includes straight-chain or branched alkynyloxy moieties. Examples of “alkynyloxy” include HC≡CCH₂O and CH₃C≡CCH₂O. “Alkylthio” includes branched or straight-chain alkylthio moieties such as methylthio, ethylthio and the different propylthio, butylthio isomers. “Alkylthioalkyl” denotes alkylthio substitution on alkyl. Examples of “alkylthioalkyl” include CH₃SCH₂, CH₃SCH₂CH₂, CH₃CH₂SCH₂ and CH₃CH₂SCH₂CH₂. Examples of “alkylsulfonyl” include CH₃S(O)₂—, CH₃CH₂S(O)₂— and CH₃CH₂CH₂S(O)₂—, and the different butylsulfonyl isomers. The term “alkylsulfonylalkyl” denotes alkylsulfonyl substitution on alkyl. Examples of “alkylsulfonylalkyl” include CH₃SO₂CH₂, CH₃SO₂CH₂CH₂, CH₃CH₂SO₂CH₂ and CH₃CH₂SO₂CH₂CH₂. “Hydroxyalkyl” denotes an alkyl group substituted with one hydroxy group. Examples of hydroxy alkyl include HOCH₂CH₂CH₂, CH₃CH₂CH(OH)CH₂, and CH₃CH₂CH(OH)—. “Alkylamino”, “dialkylamino” and the like, are defined analogously to the above examples.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The term “haloalkoxy” and the like, is defined analogously to the term “haloalkyl”. Examples of “haloalkoxy” include CF₃O, CCl₃CH₂O, HCF₂CH₂CH₂O and CF₃CH₂O.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃C(═O)—, CH₃CH₂OC(═O)—, CH₃CH₂CH₂C(═O)—, (CH₃)₂CHOC(═O)— and the different butoxy- or pentoxycarbonyl isomers.

“Oxiranylalkyl” denotes oxirane substitution on straight-chain or branched alkyl groups. Examples of “oxiranylalkyl” include but are not limited to

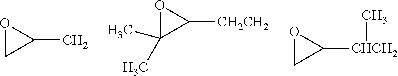

“Oxetanylalkyl” denotes oxetane substitution on straight-chain or branched alkyl groups. Examples of “oxetanylalkyl” include but are not limited to

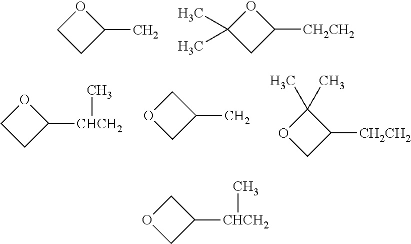

“Thietanylalkyl” denotes thietane substitution on straight-chain or branched alkyl groups. Examples of “thietanylalkyl” include but are not limited to

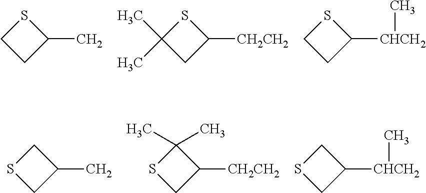

The total number of carbon atoms in a substituent group is indicated by the “Cᵢ-Cⱼ” prefix where i and j are numbers from 1 to 4. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a group contains a substituent which can be hydrogen, for example R², R³, R⁴, R⁵ and R⁶, then when this substituent is taken as hydrogen, it is recognized that this is equivalent to said group being unsubstituted. When a variable group is shown to be optionally attached to a position, for example R⁸, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” as a component of Formula 1 (e.g., substituent J) is heterocyclic. The term “ring member” refers to an atom or other moiety forming the backbone of a ring. The term “heterocyclic ring” denotes a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings and ring systems can be attached through any available carbon or nitrogen by replacement of a hydrogen on said carbon or nitrogen. “Aromatic” indicates that each of the ring atoms is essentially in the same plane and has a p-orbital perpendicular to the ring plane, and that (4n+2) π electrons, where n is a positive integer, are associated with the ring to comply with Hückel's rule.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

When J is a 5- or 6-membered nitrogen-containing heterocyclic ring, it may be attached to the remainder of Formula 1 though any available carbon or nitrogen ring atom, unless otherwise described. As noted above, J can be (among others) phenyl optionally substituted with one or more substituents selected from a group of substituents as defined in the Summary of the Invention. An example of phenyl optionally substituted with one to five substituents is the ring illustrated as U-1 in Exhibit 1, wherein R^{v} is R⁷ and R⁸ as defined in the Summary of the Invention for substitution on J and r is an integer from 0 to 3 (i.e. substituted with one R⁷ and up to two R⁸).

As noted above, J can be phenyl or a 5- or 6-membered aromatic heterocyclic ring, which may be saturated or unsaturated, optionally substituted with one or more substituents selected from a group of substituents as defined in the Summary of the Invention. Examples of a 5- or 6-membered unsaturated aromatic heterocyclic ring optionally substituted with from one or more substituents include the rings U-2 through U-61 illustrated in Exhibit 1 wherein R^{v} is any substituent as defined in the Summary of the Invention for J (i.e. R⁷, R⁸, R⁹, R¹⁰ and R¹¹) and r is an integer from 0 to 3, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

### Exhibit 1

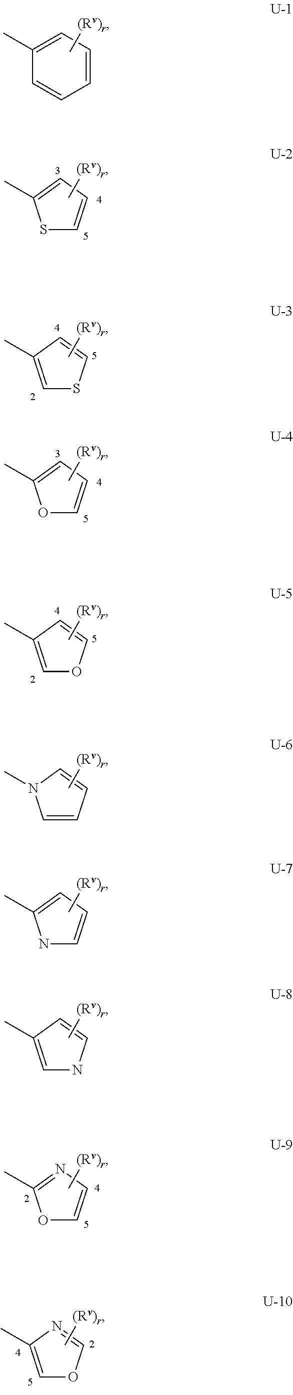

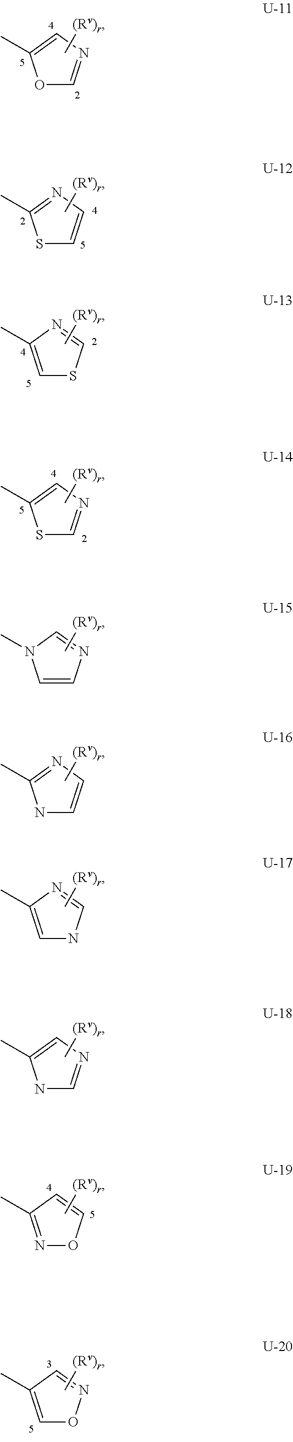

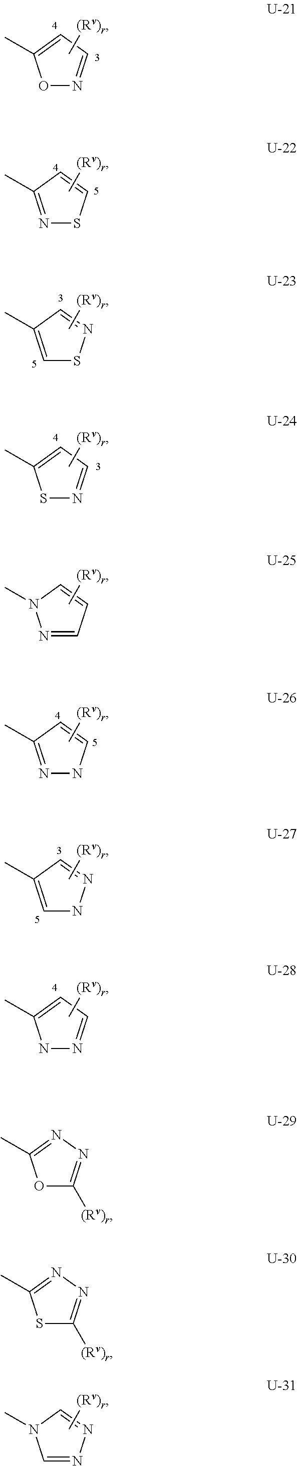

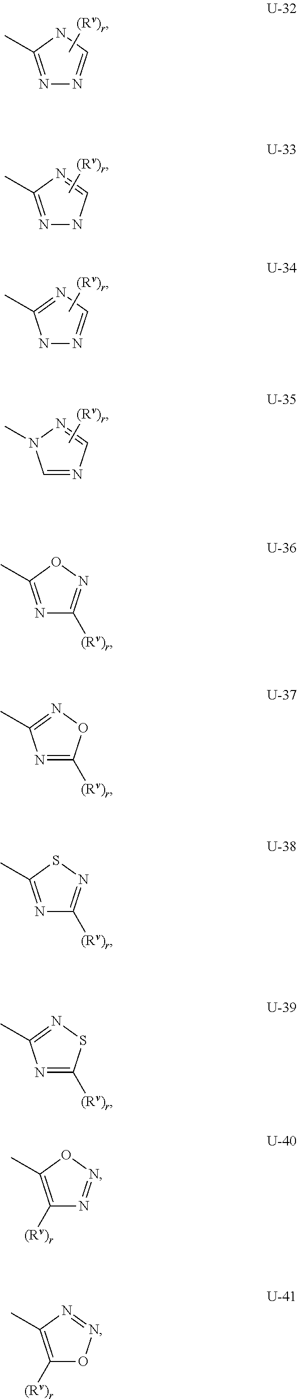

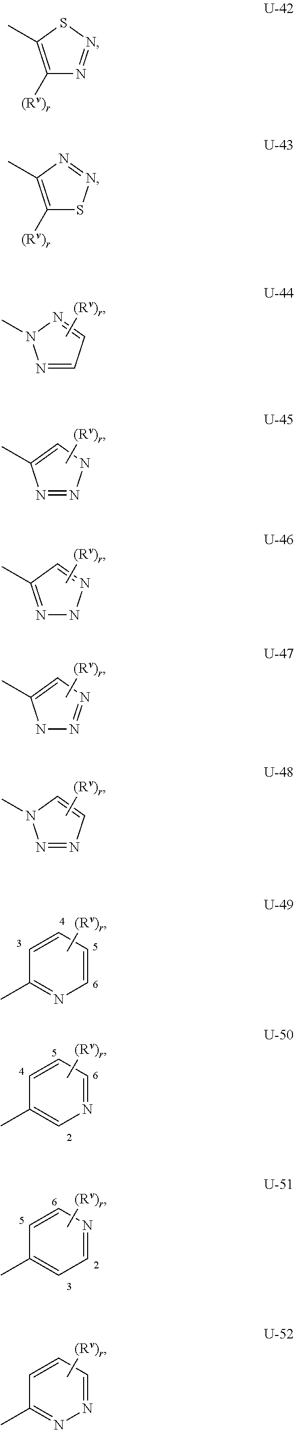

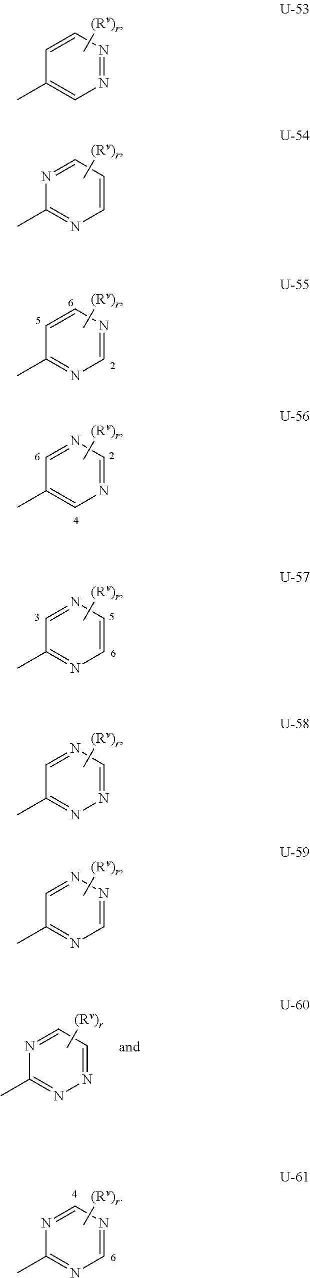

Note that when J is a 5- or 6-membered saturated or unsaturated non-aromatic heterocyclic ring optionally substituted with one or more substituents selected from the group of substituents as defined in the Summary of the Invention for J, one or two carbon ring members of the heterocycle can optionally be in the oxidized form of a carbonyl moiety.

Examples of a 5-membered carbocyclic ring containing ring members selected from up to two O atoms and up to two S atoms, and optionally substituted on carbon atom ring members with up to five halogen atoms includes the rings G-1 through G-5 as illustrated in Exhibit 2 (i.e. when R⁷ and R⁸ are taken together with two adjacent carbon atoms). Note that when the attachment point on the R^{v} group is illustrated as floating, the R^{v} group can be attached to the remainder of Formula 1 through any available carbon G group by replacement of a hydrogen atom. The optional substituents corresponding to R^{v} can be attached to any available carbon by replacing a hydrogen atom. For these G rings, r is typically an integer from 0 to 5, limited by the number of available positions on each G group.

### Exhibit 2

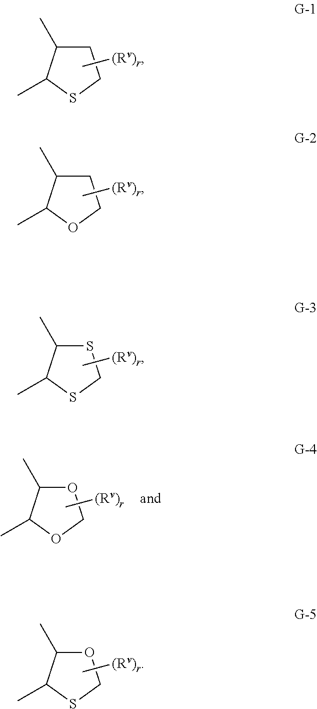

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form. For a comprehensive discussion of all aspects of stereoisomerism, see Ernest L. Eliel and Samuel H. Wilen, Stereochemistry of Organic Compounds, John Wiley & Sons, 1994.

Compounds of Formula 1 typically exist in more than one form, and Formula 1 thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Embodiments of the present invention as described in the Summary of the Invention include (where Formula 1 as used in the following Embodiments includes N-oxides and salts thereof):

### Embodiment 1

The method of Formula 1 (including all stereoisomers), N-oxides, and salts thereof, agricultural compositions containing them and their use as herbicides as described in the Summary of the Invention.

### Embodiment 2

The method of Embodiment 1 wherein A is C(═O)N(R^{A})(R^{B}); or A is a radical selected from the group consisting of A-1, A-2 and A-3.

### Embodiment 3

The method of Embodiment 2 wherein A is C(═O)N(R^{A})(R^{B}); or A is A-1.

### Embodiment 4

The method of Embodiment 2 wherein A is C(═O)N(R^{A})(R^{B}).

### Embodiment 5

The method of any one of Embodiments 1 through 3 wherein each Y¹ and Y⁵ is independently N or CR²; and each Y², Y³ and Y⁴ is CR².

### Embodiment 6

The method of Embodiment 5 wherein Y¹ is N or CR²; and each Y², Y³, Y⁴ and Y⁵ is independently CR².

### Embodiment 7

The method of Embodiment 6 wherein Y¹ is N; and each Y², Y³, Y⁴ and Y⁵ is independently CR².

### Embodiment 8

The method of Embodiment 5 wherein each Y¹, Y², Y³ and Y⁴ and is CH; and Y⁵ is CCF₃, CBr or CF.

### Embodiment 9

The method of any one of Embodiments 1 through 2 wherein each Y⁶ and Y⁷ is independently N or CR³; and Y⁸ is CR³.

### Embodiment 10

The method of Embodiment 9 wherein each Y⁶ and Y⁷ is N; and Y⁸ is CR³.

### Embodiment 11

The method of Embodiment 10 wherein each Y⁶ and Y⁷ is N; and Y⁸ is CH.

### Embodiment 12

The method of any one of Embodiments 10 or 11 wherein each Z is S.

### Embodiment 13

The method of Embodiment 1 wherein each Y⁹ and Y¹¹ is independently N or CR⁴, and Y¹⁰ is CR⁴.

### Embodiment 14

The method of Embodiment 13 wherein Y⁹ is N or CR⁴; and each Y¹⁰ and Y¹¹ is N or CR⁴.

### Embodiment 15

The method of any one of Embodiments 1 through 4 wherein R^{A} is C₁-C₆ alkyl or C₁-C₄ alkoxy, each optionally substituted with halogen, cyano or C₁-C₄ alkoxy; or C₃-C₆ cycloalkyl or C₄-C₈ cycloalkylalkyl, each optionally substituted with halogen, cyano, C₁-C₄ alkyl or C₁-C₄ alkoxy; or —N(R^{A1})(R^{A2}); or phenyl optionally substituted with halogen, cyano, C₁-C₄ alkyl, C₁-C₃ haloalkyl or C₁-C₄ alkoxy.

### Embodiment 16

The method of Embodiment 15 wherein R^{A} is C₁-C₆ alkyl or C₁-C₄ alkoxy, each optionally substituted with halogen or C₁-C₄ alkoxy; or C₃-C₆ cycloalkyl or C₄-C₈ cycloalkylalkyl, each optionally substituted with halogen, C₁-C₄ alkyl or C₁-C₄ alkoxy; or phenyl optionally substituted with halogen, C₁-C₄ alkyl, C₁-C₃ haloalkyl or C₁-C₄ alkoxy.

### Embodiment 17

The method of Embodiment 16 wherein R^{A} is C₁-C₆ alkyl or C₁-C₄ alkoxy, each optionally substituted with halogen; or C₃-C₆ cycloalkyl optionally substituted with halogen or C₁-C₄ alkyl; or phenyl optionally substituted with halogen or C₁-C₃ haloalkyl.

### Embodiment 18

The method of Embodiment 17 wherein R^{A} is C₁-C₆ alkyl or C₁-C₄ alkoxy, each optionally substituted with halogen; or C₃-C₆ cycloalkyl optionally substituted with halogen or C₁-C₄ alkyl.

### Embodiment 19

The method of Embodiment 18 wherein R^{A} is C₁-C₆ alkyl or C₁-C₄ alkoxy, each optionally substituted with halogen; or cyclopropyl.

### Embodiment 20

The method of Embodiment 19 wherein R^{A} is —CH₂CF₃, —CH₂CH₂CF₃, —CH₂CF₂CF₃, OCH₂CH₃ or OCH₃; or cyclopropyl.

### Embodiment 21

The method of Embodiment 20 wherein R^{A} is cyclopropyl.

### Embodiment 22

The method of Embodiment 20 wherein R^{A} is —CH₂CF₃.

### Embodiment 23

The method of Embodiment 17 wherein R^{A} is 4-fluorophenyl, 2,4-difluorophenyl, 2,6-difluorophenyl, 2-fluoro-4-chlorophenyl or 4-chlorophenyl.

### Embodiment 24

The method of any one of Embodiments 1 through 4 or 15 through 23 wherein R^{B} is H, C₁-C₄ alkyl or C₂-C₆ alkylcarbonyl.

### Embodiment 25

The method of Embodiment 24 wherein R^{B} is H or C₁-C₄ alkyl.

### Embodiment 26

The method of Embodiment 25 wherein R^{B} is H, CH₃ or CH₂CH₃.

### Embodiment 27

The method of Embodiment 26 wherein R^{B} is H or CH₃.

### Embodiment 28

The method of Embodiment 27 wherein R^{B} is H.

### Embodiment 29

The method of any one of Embodiments 1 through 28 wherein R¹ is halogen, C₁-C₄ alkoxy, C₁-C₄ alkyl, C₁-C₄ haloalkyl or C₁-C₄ alkylamino.

### Embodiment 30

The method of Embodiment 29 wherein R¹ is C₁-C₄ alkoxy, C₁-C₄ alkyl or C₁-C₄ haloalkyl.

### Embodiment 31

The method of Embodiment 30 wherein R¹ is C₁-C₄ alkoxy or C₁-C₄ alkyl.

### Embodiment 32

The method of Embodiment 31 wherein R¹ is C₁-C₄ alkyl.

### Embodiment 33

The method of Embodiment 32 wherein R¹ is CH₃, CH₂CH₃ or CH₂CH₂CH₃.

### Embodiment 34

The method of Embodiment 33 wherein R¹ is CH₂CH₃.

### Embodiment 35

The method of Embodiment 33 wherein R¹ is CH₃.

### Embodiment 36

The method of any one of Embodiments 1 through 35 wherein Q is —O—.

### Embodiment 37

The method of any one of Embodiments 1 through 35 wherein Q is —C(R⁵)(R⁶)—.

### Embodiment 38

The method of any one of Embodiments 1 through 37 wherein J is selected from

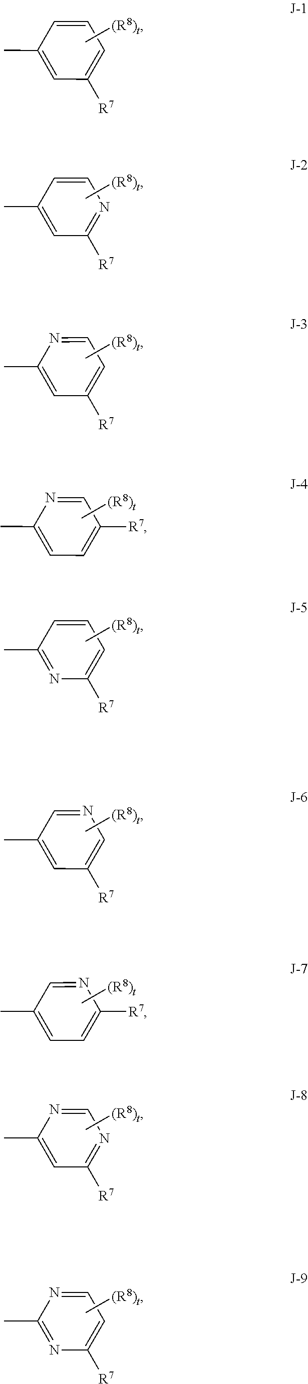

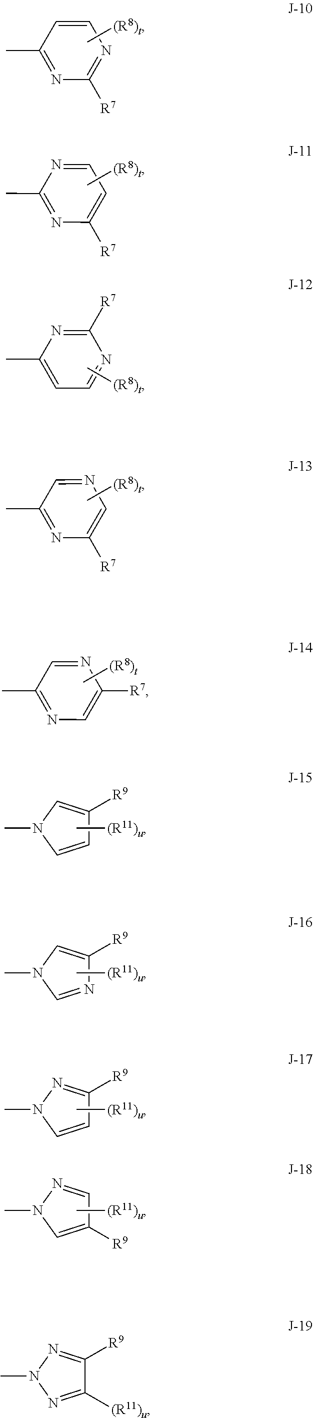

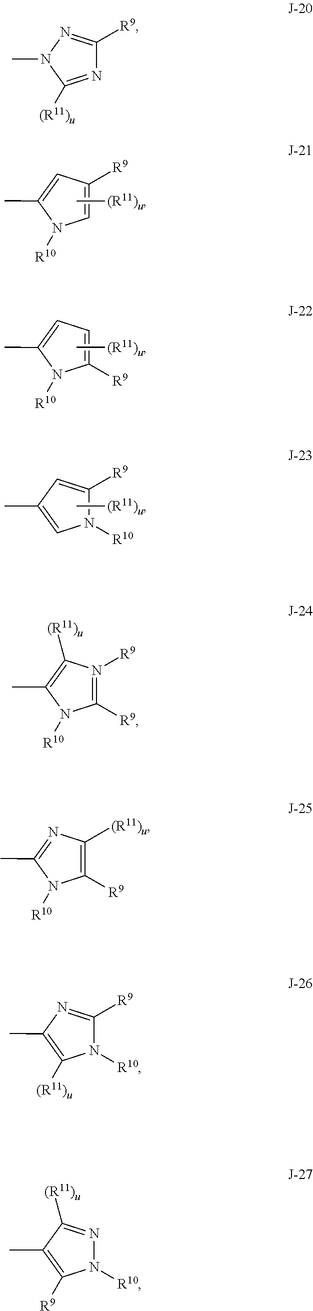

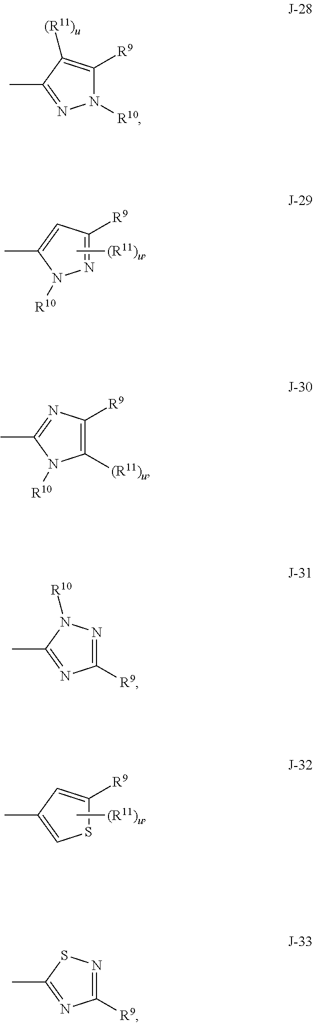

t is 0, 1 or 2; and
u is 0 or 1.

### Embodiment 39

The method of Embodiment 38 wherein J is other than J-1 (i.e. J is other than is phenyl substituted with 1 R⁷ and optionally substituted with up to 2 R⁸).

### Embodiment 40

The method of Embodiment 38 wherein J is selected from J-2 through J-14 (i.e. J is a 6-membered aromatic heterocycle selected from J-2 through J-14).

### Embodiment 41

The method of Embodiment 40 wherein J is selected from J-2 and J-10.

### Embodiment 42

The method of Embodiment 41 wherein J is J-2.

### Embodiment 43

The method of Embodiment 41 wherein J is J-10.

### Embodiment 44

The method of any one of Embodiments 38 through 43 wherein t is 0 or 1.

### Embodiment 45

The method of Embodiment 44 wherein t is 0.

### Embodiment 46

The method of Embodiment 38 wherein J is selected from J-15 through J-33 (i.e. J is a 5-membered aromatic heterocycle selected from J-15 through J-33).

### Embodiment 47

The method of Embodiment 46 wherein J is J-29.

### Embodiment 48

The method of any one of Embodiments 38 through 47 wherein u is 0.

### Embodiment 49

The method of Embodiment 38 wherein J is J-1.

### Embodiment 50

The method of any one of Embodiments 1, 2, 3, 4 through 8, or 29 through 49 wherein each R² is independently H, halogen, C₁-C₄ alkoxy, C₁-C₄ alkyl or C₁-C₄ haloalkyl.

### Embodiment 51

The method of Embodiment 50 wherein each R² is independently H, halogen, C₁-C₄ alkyl or C₁-C₄ haloalkyl.

### Embodiment 52

The method of Embodiment 51 wherein each R² is independently H, F, Cl, CH₃ or CF₃.

### Embodiment 53

The method of Embodiment 52 wherein each R² is independently H, F, Cl or CF₃.

### Embodiment 54

The method of Embodiment 53 wherein each R² is independently H or CF₃.

### Embodiment 55

The method of Embodiment 54 wherein each R² is independently H or F.

### Embodiment 56

The method of any one of Embodiments 1, 2, 9 through 11 or 28 through 49 wherein each R³ is independently H, halogen or C₁-C₄ haloalkyl.

### Embodiment 57

The method of Embodiment 56 wherein each R³ is independently H, F, Cl or CF₃.

### Embodiment 58

The method of Embodiment 57 wherein each R³ is independently H or CF₃.

### Embodiment 59

The method of any one of Embodiment 1, 13, 14 or 29 through 49 wherein each R⁴ is independently H, halogen or C₁-C₄ haloalkyl.

### Embodiment 60

The method of Embodiment 59 wherein each R⁴ is independently H, F, Cl or CF₃.

### Embodiment 61

The method of any one of Embodiments 1 through 35 or 37 through 60 wherein R⁵ is H, F or OH.

### Embodiment 62

The method of Embodiment 61 wherein R⁵ is H or F.

### Embodiment 63

The method of Embodiment 62 wherein R⁵ is H.

### Embodiment 64

The method of Embodiment 62 wherein R⁵ is F.

### Embodiment 65

The method of any one of Embodiments 1 through 35 or 36 through 60 wherein R⁶ is H or C₁-C₄ alkyl.

### Embodiment 66

The method of Embodiment 65 wherein R⁶ is CH₃.

### Embodiment 67

The method of Embodiment 65 wherein R⁶ is H.

### Embodiment 68

The method of any one of Embodiments 1 through 67 wherein R⁷ is halogen, C₁-C₄ alkyl, C₁-C₄ haloalkyl, C₁-C₄ alkoxy or C₁-C₄ haloalkoxy.

### Embodiment 69

The method of Embodiment 68 wherein R⁷ is C₁-C₄ haloalkyl or C₁-C₄ haloalkoxy.

### Embodiment 70

The method of Embodiment 69 wherein R⁷ is CF₃, OCF₃ or OCHF₂.

### Embodiment 71

The method of Embodiment 70 wherein R⁷ is CF₃ or OCHF₂.

### Embodiment 72

The method of Embodiment 71 wherein R⁷ is OCHF₂.

### Embodiment 73

The method of Embodiment 71 wherein R⁷ is CF₃.

### Embodiment 74

The method of any one of Embodiments 1 through 73 wherein each R⁸ is independently halogen or C₁-C₄ haloalkyl.

### Embodiment 75

The method of Embodiment 74 wherein each R⁸ is independently F, Cl or CF₃.

### Embodiment 76

The method of Embodiment 75 wherein each R⁸ is F.

### Embodiment 77

The method of any one of Embodiments 1 through 76 wherein R⁷ and R⁸ are taken alone (i.e. not taken together).

### Embodiment 78

The method of any one of Embodiments 1 through 67 wherein R⁷ and R⁸ are taken together with two adjacent carbon atoms to form a 5-membered carbocyclic ring containing ring members selected from up to 2 O atoms, and optionally substituted on carbon atom ring members with up to 5 halogen atoms.

### Embodiment 79

The method of any one of Embodiments 1 through 78 wherein R⁹ is halogen, C₁-C₄ alkyl, C₁-C₄ haloalkyl, C₁-C₄ alkoxy or C₁-C₄ haloalkoxy.

### Embodiment 80

The method of Embodiment 79 wherein R⁹ is C₁-C₄ haloalkyl or C₁-C₄ haloalkoxy.

### Embodiment 81

The method of Embodiment 80 wherein R⁹ is CF₃ or OCHF₂.

### Embodiment 82

The method of Embodiment 81 wherein R⁹ is OCHF₂.

### Embodiment 83

The method of Embodiment 81 wherein R⁹ is CF₃.

### Embodiment 84

The method of any one of Embodiments 1 through 83 wherein R¹⁰ is CH₃ or CH₂CF₃.

### Embodiment 85

The method of Embodiment 84 wherein R¹⁰ is CH₃.

### Embodiment 86

The method of any one of Embodiments 1 through 85 wherein R¹¹ is halogen, C₁-C₄ alkyl, C₁-C₄ haloalkyl, C₁-C₄ alkoxy or C₁-C₄ haloalkoxy.

### Embodiment 87

The method of Embodiment 86 wherein R¹¹ is C₁-C₄ haloalkyl.

### Embodiment 88

The method of Embodiment 87 wherein R¹¹ is CF₃.

### Embodiment 89

The method of any one of Embodiments 1 through 88 wherein each each R^{S1}, R^{S2}, R^{S3}, R^{S4}, R^{S5}, R^{S6}, R^{S7} and R^{S8} is independently C₁-C₄ alkyl.

### Embodiment 90

The method of Embodiment 89 wherein each R^{S1}, R^{S2}, R^{S3}, R^{S4}, R^{S5}, R^{S6}, R^{S7} and R^{S8} is independently CH₃ or CH₂CH₃.

### Embodiment 91

The method of Embodiment 89 or 90 wherein each n is independently 0 or 2.

### Embodiment 92

The method of Embodiment 91 wherein each n is independently 0.

### Embodiment 93

The method of Embodiment 91 wherein each n is independently 2.

### Embodiment 94

The method of any one of Embodiments 1 through 4 wherein R^{A1} is H or C₁-C₄ alkyl.

### Embodiment 95

The method of Embodiment 94 wherein R^{A1} is H or CH₃.

### Embodiment 96

The method of any one of Embodiments 1 through 4 wherein R^{A2} is H or C₁-C₄ alkyl.

### Embodiment 97

The method Embodiment 96 wherein R^{A2} is H or CH₃.

### Embodiment 98

The method of any one of Embodiments 1 through 4 wherein R^{A3} is C₁-C₄ alkyl.

### Embodiment 99

The method of Embodiment 98 wherein R^{A3} is CH₃.

### Embodiment 100

The method of Embodiment 98 or 99 wherein each m is independently 0 or 2.
   Embodiments of this invention, including Embodiments 1-100 above as well as any other embodiments described herein, can be combined in any manner, and the descriptions of variables in the embodiments pertain not only to the compounds of Formula 1 used in the method but also to the starting compounds and intermediate compounds useful for preparing the compounds of Formula 1 used in the method. In addition, embodiments of this invention, including Embodiments 1-100 above as well as any other embodiments described herein, and any combination thereof, pertain to the compositions and compounds of the present invention.

### Embodiment A

A method of the Summary of the Invention wherein
A is C(═O)N(R^{A})(R^{B}); or A is a radical selected from the group consisting of A-1, A-2 and A-3;
each Y¹ and Y⁵ is independently N or CR²; and each Y², Y³ and Y⁴ is CR²;
each Y⁶ and Y⁷ is independently N or CR³; and Y⁸ is CR³;
R^{A} is C₁-C₆ alkyl or C₁-C₄ alkoxy, each optionally substituted with halogen, cyano or C₁-C₄ alkoxy; or C₃-C₆ cycloalkyl or C₄-C₈ cycloalkylalkyl, each optionally substituted with halogen, cyano, C₁-C₄ alkyl or C₁-C₄ alkoxy; or —N(R^{A1})(R^{A2}); or phenyl optionally substituted with halogen, cyano, C₁-C₄ alkyl, C₁-C₃ haloalkyl or C₁-C₄ alkoxy;
R^{B} is H, C₁-C₄ alkyl or C₂-C₆ alkylcarbonyl;
R¹ is halogen, C₁-C₄ alkoxy, C₁-C₄ alkyl, C₁-C₄ haloalkyl or C₁-C₄ alkylamino;
J is selected from

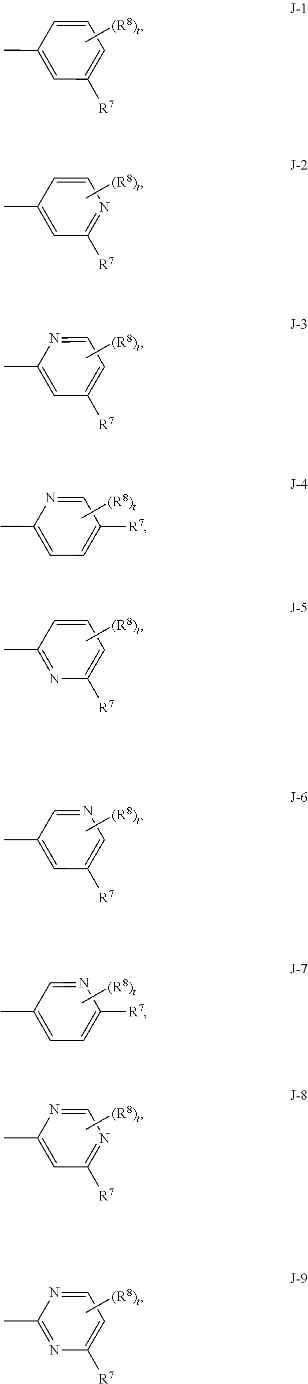

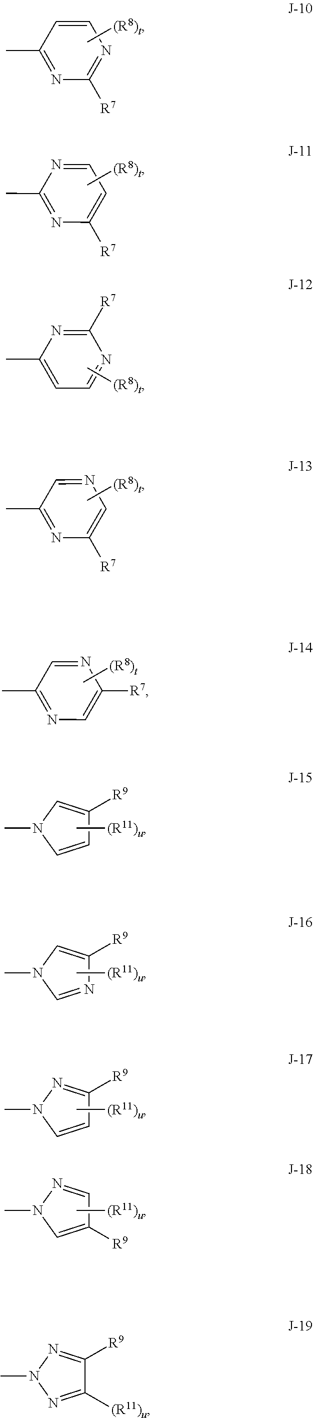

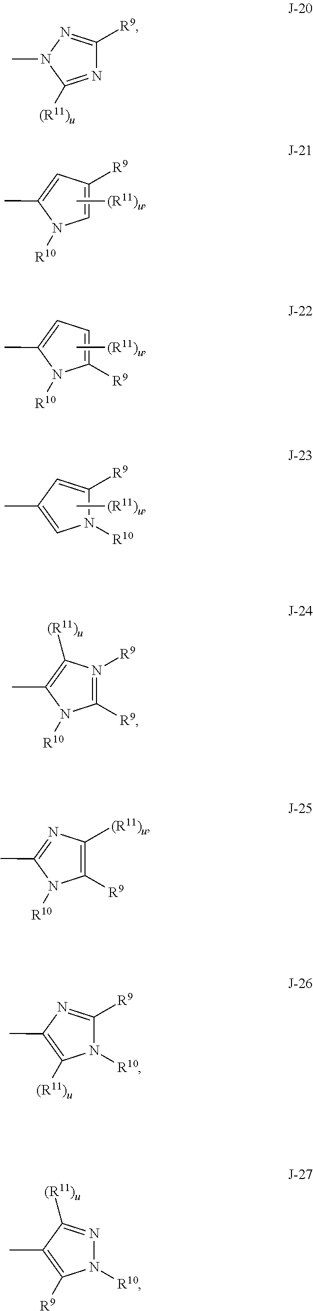

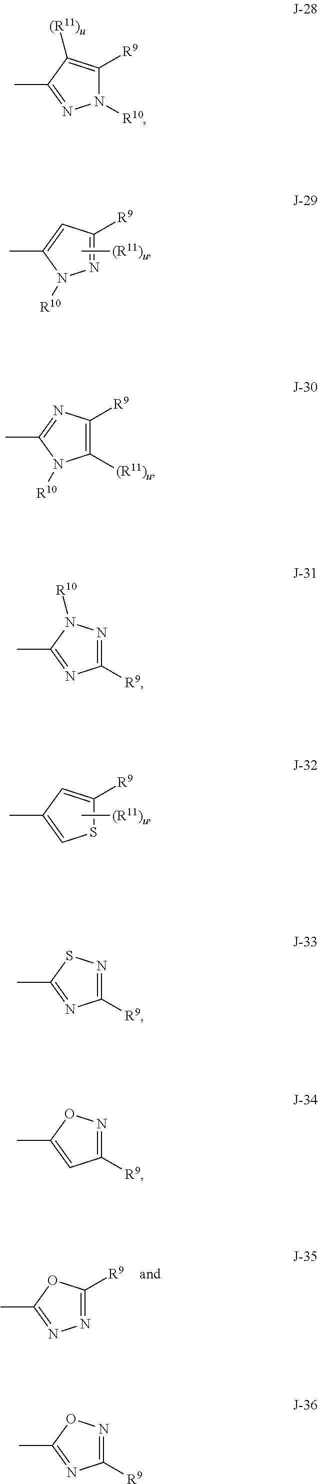

t is 0, 1 or 2; and
u is 0 or 1;
each R² is independently H, halogen, C₁-C₄ alkoxy, C₁-C₄ alkyl or C₁-C₄ haloalkyl;
each R³ is independently H, halogen or C₁-C₄ haloalkyl;
R⁵ is H, F or OH;
R⁶ is H or C₁-C₄ alkyl;
R⁷ is halogen, C₁-C₄ alkyl, C₁-C₄ haloalkyl, C₁-C₄ alkoxy or C₁-C₄ haloalkoxy;
R⁸ is independently halogen or C₁-C₄ haloalkyl; or
R⁷ and R⁸ are taken together with two adjacent carbon atoms to form a 5-membered carbocyclic ring containing ring members selected from up to 2 O atoms, and optionally substituted on carbon atom ring members with up to 5 halogen atoms;
R⁹ is halogen, C₁-C₄ alkyl, C₁-C₄ haloalkyl, C₁-C₄ alkoxy or C₁-C₄ haloalkoxy;
R¹⁰ is CH₃ or CH₂CF₃;
R¹¹ is halogen, C₁-C₄ alkyl, C₁-C₄ haloalkyl, C₁-C₄ alkoxy or C₁-C₄ haloalkoxy;
R^{A1} is H or C₁-C₄ alkyl; and
R^{A2} is H or C₁-C₄ alkyl.

### Embodiment B

A method Embodiment A wherein
A is C(═O)N(R^{A})(R^{B}); or A is A-1;
Y¹ is N or CR²; and each Y², Y³, Y⁴ and Y⁵ is independently CR²;
R^{A} is C₁-C₆ alkyl or C₁-C₄ alkoxy, each optionally substituted with halogen or C₁-C₄ alkoxy; or C₃-C₆ cycloalkyl or C₄-C₈ cycloalkylalkyl, each optionally substituted with halogen, C₁-C₄ alkyl or C₁-C₄ alkoxy; or phenyl optionally substituted with halogen, C₁-C₄ alkyl, C₁-C₃ haloalkyl or C₁-C₄ alkoxy;
R^{B} is H or C₁-C₄ alkyl;
R¹ is C₁-C₄ alkoxy, C₁-C₄ alkyl or C₁-C₄ haloalkyl;
Q is —C(R⁵)(R⁶)—;
J is selected from J-2 through J-14;
t is 0 or 1;
each R² is independently H, halogen, C₁-C₄ alkyl or C₁-C₄ haloalkyl;
R⁵ is H or F;
R⁶ is H;
R⁷ is C₁-C₄ haloalkyl or C₁-C₄ haloalkoxy; and
each R⁸ is independently F, Cl or CF₃.

### Embodiment C

A method Embodiment B wherein
A is C(═O)N(R^{A})(R^{B});
R^{A} is C₁-C₆ alkyl or C₁-C₄ alkoxy, each optionally substituted with halogen; or C₃-C₆ cycloalkyl optionally substituted with halogen or C₁-C₄ alkyl; or phenyl optionally substituted with halogen or C₁-C₃ haloalkyl;
R^{B} is H, CH₃ or CH₂CH₃;
R¹ is C₁-C₄ alkoxy or C₁-C₄ alkyl;
J is selected from J-2 and J-5;
t is 0;
each R² is independently H, F, Cl, CH₃ or CF₃;
R⁵ is H; and
R⁷ is CF₃, OCF₃ or OCHF₂.

### Embodiment D

A method Embodiment C wherein
R^{A} is C₁-C₆ alkyl or C₁-C₄ alkoxy, each optionally substituted with halogen; or C₃-C₆ cycloalkyl optionally substituted with halogen or C₁-C₄ alkyl;
R^{B} is H or CH₃;
R¹ is C₁-C₄ alkyl;
J is J-2; and
R⁷ is CF₃.

### Embodiment E

A method Embodiment A wherein
A is C(═O)N(R^{A})(R^{B});
R^{A} is C₁-C₆ alkyl or C₁-C₄ alkoxy, each optionally substituted with halogen; or cyclopropyl;
R^{B} is H;
R¹ is CH₃, CH₂CH₃ or CH₂CH₂CH₃;
Q is —C(R⁵)(R⁶)—;
J is selected from J-15 through J-33;
u is 0;
R⁵ is H;
R⁶ is H;
R⁹ is C₁-C₄ haloalkyl or C₁-C₄ haloalkoxy; and
R¹⁰ is CH₃.

### Embodiment F

A method Embodiment E wherein
R^{A} is —CH₂CF₃;
R¹ is CH₂CH₃;
J is J-29; and
R⁹ is F, CH₃ or CF₃.

Specific embodiments include methods of Formula 1 selected from the group consisting of:
5-ethyl-N-(2,2,2-trifluoroethyl)-1-[[(2-trifluoromethyl)-4-pyridinyl]methyl]-1H-pyrazole-3-carboxamide (Compound 78);
N-cyclopropyl-5-ethyl-1-[[2-trifluoromethyl)-4-pyridinyl]methyl]-1H-pyrazole-3-carboxamide (Compound 76); and
5-ethyl-1-[[1-methyl-3-(trifluoromethyl)-1H-pyrazole-5-yl]methyl]-N-(2,2,2-trifluoroethyl)-1H-pyrazole-3-carboxamide (Compound 108).

Also noteworthy as embodiments are herbicidal compositions of the present invention comprising the compounds as described in the embodiments above. Of note as embodiments relating to methods of use are those involving the compounds of embodiments described above. Compounds of the invention are particularly useful for selective control of weeds in cereal crops such as wheat, barley, maize, soybean, sunflower, cotton, oilseed rape and rice, and specialty crops such as sugarcane, citrus, fruit and nut crops.

Embodiments of the present invention as described in the Summary of the Invention include (where Formula 1 as used in the following Embodiments includes N-oxides and salts thereof):

### Embodiment 101

A compound of Formula 1 (including all stereoisomers), N-oxides, and salts thereof, agricultural compositions containing them and their use as herbicides as described in the Summary of the Invention.

### Embodiment 102

A compound of Embodiment 101 wherein A is C(═O)N(R^{A})(R^{B}); or A is a radical selected from the group consisting of A-1, A-2 and A-3.

### Embodiment 103

A compound of Embodiment 102 wherein A is C(═O)N(R^{A})(R^{B}); or A is A-1.

### Embodiment 104

A compound of Embodiment 102 wherein A is C(═O)N(R^{A})(R^{B}).

### Embodiment 105

A compound of any one of Embodiments 101 through 103 wherein each Y¹ and Y⁵ is independently N or CR²; and each Y², Y³ and Y⁴ is CR².

### Embodiment 106

A compound of Embodiment 105 wherein Y¹ is N or CR²; and each Y², Y³, Y⁴ and Y⁵ is independently CR².

### Embodiment 107

A compound of Embodiment 106 wherein Y¹ is N; and each Y², Y³, Y⁴ and Y⁵ is independently CR².

### Embodiment 108

A compound of Embodiment 105 wherein each Y¹, Y², Y³ and Y⁴ and is CH; and Y⁵ is CCF₃, CBr or CF.

### Embodiment 109

A compound of any one of Embodiments 101 through 102 wherein each Y⁶ and Y⁷ is independently N or CR³; and Y⁸ is CR³.

### Embodiment 110

A compound of Embodiment 109 wherein each Y⁶ and Y⁷ is N; and Y⁸ is CR³.

### Embodiment 111

A compound of Embodiment 110 wherein each Y⁶ and Y⁷ is N; and Y⁸ is CH.

### Embodiment 112

A compound of any one of Embodiments 110 or 111 wherein each Z is S.

### Embodiment 113

A compound of Embodiment 101 wherein each Y⁹ and Y¹¹ is independently N or CR⁴, and Y¹⁰ is CR⁴.

### Embodiment 114

A compound of Embodiment 113 wherein Y⁹ is N or CR⁴; and each Y¹⁰ and Y¹¹ is N or CR⁴.

### Embodiment 115

A compound of any one of Embodiments 101 through 104 wherein R^{A} is C₁-C₆ alkyl or C₁-C₄ alkoxy, each optionally substituted with halogen, cyano or C₁-C₄ alkoxy; or C₃-C₆ cycloalkyl or C₄-C₈ cycloalkylalkyl, each optionally substituted with halogen, cyano, C₁-C₄ alkyl or C₁-C₄ alkoxy; or —N(R^{A1})(R^{A2}); or phenyl optionally substituted with halogen, cyano, C₁-C₄ alkyl, C₁-C₃ haloalkyl or C₁-C₄ alkoxy.

### Embodiment 116

A compound of Embodiment 115 wherein R^{A} is C₁-C₆ alkyl or C₁-C₄ alkoxy, each optionally substituted with halogen or C₁-C₄ alkoxy; or C₃-C₆ cycloalkyl or C₄-C₈ cycloalkylalkyl, each optionally substituted with halogen, C₁-C₄ alkyl or C₁-C₄ alkoxy; or phenyl optionally substituted with halogen, C₁-C₄ alkyl, C₁-C₃ haloalkyl or C₁-C₄ alkoxy.

### Embodiment 117

A compound of Embodiment 116 wherein R^{A} is C₁-C₆ alkyl or C₁-C₄ alkoxy, each optionally substituted with halogen; or C₃-C₆ cycloalkyl optionally substituted with halogen or C₁-C₄ alkyl; or phenyl optionally substituted with halogen or C₁-C₃ haloalkyl.

### Embodiment 118

A compound of Embodiment 117 wherein R^{A} is C₁-C₆ alkyl or C₁-C₄ alkoxy, each optionally substituted with halogen; or C₃-C₆ cycloalkyl optionally substituted with halogen or C₁-C₄ alkyl.

### Embodiment 119

A compound of Embodiment 118 wherein R^{A} is C₁-C₆ alkyl or C₁-C₄ alkoxy, each optionally substituted with halogen; or cyclopropyl.

### Embodiment 120

A compound of Embodiment 119 wherein R^{A} is —CH₂CF₃, —CH₂CH₂CF₃, —CH₂CF₂CF₃, OCH₂CH₃ or OCH₃; or cyclopropyl.

### Embodiment 121

A compound of Embodiment 120 wherein R^{A} is cyclopropyl.

### Embodiment 122

A compound of Embodiment 120 wherein R^{A} is —CH₂CF₃.

### Embodiment 123

A compound of Embodiment 117 wherein R^{A} is 4-fluorophenyl, 2,4-difluorophenyl, 2,6-difluorophenyl, 2-fluoro-4-chlorophenyl or 4-chlorophenyl.

### Embodiment 124

A compound of any one of Embodiments 101 through 104 or 115 through 123 wherein R^{B} is H, C₁-C₄ alkyl or C₂-C₆ alkylcarbonyl.

### Embodiment 125

A compound of Embodiment 124 wherein R^{B} is H or C₁-C₄ alkyl.

### Embodiment 126

A compound of Embodiment 125 wherein R^{B} is H, CH₃ or CH₂CH₃.

### Embodiment 127

A compound of Embodiment 126 wherein R^{B} is H or CH₃.

### Embodiment 128

A compound of Embodiment 127 wherein R^{B} is H.

### Embodiment 129

A compound of any one of Embodiments 101 through 128 wherein R¹ is halogen, C₁-C₄ alkoxy, C₁-C₄ alkyl, C₁-C₄ haloalkyl or C₁-C₄ alkylamino.

### Embodiment 130

A compound of Embodiment 129 wherein R¹ is C₁-C₄ alkoxy, C₁-C₄ alkyl or C₁-C₄ haloalkyl.

### Embodiment 131

A compound of Embodiment 130 wherein R¹ is C₁-C₄ alkoxy or C₁-C₄ alkyl.

### Embodiment 132

A compound of Embodiment 131 wherein R¹ is C₁-C₄ alkyl.

### Embodiment 133

A compound of Embodiment 132 wherein R¹ is CH₃, CH₂CH₃ or CH₂CH₂CH₃.

### Embodiment 134

A compound of Embodiment 133 wherein R¹ is CH₂CH₃.

### Embodiment 135

A compound of Embodiment 133 wherein R¹ is CH₃.

### Embodiment 136

A compound of any one of Embodiments 101 through 135 wherein Q is —O—.

### Embodiment 137

A compound of any one of Embodiments 101 through 135 wherein Q is —C(R⁵)(R⁶)—.

### Embodiment 138

A compound of any one of Embodiments 101 through 137 wherein J is selected from

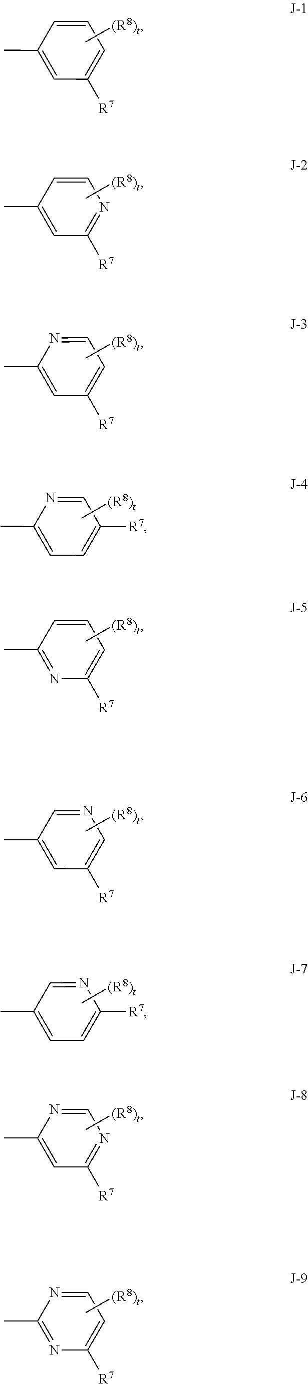

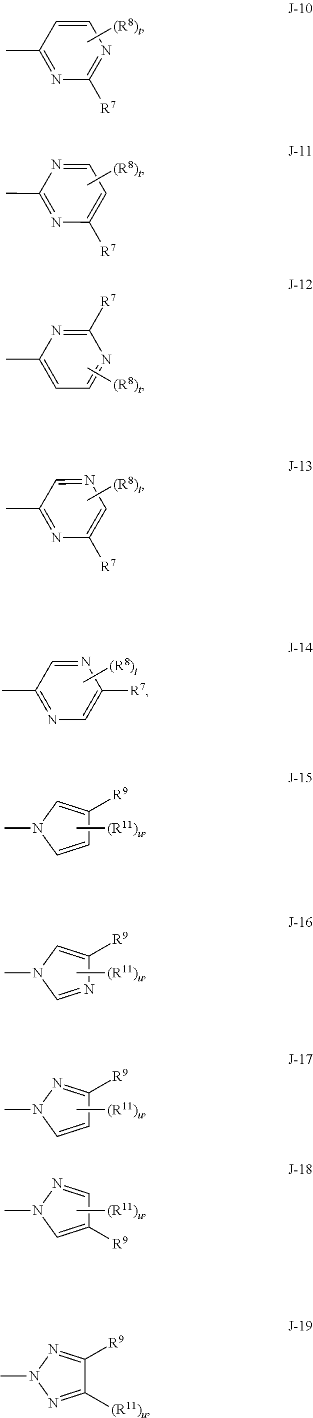

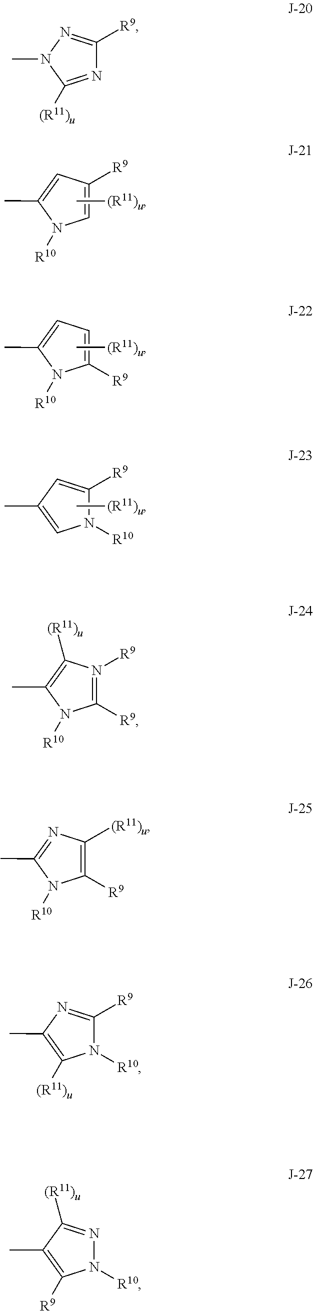

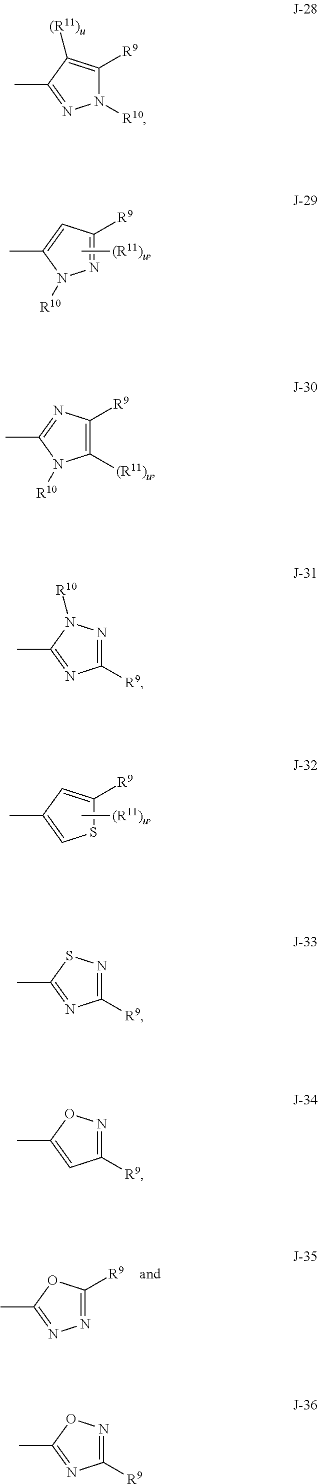

t is 0, 1 or 2; and
u is 0 or 1.

### Embodiment 139

A compound of Embodiment 138 wherein J is other than J-1 (i.e. J is other than is phenyl substituted with 1 R⁷ and optionally substituted with up to 2 R⁸).

### Embodiment 140

A compound of Embodiment 138 wherein J is selected from J-2 through J-14 (i.e. J is a 6-membered aromatic heterocycle selected from J-2 through J-14).

### Embodiment 141

A compound of Embodiment 140 wherein J is selected from J-2 and J-10.

### Embodiment 142

A compound of Embodiment 141 wherein J is J-2.

### Embodiment 143

A compound of Embodiment 141 wherein J is J-10.

### Embodiment 144

A compound of any one of Embodiments 101 through 143 wherein t is 0 or 1.

### Embodiment 145

A compound of Embodiment 144 wherein t is 0.

### Embodiment 146

A compound of Embodiment 138 wherein J is selected from J-15 through J-33 (i.e. J is a 5-membered aromatic heterocycle selected from J-15 through J-33).

### Embodiment 147

A compound of Embodiment 146 wherein J is J-29.

### Embodiment 148

A compound of any one of Embodiments 138 through 147 wherein u is O.

### Embodiment 149

A compound of Embodiment 138 wherein J is J-1.

### Embodiment 150

A compound of any one of Embodiments 101, 102, 103, 104 through 108, or 129 through 149 wherein each R² is independently H, halogen, C₁-C₄ alkoxy, C₁-C₄ alkyl or C₁-C₄ haloalkyl.

### Embodiment 151

A compound of Embodiment 150 wherein each R² is independently H, halogen, C₁-C₄ alkyl or C₁-C₄ haloalkyl.

### Embodiment 152

A compound of Embodiment 151 wherein each R² is independently H, F, Cl, CH₃ or CF₃.

### Embodiment 153

A compound of Embodiment 152 wherein each R² is independently H, F, Cl or CF₃.

### Embodiment 154

A compound of Embodiment 153 wherein each R² is independently H or CF₃.

### Embodiment 155

A compound of Embodiment 154 wherein each R² is independently H or F.

### Embodiment 156

A compound of any one of Embodiments 101, 102, 109 through 111 or 128 through 149 wherein each R³ is independently H, halogen or C₁-C₄ haloalkyl.

### Embodiment 157

A compound of Embodiment 156 wherein each R³ is independently H, F, Cl or CF₃.

### Embodiment 158

A compound of Embodiment 157 wherein each R³ is independently H or CF₃.

### Embodiment 159

A compound of any one of Embodiment 101, 113, 114 or 129 through 149 wherein each R⁴ is independently H, halogen or C₁-C₄ haloalkyl.

### Embodiment 160

A compound of Embodiment 159 wherein each R⁴ is independently H, F, Cl or CF₃.

### Embodiment 161

A compound of any one of Embodiments 101 through 135 or 137 through 160 wherein R⁵ is F or OH.

### Embodiment 162

A compound of Embodiment 161 wherein R⁵ is F.

### Embodiment 163

A compound of Embodiment 160 wherein R⁵ is OH.

### Embodiment 164

A compound of any one of Embodiments 101 through 135 or 136 through 160 wherein R⁶ is H or C₁-C₄ alkyl.

### Embodiment 165

A compound of Embodiment 164 wherein R⁶ is H or CH₃.

### Embodiment 166

A compound of any one of Embodiments 1 through 165 wherein R⁷ is halogen, C₁-C₄ alkyl, C₁-C₄ haloalkyl, C₁-C₄ alkoxy or C₁-C₄ haloalkoxy.

### Embodiment 167

A compound of Embodiment 166 wherein R⁷ is C₁-C₄ haloalkyl or C₁-C₄ haloalkoxy.

### Embodiment 168

A compound of Embodiment 69 wherein R⁷ is CF₃, OCF₃ or OCHF₂.

### Embodiment 169

A compound of Embodiment 168 wherein R⁷ is CF₃ or OCHF₂.

### Embodiment 170

A compound of Embodiment 169 wherein R⁷ is OCHF₂.

### Embodiment 171

A compound of Embodiment 171 wherein R⁷ is CF₃.

### Embodiment 172

A compound of any one of Embodiments 101 through 171 wherein each R⁸ is independently halogen or C₁-C₄ haloalkyl.

### Embodiment 173

A compound of Embodiment 172 wherein each R⁸ is independently F, Cl or CF₃.

### Embodiment 174

A compound of Embodiment 173 wherein each R⁸ is F.

### Embodiment 175

A compound of any one of Embodiments 1 through 174 wherein R⁷ and R⁸ are taken alone (i.e. not taken together).

### Embodiment 176

A compound of any one of Embodiments 101 through 165 wherein R⁷ and R⁸ are taken together with two adjacent carbon atoms to form a 5-membered carbocyclic ring containing ring members selected from up to 2 O atoms, and optionally substituted on carbon atom ring members with up to 5 halogen atoms.

### Embodiment 177

A compound of any one of Embodiments 1 through 176 wherein R⁹ is halogen, C₁-C₄ alkyl, C₁-C₄ haloalkyl, C₁-C₄ alkoxy or C₁-C₄ haloalkoxy.

### Embodiment 178

A compound of Embodiment 177 wherein R⁹ is C₁-C₄ haloalkyl or C₁-C₄ haloalkoxy.

### Embodiment 179

A compound of Embodiment 178 wherein R⁹ is CF₃ or OCHF₂.

### Embodiment 180

A compound of Embodiment 179 wherein R⁹ is OCHF₂.

### Embodiment 181

A compound of Embodiment 179 wherein R⁹ is CF₃.

### Embodiment 182

A compound of any one of Embodiments 101 through 181 wherein R¹⁰ is CH₃ or CH₂CF₃.

### Embodiment 183

A compound of Embodiment 182 wherein R¹⁰ is CH₃.

### Embodiment 184

A compound of any one of Embodiments 1 through 183 wherein R¹¹ is halogen, C₁-C₄ alkyl, C₁-C₄ haloalkyl, C₁-C₄ alkoxy or C₁-C₄ haloalkoxy.

### Embodiment 185

A compound of Embodiment 184 wherein R¹¹ is C₁-C₄ haloalkyl.

### Embodiment 186

A compound of Embodiment 185 wherein R¹¹ is CF₃.

### Embodiment 187

A compound of any one of Embodiments 101 through 186 wherein each R^{S1}, R^{S2}, R^{S3}, R^{S4}, R^{S5}, R^{S6}, R^{S7} and R^{S8} is independently C₁-C₄ alkyl.

### Embodiment 188

A compound of Embodiment 187 wherein each R^{S1}, R^{S2}, R^{S3}, R^{S4}, R^{S5}, R^{S6}, R^{S7} and R^{S8} is independently CH₃ or CH₂CH₃.

### Embodiment 189

A compound of Embodiment 187 or 188 wherein each n is independently 0 or 2.

### Embodiment 190

A compound of Embodiment 189 wherein each n is independently 0.

### Embodiment 191

A compound of Embodiment 189 wherein each n is independently 2.

### Embodiment 192

A compound of any one of Embodiments 101 through 104 wherein R^{A1} is H or C₁-C₄ alkyl.

### Embodiment 193

A compound of Embodiment 192 wherein R^{A1} is H or CH₃.

### Embodiment 194

A compound of any one of Embodiments 101 through 104 wherein R^{A2} is H or C₁-C₄ alkyl.

### Embodiment 195

A compound Embodiment 194 wherein R^{A2} is H or CH₃.

### Embodiment 196

A compound of any one of Embodiments 101 through 104 wherein R^{A3} is C₁-C₄ alkyl.

### Embodiment 197

A compound of Embodiment 196 wherein R^{A3} is CH₃.

### Embodiment 198

A compound of Embodiment 196 or 197 wherein each m is independently 0 or 2.

### Embodiment 199

A compound of any one of Embodiments 101 through 131 or 136 through 198 wherein R¹ is other than C₁-C₄ alkoxy, C₁-C₄ haloalkoxy, C₃-C₄ alkenyloxy or C₃-C₄ alkynyloxy.

### Embodiment 200

A compound of Embodiment 199 wherein R¹ is other than methoxy (i.e. —OCH₃).

### Embodiment 201

A compound of Embodiment 199 wherein R¹ is other than n-butoxy (i.e. —OCH₂CH₂CH₂CH₃).

### Embodiment 202

A compound of Formula 1 wherein R¹ is other than i-Pr.

### Embodiment 202

A compound of Formula 1 wherein R¹ is other than SOₙ(R^{S1}).

### Embodiment 203

A compound of Formula 1 wherein R¹ is other than cyano.

### Embodiment 204

A compound of any one of Embodiments 101 through 203 wherein when A is A-1, and Y¹ and Y² are both CR² than R² is other than halogen or C₁-C₄ alkoxy.

### Embodiment 205

A compound of any one of Embodiments 101 through 203 wherein when A is A-2, and Y⁶ and Y⁷ are both CR³ then R³ is other halogen, cyano or C₁-C₄ alkoxy.
   Embodiments of this invention, including Embodiments 101-205 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 101-205 above as well as any other embodiments described herein, and any combination thereof, pertain to the compositions and methods of the present invention.

### Embodiment G

A compound of the Summary of the Invention wherein
A is C(═O)N(R^{A})(R^{B}); or A is a radical selected from the group consisting of A-1, A-2 and A-3;
each Y¹ and Y⁵ is independently N or CR²; and each Y², Y³ and Y⁴ is CR²;
each Y⁶ and Y⁷ is independently N or CR³; and Y⁸ is CR³;
R^{A} is C₁-C₆ alkyl or C₁-C₄ alkoxy, each optionally substituted with halogen, cyano or C₁-C₄ alkoxy; or C₃-C₆ cycloalkyl or C₄-C₈ cycloalkylalkyl, each optionally substituted with halogen, cyano, C₁-C₄ alkyl or C₁-C₄ alkoxy; or —N(R^{A1})(R^{A2});
or phenyl optionally substituted with halogen, cyano, C₁-C₄ alkyl, C₁-C₃ haloalkyl or C₁-C₄ alkoxy;
R^{B} is H, C₁-C₄ alkyl or C₂-C₆ alkylcarbonyl;
R¹ is halogen, C₁-C₄ alkoxy, C₁-C₄ alkyl, C₁-C₄ haloalkyl or C₁-C₄ alkylamino;
J is selected from

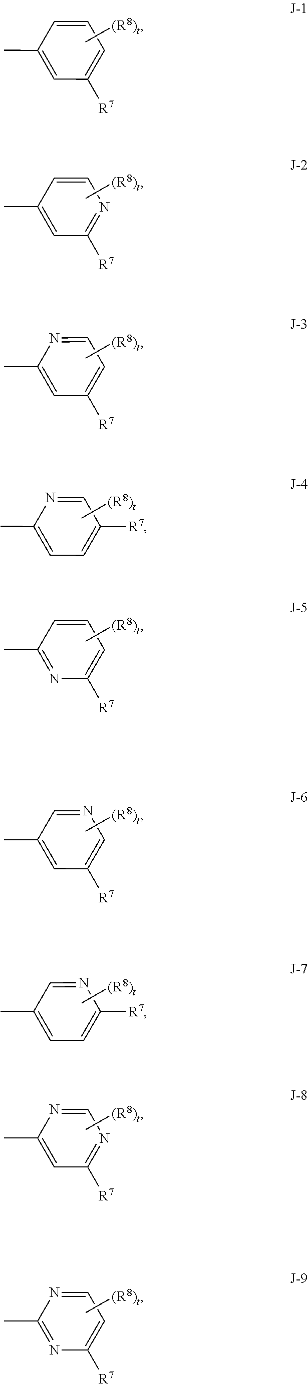

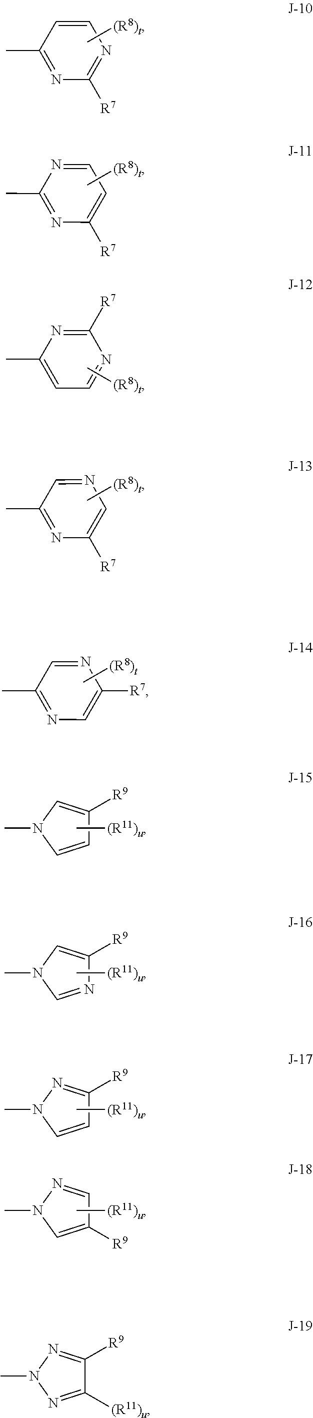

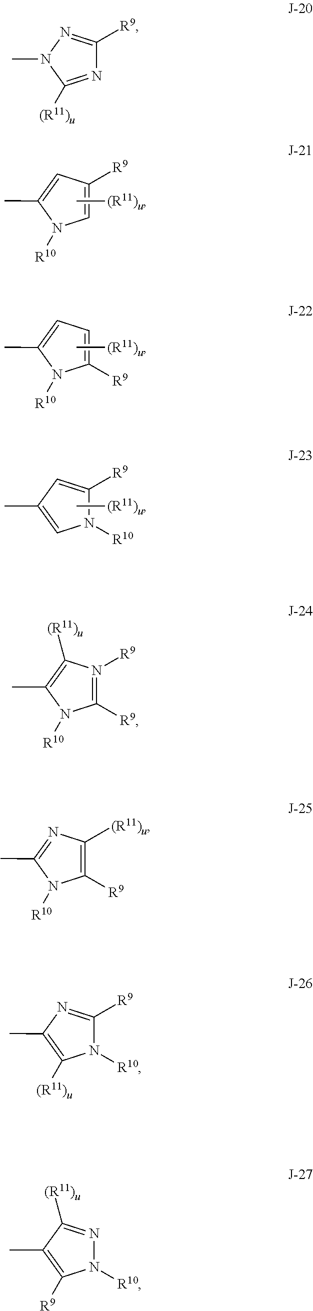

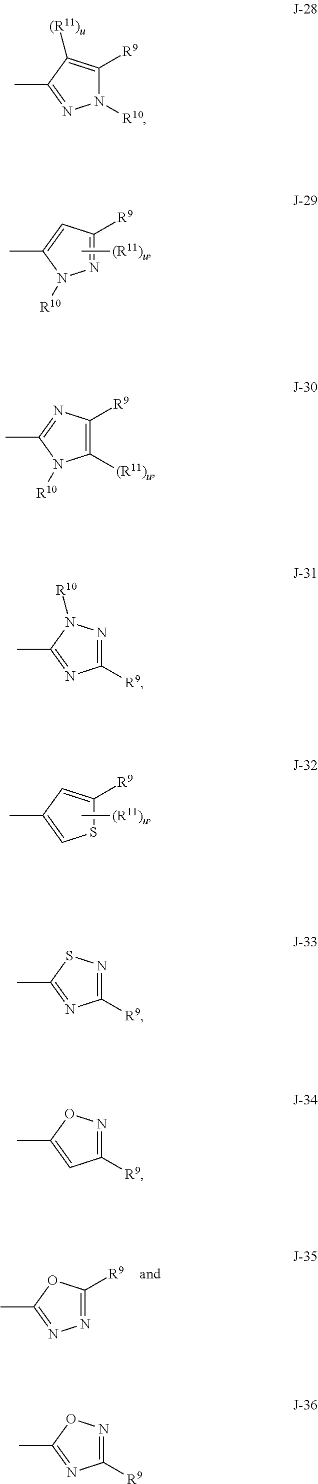

t is 0, 1 or 2; and
u is 0 or 1;
each R² is independently H, halogen, C₁-C₄ alkoxy, C₁-C₄ alkyl or C₁-C₄ haloalkyl;
each R³ is independently H, halogen or C₁-C₄ haloalkyl;
R⁵ is F or OH;
R⁶ is C₁-C₄ alkyl;
R⁷ is halogen, C₁-C₄ alkyl, C₁-C₄ haloalkyl, C₁-C₄ alkoxy or C₁-C₄ haloalkoxy;
R⁸ is independently halogen or C₁-C₄ haloalkyl; or
R⁷ and R⁸ are taken together with two adjacent carbon atoms to form a 5-membered carbocyclic ring containing ring members selected from up to 2 O atoms, and optionally substituted on carbon atom ring members with up to 5 halogen atoms;
R⁹ is halogen, C₁-C₄ alkyl, C₁-C₄ haloalkyl, C₁-C₄ alkoxy or C₁-C₄ haloalkoxy;
R¹⁰ is CH₃ or CH₂CF₃;
R¹¹ is halogen, C₁-C₄ alkyl, C₁-C₄ haloalkyl, C₁-C₄ alkoxy or C₁-C₄ haloalkoxy;
R^{A1} is H or C₁-C₄ alkyl; and
R^{A2} is H or C₁-C₄ alkyl.

### Embodiment H

A compound Embodiment G wherein
A is C(═O)N(R^{A})(R^{B}); or A is A-1;
Y¹ is N or CR²; and each Y², Y³, Y⁴ and Y⁵ is independently CR²;
R^{A} is C₁-C₆ alkyl or C₁-C₄ alkoxy, each optionally substituted with halogen or C₁-C₄ alkoxy; or C₃-C₆ cycloalkyl or C₄-C₈ cycloalkylalkyl, each optionally substituted with halogen, C₁-C₄ alkyl or C₁-C₄ alkoxy; or phenyl optionally substituted with halogen, C₁-C₄ alkyl, C₁-C₃ haloalkyl or C₁-C₄ alkoxy;
R^{B} is H or C₁-C₄ alkyl;
R¹ is C₁-C₄ alkoxy, C₁-C₄ alkyl or C₁-C₄ haloalkyl;
Q is —C(R⁵)(R⁶)—;
J is selected from J-2 through J-14;
t is 0 or 1;
each R² is independently H, halogen, C₁-C₄ alkyl or C₁-C₄ haloalkyl;
R⁷ is C₁-C₄ haloalkyl or C₁-C₄ haloalkoxy; and
each R⁸ is independently F, Cl or CF₃.

### Embodiment I

A compound Embodiment H wherein
A is C(═O)N(R^{A})(R^{B});
R^{A} is C₁-C₆ alkyl or C₁-C₄ alkoxy, each optionally substituted with halogen; or C₃-C₆ cycloalkyl optionally substituted with halogen or C₁-C₄ alkyl; or phenyl optionally substituted with halogen or C₁-C₃ haloalkyl;
R^{B} is H, CH₃ or CH₂CH₃;
R¹ is C₁-C₄ alkoxy or C₁-C₄ alkyl;
J is selected from J-2 and J-5;
t is 0;
wherein each R² is independently H, F, Cl, CH₃ or CF₃;
R⁷ is CF₃, OCF₃ or OCHF₂.

### Embodiment J

A compound Embodiment I wherein
R^{A} is C₁-C₆ alkyl or C₁-C₄ alkoxy, each optionally substituted with halogen; or C₃-C₆ cycloalkyl optionally substituted with halogen or C₁-C₄ alkyl;
R^{B} is H or CH₃;
R¹ is C₁-C₄ alkyl;
J is J-2; and
R⁷ is CF₃.

### Embodiment K

A compound Embodiment G wherein
A is C(═O)N(R^{A})(R^{B});
R^{A} is C₁-C₆ alkyl or C₁-C₄ alkoxy, each optionally substituted with halogen; or cyclopropyl;
R^{B} is H;
R¹ is CH₃, CH₂CH₃ or CH₂CH₂CH₃;
Q is —C(R⁵)(R⁶)—;
J is selected from J-15 through J-33;
u is 0;
R⁹ is C₁-C₄ haloalkyl or C₁-C₄ haloalkoxy; and
R¹⁰ is CH₃.

### Embodiment L

A compound Embodiment J wherein
R^{A} is —CH₂CF₃;
R¹ is CH₂CH₃;
J is J-29; and
R⁹ is F, CH₃ or CF₃.

Compounds of the invention are particularly useful for selective control of weeds in cereal crops such as wheat, barley, maize, soybean, sunflower, cotton, oilseed rape and rice, and specialty crops such as sugarcane, citrus, fruit and nut crops.

Also noteworthy as embodiments are herbicidal compositions of the present invention comprising the compounds as described in the embodiments above.

This invention also includes a method of using a herbicidal mixture comprising (a) a compound selected from Formula 1, N-oxides, and salts thereof, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics and (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solenesyltransererase (HST) inhibitors, (b14) cellulose biosynthesis inhibitors, (b15) other herbicides including mitotic disruptors, organic arsenicals, asulam, difenzoquat, bromobutide, flurenol, cinmethylin, cumyluron, dazomet, dymron, methyldymron, etobenzanid, fosamine, fosamine-ammonium, metam, oxaziclomefone, oleic acid, pelargonic acid and pyributicarb, and (b16) herbicide safeners; and salts of compounds of (b1) through (b16).

“Photosystem II inhibitors” (b1) are chemical compounds that bind to the D-1 protein at the Q_{B}-binding niche and thus block electron transport from Q_{A} to Q_{B} in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The Q_{B}-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem II inhibitors include ametryn, atrazine, cyanazine, desmetryne, dimethametryn, prometon, prometryne, propazine, simazine, simetryn, terbumeton, terbuthylazine, terbutryne, trietazine, hexazinone, metamitron, metribuzin, amicarbazone, bromacil, lenacil, terbacil, chloridazon, desmedipham, phenmedipham, chlorobromuron, chlorotoluron, chloroxuron, dimefuron, diuron, ethidimuron, fenuron, fluometuron, isoproturon, isouron, linuron, methabenzthiazuron, metobromuron, metoxuron, monolinuron, neburon, siduron, tebuthiuron, propanil, pentanochlor, bromofenoxim, bromoxynil, ioxynil, bentazon, pyridate and pyridafol.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl (b2a), chlorimuron-ethyl, chlorsulfuron, cinosulfuron, cyclosulfamuron, ethametsulfuron-methyl, ethoxysulfuron, flazasulfuron, flupyrsulfuron-methyl (b2b), flupyrsulfuron-sodium, foramsulfuron, halosulfuron-methyl, imazosulfuron, iodosulfuron-methyl (including sodium salt), mesosulfuron-methyl, metazosulfuron, metsulfuron-methyl, nicosulfuron, oxasulfuron, primisulfuron-methyl, propyrisulfuron, prosulfuron, pyrazosulfuron-ethyl, rimsulfuron, sulfometuron-methyl, sulfosulfuron, thifensulfuron-methyl (b2c), triasulfuron, tribenuron-methyl, trifloxysulfuron (including sodium salt), triflusulfuron-methyl, tritosulfuron, imazapic, imazamethabenz-methyl, imazamox, imazapyr, imazaquin, imazethapyr, cloransulam-methyl, diclosulam, florasulam, flumetsulam, metosulam, penoxsulam, bispyribac-sodium, pyribenzoxim, pyriftalid, pyrithiobac-sodium, pyriminobac-methyl, thiencarbazone, flucarbazone-sodium and propoxycarbazone-sodium.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl CoA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include cyclopyrimorate, clodinafop, cyhalofop, diclofop, fenoxaprop, fluazifop, haloxyfop, propaquizafop, quizalofop, alloxydim, butroxydim, clethodim, cycloxydim, pinoxaden, profoxydim, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b4) are chemical compounds mimicking the plant growth hormone auxin, thus causing uncontrolled and disorganized growth leading to plant death in susceptible species. Examples of auxin mimics include aminocyclopyrachlor and its methyl and ethyl esters and its sodium and potassium salts, aminopyralid benazolin-ethyl, chloramben, clacyfos, clomeprop, clopyralid, dicamba, 2,4-D, 2,4-DB, dichlorprop, fluoroxypyr, halauxifen, halauxifen-methyl, mecoprop, MCPA, MCPB, 2,3,6-TBA, picloram, triclopyr, quinclorac and quinmerac.

“EPSP (5-enol-pyruvylshikimate-3-phosphate) synthase inhibitors” (b5) are chemical compounds that inhibit the enzyme, 5-enol-pyruvylshikimate-3-phosphate synthase, which is involved in the synthesis of aromatic amino acids such as tyrosine, tryptophan and phenylalanine EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosystem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include paraquat and diquat.

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, bifenox, chlomethoxyfen, fluoroglycofen-ethyl, fomesafen, halosafen, lactofen, oxyfluorfen, fluazolate, pyraflufen-ethyl, cinidon-ethyl, flumioxazin, flumiclorac-pentyl, fluthiacet-methyl, thidiazimin, oxadiazon, oxadiargyl, saflufencil, azafenidin, carfentrazone carfentrazone-ethyl, sulfentrazone, pentoxazone, benzfendizone, butafenacil, pyraclonil, profluazol, flufenpyr-ethyl and tiafenacil.

“GS (glutamine synthase) inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P and bilanaphos.

“VLCFA (very long chain fatty acid) elongase inhibitors” (b9) are herbicides having a wide variety of chemical structures, which inhibit the elongase. Elongase is one of the enzymes located in or near chloroplasts which are involved in biosynthesis of VLCFAs. In plants, very-long-chain fatty acids are the main constituents of hydrophobic polymers that prevent desiccation at the leaf surface and provide stability to pollen grains. Such herbicides include acetochlor, alachlor, butachlor, dimethachlor, dimethanamid, metazachlor, metolachlor, pethoxamid, pretilachlor, propachlor, propisochlor, pyroxasulfone, thenylchlor, diphenamid, napropamide, naproanilide, fenoxasulfone, flufenacet, indanofan, mefenacet, fentrazamide, anilofos, cafenstrole, piperophos including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include naptalam (also known as N-(1-naphthyl)phthalamic acid and 2-[(1-naphthalenylamino)carbonyl]benzoic acid) and diflufenzopyr.

“PDS (phytoene desaturase inhibitors) (b11) are chemical compounds that inhibit carotenoid biosynthesis pathway at the phytoene desaturase step. Examples of PDS inhibitors include norflurzon, diflufenican, picolinafen, beflubutamide, fluridone, fluorochloridone and flurtamone.

“HPPD (4-hydroxyphenyl-pyruvate dioxygenase) inhibitors” (b12) are chemical substances that inhibit the biosynthesis of synthesis of 4-hydroxyphenyl-pyruvate dioxygenase. Examples of HPPD inhibitors include mesotrione, sulcotrione, topramezone, tembotrione, tefuryltrione, isoxachlortole, isoxaflutole, benzofenap, pyrasulfatole, pyrazolynate, pyrazoxyfen, bicyclopyrone, benzobicyclon, fenquinotrione and 5-[(2-hydroxy-6-oxo-1-cyclohexen-1-yl)carbonyl]-2-(3-methoxyphenyl)-3-(3-methoxypropyl)-4(3H)-pyrimidinone (b12a).

HST (homogentisate solenesyltransererase) inhibitors (b13) disrupt a plant's ability to convert homogentisate to 2-methyl-6-solanyl-1,4-benzoquinone, thereby disrupting carotenoid biosynthesis. Examples of HST inhibitors include haloxydine, pyriclor and the compounds of Formulae A, B and C.

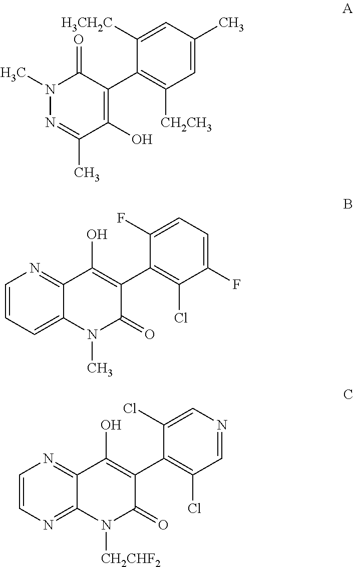

HST inhibitors also include compounds of Formulae D and E.

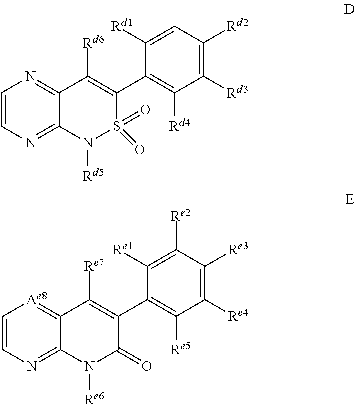

wherein R^{d1} is H, Cl or CF₃; R^{d2} is H, Cl or Br; R^{d3} is H or Cl; R^{d4} is H, Cl or CF₃; R^{d5} is CH₃, CH₂CH₃ or CH₂CHF₂; and R^{d6} is OH, or —OC(═O)-i-Pr; and R^{e1} is H, F, Cl, CH₃ or CH₂CH₃; R^{e2} is H or CF₃; R^{e3} is H, CH₃ or CH₂CH₃; R^{e4} is H, F or Br; R^{e5} is Cl, CH₃, CF₃, OCF₃ or CH₂CH₃; R^{e6} is H, CH₃, CH₂CHF₂ or C≡CH; R^{e7} is OH, —OC(═O)Et, —OC(═O)-i-Pr or —OC(═O)-t-Bu; and A^{e8} is N or CH.

Cellulose biosynthesis inhibitors (b14) inhibit the biosynthesis of cellulose in certain plants. They are most effective when applied preemergence or early postemergence on young or rapidly growing plants. Examples of cellulose biosynthesis inhibitors include chlorthiamid, diclobenil, flupoxam, indaziflam, isoxaben and triaziflam.

Other herbicides (b15) include herbicides that act through a variety of different modes of action such as mitotic disruptors (e.g., flamprop-M-methyl and flamprop-M-isopropyl) organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b14) or act through a combination of modes of action listed above. Examples of other herbicides include aclonifen, asulam, amitrole, clomezone, fluometuron, difenzoquat, bromobutide, flurenol, cinmethylin, cumyluron, dazomet, dymron, methyldymron, methiozolon, ipfencarbazone, etobenzanid, fosamine, fosamine-ammonium, metam, oxaziclomefone, oleic acid, pelargonic acid and pyributicarb.

“Herbicide safeners” (b16) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, 1-bromo-4-[(chloromethyl)sulfonyl]benzene, cloquintocet-mexyl, cumyluron, cyometrinil, cyprosulfamide, daimuron, dichlormid, dicyclonon, 4-(dichloroacetyl)-1-oxa-4-azospiro[4.5]decane (MON 4660), 2-(dichloromethyl)-2-methyl-1,3-dioxolane (MG 191), dimepiperate, fenchlorazole-ethyl, fenclorim, flurazole, fluxofenim, furilazole, isoxadifen-ethyl, mefenpyr-diethyl, mephenate, methoxyphenone, naphthalic anhydride and oxabetrinil.

One or more of the following methods and variations as described in Schemes 1-10 can be used to prepare the compounds of Formula 1. The definitions of A, Q, J, R¹, R², R³, R⁴, R⁵, R⁶, R⁷, R⁸, R⁹, R¹⁰ and R¹¹ in the compounds of Formulae 1 through 19 below are as defined above in the Summary of the Invention unless otherwise noted. Formulae 1a-1b and 4a-b are various subsets of a compound of Formulae 1 and 4, respectively. All substituents for Formulae 1a-1b are as defined above for Formula 1 unless otherwise noted.

Compounds of Formula 1a (i.e. Formula 1 wherein A is C(═O)N(R^{A})(R^{B})) can be prepared by reaction of compounds of Formulae 2-4 with an amine of Formula 5 under a variety of conditions as shown in Scheme 1. Reaction of acid chloride 2 is carried out in the presence of an acid scavenger. Typical acid scavengers include amine bases such as triethylamine, N,N-diisopropylethylamine and pyridine. Other scavengers include hydroxides such as sodium and potassium hydroxide and carbonates such as sodium carbonate and potassium carbonate. In certain instances it is useful to use polymer-supported acid scavengers such as polymer-bound N,N-diisopropylethylamine and polymer-bound 4-(dimethylamino)pyridine. Reaction of acid 3 with an amine of Formula 5 (or its acid salt) is carried out in the presence of a dehydrative coupling reagent such as dicyclohexylcarbodiimide (DCC), 1-(3-dimethylaminopropyl)-3-ethylcarbodiimide hydrochloride (EDC) or O-benzotriazol-1-yl-N,N,N′,N′-tetramethyluronium hexafluorophosphate (HBTU). Polymer-supported reagents are again useful here, such as polymer-bound cyclohexylcarbodiimide. These reactions are typically run at 0-40° C. in a solvent such as dichloromethane or acetonitrile in the presence of a base such as triethylamine or N,N-diisopropylethylamine. Reaction of ester 4 with an amine of Formula 5 is typically carried by heating the ester with an excess of the amine (2 to 50 equivalents) of Formula 5 at temperatures in the range from 20-250° C. It is convenient to carry the reaction out in a microwave apparatus where the temperature of the reaction can exceed the boiling point of the amine. The reaction may be carried out in the presence or absence of solvent. A wide variety of solvents are suitable for the reaction including, for example but are not limited, to C₁-C₆ alcohols, tetrahydrofuran, dichloromethane, dioxane, N,N-dimethylformamide, N,N-dimethylacetamide, N-methylpyrrolidinone, water, and acetonitrile as well as mixtures of these solvents.

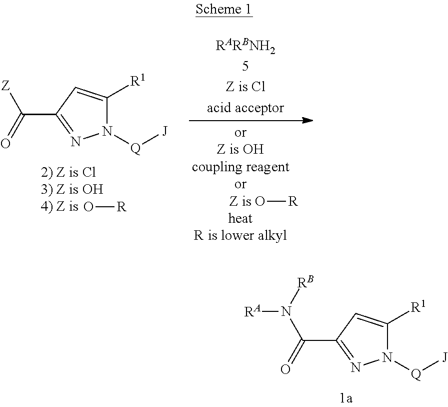

As shown in Scheme 2 esters of Formula 4a (i.e. Formula 4 wherein Q is —C(R⁵)(R⁶)— and R⁵ and R⁶ are H) can be prepared by the alkylation of compounds of Formula 5 with alkylating agents of Formula 6 in the presence of an acid acceptor. Alkylating agents of Formula 6 typically have leaving groups (Lg) selected from halides such as Cl, Br or I and sulfonates. Suitable acid acceptors for the reaction include inorganic bases such as alkali or alkaline earth metal (e.g., lithium, sodium, potassium and cesium) hydrides, alkoxides, carbonates, phosphates and hydroxides, and organic bases such as triethylamine, N,N-diisopropylethylamine and 1,8-diazabicyclo[5.4.0]undec-7-ene. A wide variety of solvents are suitable for the reaction including, for example but are not limited, to tetrahydrofuran, dichloromethane, N,N-dimethylformamide, N,N-dimethylacetamide, N-methylpyrrolidinone, acetonitrile, C₂-C₆ alcohols and acetone as well as mixtures of these solvents. This reaction is conducted between about −20 and 200° C., and typically between 0 and 50° C.

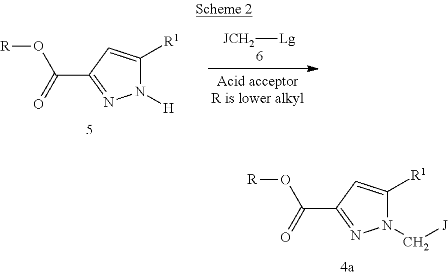

Alternatively, as shown in Scheme 3, compounds of Formula 4a may be prepared by reaction of compounds of Formula 7 with substituted hydrazines of Formula 8. This reaction may be carried out in a variety of solvents for example but are not limited, to tetrahydrofuran, N,N-dimethylformamide, N,N-dimethylacetamide, N-methylpyrrolidinone, acetonitrile, C₂-C₆ alcohols and water as well as mixtures of these solvents. This reaction is conducted between about −20 and 200° C., and typically between 0 and 80° C. It is recognized by one skilled in the art that this reaction may produce regioisomers and the compounds of Formula 4a can be separated from the other isomer by a variety of means. An alternative approach to compounds of Formula 4a as well as the relationship between solvent choice and regiochemistry of pyrazole formation is discussed in J. Org. Chem. 2008, 73, 3523-29 and references cited therein.

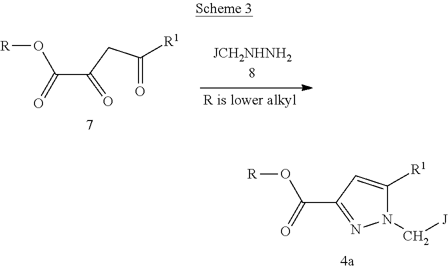

As shown in Scheme 4, compounds of Formula 5 may be prepared by the reaction of compounds of Formula 7 with hydrazine or its salts using the method described in Scheme 3. As also shown in Scheme 4, compounds of Formula 5 may be made by the reaction of diazoacetate esters of Formula 9 with aldehydes of Formula 10 in the presence of secondary amines. Appropriate conditions for carrying out this transformation have been described for ethyl diazoacetate and a variety of different aldehydes of Formula 10 in Chem. Eur. J. 2013, 19, 7555-7560.

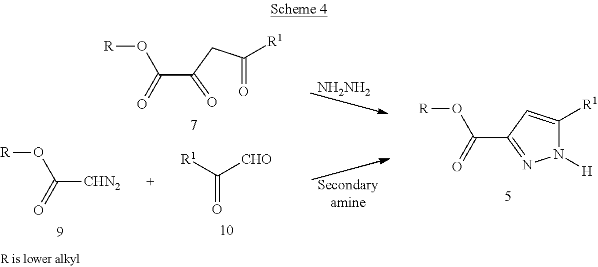

Compounds of Formula 7 may be prepared by the reaction of oxalate esters of Formula 11 with ketones of Formula 12 in the presence of an acid acceptor as shown in Scheme 5. Suitable acid acceptors for the reaction include inorganic bases such as alkali or alkaline earth metal (e.g., lithium, sodium, potassium and cesium) hydrides, alkoxides, carbonates, phosphates and hydroxides, and organic bases such as triethylamine, N,N-diisopropylethylamine and 1,8-diazabicyclo[5.4.0]undec-7-ene. Sodium hydride, sodium methoxide, sodium ethoxide, and potassium t-butoxide are preferred acid acceptors. A wide variety of solvents are suitable for the reaction including, for example but are not limited, to tetrahydrofuran, N,N-dimethylformamide, N,N-dimethylacetamide, N-methylpyrrolidinone, acetonitrile, C₂-C₆ alcohols and dioxane as well as mixtures of these solvents. This reaction is conducted between about −20 and 200° C., and typically between 0 and 50° C.

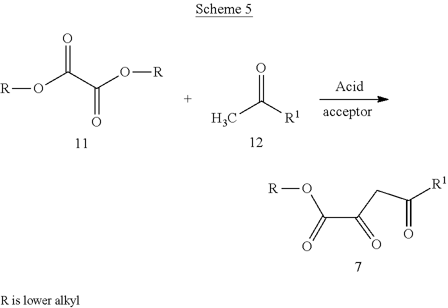

As shown in Scheme 6 compounds of Formula 4b (i.e. Formula 4 wherein Q is O) can be prepared by arylation of N-hydroxypyrazoles of Formula 13 with compounds of Formula 14 in the presence of an acid acceptor. Arylating agents of Formula 14 typically have leaving groups (Lg) selected from halides such as Cl, Br or I and sulfonates. Suitable acid acceptors for the reaction include inorganic bases such as alkali or alkaline earth metal (e.g., lithium, sodium, potassium and cesium) hydrides, alkoxides, carbonates, phosphates and hydroxides, and organic bases such as triethylamine, N,N-diisopropylethylamine and 1,8-diazabicyclo[5.4.0]undec-7-ene. A wide variety of solvents are suitable for the reaction including, for example but are not limited, to tetrahydrofuran, dichloromethane, N,N-dimethylformamide, N,N-dimethylacetamide, N-methylpyrrolidinone, acetonitrile, C₂-C₆ alcohols and acetone as well as mixtures of these solvents. This reaction is conducted between about −20 and 200° C., and typically between 0 and 50° C. Compounds of Formula 13 are described in J. Chem. Research 1996, 0570-0581. Compounds of Formula 14 are generally commercially available or can readily be prepared by methods known to those skilled in the art.

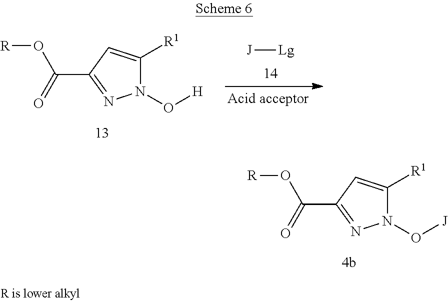

As shown in Scheme 7, compounds of Formula 1b (i.e. Formula wherein Q is —C(R⁵)(R⁶)— and R⁵ and R⁶ are H) in which A is A-1, A-2, A-3 or A-4 can be prepared by the alkylation of compounds of Formula 15 with alkylating agents of Formula 6 in the presence of an acid acceptor, by the method described in Scheme 2. Pyrazoles of Formula 15 can be prepared as disclosed in PCT Patent Publication WO 2009/086041 and references cited therein.

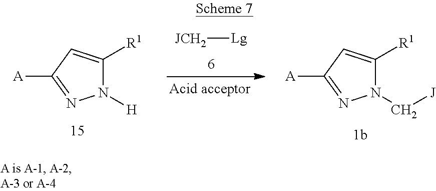

Alternatively, as shown in Scheme 8, compounds of Formula 1b in which A is A-1, A-2, A-3 or A-4 can be prepared from compounds of Formula 16 by Suzuki, Stille, Kumada and Negishi couplings with organometallic compounds of Formula 17 using transition metal catalysts. This chemistry applies to both aromatic and heteroaromatic metal species and is well known to those skilled in the art. A wide variety of conditions, organometallic species and transition metal catalysts have been surveyed by many authors. See for example: E. Negishi in Handbook of Organopalladium Chemistry for Organic Synthesis, John Wiley and Sons, 2002; N. Miyaura in Cross-Coupling Reactions: A Practical Guide, Springer, 2002; H. C. Brown et al., Organic Synthesis via Boranes, Aldrich Chemical Co., Vol. 3, 2002; Suzuki et al., Chemical Reviews 1995 95, 2457-2483; and Molander et al., Accounts of Chemical Research 2007 40, 275-286. Also see Gribble and Li Eds., Palladium in Heterocyclic Chemistry Volume 1, Pergamon Press, 2000 and Gribble and Li, Eds., Palladium in Heterocyclic Chemistry Volume 2, Pergamon Press, 2007 as well as deMeijere and Diederich Eds., Metal-Catalyzed Cross-Coupling Reactions, Second Edition, John Wiley and Sons, 2004.

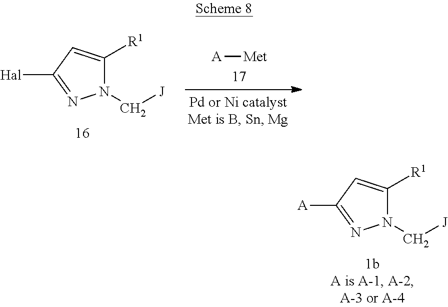

Compounds of Formula 1b wherein A is A-4 can be synthesized as depicted in Scheme 9 by reaction of compounds of Formula 16 with azoles of Formula 18 in the presence of copper catalysts. A variety of copper sources may be utilized in this reaction and the presence of a ligand which can chelate copper is also desirable. Bidentate ligands are preferred in this reaction. These coupling reactions are typically conducted in an inert solvent in the presence of a suitable ligand, a Cu(I) salt such as copper(I) iodide or copper(I) bromide, and a base such as sodium or potassium carbonate. Typical ligands include 1,2-diaminocyclohexane and phenanthroline. Suitable solvents for the reaction are dioxane, 1,2-diethoxyethane or toluene, and the reaction is carried out at temperatures ranging from room temperature to reflux for a period ranging from 1-48 h. This transformation can also be accomplished by reaction with palladium catalysis as well. For references which disclose conditions for this transformation see Sorokin, Mini-Reviews in Organic Chemistry 2008 5(4), 323-330; Bellina and Rossi, Advanced Synthesis & Catalysis 2010, 352(8), 1223-1276; Surry and Buchwald, Chemical Science 2010 1(1), 13-31; and Beletskaya and Cheprakov, Organometallics 2012, 31(22), 7753-7808.

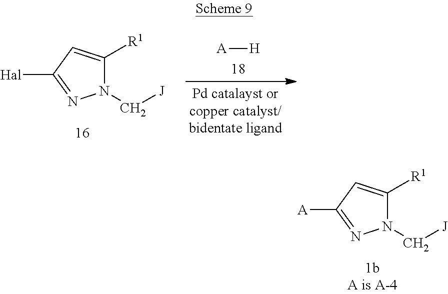

As shown in Scheme 10 compounds of Formula 16 can be prepared by the alkylation of compounds of Formula 19 with alkylating agents of Formula 6 in the presence of an acid acceptor by the method described in Scheme 2. Compounds of Formula 19 can be prepared by methods disclosed in U.S. Pat. No. 8,314,138, PCT Patent Publications WO 2008/129280 and WO 2010/15656, S. Guillou et. al. Tetrahedron 2011 67, 8451-8457 and Elguero et. al. Bull. Chim. Soc. France 1966, 293-302 as well as references cited therein.

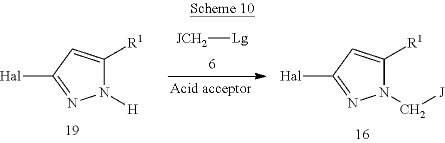

Alkylating agents of Formula 6 are often commercially available, but can readily be prepared from esters and aldehydes by reduction and conversion of the alcohol products to leaving groups by methods that are well known in the art. Alternatively preparation of compounds of Formula 6 by halogenation of alkyl benzenes and heterocycles by bromine or N-bromosuccinimide is also well known to one skilled in the art.

Conversion of esters of Formula 4 into acids of Formula 3 by alkaline hydrolysis and subsequent transformation into acid chlorides of Formula 2 by thionyl chloride or oxalyl chloride is also well precedented and familiar to one skilled in the art.

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular order presented to prepare the compounds of Formula 1.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “dd” means doublet of doublets, “dq” means doublet of quartets, “t” means triplet, “q” means quartet, “m” means multiplet and “br s” means broad singlet.

### Synthesis Example 1

### Preparation of N-cyclopropyl-5-ethyl-1-[[(2-trifluoromethyl)-4-pyridinyl]oxy]-1H-pyrazole-3-carboxamide (Compound 83)

### Step A: Preparation of ethyl 5-ethyl-1-hydroxy-1H-pyrazole-3-carboxylate

A solution of N,N-dimethyl-2-nitro-1-buten-1-amine (8.00 g crude, 54.7 mmol) in tetrahydrofuran (100 mL) was treated with 1,8-diazabicycloundec-7-ene (6.80 mL, 60.2 mmol) and ethyl isocyanoacetate (4.95 mL, 60.2 mmol). The mixture was stirred at 23° C. for 12 h. The reaction mixture was concentrated under reduced pressure, quenched with 10% aqueous hydrochloric acid (50 ml) and extracted with ethyl acetate (2×50 mL). The combined organic extracts were washed with brine (20 mL), dried (Na₂SO₄) and concentrated under reduced pressure. The residue was chromatographed on silica gel eluting with 0% to 100% ethyl acetate in hexanes to afford the title compound (1.10 g) as a yellow solid.

¹H NMR δ 6.54 (s, 1H), 4.34 (q, 2H), 2.72 (dq, 2H), 1.37 (t, 3H), 1.27 (t, 1H).

### Step B: Preparation of ethyl 5-ethyl-1-[[(2-(trifluoromethyl)-4-pyridinyl]oxy]-1H-pyrazole-3-carboxylate

A solution of ethyl 5-ethyl-1-hydroxy-1H-pyrazole-3-carboxylate (i.e. the product of Step A, 1.00 g, 5.43 mmol) in N,N-dimethylformamide (10 mL) was treated with 4-chloro-2-(trifluoromethyl)pyridine (0.98 g, 5.97 mmol), potassium carbonate (1.51 g, 10.86 mmol) and copper powder (0.015 g, 0.1 mmol). The mixture was irradiated under microwave conditions at 100° C. for 10 min. The reaction mixture was quenched with water (20 mL), extracted with ethyl acetate (2×30 mL) and the combined organic extracts washed with water (100 mL), brine (100 mL), dried (Na₂SO₄) and concentrated under reduced pressure. The residue was chromatographed on silica gel eluting with 0% to 100% ethyl acetate in hexanes to give the title compound as an off-white solid (550 mg).

¹H NMR δ 8.68 (d, 1H), 6.79 (dd, 1H), 6.76 (s, 1H), 4.41 (s, 3H), 2.61 (d, 2H), 1.39 (t, 3H), 1.37 (t, 3H).

### Step C: Preparation of 5-ethyl-1-[[(2-(trifluoromethyl)-4-pyridinyl)]oxy]-1H-pyrazole-3-carboxylic acid

A solution of ethyl 5-ethyl-1-[[(2-(trifluoromethyl)-4-pyridinyl]oxy]-1H-pyrazole-3-carboxylate (i.e. the product of Step B, 600 mg, 1.18 mmol) in ethanol (9 mL) and water (1 mL) was treated with lithium hydroxide monohydrate (133 mg, 1.77 mmol) and the mixture stirred at 23° C. for 1 h. The mixture was concentrated under reduced pressure, acidified with 1N aqueous hydrochloric acid (6 mL) to pH −1 and extracted with ethyl acetate (2×15 mL). The combined organic extracts were washed with brine (8 mL), dried (Na₂SO₄) and concentrated under reduced pressure to give the title compound (300 mg) as a white solid.

¹H NMR δ 8.68 (d, 1H) 7.13 (d, 1H), 6.80 (dd, 1H), 6.82 (s, 1H), 2.63 (q, 2H), 1.28 (t, 3H).

### Step D: Preparation of N-cyclopropyl-5-ethyl-1-[[(2-trifluoromethyl)-4-pyridinyl]oxy]-1H-pyrazole-3-carboxamide

A solution of 5-ethyl-1-[[(2-(trifluoromethyl)-4-pyridinyl)]oxy]-1H-pyrazole-3-carboxylic acid (i.e. the product of Step C, 75 mg, 0.24 mmol) in N,N-dimethylformamide (3 mL) was treated with 1-[bis(dimethylamino)methylene]-1H-1,2,3-triazolo[4,5-b]pyridinium 3-oxid hexafluorophosphate (HATU) (209 mg, 0.55 mmol) and N,N-diisopropylethylamine (92.8 mg, 72 mmol) and stirred at 23° C. for 10 min. Cyclopropylamine (28 mg, 0.48 mmol) was added and the reaction mixture was stirred at 23° C. for 5 h. The reaction mixture was quenched with water (6 mL), extracted with ethyl acetate (15 mL) and the combined organic extracts washed with water (8 mL), brine (8 mL), dried (Na₂SO₄) and concentrated under reduced pressure. The residue was chromatographed on silica gel eluting with 0% to 40% ethyl acetate in hexanes to give the title compound as an off-white semi-solid (25.6 mg).

¹H NMR δ 8.67 (d, 1H), 7.13 (d, 1H), 6.74 (dd, 1H), 6.71 (br s, 1H), 2.84 (m, 1H), 2.59 (dq, 2H), 1.26 (t, 3H), 0.83 (m, 2H), 0.60 (m, 2H).

### Synthesis Example 2

### Preparation of 5-ethyl-N-(2,2,2-trifluoroethyl)-1-[[(2-trifluoromethyl)-4-pyridinyl]methyl]-1H-pyrazole-3-carboxamide (Compound 78)

### Step A: Preparation of 4-(bromomethyl)-2-(trifluoromethyl)pyridine

To a stirred solution of [2-(trifluoromethyl)-pyridin-4-yl]methanol (56.5 mmol, 10.0 g) in dichloromethane (100 mL) under nitrogen was added dropwise over 15 min. a solution of phosphorous tribromide (113.0 mmol, 10.6 mL) in dichloromethane (40 mL). The resulting reaction mixture was allowed to stir at 23° C. overnight. The reaction mixture was then poured onto ice (150 g) and the pH adjusted to 6-7 by addition of saturated aqueous sodium bicarbonate (approximately 60 mL). The mixture was allowed to stir for 1 h at 23° C., during which time aqueous saturated sodium bicarbonate was added as needed to maintain the pH at 6-7. The aqueous solution was then extracted with ethyl acetate (4×100 mL), and the combined organic layers washed with brine, dried (MgSO₄) and concentrated under reduced pressure. The residue was chromatographed on silica gel eluting with 10-100% ethyl acetate/hexane to afford the title compound (11.14 g) as a clear oil.

¹H NMR δ 8.70-8.74 (m, 1H), 7.70 (s, 1H), 7.47-7.54 (m, 1H), 4.45 (s, 2H).

### Step B: Preparation of methyl 5-ethyl-1-[[(2-trifluoromethyl)-4-pyridinyl]methyl]-1H-pyrazole-3-carboxylate

To a solution of potassium tert-butoxide (1.10 g, 9.8 mmol) in tetrahydrofuran (20 mL) at 23° C. was added 3-ethyl-5-pyrazolecarboxylic acid methyl ester (1.25 g, 8.1 mmol) in tetrahydrofuran (2 mL), followed by 4-(bromomethyl)-2-(trifluoromethyl)pyridine (i.e. the product of Step A, 1.95 g, 8.1 mmol). The reaction mixture was stirred at 23° C. for 4 h and then poured into a 1:1 mixture of water and ethyl acetate (100 mL). The layers were separated and the aqueous layer extracted with ethyl acetate (2×50 mL). The combined organic extracts were washed with brine, dried (MgSO₄) and concentrated under reduced pressure. The residue was chromatographed on silica gel, eluting with 0-100% ethyl acetate/hexane to afford the title compound (1.68 g) as a pale yellow oil.

¹H NMR δ 8.66-8.70 (m, 1H), 7.36-7.40 (m, 1H), 7.06-7.13 (m, 1H), 6.73 (s, 1H), 5.46 (s, 2H), 3.94 (s, 3H), 2.48-2.55 (m, 2H), 1.24-1.27 (m, 3H).

### Step C: Preparation of 5-ethyl-1-[[2-(trifluoromethyl)-4-pyridinyl]methyl]-1H-pyrazole-3-carboxylic acid

A mixture of methyl 5-ethyl-1-[[(2-trifluoromethyl)-4-pyridinyl]methyl]-1H-pyrazole-3-carboxylate (i.e. the product of Step B, 1.68 g) and potassium hydroxide (1.08 g, 19.2 mmol) in 2:1 methanol/water (30 mL) was stirred at 23° C. for 3 h. The volume of the reaction mixture was reduced by 50% under reduced pressure and then poured into 1N aqueous hydrochloric acid (100 mL). The acidic solution was then extracted with ethyl acetate (3×50 mL) and the combined organic extracts dried (MgSO₄) and concentrated under reduced pressure to afford the title compound (1.5 g) as a pale yellow oil that solidified and was used in Step D below without further purification.

¹H NMR δ 9.73 (s, 1H), 7.73-7.80 (m, 2H), 7.12-7.19 (m, 2H), 2.33 (s, 3H).

### Step D: Preparation of 5-ethyl-N-(2,2,2-trifluoroethyl)-1-[[(2-trifluoromethyl)-4-pyridinyl]methyl]-1H-pyrazole-3-carboxamide

To a solution of 5-ethyl-1-[[2-(trifluoromethyl)-4-pyridinyl]methyl]-1H-pyrazole-3-carboxylic acid (1.5 g, 5.0 mmol) in methylene chloride containing a catalytic amount of N,N-dimethylformamide was added oxalyl chloride (0.645 mL, 7.5 mmol). The resulting mixture was allowed to stir at 23° C. for 1 h and then concentrated under reduced pressure. The yellow residue was dissolved in dichloromethane (1 mL) and added in one portion to a mixture of 2,2,2-trifluoroethylamine (0.492 mL, 6.0 mmol) and triethylamine (1.05 mL, 7.5 mmol). The solution was stirred for 1 h at 23° C. and then concentrated under reduced pressure. The residue was chromatographed on silica gel, eluting with 20-100% ethyl acetate/hexane to afford a clear oil which crystallized on standing to afford the title compound (1.72 g) as a solid. M.P.=81.2-84.6° C.

¹H NMR δ 8.68-8.72 (m, 1H), 7.38-7.42 (m, 1H), 7.04-7.12 (m, 2H), 6.75 (br s, 1H), 5.38 (s, 2H), 4.03-4.11 (m, 2H), 2.50-2.57 (m, 2H), 1.25-1.28 (m, 3H).

By the procedures described herein together with methods known in the art, the following compounds of Tables 1 to 187 can be prepared. The following abbreviations are used in the Tables which follow: n means normal, i means iso, c means cyclo, Me means methyl, Et means ethyl, Pr means propyl, Bu means butyl, i-Pr means isopropyl, c-Pr means cyclopropyl, c-Bu means cyclobutyl, Ph means phenyl, OMe means methoxy, OEt means ethoxy, SMe means methylthio, NHMe means methylamino and NH-c-Pr means cyclopropylamino.

In the following Tables 1 to 132, J-2A, J-2B, J-2C, J-2D, J-2E, J-2F, J-5A, J-10A, J-29A, J-29B, J-31A and J-32A refer to the following structures:

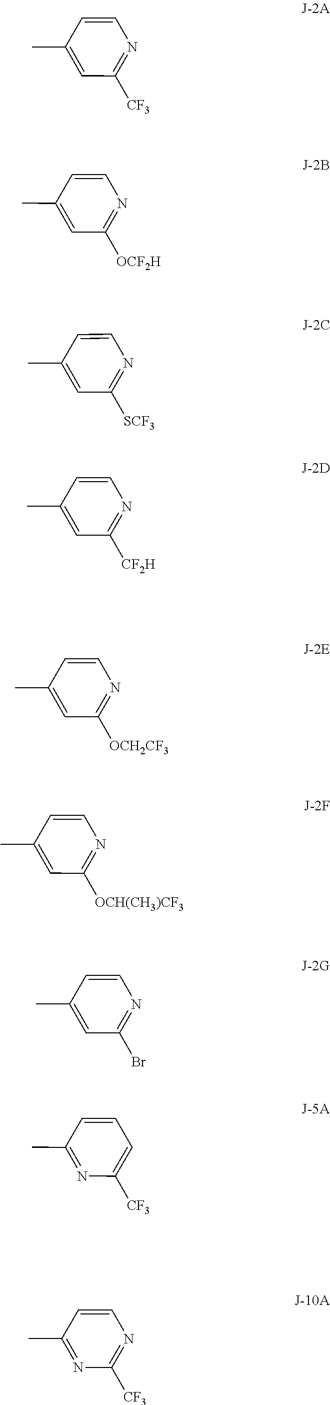

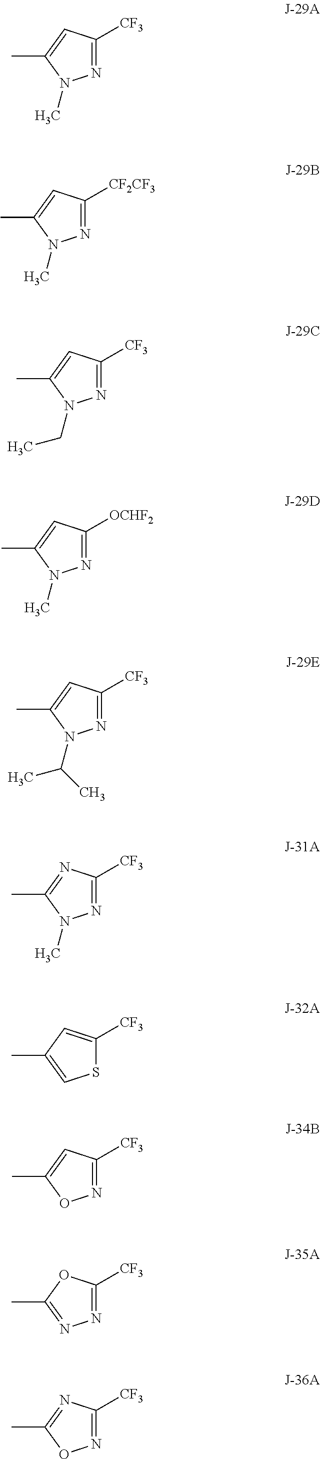

TABLE 1
1

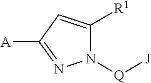

J is J-2A; Q is CH₂; R¹ is Me; and A is
A

Ph(4-F)
Ph(4-CF₃)
Ph(4-Cl)
Ph(4-Br)
Ph(4-SF₅)
Ph(4-OCF₃)
Ph(4-SCH₃)
Ph(4-OCH₃)
Ph(2-Br)
Ph(2,4-di-F)
Ph(3,4-di-F)
Ph(2,5-di-F)
Ph(2,4,6-tri-F)
Ph(2-Cl,4-F)
Ph(3-Cl,4-F)
Ph(4-F,3-CF₃)
Ph(2-F,4-CF₃)
Ph(3-F,4-CF₃)
Ph(3-Cl,4-CF₃)
Ph(2-Cl,4-CF₃)
Ph(4-Cl,3-CF₃)
1H-Imidazol-1-yl(5-CF₃)
1H-Pyrazol-1-yl(3-CF₃)
1H-Pyrazol-1-yl(4-CF₃)
2-Pyridinyl
3-Pyridinyl
4-Pyridinyl
2-Pyridinyl(5-CF₃)
2-Pyridinyl(5-Cl)
4-Pyridinyl(2-CF₃)
4-Pyridinyl(2-Cl)
2-Pyridinyl(6-CF₃)
3-Pyridinyl(5-CF₃)
Ph(4-C≡N)
Ph(4-Me)
Ph
Ph(3-F)
Ph(3-CF₃)
Ph(3-Cl)
Ph(3-Br)
Ph(3-SF₅)
5-Pyridinyl(2-CF₃)
2-Pyrazinyl(5-CF₃)
3-Pyridazinyl(6-CF₃)
2-Pyridinyl(5-F)
4-Pyridinyl(2-F)
2-Pyridinyl(6-F)
3-Pyridinyl(5-F)
3-Pyridinyl(6-F)
2-Pyrimidinyl(5-Cl)
2-Pyrimidinyl(5-CF₃)
4-Pyrimidinyl(2-CF₃)
2-Pyrimidinyl(4-CF₃)
2-Thienyl(5-Cl)
2-Thienyl(5-CF₃)
2-Thiazolyl(4-CF₃)
1,2,4-Thiadiazol-5-yl(3-CF₃)
1H-1,2,3-Triazol-2-yl(4-CF₃)
1H-1,2,4-Triazol-1-yl(3-CF₃)
C(═O)NH(CH₃)
C(═O)NH(CH₂CH₃)
C(═O)NH(CH₂CH₂CH₃)
C(═O)NH(c-Pr)
C(═O)NH(CH(CH₃)₂)
C(═O)NH(CH₂CF₃)
Ph(3-OCF₃)
Ph(3-SMe)
Ph(3-OMe)
Ph(3-C≡N)
Ph(3-Me)
Ph(2-F)
Ph(2-CF₃)
Ph(2-Cl)
C(═O)NH(OCH₃)
C(═O)NH(OCH₂CH₃)
C(═O)NH(OCH₂CH₂CH₃)
C(═O)NH(OCH(CH₃)₂)
C(═O)NH(OC(CH₃)₃)
C(═O)NH(OCH₂CH₂CH₂CH₃)
C(═O)NH(OCH(CH₃)CH₂CH₃))
C(═O)NH(OCH(CH₃(CH₂CH₃))
C(═O)NH(CH₂-c-Pr)
C(═O)NH(CH₂CF₂CF₃)
C(═O)NH(c-Bu)
C(═O)NH(c-Pentyl)
C(═O)NH(c-Hexyl)
C(═O)NH(C(CH₃)₃)
C(═O)NH(CH₂CH₂OCH₃)
C(═O)NCH₃(CH₃)
C(═O)NCH₃(OCH₃)
C(═O)NCH₃(CH₂CH₂CH₃)
C(═O)NH(CH₂CH₂CF₃)
C(═O)NH(CH₂CHF₂)
C(═O)NH(CH₂CH₂CHF₂)
C(═O)NH(CH₂CH₂SCH₃)
C(═O)NH(CH₂C(CH₃)₃)

Table 2 is constructed in the same manner except that the Row Heading “J is J-2A; Q is CH₂, R¹ is Me; and A is” is replaced with the Row Heading listed for Table 2 below (i.e. “J is J-2A; Q is CH₂; R¹ is Et; and A is”). Therefore the first entry in Table 2 is a compound of Formula 1 wherein R¹ is Et; Q is CH₂; A is Ph(4-F) (i.e. 4-fluorophenyl); and J is J-2A. Tables 3 through 132 are constructed similarly.

| Table | Row Heading |
|---|---|
| 2 | J is J-2A; Q is CH₂; R¹ is Et; and A is |
| 3 | J is J-2A; Q is CH₂; R¹ is n-Pr; and A is |
| 4 | J is J-2A; Q is CH₂; R¹ is n-Bu; and A is |
| 5 | J is J-2A; Q is CH₂; R¹ is OMe; and A is |
| 6 | J is J-2A; Q is CH₂; R¹ is SMe; and A is |
| 7 | J is J-2A; Q is CH₂; R¹ is NHMe; and A is |
| 8 | J is J-2A; Q is CH₂; R¹ is CH₂OCH₃; and A is |
| 9 | J is J-2A; Q is CH₂; R¹ is OEt; and A is |
| 10 | J is J-2A; Q is CH₂; R¹ is OCHF₂; and A is |
| 11 | J is J-2A; Q is CH₂; R¹ is Cl; and A is |
| 12 | J is J-2B; Q is CH₂; R¹ is Me; and A is |
| 13 | J is J-2B; Q is CH₂; R¹ is Et; and A is |
| 14 | J is J-2B; Q is CH₂; R¹ is n-Pr; and A is |
| 15 | J is J-2B; Q is CH₂; R¹ is n-Bu; and A is |
| 16 | J is J-2B; Q is CH₂; R¹ is OMe; and A is |
| 17 | J is J-2B; Q is CH₂; R¹ is SMe; and A is |
| 18 | J is J-2B; Q is CH₂; R¹ is NHMe; and A is |
| 19 | J is J-2B; Q is CH₂; R¹ is CH₂OCH₃; and A is |
| 20 | J is J-2B; Q is CH₂; R¹ is OEt; and A is |
| 21 | J is J-2B; Q is CH₂; R¹ is OCHF₂; and A is |
| 22 | J is J-2B; Q is CH₂; R¹ is Cl; and A is |
| 23 | J is J-2C; Q is CH₂; R¹ is Me; and A is |
| 24 | J is J-2C; Q is CH₂; R¹ is Et; and A is |
| 25 | J is J-2C; Q is CH₂; R¹ is n-Pr; and A is |
| 26 | J is J-2C; Q is CH₂; R¹ is n-Bu; and A is |
| 27 | J is J-2C; Q is CH₂; R¹ is OMe; and A is |
| 28 | J is J-2C; Q is CH₂; R¹ is SMe; and A is |
| 29 | J is J-2C; Q is CH₂; R¹ is NHMe; and A is |
| 30 | J is J-2C; Q is CH₂; R¹ is CH₂OCH₃; and A is |
| 31 | J is J-2C; Q is CH₂; R¹ is OEt; and A is |
| 32 | J is J-2C; Q is CH₂; R¹ is OCHF₂; and A is |
| 33 | J is J-2C; Q is CH₂; R¹ is Cl; and A is |
| 34 | J is J-2D; Q is CH₂; R¹ is Me; and A is |
| 35 | J is J-2D; Q is CH₂; R¹ is Et; and A is |
| 36 | J is J-2D; Q is CH₂; R¹ is n-Pr; and A is |
| 37 | J is J-2D; Q is CH₂; R¹ is n-Bu; and A is |
| 38 | J is J-2D; Q is CH₂; R¹ is OMe; and A is |
| 39 | J is J-2D; Q is CH₂; R¹ is SMe; and A is |
| 40 | J is J-2D; Q is CH₂; R¹ is NHMe; and A is |
| 41 | J is J-2D; Q is CH₂; R¹ is CH₂OCH₃; and A is |
| 42 | J is J-2D; Q is CH₂; R¹ is OEt; and A is |
| 43 | J is J-2D; Q is CH₂; R¹ is OCHF₂; and A is |
| 44 | J is J-2D Q is CH₂; R¹ is Cl; and A is |
| 45 | J is J-2E; Q is CH₂; R¹ is Me; and A is |
| 46 | J is J-2E; Q is CH₂; R¹ is Et; and A is |
| 47 | J is J-2E; Q is CH₂; R¹ is n-Pr; and A is |
| 48 | J is J-2E; Q is CH₂; R¹ is n-Bu; and A is |
| 49 | J is J-2E; Q is CH₂; R¹ is OMe; and A is |
| 50 | J is J-2E; Q is CH₂; R¹ is SMe; and A is |
| 51 | J is J-2E; Q is CH₂; R¹ is NHMe; and A is |
| 52 | J is J-2E; Q is CH₂; R¹ is CH₂OCH₃; and A is |
| 53 | J is J-2E; Q is CH₂; R¹ is OEt; and A is |
| 54 | J is J-2E; Q is CH₂; R¹ is OCHF₂; and A is |
| 55 | J is J-2E Q is CH₂; R¹ is Cl; and A is |
| 56 | J is J-2F; Q is CH₂; R¹ is Me; and A is |
| 57 | J is J-2F; Q is CH₂; R¹ is Et; and A is |
| 58 | J is J-2F; Q is CH₂; R¹ is n-Pr; and A is |
| 59 | J is J-2F; Q is CH₂; R¹ is n-Bu; and A is |
| 60 | J is J-2F; Q is CH₂; R¹ is OMe; and A is |
| 61 | J is J-2F; Q is CH₂; R¹ is SMe; and A is |
| 62 | J is J-2F; Q is CH₂; R¹ is NHMe; and A is |
| 63 | J is J-2F; Q is CH₂; R¹ is CH₂OCH₃; and A is |
| 64 | J is J-2F; Q is CH₂; R¹ is OEt; and A is |
| 65 | J is J-2F; Q is CH₂; R¹ is OCHF₂; and A is |
| 66 | J is J-2F; Q is CH₂; R¹ is Cl; and A is |
| 67 | J is J-5A; Q is CH₂; R¹ is Me; and A is |
| 68 | J is J-5A; Q is CH₂; R¹ is Et; and A is |
| 69 | J is J-5A; Q is CH₂; R¹ is n-Pr; and A is |
| 70 | J is J-5A; Q is CH₂; R¹ is n-Bu; and A is |
| 71 | J is J-5A; Q is CH₂; R¹ is OMe; and A is |
| 72 | J is J-5A; Q is CH₂; R¹ is SMe; and A is |
| 73 | J is J-5A; Q is CH₂; R¹ is NHMe; and A is |
| 74 | J is J-5A Q is CH₂; R¹ is CH₂OCH₃; and A is |
| 75 | J is J-5A; Q is CH₂; R¹ is OEt; and A is |
| 76 | J is J-5A; Q is CH₂; R¹ is OCHF₂; and A is |
| 77 | J is J-5A; Q is CH₂; R¹ is Cl; and A is |
| 78 | J is J-10A; Q is CH₂; R¹ is Me; and A is |
| 79 | J is J-10A; Q is CH₂; R¹ is Et; and A is |
| 80 | J is J-10A; Q is CH₂; R¹ is n-Pr; and A is |
| 81 | J is J-10A; Q is CH₂; R¹ is n-Bu; and A is |
| 82 | J is J-10A; Q is CH₂; R¹ is OMe; and A is |
| 83 | J is J-10A; Q is CH₂; R¹ is SMe; and A is |
| 84 | J is J-10A; Q is CH₂; R¹ is NHMe; and A is |
| 85 | J is J-10A; Q is CH₂; R¹ is CH₂OCH₃; and A is |
| 86 | J is J-10A; Q is CH₂; R¹ is OEt; and A is |
| 87 | J is J-10A; Q is CH₂; R¹ is OCHF₂; and A is |
| 88 | J is J-10A; Q is CH₂; R¹ is Cl; and A is |
| 89 | J is J-29A; Q is CH₂; R¹ is Me; and A is |
| 90 | J is J-29A; Q is CH₂; R¹ is Et; and A is |
| 91 | J is J-29A; Q is CH₂; R¹ is n-Pr; and A is |
| 92 | J is J-29A; Q is CH₂; R¹ is n-Bu; and A is |
| 93 | J is J-29A; Q is CH₂; R¹ is OMe; and A is |
| 94 | J is J-29A; Q is CH₂; R¹ is SMe; and A is |
| 95 | J is J-29A; Q is CH₂; R¹ is NHMe; and A is |
| 96 | J is J-29A; Q is CH₂; R¹ is CH₂OCH₃; and A is |
| 97 | J is J-29A; Q is CH₂; R¹ is OEt; and A is |
| 98 | J is J-29A; Q is CH₂; R¹ is OCHF₂; and A is |
| 99 | J is J-29A; Q is CH₂; R¹ is Cl; and A is |
| 100 | J is J-29B; Q is CH₂; R¹ is Me; and A is |
| 101 | J is J-29B; Q is CH₂; R¹ is Et; and A is |
| 102 | J is J-29B; Q is CH₂; R¹ is n-Pr; and A is |
| 103 | J is J-29B; Q is CH₂; R¹ is n-Bu; and A is |
| 104 | J is J-29B; Q is CH₂; R¹ is OMe; and A is |
| 105 | J is J-29B; Q is CH₂; R¹ is SMe; and A is |
| 106 | J is J-29B; Q is CH₂; R¹ is NHMe; and A is |
| 107 | J is J-29B; Q is CH₂; R¹ is CH₂OCH₃; and A is |
| 108 | J is J-29B; Q is CH₂; R¹ is OEt; and A is |
| 109 | J is J-29B; Q is CH₂; R¹ is OCHF₂; and A is |
| 110 | J is J-29B; Q is CH₂; R¹ is Cl; and A is |
| 111 | J is J-31A; Q is CH₂; R¹ is Me; and A is |
| 112 | J is J-31A; Q is CH₂; R¹ is Et; and A is |
| 113 | J is J-31A; Q is CH₂; R¹ is n-Pr; and A is |
| 114 | J is J-31A; Q is CH₂; R¹ is n-Bu; and A is |
| 115 | J is J-31A; Q is CH₂; R¹ is OMe; and A is |
| 116 | J is J-31A; Q is CH₂; R¹ is SMe; and A is |
| 117 | J is J-31A; Q is CH₂; R¹ is NHMe; and A is |
| 118 | J is J-31A; Q is CH₂; R¹ is CH₂OCH₃; and A is |
| 119 | J is J-31A; Q is CH₂; R¹ is OEt; and A is |
| 120 | J is J-31A; Q is CH₂; R¹ is OCHF₂; and A is |
| 121 | J is J-32A; Q is CH₂; R¹ is Cl; and A is |
| 122 | J is J-32A; Q is CH₂; R¹ is Me; and A is |
| 123 | J is J-32A; Q is CH₂; R¹ is Et; and A is |
| 124 | J is J-32A; Q is CH₂; R¹ is n-Pr; and A is |
| 125 | J is J-32A; Q is CH₂; R¹ is n-Bu; and A is |
| 126 | J is J-32A; Q is CH₂; R¹ is OMe; and A is |
| 127 | J is J-32A; Q is CH₂; R¹ is SMe; and A is |
| 128 | J is J-32; Q is CH₂; R¹ is NHMe; and A is |
| 129 | J is J-32A; Q is CH₂; R¹ is CH₂OCH₃; and A is |
| 130 | J is J-32A; Q is CH₂; R¹ is OEt; and A is |
| 131 | J is J-32A; Q is CH₂; R¹ is OCHF₂; and A is |
| 132 | J is J-32A; Q is CH₂; R¹ is Cl; and A is |

In the following Tables 133 to 187, J-1A, J-1B, J-1C, J-1D and J-1E refer to the following structures:

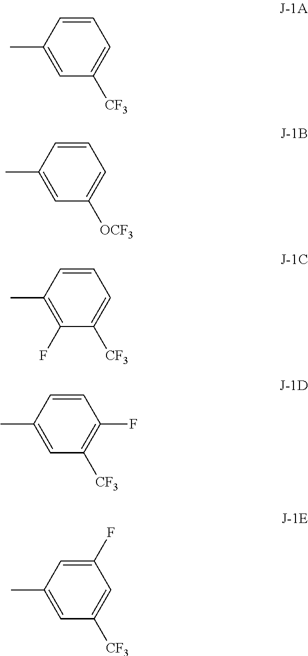

TABLE 133
1

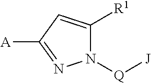

J is J-1A; Q is CH₂; R¹ is Me; and A is
A

1H-Imidazol-1-yl(5-CF₃)
1H-Pyrazo-1-yl(3-CF₃)
1H-Pyrazol-1-yl(4-CF₃)
2-Pyridinyl
3-Pyridinyl
4-Pyridinyl
3-Pyridinyl(5-F)
3-Pyridinyl(6-F)
2-Pyrimidinyl(5-Cl)
2-Pyrimidinyl(5-CF₃)
4-Pyrimidinyl(2-CF₃)
2-Pyrimidinyl(4-CF₃)
2-Thienyl(5-Cl)
2-Thienyl(5-CF₃)
2-Thiazolyl(4-CF₃)
1,2,4-Thiadiazol-5-yl(3-CF₃)
1,2,3-1H-Triazol-2-yl(4-CF₃)
1,2,4-1H-Triazol-1-yl(3-CF₃)
C(═O)NH(CH₃)
C(═O)NH(CH₂CH₃)
2-Pyridinyl(5-CF₃)
2-Pyridinyl(5-Cl)
4-Pyridinyl(2-CF₃)
4-Pyridinyl(2-Cl)
2-Pyridinyl(6-CF₃)
3-Pyridinyl(5-CF₃)
C(═O)NH(CH₂CH₂CH₃)
C(═O)NH(c-Pr)
C(═O)NH(CH(CH₃)₂)
C(═O)NH(CH₂CF₃)
C(═O)NHN(CH₃)₂
C(═O)NH(OCH₃)
C(═O)NH(OCH₂CH₃)
C(═O)NH(OCH₂CH₂CH₃)
C(═O)NH(OCH(CH₃)₂)
C(═O)NH(OC(CH₃)₃)
C(═O)NH(OCH₂CH₂CH₂CH₃)
C(═O)NH(OCH(CH₃)CH₂CH₃))
C(═O)NH(OCH(CH₃)(CH₂CH₃))
C(═O)NH(CH₂-c-Pr)
5-Pyridinyl(2-CF₃)
2-Pyrazinyl(5-CF₃)
3-Pyridazinyl(6-CF₃)
2-Pyridinyl(5-F)
4-Pyridinyl(2-F)
2-Pyridinyl(6-F)
C(═O)NH(CH₂CF₂CF₃)
C(═O)NH(c-Bu)
C(═O)NH(c-Pentyl)
C(═O)NH(c-Hexyl)
C(═O)NH(C(CH₃)₃)
C(═O)NH(CH₂CH₂OCH₃)
C(═O)NCH₃(CH₃)
C(═O)NCH₃(OCH₃)
C(═O)NCH₃(CH₂CH₂CH₃)
C(═O)NH(CH₂CH₂CF₃)
C(═O)NH(CH₂CHF₂)
C(═O)NH(CH₂CH₂CHF₂)
C(═O)NH(CH₂CH₂SCH₃)
C(═O)NH(CH₂C(CH₃)₃)

Table 134 is constructed in the same manner as Table 133 except that the Row Heading (i.e. “J is J-1A; Q is CH₂, R¹ is Me; and A is”) is replaced with the Row Heading listed for Table 134 below (i.e. “J is J-1A; Q is CH₂; R¹ is Et; and A is”). Therefore the first entry in Table 134 is a compound of Formula 1 wherein R¹ is Et; Q is CH₂; A is 1H-Imidazol-1-yl(5-CF₃) and J is J-1A. Tables 135 through 187 are constructed similarly.

| Table | Row Heading |
|---|---|
| 134 | J is J-1A; Q is CH₂; R¹ is Et; and A is |
| 135 | J is J-1A; Q is CH₂; R¹ is n-Pr; and A is |
| 136 | J is J-1A; Q is CH₂; R¹ is n-Bu; and A is |
| 137 | J is J-1A; Q is CH₂; R¹ is OMe; and A is |
| 138 | J is J-1A; Q is CH₂; R¹ is SMe; and A is |
| 139 | J is J-1A; Q is CH₂; R¹ is NHMe; and A is |
| 140 | J is J-1A; Q is CH₂; R¹ is CH₂OCH₃; and A is |
| 141 | J is J-1A; Q is CH₂; R¹ is OEt; and A is |
| 142 | J is J-1A; Q is CH₂; R¹ is OCHF₂; and A is |
| 143 | J is J-1A; Q is CH₂; R¹ is Cl; and A is |
| 144 | J is J-1B; Q is CH₂; R¹ is Me; and A is |
| 145 | J is J-1B; Q is CH₂; R¹ is Et; and A is |
| 146 | J is J-1B; Q is CH₂; R¹ is n-Pr; and A is |
| 147 | J is J-1B; Q is CH₂; R¹ is n-Bu; and A is |
| 148 | J is J-1B; Q is CH₂; R¹ is OMe; and A is |
| 149 | J is J-1B; Q is CH₂; R¹ is SMe; and A is |
| 150 | J is J-1B; Q is CH₂; R¹ is NHMe; and A is |
| 151 | J is J-1B; Q is CH₂; R¹ is CH₂OCH₃; and A is |
| 152 | J is J-1B; Q is CH₂; R¹ is OEt; and A is |
| 153 | J is J-1B; Q is CH₂; R¹ is OCHF₂; and A is |
| 154 | J is J-1B; Q is CH₂; R¹ is Cl; and A is |
| 155 | J is J-1C; Q is CH₂; R¹ is Me; and A is |
| 156 | J is J-1C; Q is CH₂; R¹ is Et; and A is |
| 157 | J is J-1C; Q is CH₂; R¹ is n-Pr; and A is |
| 158 | J is J-1C; Q is CH₂; R¹ is n-Bu; and A is |
| 159 | J is J-1C; Q is CH₂; R¹ is OMe; and A is |
| 160 | J is J-1C; Q is CH₂; R¹ is SMe; and A is |
| 161 | J is J-1C; Q is CH₂; R¹ is NHMe; and A is |
| 162 | J is J-1C; Q is CH₂; R¹ is CH₂OCH₃; and A is |
| 163 | J is J-1C; Q is CH₂; R¹ is OEt; and A is |
| 164 | J is J-1C; Q is CH₂; R¹ is OCHF₂; and A is |
| 165 | J is J-1C; Q is CH₂; R¹ is Cl; and A is |
| 166 | J is J-1D; Q is CH₂; R¹ is Me; and A is |
| 167 | J is J-1D; Q is CH₂; R¹ is Et; and A is |
| 168 | J is J-1D; Q is CH₂; R¹ is n-Pr; and A is |
| 169 | J is J-1D; Q is CH₂; R¹ is n-Bu; and A is |
| 170 | J is J-1D; Q is CH₂; R¹ is OMe; and A is |
| 171 | J is J-1D; Q is CH₂; R¹ is SMe; and A is |
| 172 | J is J-1D; Q is CH₂; R¹ is NHMe; and A is |
| 173 | J is J-1D; Q is CH₂; R¹ is CH₂OCH₃; and A is |
| 174 | J is J-1D; Q is CH₂; R¹ is OEt; and A is |
| 175 | J is J-1D; Q is CH₂; R¹ is OCHF₂; and A is |
| 176 | J is J-1D Q is CH₂; R¹ is Cl; and A is |
| 177 | J is J-1E; Q is CH₂; R¹ is Me; and A is |
| 178 | J is J-1E; Q is CH₂; R¹ is Et; and A is |
| 179 | J is J-1E; Q is CH₂; R¹ is n-Pr; and A is |
| 180 | J is J-1E; Q is CH₂; R¹ is n-Bu; and A is |
| 181 | J is J-1E; Q is CH₂; R¹ is OMe; and A is |
| 182 | J is J-1E; Q is CH₂; R¹ is SMe; and A is |
| 183 | J is J-1E; Q is CH₂; R¹ is NHMe; and A is |
| 184 | J is J-1E; Q is CH₂; R¹ is CH₂OCH₃; and A is |
| 185 | J is J-1E; Q is CH₂; R¹ is OEt; and A is |
| 186 | J is J-1E; Q is CH₂; R¹ is OCHF₂; and A is |
| 187 | J is J-1E; Q is CH₂; R¹ is Cl; and A is |

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Active | Weight Percent | |
|---|---|---|---|
| | Ingredient | Diluent | Surfactant |
| Water-Dispersible and Water-soluble Granules, | 0.001-90 | 0-99.999 | 0-15 |
| Tablets and Powders | | | |
| Oil Dispersions, Suspensions, Emulsions, Solutions | 1-50 | 40-99 | 0-50 |
| (including Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A and B. Without further elaboration, it is believed that one skilled in the art using the preceding description can utilize the present invention to its fullest extent. The following non-limiting Examples are illustrative of the invention. Percentages are by weight except where otherwise indicated.

### Example A

High Strength Concentrate

| | |
|---|---|
| Compound 105 | 98.5% |
| silica aerogel | 0.5% |
| synthetic amorphous fine silica | 1.0% |

### Example B

Wettable Powder

| | |
|---|---|
| Compound 78 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 104 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 106 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 108 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 98 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Suspension Concentrate

| | |
|---|---|
| Compound 78 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example H

Emulsion in Water

| | |
|---|---|
| Compound 104 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example I

Oil Dispersion

| | |
|---|---|
| Compound 106 | 25% |
| polyoxyethylene sorbitol hexaoleate | 15% |
| organically modified bentonite clay | 2.5% |
| fatty acid methyl ester | 57.5% |

### Example J

Suspoemulsion

| | |
|---|---|
| Compound 98 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Test results indicate that the compounds of the present invention are highly active preemergent and/or postemergent herbicides and/or plant growth regulants. The compounds of the invention generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation.

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.001 to 20 kg/ha with a preferred range of about 0.004 to 1 kg/ha. One skilled in the art can easily determine the herbicidally effective amount necessary for the desired level of weed control.

In one common embodiment, a compound of the invention is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Plant varieties and cultivars of the desired vegetation in the locus treated with a compound of the invention can be obtained by conventional propagation and breeding methods or by genetic engineering methods. Genetically modified plants (transgenic plants) are those in which a heterologous gene (transgene) has been stably integrated into the plant's genome. A transgene that is defined by its particular location in the plant genome is called a transformation or transgenic event.

Genetically modified plant cultivars in the locus which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance. Useful genetically modified plants containing single gene transformation events or combinations of transformation events are listed in Exhibit C. Additional information for the genetic modifications listed in Exhibit C can be obtained from publicly available databases maintained, for example, by the U.S. Department of Agriculture.

The following abbreviations are used in Exhibit C which follows: “tol.” is tolerance, “ins.” is insect, “res.” is resistance, “herb.” is herbicide, “SU” is sulfonylurea, “ALS” is acetolactate synthase, “HPPD” is 4-hydroxyphenylpyruvate dioxygenase, “Gly.” is glyphosate, “Glu.” is glufosinate, “Aoa” is Aryloxyalkanoate, “Mofa” is Modified oil/fatty acid, “Poll. Cntrl.” Is pollination control, “Imid.” is imidazolinone, “Phy.” phytate breakdown, A “-” means the entry is not available.

### Exhibit C

| Crop | Event Name | Event Code | Trait(s) | Gene(s) |
|---|---|---|---|---|
| Alfalfa | J101 | MON-00101- | Gly. tol. | cp4 epsps (aroA:CP4) |
| | | 8 | | |
| Alfalfa | J163 | MON- | Gly. tol. | cp4 epsps (aroA:CP4) |
| | | ØØ163-7 | | |
| Canola* | 23-18-17 (Event 18) | CGN-89465- | High lauric acid | te |
| | | 2 | oil | |
| Canola* | 23-198 (Event 23) | CGN-89465- | High lauric acid | te |
| | | 2 | oil | |
| Canola* | 61061 | DP-Ø61Ø61- | Gly. tol. | gat4621 |
| | | 7 | | |
| Canola* | 73496 | DP-Ø73496- | Gly. tol. | gat4621 |
| | | 4 | | |
| Canola* | GT200 (RT200) | MON-89249- | Gly. tol. | cp4 epsps (aroA:CP4); goxv247 |
| | | 2 | | |
| Canola* | GT73 (RT73) | MON- | Gly. tol. | cp4 epsps (aroA:CP4); goxv247 |
| | | ØØØ73-7 | | |
| Canola* | HCN10 (Topas 19/2) | — | Glu. tol. | bar |
| Canola* | HCN28 (T45) | ACS- | Glu. tol. | pat (syn) |
| | | BNØØ8-2 | | |
| Canola* | HCN92 (Topas 19/2) | ACS- | Glu. tol. | bar |
| | | BNØØ7-1 | | |
| Canola* | MON88302 | MON- | Gly. tol. | cp4 epsps (aroA:CP4) |
| | | 883Ø2-9 | | |
| Canola* | MPS961 | — | Phy. | phyA |
| Canola* | MPS962 | — | Phy. | phyA |
| Canola* | MPS963 | — | Phy. | phyA |
| Canola* | MPS964 | — | Phy. | phyA |
| Canola* | MPS965 | — | Phy. | phyA |
| Canola* | MS1 (B91-4) | ACS- | Glu. tol. | bar |
| | | BNØØ4-7 | | |
| Canola* | MS8 | ACS- | Glu. tol. | bar |
| | | BNØØ5-8 | | |
| Canola* | OXY-235 | ACS- | Oxynil tol. | bxn |
| | | BNØ11-5 | | |
| Canola* | PHY14 | — | Glu. tol. | bar |
| Canola* | PHY23 | — | Glu. tol. | bar |
| Canola* | PHY35 | — | Glu. tol. | bar |
| Canola* | PHY36 | — | Glu. tol. | bar |
| Canola* | RF1 (B93-101) | ACS- | Glu. tol. | bar |
| | | BNØØ1-4 | | |
| Canola* | RF2 (B94-2) | ACS- | Glu. tol. | bar |
| | | BNØØ2-5 | | |
| Canola* | RF3 | ACS- | Glu. tol. | bar |
| | | BNØØ3-6 | | |
| Bean | EMBRAPA 5.1 | EMB- | Disease res. | ac1 (sense and antisense) |
| | | PV051-1 | | |
| Brinjal | EE-1 | — | Ins. res. | cry1Ac |
| (Eggplant) | | | | |
| Cotton | 19-51a | DD- | ALS herb. tol. | S4-HrA |
| | | Ø1951A-7 | | |
| Cotton | 281-24-236 | DAS-24236- | Glu. tol.; ins. res. | pat (syn); cry1F |
| | | 5 | | |
| Cotton | 3006-210-23 | DAS-21Ø23- | Glu. tol.; ins. res. | pat (syn); cry1Ac |
| | | 5 | | |
| Cotton | 31707 | — | Oxynil tol.; ins. | bxn; cry1Ac |
| | | | res. | |
| Cotton | 31803 | — | Oxynil tol.; ins. | bxn; cry1Ac |
| | | | res. | |
| Cotton | 31807 | — | Oxynil tol.; ins. | bxn; cry1Ac |
| | | | res. | |
| Cotton | 31808 | — | Oxynil tol.; ins. | bxn; cry1Ac |
| | | | res. | |
| Cotton | 42317 | — | Oxynil tol.; ins. | bxn; cry1Ac |
| | | | res. | |
| Cotton | BNLA-601 | — | Ins. res. | cry1Ac |
| Cotton | BXN10211 | BXN10211-9 | Oxynil tol. | bxn; cry1Ac |
| Cotton | BXN10215 | BXN10215-4 | Oxynil tol. | bxn; cry1Ac |
| Cotton | BXN10222 | BXN10222-2 | Oxynil tol. | bxn; cry1Ac |
| Cotton | BXN10224 | BXN10224-4 | Oxynil tol. | bxn; cry1Ac |
| Cotton | COT102 | SYN-IR102- | Ins. res. | vip3A(a) |
| | | 7 | | |
| Cotton | COT67B | SYN-IR67B- | Ins. res. | cry1Ab |
| | | 1 | | |
| Cotton | COT202 | — | Ins. res. | vip3A |
| Cotton | Event 1 | — | Ins. res. | cry1Ac |
| Cotton | GMF Cry1A | GTL- | Ins. res. | cry1Ab-Ac |
| | | GMF311-7 | | |
| Cotton | GHB119 | BCS-GH005- | Ins. res. | cry2Ae |
| | | 8 | | |
| Cotton | GHB614 | BCS-GH002- | Gly. tol. | 2mepsps |
| | | 5 | | |
| Cotton | GK12 | — | Ins. res. | cry1Ab-Ac |
| Cotton | LLCotton25 | ACS- | Glu. tol. | bar |
| | | GH001-3 | | |
| Cotton | MLS 9124 | — | Ins. res. | cry1C |
| Cotton | MON1076 | MON-89924- | Ins. res. | cry1Ac |
| | | 2 | | |
| Cotton | MON1445 | MON-01445- | Gly. tol. | cp4 epsps (aroA:CP4) |
| | | 2 | | |
| Cotton | MON15985 | MON-15985- | Ins. res. | cry1Ac; cry2Ab2 |
| | | 7 | | |
| Cotton | MON1698 | MON-89383- | Gly. tol. | cp4 epsps (aroA:CP4) |
| | | 1 | | |
| Cotton | MON531 | MON-00531- | Ins. res. | cry1Ac |
| | | 6 | | |
| Cotton | MON757 | MON-00757- | Ins. res. | cry1Ac |
| | | 7 | | |
| Cotton | MON88913 | MON-88913- | Gly. tol. | cp4 epsps (aroA:CP4) |
| | | 8 | | |
| Cotton | Nqwe Chi 6 Bt | — | Ins. res. | — |
| Cotton | SKG321 | — | Ins. res. | cry1A; CpTI |
| Cotton | T303-3 | BCS-GH003- | Ins. res.; Glu. tol. | cry1Ab; bar |
| | | 6 | | |
| Cotton | T304-40 | BCS-GH004- | Ins. res.; Glu. tol. | cry1Ab; bar |
| | | 7 | | |
| Cotton | CE43-67B | — | Ins. res. | cry1Ab |
| Cotton | CE46-02A | — | Ins. res. | cry1Ab |
| Cotton | CE44-69D | — | Ins. res. | cry1Ab |
| Cotton | 1143-14A | — | Ins. res. | cry1Ab |
| Cotton | 1143-51B | — | Ins. res. | cry1Ab |
| Cotton | T342-142 | — | Ins. res. | cry1Ab |
| Cotton | PV-GHGT07 (1445) | — | Gly. tol. | cp4 epsps (aroA:CP4) |
| Cotton | EE-GH3 | — | Gly. tol. | mepsps |
| Cotton | EE-GH5 | — | Ins. res. | cry1Ab |
| Cotton | MON88701 | MON-88701- | Dicamba & Glu. | Modified dmo; bar |
| | | 3 | tol. | |
| Cotton | OsCr11 | — | Anti-allergy | Modified Cry j |
| Flax | FP967 | CDC-FL001- | ALS herb. tol. | als |
| | | 2 | | |
| Lentil | RH44 | | Imid. tol. | als |
| Maize | 3272 | SYN-E3272- | Modified alpha- | amy797E |
| | | 5 | amylase | |
| Maize | 5307 | SYN-05307- | Ins. res. | ecry3.1Ab |
| | | 1 | | |
| Maize | 59122 | DAS-59122- | Ins. res.; Glu. tol. | cry34Ab1; cry35Ab1; pat |
| | | 7 | | |
| Maize | 676 | PH-000676-7 | Glu. tol.; Poll. | pat; dam |
| | | | Cntrl. | |
| Maize | 678 | PH-000678-9 | Glu. tol.; Poll. | pat; dam |
| | | | Cntrl. | |
| Maize | 680 | PH-000680-2 | Glu. tol.; Poll. | pat; dam |
| | | | Cntrl. | |
| Maize | 98140 | DP-098140-6 | Gly. tol.; ALS | gat4621; zm-hra |
| | | | herb. tol. | |
| Maize | Bt10 | — | Ins. res.; Glu. tol. | cry1Ab; pat |
| Maize | Bt176 (176) | SYN-EV176- | Ins. res.; Glu. tol. | cry1Ab; bar |
| | | 9 | | |
| Maize | BVLA430101 | — | Phy. | phyA2 |
| Maize | CBH-351 | ACS- | Ins. res.; Glu. tol. | cry9C; bar |
| | | ZM004-3 | | |
| Maize | DAS40278-9 | DAS40278-9 | 2,4-D tol. | aad-1 |
| Maize | DBT418 | DKB-89614- | Ins. res.; Glu. tol. | cry1Ac; pinII; bar |
| | | 9 | | |
| Maize | DLL25 (B16) | DKB-89790- | Glu. tol. | bar |
| | | 5 | | |
| Maize | GA21 | MON-00021- | Gly. tol. | mepsps |
| | | 9 | | |
| Maize | GG25 | — | Gly. tol. | mepsps |
| Maize | GJ11 | — | Gly. tol. | mepsps |
| Maize | Fl117 | — | Gly. tol. | mepsps |
| Maize | GAT-ZM1 | — | Glu. tol. | pat |
| Maize | LY038 | REN-00038- | Increased lysine | cordapA |
| | | 3 | | |
| Maize | MIR162 | SYN-IR162- | Ins. res. | vip3Aa20 |
| | | 4 | | |
| Maize | MIR604 | SYN-IR604- | Ins. res. | mcry3A |
| | | 5 | | |
| Maize | MON801 | MON801 | Ins. res.; Gly. tol. | cry1Ab; cp4 epsps (aroA:CP4); |
| | (MON80100) | | | goxv247 |
| Maize | MON802 | MON-80200- | Ins. res.; Gly. tol. | cry1Ab; cp4 epsps (aroA:CP4); |
| | | 7 | | goxv247 |
| Maize | MON809 | PH-MON- | Ins. res.; Gly. tol. | cry1Ab; cp4 epsps (aroA:CP4); |
| | | 809-2 | | goxv247 |
| Maize | MON810 | MON-00810- | Ins. res.; Gly. tol. | cry1Ab; cp4 epsps (aroA:CP4); |
| | | 6 | | goxv247 |
| Maize | MON832 | — | Gly. tol. | cp4 epsps (aroA:CP4); goxv247 |
| Maize | MON863 | MON-00863- | Ins. res. | cry3Bb1 |
| | | 5 | | |
| Maize | MON87427 | MON-87427- | Gly. tol. | cp4 epsps (aroA:CP4) |
| | | 7 | | |
| Maize | MON87460 | MON-87460- | Drought tol. | cspB |
| | | 4 | | |
| Maize | MON88017 | MON-88017- | Ins. res.; Gly. tol. | cry3Bb1; cp4 epsps (aroA:CP4) |
| | | 3 | | |
| Maize | MON89034 | MON-89034- | Ins. res. | cry2Ab2; cry1A.105 |
| | | 3 | | |
| Maize | MS3 | ACS- | Glu. tol.; Poll. | bar; barnase |
| | | ZM001-9 | Cntrl. | |
| Maize | MS6 | ACS- | Glu. tol.; Poll. | bar; barnase |
| | | ZM005-4 | Cntrl. | |
| Maize | NK603 | MON-00603- | Gly. tol. | cp4 epsps (aroA:CP4) |
| | | 6 | | |
| Maize | T14 | ACS- | Glu. tol. | pat (syn) |
| | | ZM002-1 | | |
| Maize | T25 | ACS- | Glu. tol. | pat (syn) |
| | | ZM003-2 | | |
| Maize | TC1507 | DAS-01507- | Ins. res.; Glu. tol. | cry1Fa2; pat |
| | | 1 | | |
| Maize | TC6275 | DAS-06275- | Ins. res.; Glu. tol. | mocry1F; bar |
| | | 8 | | |
| Maize | VIP1034 | — | Ins. res.; Glu. tol. | vip3A; pat |
| Maize | 43A47 | DP-043A47- | Ins. res.; Glu. tol. | cry1F; cry34Ab1; cry35Ab1; pat |
| | | 3 | | |
| Maize | 40416 | DP-040416-8 | Ins. res.; Glu. tol. | cry1F; cry34Ab1; cry35Ab1; pat |
| Maize | 32316 | DP-032316-8 | Ins. res.; Glu. tol. | cry1F; cry34Ab1; cry35Ab1; pat |
| Maize | 4114 | DP-004114-3 | Ins. res.; Glu. tol. | cry1F; cry34Ab1; cry35Ab1; pat |
| Papaya | 55-1 | CUH-CP551- | Disease res. | prsv cp |
| | | 8 | | |
| Papaya | 63-1 | CUH-CP631- | Disease res. | prsv cp |
| | | 7 | | |
| Papaya | Huanong No. 1 | — | Disease res. | prsv rep |
| Papaya | X17-2 | UFL-X17CP- | Disease res. | prsv cp |
| | | 6 | | |
| Canola** | ZSR500 | — | Gly. tol. | cp4 epsps (aroA:CP4); goxv247 |
| Canola** | ZSR502 | — | Gly. tol. | cp4 epsps (aroA:CP4); goxv247 |
| Canola** | ZSR503 | — | Gly. tol. | cp4 epsps (aroA:CP4); goxv247 |
| Canola** | ZSR500 | — | Gly. tol. | cp4 epsps (aroA:CP4); goxv247 |
| Canola** | ZSR502 | — | Gly. tol. | cp4 epsps (aroA:CP4); goxv247 |
| Rice | 7Crp#242-95-7 | — | Anti-allergy | 7crp |
| Rice | 7Crp#10 | — | Anti-allergy | 7crp |
| Rice | GM Shanyou 63 | — | Ins. res. | cry1Ab; cry1Ac |
| Rice | Huahui-1/TT51 -1 | — | Ins. res. | cry1Ab; cry1Ac |
| Rice | LLRICE06 | ACS-OS001- | Glu. tol. | bar |
| | | 4 | | |
| Rice | LLRICE601 | BCS-OS003- | Glu. tol. | bar |
| | | 7 | | |
| Rice | LLRICE62 | ACS-OS002- | Glu. tol. | bar |
| | | 5 | | |
| Rice | Tarom molaii + | — | Ins. res. | cry1Ab (truncated) |
| | cry1Ab | | | |
| Rice | GAT-OS2 | — | Glu. tol. | bar |
| Rice | GAT-OS3 | — | Glu. tol. | bar |
| Rice | PE-7 | — | Ins. res. | Cry1Ac |
| Rice | 7Crp#10 | — | Anti-allergy | 7crp |
| Rice | KPD627-8 | — | High tryptophan | OASA1D |
| Rice | KPD722-4 | — | High tryptophan | OASA1D |
| Rice | KA317 | — | High tryptophan | OASA1D |
| Rice | HW5 | — | High tryptophan | OASA1D |
| Rice | HW1 | — | High tryptophan | OASA1D |
| Rice | B-4-1-18 | — | Erect leaves | Δ OsBRI1 |
| | | | semidwarf | |
| Rice | G-3-3-22 | — | Semidwarf | OSGA2ox1 |
| Rice | AD77 | — | Disease res. | DEF |
| Rice | AD51 | — | Disease res. | DEF |
| Rice | AD48 | — | Disease res. | DEF |
| Rice | AD41 | — | Disease res. | DEF |
| Rice | 13pNasNaatAprt1 | — | Low iron tol. | HvNAS1; HvNAAT-A; APRT |
| Rice | 13pAprt1 | — | Low iron tol. | APRT |
| Rice | gHvNAS1- | — | Low iron tol. | HvNAS1; HvNAAT-A; |
| | gHvNAAT-1 | | | HvNAAT-B |
| Rice | gHvIDS3-1 | — | Low iron tol. | HvIDS3 |
| Rice | gHvNAAT1 | — | Low iron tol. | HvNAAT-A; HvNAAT-B |
| Rice | gHvNAS1-1 | — | Low iron tol. | HvNAS1 |
| Rice | NIA-OS006-4 | — | Disease res. | WRKY45 |
| Rice | NIA-OS005-3 | — | Disease res. | WRKY45 |
| Rice | NIA-OS004-2 | — | Disease res. | WRKY45 |
| Rice | NIA-OS003-1 | — | Disease res. | WRKY45 |
| Rice | NIA-OS002-9 | — | Disease res. | WRKY45 |
| Rice | NIA-OS001-8 | — | Disease res. | WRKY45 |
| Rice | OsCr11 | — | Anti-allergy | Modified Cry j |
| Rice | 17053 | — | Gly. tol. | cp4 epsps (aroA:CP4) |
| Rice | 17314 | — | Gly. tol. | cp4 epsps (aroA:CP4) |
| Soybean | 260-05 (G94-1, G94- | — | Mofa | gm-fad2-1 (silencing locus) |
| | 19, G168) | | | |
| Soybean | A2704-12 | ACS- | Glu. tol. | pat |
| | | GM005-3 | | |
| Soybean | A2704-21 | ACS- | Glu. tol. | pat |
| | | GM004-2 | | |
| Soybean | A5547-127 | ACS- | Glu. tol. | pat |
| | | GM006-4 | | |
| Soybean | A5547-35 | ACS- | Glu. tol. | pat |
| | | GM008-6 | | |
| Soybean | CV127 | BPS-CV127- | Imid. tol. | csr1-2 |
| | | 9 | | |
| Soybean | DAS68416-4 | DAS68416-4 | Glu. tol. | pat |
| Soybean | DP305423 | DP-305423-1 | Mofa; ALS herb. | gm-fad2-1 (silencing locus); gm- |
| | | | tol. | hra |
| Soybean | DP356043 | DP-356043-5 | Mofa; Gly. tol. | gm-fad2-1 (silencing locus); |
| | | | | gat4601 |
| Soybean | FG72 | MST-FG072- | Gly. & HPPD | 2mepsps; hppdPF W336 |
| | | 3 | tol. | |
| Soybean | GTS 40-3-2 (40-3-2) | MON-04032- | Gly. tol. | cp4 epsps (aroA:CP4) |
| | | 6 | | |
| Soybean | GU262 | ACS- | Glu. tol. | pat |
| | | GM003-1 | | |
| Soybean | MON87701 | MON-87701- | Ins. res. | cry1Ac |
| | | 2 | | |
| Soybean | MON87705 | MON-87705- | Mofa; Gly. tol. | fatb1-A (sense & antisense); |
| | | 6 | | fad2-1A (sense & antisense); cp4 |
| | | | | epsps (aroA:CP4) |
| Soybean | MON87708 | MON-87708- | Dicamba & Gly. | dmo; cp4 epsps (aroA:CP4) |
| | | 9 | tol. | |
| Soybean | MON87769 | MON-87769- | Mofa; Gly. tol. | Pj.D6D; Nc.Fad3; cp4 epsps |
| | | 7 | | (aroA:CP4) |
| Soybean | MON89788 | MON-89788- | Gly. tol. | cp4 epsps (aroA:CP4) |
| | | 1 | | |
| Soybean | W62 | ACS- | Glu. tol. | bar |
| | | GM002-9 | | |
| Soybean | W98 | ACS- | Glu. tol. | bar |
| | | GM001-8 | | |
| Soybean | MON87754 | MON-87754- | High oil | dgat2A |
| | | 1 | | |
| Soybean | DAS21606 | DAS-21606 | Aoa & Glu. tol. | Modified aad-12; pat |
| Soybean | DAS44406 | DAS-44406- | Aoa, Gly. & Glu. | Modified aad-12; 2mepsps; pat |
| | | 6 | tol. | |
| Soybean | SYHT04R | SYN-0004R- | Mesotrione tol. | Modified avhppd |
| | | 8 | | |
| Soybean | 9582.814.19.1 | — | Ins. res. & Glu. | cry1Ac, cry1F, PAT |
| | | | tol. | |
| Squash | CZW3 | SEM- | Disease res. | cmv cp, zymv cp, wmv cp |
| | | ØCZW3-2 | | |
| Squash | ZW20 | SEM- | Disease res. | zymv cp, wmv cp |
| | | 0ZW20-7 | | |
| Sugar Beet | GTSB77 (T9100152) | SY-GTSB77- | Gly. tol. | cp4 epsps (aroA:CP4); goxv247 |
| | | 8 | | |
| Sugar Beet | H7-1 | KM-000H71- | Gly. tol. | cp4 epsps (aroA:CP4) |
| | | 4 | | |
| Sugar Beet | T120-7 | ACS-BV001- | Glu. tol. | pat |
| | | 3 | | |
| Sugar Beet | T227-1 | — | Gly. tol. | cp4 epsps (aroA:CP4) |
| Sugarcane | NXI-1T | — | Drought tol. | EcbetA |
| Sunflower | X81359 | — | Imid. tol. | als |
| Sweet | PK-SP01 | — | Disease res. | cmv cp |
| Pepper | | | | |
| Sunflower | X81359 | — | Imid. tol. | als |
| Wheat | MON71800 | MON- | Gly. tol. | cp4 epsps (aroA:CP4) |
| | | 718ØØ-3 | | |

*Argentine
**Polish

Although most typically, compounds of the invention are used to control undesired vegetation, contact of desired vegetation in the treated locus with compounds of the invention may result in super-additive or synergistic effects with genetic traits in the desired vegetation, including traits incorporated through genetic modification. For example, resistance to phytophagous insect pests or plant diseases, tolerance to biotic/abiotic stresses or storage stability may be greater than expected from the genetic traits in the desired vegetation.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its esters (e.g., methyl, ethyl) and salts (e.g., sodium, potassium), aminopyralid, amitrole, ammonium sulfamate, anilofos, asulam, atrazine, azimsulfuron, beflubutamid, benazolin, benazolin-ethyl, bencarbazone, benfluralin, benfuresate, bensulfuron-methyl, bensulide, bentazone, benzobicyclon, benzofenap, bicyclopyrone, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clacyfos, clefoxydim, clethodim, cyclopyrimorate (6-chloro-3-(2-cyclopropyl-6-methylphenoxy)-4-pyridazinyl 4-morpholinecarboxylate), clodinafop-propargyl, clomazone, clomeprop, clopyralid, clopyralid-olamine, cloransulam-methyl, cumyluron, cyanazine, cycloate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenquinotrione (2-[[8-chloro-3,4-dihydro-4-(4-methoxyphenyl)-3-oxo-2-quinoxalinyl]carbonyl]-1,3-cyclohexanedione), fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, fluorochloridone, fluoroxypyr, flurtamone, fluthiacet-methyl, fomesafen, foramsulfuron, fosamine-ammonium, glufosinate, glufosinate-ammonium, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, imazamethabenz-methyl, imazamox, imazapic, imazapyr, imazaquin, imazaquin-ammonium, imazethapyr, imazethapyr-ammonium, imazosulfuron, indanofan, indaziflam, iofensulfuron, iodosulfuron-methyl, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methiozolin, methylarsonic acid and its calcium, monoammonium, monosodium and disodium salts, methyldymron, metobenzuron, metobromuron, metolachlor, S-metolachlor, metosulam, metoxuron, metribuzin, metsulfuron-methyl, molinate, monolinuron, naproanilide, napropamide, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thifensulfuron-methyl, thiobencarb, tiafenacil (methyl N-[2-[[2-chloro-5-[3,6-dihydro-3-methyl-2,6-dioxo-4-(trifluoromethyl)-1(2H)-pyrimidinyl]-4-fluorophenyl]thio]-1-oxopropyl]-(β-alaninate), tiocarbazil, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifluralin, triflusulfuron-methyl, tritosulfuron and vernolate. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butyl.) Butyl. and Puccinia thlaspeos Schub.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

Compounds of this invention can also be mixed with RNA to enhance effectiveness or to confer safening properties. Accordingly, a compound of Formula 1 can be mixed with polynucleotides including but not limited to DNA, RNA, and/or chemically modified nucleotides influencing the amount of a particular target through down regulation, interference, suppression or silencing of the genetically derived transcript that render a herbicidal effect. Alternatively, a compound of Formula 1 can be mixed with polynucleotides including but not limited to DNA, RNA, and/or chemically modified nucleotides influencing the amount of a particular target through down regulation, interference, suppression or silencing of the genetically derived transcript that render a safening effect.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, active ingredients are often applied at an application rate between one-half and the full application rate specified on product labels for use of the active ingredient alone. The amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the invention with at least one other herbicidal active ingredient. Of particular note is such a combination where the other herbicidal active ingredient has different site of action from the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners such as allidochlor, N-(aminocarbonyl)-2-methylbenzenesulfonamide, benoxacor, BCS (1-bromo-4-[(chloromethyl)sulfonyl]benzene), cloquintocet-mexyl, cyometrinil, cyprosulfonamide, dichlormid, 4-(dichloroacetyl)-1-oxa-4-azospiro[4.5]decane (MON 4660), 2-(dichloromethyl)-2-methyl-1,3-dioxolane (MG 191), dicyclonon, dietholate, ethyl 1,6-dihydro-1-(2-methoxyphenyl)-6-oxo-2-phenyl-5-pyrimidinecarboxylate, fenchlorazole-ethyl, fenclorim, flurazole, fluxofenim, furilazole, 2-hydroxy-N,N-dimethyl-6-(trifluoromethyl)pyridine-3-carboxamide, isoxadifen-ethyl, mefenpyr-diethyl, mephenate, methoxyphenone ((4-methoxy-3-methylphenyl)(3-methylphenyl)methanone), naphthalic anhydride (1,8-naphthalic anhydride) oxabetrinil and 3-oxo-1-cyclohexen-1-yl 1-(3,4-dimethylphenyl)-1,6-dihydro-6-oxo-2-phenyl-5-pyrimidinecarboxylate to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Of note is a composition comprising a compound of the invention (in a herbicidally effective amount), at least one additional active ingredient selected from the group consisting of other herbicides and herbicide safeners (in an effective amount), and at least one component selected from the group consisting of surfactants, solid diluents and liquid diluents.

Preferred for better control of undesired vegetation (e.g., lower use rate such as from synergism, broader spectrum of weeds controlled, or enhanced crop safety) or for preventing the development of resistant weeds are mixtures of a compound of this invention with another herbicide. Table A1 lists specific combinations of a Component (a) with Component (b) illustrative of the mixtures, compositions and methods of the present invention. Compound 105 in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 105 in Index Table A) with 2,4-D is typically applied in a weight ratio between 1:192 to 6:1. The remaining lines of Table A1 are to be construed similarly.

TABLE A1

| | | Typical Weight | More Typical | Most Typical |
|---|---|---|---|---|
| Component (a) | Component (b) | Ratio | Weight Ratio | Weight Ratio |
| Compound 105 | 2,4-D | 1:192 to 6:1 | 1:64 to 2:1 | 1:24 to 1:3 |
| Compound 105 | halauxifen | 1:20 to 56:1 | 1:6 to 19:1 | 1:2 to 4:1 |
| Compound 105 | halauxifen methyl | 1:20 to 56:1 | 1:6 to 19:1 | 1:2 to 4:1 |
| Compound 105 | Acetochlor | 1:768 to 2:1 | 1:256 to 1:2 | 1:96 to 1:11 |
| Compound 105 | Acifluorfen | 1:96 to 12:1 | 1:32 to 4:1 | 1:12 to 1:2 |
| Compound 105 | Aclonifen | 1:857 to 2:1 | 1:285 to 1:3 | 1:107 to 1:12 |
| Compound 105 | Alachlor | 1:768 to 2:1 | 1:256 to 1:2 | 1:96 to 1:11 |
| Compound 105 | Ametryn | 1:384 to 3:1 | 1:128 to 1:1 | 1:48 to 1:6 |
| Compound 105 | Amicarbazone | 1:192 to 6:1 | 1:64 to 2:1 | 1:24 to 1:3 |
| Compound 105 | Amidosulfuron | 1:6 to 168:1 | 1:2 to 56:1 | 1:1 to 11:1 |
| Compound 105 | Aminocyclopyrachlor | 1:48 to 24:1 | 1:16 to 8:1 | 1:6 to 2:1 |
| Compound 105 | Aminopyralid | 1:20 to 56:1 | 1:6 to 19:1 | 1:2 to 4:1 |
| Compound 105 | Amitrole | 1:768 to 2:1 | 1:256 to 1:2 | 1:96 to 1:11 |
| Compound 105 | Anilofos | 1:96 to 12:1 | 1:32 to 4:1 | 1:12 to 1:2 |
| Compound 105 | Asulam | 1:960 to 2:1 | 1:320 to 1:3 | 1:120 to 1:14 |
| Compound 105 | Atrazine | 1:192 to 6:1 | 1:64 to 2:1 | 1:24 to 1:3 |
| Compound 105 | Azimsulfuron | 1:6 to 168:1 | 1:2 to 56:1 | 1:1 to 11:1 |
| Compound 105 | Beflubutamid | 1:342 to 4:1 | 1:114 to 2:1 | 1:42 to 1:5 |
| Compound 105 | Benfuresate | 1:617 to 2:1 | 1:205 to 1:2 | 1:77 to 1:9 |
| Compound 105 | Bensulfuron-methyl | 1:25 to 45:1 | 1:8 to 15:1 | 1:3 to 3:1 |
| Compound 105 | Bentazone | 1:192 to 6:1 | 1:64 to 2:1 | 1:24 to 1:3 |
| Compound 105 | Benzobicyclon | 1:85 to 14:1 | 1:28 to 5:1 | 1:10 to 1:2 |
| Compound 105 | Benzofenap | 1:257 to 5:1 | 1:85 to 2:1 | 1:32 to 1:4 |
| Compound 105 | Bicyclopyrone | 1:42 to 27:1 | 1:14 to 9:1 | 1:5 to 2:1 |
| Compound 105 | Bifenox | 1:257 to 5:1 | 1:85 to 2:1 | 1:32 to 1:4 |
| Compound 105 | Bispyribac-sodium | 1:10 to 112:1 | 1:3 to 38:1 | 1:1 to 7:1 |
| Compound 105 | Bromacil | 1:384 to 3:1 | 1:128 to 1:1 | 1:48 to 1:6 |
| Compound 105 | Bromobutide | 1:384 to 3:1 | 1:128 to 1:1 | 1:48 to 1:6 |
| Compound 105 | Bromoxynil | 1:96 to 12:1 | 1:32 to 4:1 | 1:12 to 1:2 |
| Compound 105 | Butachlor | 1:768 to 2:1 | 1:256 to 1:2 | 1:96 to 1:11 |
| Compound 105 | Butafenacil | 1:42 to 27:1 | 1:14 to 9:1 | 1:5 to 2:1 |
| Compound 105 | Butylate | 1:1542 to 1:2 | 1:514 to 1:5 | 1:192 to 1:22 |
| Compound 105 | Carfenstrole | 1:192 to 6:1 | 1:64 to 2:1 | 1:24 to 1:3 |
| Compound 105 | Carfentrazone-ethyl | 1:128 to 9:1 | 1:42 to 3:1 | 1:16 to 1:2 |
| Compound 105 | Chlorimuron-ethyl | 1:8 to 135:1 | 1:2 to 45:1 | 1:1 to 9:1 |
| Compound 105 | Chlorotoluron | 1:768 to 2:1 | 1:256 to 1:2 | 1:96 to 1:11 |
| Compound 105 | Chlorsulfuron | 1:6 to 168:1 | 1:2 to 56:1 | 1:1 to 11:1 |
| Compound 105 | Cincosulfuron | 1:17 to 68:1 | 1:5 to 23:1 | 1:2 to 5:1 |
| Compound 105 | Cinidon-ethyl | 1:384 to 3:1 | 1:128 to 1:1 | 1:48 to 1:6 |
| Compound 105 | Cinmethylin | 1:34 to 34:1 | 1:11 to 12:1 | 1:4 to 3:1 |
| Compound 105 | Clacyfos | 1:34 to 34:1 | 1:11 to 12:1 | 1:4 to 3:1 |
| Compound 105 | Clethodim | 1:48 to 24:1 | 1:16 to 8:1 | 1:6 to 2:1 |
| Compound 105 | Clodinafop-propargyl | 1:20 to 56:1 | 1:6 to 19:1 | 1:2 to 4:1 |
| Compound 105 | Clomazone | 1:384 to 3:1 | 1:128 to 1:1 | 1:48 to 1:6 |
| Compound 105 | Clomeprop | 1:171 to 7:1 | 1:57 to 3:1 | 1:21 to 1:3 |
| Compound 105 | Clopyralid | 1:192 to 6:1 | 1:64 to 2:1 | 1:24 to 1:3 |
| Compound 105 | Cloransulam-methyl | 1:12 to 96:1 | 1:4 to 32:1 | 1:1 to 6:1 |
| Compound 105 | Cumyluron | 1:384 to 3:1 | 1:128 to 1:1 | 1:48 to 1:6 |
| Compound 105 | Cyanazine | 1:384 to 3:1 | 1:128 to 1:1 | 1:48 to 1:6 |
| Compound 105 | Cyclopyrimorate | 1:17 to 68:1 | 1:5 to 23:1 | 1:2 to 5:1 |
| Compound 105 | Cyclosulfamuron | 1:17 to 68:1 | 1:5 to 23:1 | 1:2 to 5:1 |
| Compound 105 | Cycloxydim | 1:96 to 12:1 | 1:32 to 4:1 | 1:12 to 1:2 |
| Compound 105 | Cyhalofop | 1:25 to 45:1 | 1:8 to 15:1 | 1:3 to 3:1 |
| Compound 105 | Daimuron | 1:192 to 6:1 | 1:64 to 2:1 | 1:24 to 1:3 |
| Compound 105 | Desmedipham | 1:322 to 4:1 | 1:107 to 2:1 | 1:40 to 1:5 |
| Compound 105 | Dicamba | 1:192 to 6:1 | 1:64 to 2:1 | 1:24 to 1:3 |
| Compound 105 | Dichlobenil | 1:1371 to 1:2 | 1:457 to 1:4 | 1:171 to 1:20 |
| Compound 105 | Dichlorprop | 1:925 to 2:1 | 1:308 to 1:3 | 1:115 to 1:13 |
| Compound 105 | Diclofop-methyl | 1:384 to 3:1 | 1:128 to 1:1 | 1:48 to 1:6 |
| Compound 105 | Diclosulam | 1:10 to 112:1 | 1:3 to 38:1 | 1:1 to 7:1 |
| Compound 105 | Difenzoquat | 1:288 to 4:1 | 1:96 to 2:1 | 1:36 to 1:4 |
| Compound 105 | Diflufenican | 1:857 to 2:1 | 1:285 to 1:3 | 1:107 to 1:12 |
| Compound 105 | Diflufenzopyr | 1:12 to 96:1 | 1:4 to 32:1 | 1:1 to 6:1 |
| Compound 105 | Dimethachlor | 1:768 to 2:1 | 1:256 to 1:2 | 1:96 to 1:11 |
| Compound 105 | Dimethametryn | 1:192 to 6:1 | 1:64 to 2:1 | 1:24 to 1:3 |
| Compound 105 | Dimethenamid-P | 1:384 to 3:1 | 1:128 to 1:1 | 1:48 to 1:6 |
| Compound 105 | Dithiopyr | 1:192 to 6:1 | 1:64 to 2:1 | 1:24 to 1:3 |
| Compound 105 | Diuron | 1:384 to 3:1 | 1:128 to 1:1 | 1:48 to 1:6 |
| Compound 105 | EPTC | 1:768 to 2:1 | 1:256 to 1:2 | 1:96 to 1:11 |
| Compound 105 | Esprocarb | 1:1371 to 1:2 | 1:457 to 1:4 | 1:171 to 1:20 |
| Compound 105 | Ethalfluralin | 1:384 to 3:1 | 1:128 to 1:1 | 1:48 to 1:6 |
| Compound 105 | Ethametsulfuron-methyl | 1:17 to 68:1 | 1:5 to 23:1 | 1:2 to 5:1 |
| Compound 105 | Ethoxyfen | 1:8 to 135:1 | 1:2 to 45:1 | 1:1 to 9:1 |
| Compound 105 | Ethoxysulfuron | 1:20 to 56:1 | 1:6 to 19:1 | 1:2 to 4:1 |
| Compound 105 | Etobenzanid | 1:257 to 5:1 | 1:85 to 2:1 | 1:32 to 1:4 |
| Compound 105 | Fenoxaprop-ethyl | 1:120 to 10:1 | 1:40 to 4:1 | 1:15 to 1:2 |
| Compound 105 | Fenoxasulfone | 1:85 to 14:1 | 1:28 to 5:1 | 1:10 to 1:2 |
| Compound 105 | Fenquinotrione | 1:17 to 68:1 | 1:5 to 23:1 | 1:2 to 5:1 |
| Compound 105 | Fentrazamide | 1:17 to 68:1 | 1:5 to 23:1 | 1:2 to 5:1 |
| Compound 105 | Flazasulfuron | 1:17 to 68:1 | 1:5 to 23:1 | 1:2 to 5:1 |
| Compound 105 | Florasulam | 1:2 to 420:1 | 1:1 to 140:1 | 2:1 to 27:1 |
| Compound 105 | Fluazifop-butyl | 1:192 to 6:1 | 1:64 to 2:1 | 1:24 to 1:3 |
| Compound 105 | Flucarbazone | 1:8 to 135:1 | 1:2 to 45:1 | 1:1 to 9:1 |
| Compound 105 | Flucetosulfuron | 1:8 to 135:1 | 1:2 to 45:1 | 1:1 to 9:1 |
| Compound 105 | Flufenacet | 1:257 to 5:1 | 1:85 to 2:1 | 1:32 to 1:4 |
| Compound 105 | Flumetsulam | 1:24 to 48:1 | 1:8 to 16:1 | 1:3 to 3:1 |
| Compound 105 | Flumiclorac-pentyl | 1:10 to 112:1 | 1:3 to 38:1 | 1:1 to 7:1 |
| Compound 105 | Flumioxazin | 1:25 to 45:1 | 1:8 to 15:1 | 1:3 to 3:1 |
| Compound 105 | Fluometuron | 1:384 to 3:1 | 1:128 to 1:1 | 1:48 to 1:6 |
| Compound 105 | Flupyrsulfuron-methyl | 1:3 to 336:1 | 1:1 to 112:1 | 2:1 to 21:1 |
| Compound 105 | Fluridone | 1:384 to 3:1 | 1:128 to 1:1 | 1:48 to 1:6 |
| Compound 105 | Fluroxypyr | 1:96 to 12:1 | 1:32 to 4:1 | 1:12 to 1:2 |
| Compound 105 | Flurtamone | 1:857 to 2:1 | 1:285 to 1:3 | 1:107 to 1:12 |
| Compound 105 | Fluthiacet-methyl | 1:48 to 42:1 | 1:16 to 14:1 | 1:3 to 3:1 |
| Compound 105 | Fomesafen | 1:96 to 12:1 | 1:32 to 4:1 | 1:12 to 1:2 |
| Compound 105 | Foramsulfuron | 1:13 to 84:1 | 1:4 to 28:1 | 1:1 to 6:1 |
| Compound 105 | Glufosinate | 1:288 to 4:1 | 1:96 to 2:1 | 1:36 to 1:4 |
| Compound 105 | Glyphosate | 1:288 to 4:1 | 1:96 to 2:1 | 1:36 to 1:4 |
| Compound 105 | Halosulfuron-methyl | 1:17 to 68:1 | 1:5 to 23:1 | 1:2 to 5:1 |
| Compound 105 | Haloxyfop-methyl | 1:34 to 34:1 | 1:11 to 12:1 | 1:4 to 3:1 |
| Compound 105 | Hexazinone | 1:192 to 6:1 | 1:64 to 2:1 | 1:24 to 1:3 |
| Compound 105 | Imazamox | 1:13 to 84:1 | 1:4 to 28:1 | 1:1 to 6:1 |
| Compound 105 | Imazapic | 1:20 to 56:1 | 1:6 to 19:1 | 1:2 to 4:1 |
| Compound 105 | Imazapyr | 1:85 to 14:1 | 1:28 to 5:1 | 1:10 to 1:2 |
| Compound 105 | Imazaquin | 1:34 to 34:1 | 1:11 to 12:1 | 1:4 to 3:1 |
| Compound 105 | Imazethabenz-methyl | 1:171 to 7:1 | 1:57 to 3:1 | 1:21 to 1:3 |
| Compound 105 | Imazethapyr | 1:24 to 48:1 | 1:8 to 16:1 | 1:3 to 3:1 |
| Compound 105 | Imazosulfuron | 1:27 to 42:1 | 1:9 to 14:1 | 1:3 to 3:1 |
| Compound 105 | Indanofan | 1:342 to 4:1 | 1:114 to 2:1 | 1:42 to 1:5 |
| Compound 105 | Indaziflam | 1:25 to 45:1 | 1:8 to 15:1 | 1:3 to 3:1 |
| Compound 105 | Iodosulfuron-methyl | 1:3 to 336:1 | 1:1 to 112:1 | 2:1 to 21:1 |
| Compound 105 | Ioxynil | 1:192 to 6:1 | 1:64 to 2:1 | 1:24 to 1:3 |
| Compound 105 | Ipfencarbazone | 1:85 to 14:1 | 1:28 to 5:1 | 1:10 to 1:2 |
| Compound 105 | Isoproturon | 1:384 to 3:1 | 1:128 to 1:1 | 1:48 to 1:6 |
| Compound 105 | Isoxaben | 1:288 to 4:1 | 1:96 to 2:1 | 1:36 to 1:4 |
| Compound 105 | Isoxaflutole | 1:60 to 20:1 | 1:20 to 7:1 | 1:7 to 2:1 |
| Compound 105 | Lactofen | 1:42 to 27:1 | 1:14 to 9:1 | 1:5 to 2:1 |
| Compound 105 | Lenacil | 1:384 to 3:1 | 1:128 to 1:1 | 1:48 to 1:6 |
| Compound 105 | Linuron | 1:384 to 3:1 | 1:128 to 1:1 | 1:48 to 1:6 |
| Compound 105 | MCPA | 1:192 to 6:1 | 1:64 to 2:1 | 1:24 to 1:3 |
| Compound 105 | MCPB | 1:288 to 4:1 | 1:96 to 2:1 | 1:36 to 1:4 |
| Compound 105 | Mecoprop | 1:768 to 2:1 | 1:256 to 1:2 | 1:96 to 1:11 |
| Compound 105 | Mefenacet | 1:384 to 3:1 | 1:128 to 1:1 | 1:48 to 1:6 |
| Compound 105 | Mefluidide | 1:192 to 6:1 | 1:64 to 2:1 | 1:24 to 1:3 |
| Compound 105 | Mesosulfuron-methyl | 1:5 to 224:1 | 1:1 to 75:1 | 1:1 to 14:1 |
| Compound 105 | Mesotrione | 1:42 to 27:1 | 1:14 to 9:1 | 1:5 to 2:1 |
| Compound 105 | Metamifop | 1:42 to 27:1 | 1:14 to 9:1 | 1:5 to 2:1 |
| Compound 105 | Metazachlor | 1:384 to 3:1 | 1:128 to 1:1 | 1:48 to 1:6 |
| Compound 105 | Metazosulfuron | 1:25 to 45:1 | 1:8 to 15:1 | 1:3 to 3:1 |
| Compound 105 | Methabenzthiazuron | 1:768 to 2:1 | 1:256 to 1:2 | 1:96 to 1:11 |
| Compound 105 | Metolachlor | 1:768 to 2:1 | 1:256 to 1:2 | 1:96 to 1:11 |
| Compound 105 | Metosulam | 1:8 to 135:1 | 1:2 to 45:1 | 1:1 to 9:1 |
| Compound 105 | Metribuzin | 1:192 to 6:1 | 1:64 to 2:1 | 1:24 to 1:3 |
| Compound 105 | Metsulfuron-methyl | 1:2 to 560:1 | 1:1 to 187:1 | 3:1 to 35:1 |
| Compound 105 | Molinate | 1:1028 to 2:1 | 1:342 to 1:3 | 1:128 to 1:15 |
| Compound 105 | Napropamide | 1:384 to 3:1 | 1:128 to 1:1 | 1:48 to 1:6 |
| Compound 105 | Naptalam | 1:192 to 6:1 | 1:64 to 2:1 | 1:24 to 1:3 |
| Compound 105 | Nicosulfuron | 1:12 to 96:1 | 1:4 to 32:1 | 1:1 to 6:1 |
| Compound 105 | Norflurazon | 1:1152 to 1:1 | 1:384 to 1:3 | 1:144 to 1:16 |
| Compound 105 | Orbencarb | 1:1371 to 1:2 | 1:457 to 1:4 | 1:171 to 1:20 |
| Compound 105 | Orthosulfamuron | 1:20 to 56:1 | 1:6 to 19:1 | 1:2 to 4:1 |
| Compound 105 | Oryzalin | 1:514 to 3:1 | 1:171 to 1:2 | 1:64 to 1:8 |
| Compound 105 | Oxadiargyl | 1:384 to 3:1 | 1:128 to 1:1 | 1:48 to 1:6 |
| Compound 105 | Oxadiazon | 1:548 to 3:1 | 1:182 to 1:2 | 1:68 to 1:8 |
| Compound 105 | Oxasulfuron | 1:27 to 42:1 | 1:9 to 14:1 | 1:3 to 3:1 |
| Compound 105 | Oxaziclomefone | 1:42 to 27:1 | 1:14 to 9:1 | 1:5 to 2:1 |
| Compound 105 | Oxyfluorfen | 1:384 to 3:1 | 1:128 to 1:1 | 1:48 to 1:6 |
| Compound 105 | Paraquat | 1:192 to 6:1 | 1:64 to 2:1 | 1:24 to 1:3 |
| Compound 105 | Pendimethalin | 1:384 to 3:1 | 1:128 to 1:1 | 1:48 to 1:6 |
| Compound 105 | Penoxsulam | 1:10 to 112:1 | 1:3 to 38:1 | 1:1 to 7:1 |
| Compound 105 | Penthoxamid | 1:384 to 3:1 | 1:128 to 1:1 | 1:48 to 1:6 |
| Compound 105 | Pentoxazone | 1:102 to 12:1 | 1:34 to 4:1 | 1:12 to 1:2 |
| Compound 105 | Phenmedipham | 1:102 to 12:1 | 1:34 to 4:1 | 1:12 to 1:2 |
| Compound 105 | Picloram | 1:96 to 12:1 | 1:32 to 4:1 | 1:12 to 1:2 |
| Compound 105 | Picolinafen | 1:34 to 34:1 | 1:11 to 12:1 | 1:4 to 3:1 |
| Compound 105 | Pinoxaden | 1:25 to 45:1 | 1:8 to 15:1 | 1:3 to 3:1 |
| Compound 105 | Pretilachlor | 1:192 to 6:1 | 1:64 to 2:1 | 1:24 to 1:3 |
| Compound 105 | Primisulfuron-methyl | 1:8 to 135:1 | 1:2 to 45:1 | 1:1 to 9:1 |
| Compound 105 | Prodiamine | 1:384 to 3:1 | 1:128 to 1:1 | 1:48 to 1:6 |
| Compound 105 | Profoxydim | 1:42 to 27:1 | 1:14 to 9:1 | 1:5 to 2:1 |
| Compound 105 | Prometryn | 1:384 to 3:1 | 1:128 to 1:1 | 1:48 to 1:6 |
| Compound 105 | Propachlor | 1:1152 to 1:1 | 1:384 to 1:3 | 1:144 to 1:16 |
| Compound 105 | Propanil | 1:384 to 3:1 | 1:128 to 1:1 | 1:48 to 1:6 |
| Compound 105 | Propaquizafop | 1:48 to 24:1 | 1:16 to 8:1 | 1:6 to 2:1 |
| Compound 105 | Propoxycarbazone | 1:17 to 68:1 | 1:5 to 23:1 | 1:2 to 5:1 |
| Compound 105 | Propyrisulfuron | 1:17 to 68:1 | 1:5 to 23:1 | 1:2 to 5:1 |
| Compound 105 | Propyzamide | 1:384 to 3:1 | 1:128 to 1:1 | 1:48 to 1:6 |
| Compound 105 | Prosulfocarb | 1:1200 to 1:2 | 1:400 to 1:4 | 1:150 to 1:17 |
| Compound 105 | Prosulfuron | 1:6 to 168:1 | 1:2 to 56:1 | 1:1 to 11:1 |
| Compound 105 | Pyraclonil | 1:42 to 27:1 | 1:14 to 9:1 | 1:5 to 2:1 |
| Compound 105 | Pyraflufen-ethyl | 1:5 to 224:1 | 1:1 to 75:1 | 1:1 to 14:1 |
| Compound 105 | Pyrasulfotole | 1:13 to 84:1 | 1:4 to 28:1 | 1:1 to 6:1 |
| Compound 105 | Pyrazolynate | 1:857 to 2:1 | 1:285 to 1:3 | 1:107 to 1:12 |
| Compound 105 | Pyrazosulfuron-ethyl | 1:10 to 112:1 | 1:3 to 38:1 | 1:1 to 7:1 |
| Compound 105 | Pyrazoxyfen | 1:5 to 224:1 | 1:1 to 75:1 | 1:1 to 14:1 |
| Compound 105 | Pyribenzoxim | 1:10 to 112:1 | 1:3 to 38:1 | 1:1 to 7:1 |
| Compound 105 | Pyributicarb | 1:384 to 3:1 | 1:128 to 1:1 | 1:48 to 1:6 |
| Compound 105 | Pyridate | 1:288 to 4:1 | 1:96 to 2:1 | 1:36 to 1:4 |
| Compound 105 | Pyriftalid | 1:10 to 112:1 | 1:3 to 38:1 | 1:1 to 7:1 |
| Compound 105 | Pyriminobac-methyl | 1:20 to 56:1 | 1:6 to 19:1 | 1:2 to 4:1 |
| Compound 105 | Pyrimisulfan | 1:17 to 68:1 | 1:5 to 23:1 | 1:2 to 5:1 |
| Compound 105 | Pyrithiobac | 1:24 to 48:1 | 1:8 to 16:1 | 1:3 to 3:1 |
| Compound 105 | Pyroxasulfone | 1:85 to 14:1 | 1:28 to 5:1 | 1:10 to 1:2 |
| Compound 105 | Pyroxsulam | 1:5 to 224:1 | 1:1 to 75:1 | 1:1 to 14:1 |
| Compound 105 | Quinclorac | 1:192 to 6:1 | 1:64 to 2:1 | 1:24 to 1:3 |
| Compound 105 | Quizalofop-ethyl | 1:42 to 27:1 | 1:14 to 9:1 | 1:5 to 2:1 |
| Compound 105 | Rimsulfuron | 1:13 to 84:1 | 1:4 to 28:1 | 1:1 to 6:1 |
| Compound 105 | Saflufenacil | 1:25 to 45:1 | 1:8 to 15:1 | 1:3 to 3:1 |
| Compound 105 | Sethoxydim | 1:96 to 12:1 | 1:32 to 4:1 | 1:12 to 1:2 |
| Compound 105 | Simazine | 1:384 to 3:1 | 1:128 to 1:1 | 1:48 to 1:6 |
| Compound 105 | Sulcotrione | 1:120 to 10:1 | 1:40 to 4:1 | 1:15 to 1:2 |
| Compound 105 | Sulfentrazone | 1:147 to 8:1 | 1:49 to 3:1 | 1:18 to 1:3 |
| Compound 105 | Sulfometuron-methyl | 1:34 to 34:1 | 1:11 to 12:1 | 1:4 to 3:1 |
| Compound 105 | Sulfosulfuron | 1:8 to 135:1 | 1:2 to 45:1 | 1:1 to 9:1 |
| Compound 105 | Tebuthiuron | 1:384 to 3:1 | 1:128 to 1:1 | 1:48 to 1:6 |
| Compound 105 | Tefuryltrione | 1:42 to 27:1 | 1:14 to 9:1 | 1:5 to 2:1 |
| Compound 105 | Tembotrione | 1:31 to 37:1 | 1:10 to 13:1 | 1:3 to 3:1 |
| Compound 105 | Tepraloxydim | 1:25 to 45:1 | 1:8 to 15:1 | 1:3 to 3:1 |
| Compound 105 | Terbacil | 1:288 to 4:1 | 1:96 to 2:1 | 1:36 to 1:4 |
| Compound 105 | Terbuthylatrazine | 1:857 to 2:1 | 1:285 to 1:3 | 1:107 to 1:12 |
| Compound 105 | Terbutryn | 1:192 to 6:1 | 1:64 to 2:1 | 1:24 to 1:3 |
| Compound 105 | Thenylchlor | 1:85 to 14:1 | 1:28 to 5:1 | 1:10 to 1:2 |
| Compound 105 | Thiazopyr | 1:384 to 3:1 | 1:128 to 1:1 | 1:48 to 1:6 |
| Compound 105 | Thiencarbazone | 1:3 to 336:1 | 1:1 to 112:1 | 2:1 to 21:1 |
| Compound 105 | Thifensulfuron-methyl | 1:5 to 224:1 | 1:1 to 75:1 | 1:1 to 14:1 |
| Compound 105 | Tiafenacil | 1:17 to 68:1 | 1:5 to 23:1 | 1:2 to 5:1 |
| Compound 105 | Thiobencarb | 1:768 to 2:1 | 1:256 to 1:2 | 1:96 to 1:11 |
| Compound 105 | Topramazone | 1:6 to 168:1 | 1:2 to 56:1 | 1:1 to 11:1 |
| Compound 105 | Tralkoxydim | 1:68 to 17:1 | 1:22 to 6:1 | 1:8 to 2:1 |
| Compound 105 | Triallate | 1:768 to 2:1 | 1:256 to 1:2 | 1:96 to 1:11 |
| Compound 105 | Triasulfuron | 1:5 to 224:1 | 1:1 to 75:1 | 1:1 to 14:1 |
| Compound 105 | Triaziflam | 1:171 to 7:1 | 1:57 to 3:1 | 1:21 to 1:3 |
| Compound 105 | Tribenuron-methyl | 1:3 to 336:1 | 1:1 to 112:1 | 2:1 to 21:1 |
| Compound 105 | Triclopyr | 1:192 to 6:1 | 1:64 to 2:1 | 1:24 to 1:3 |
| Compound 105 | Trifloxysulfuron | 1:2 to 420:1 | 1:1 to 140:1 | 2:1 to 27:1 |
| Compound 105 | Trifluralin | 1:288 to 4:1 | 1:96 to 2:1 | 1:36 to 1:4 |
| Compound 105 | Triflusulfuron-methyl | 1:17 to 68:1 | 1:5 to 23:1 | 1:2 to 5:1 |
| Compound 105 | Tritosulfuron | 1:13 to 84:1 | 1:4 to 28:1 | 1:1 to 6:1 |

Table A2 is constructed the same as Table A1 above except that entries below the “Component (a)” column heading are replaced with the respective Component (a) Column Entry shown below. Compound 78 in the Component (a) column is identified in Index Table A. Thus, for example, in Table A2 the entries below the “Component (a)” column heading all recite “Compound 78” (i.e. Compound 78 identified in Index Table A), and the first line below the column headings in Table A2 specifically discloses a mixture of Compound 78 with 2,4-D. Tables A3 through A7 are constructed similarly.

| Table | Component (a) |
|---|---|
| Number | Column Entries |
| A2 | Compound 78 |
| A3 | Compound 104 |
| A4 | Compound 106 |
| A5 | Compound 108 |
| A6 | Compound 98 |
| A7 | Compound 76 |

Preferred for better control of undesired vegetation (e.g., lower use rate such as from synergism, broader spectrum of weeds controlled, or enhanced crop safety) or for preventing the development of resistant weeds are mixtures of a compound of this invention with a herbicide selected from the group consisting of glyphosate, chlorimuron-ethyl, nicosulfuron, mesotrione, thifensulfuron-methyl, flupyrsulfuron-methyl, tribenuron, pyroxasulfone. pinoxaden, tembotrione, florasulam, pyroxsulam, metolachlor and S-metolachlor.

The following Tests demonstrate the control efficacy of the compounds of this invention against specific weeds. The weed control afforded by the compounds is not limited, however, to these species. See Index Tables A-B for compound descriptions. The following abbreviations are used in the Index Tables which follow: t is tertiary, s is secondary, n is normal, i is iso, c is cyclo, Pr is propyl, Bu is butyl, c-Pr is cyclopropyl, t-Bu is tert-butyl and Ph is phenyl. The abbreviation “Ex.” stands for “Example” and is followed by a number indicating in which example the compound is prepared. Structures for J are shown described as shown below.

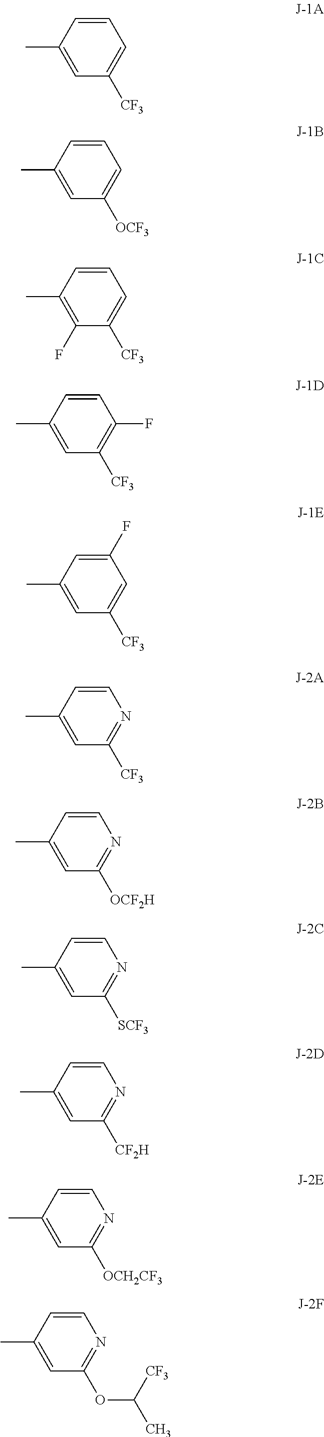

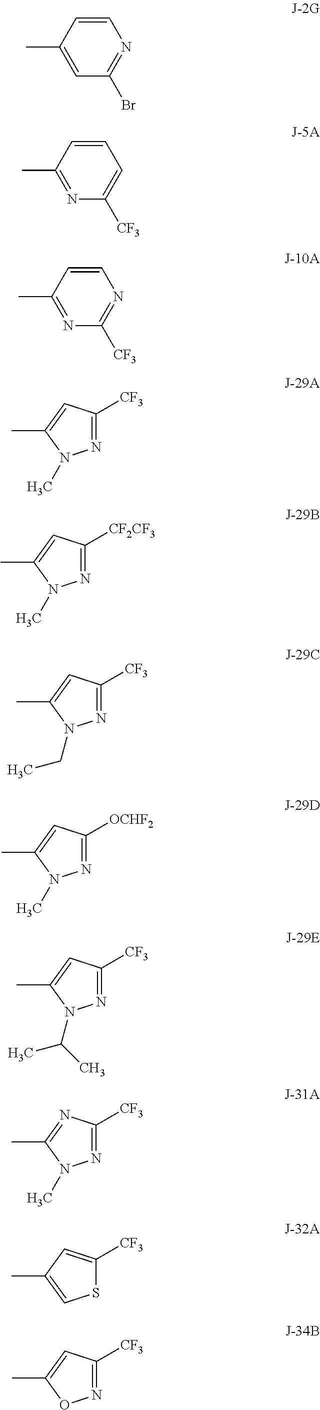

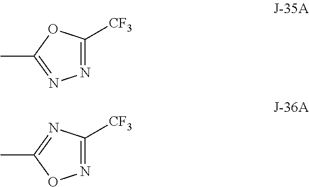

INDEX TABLE A
1

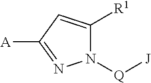

| Cmpd. | | | | | | |
|---|---|---|---|---|---|---|
| No. | A | R¹ | Q | J | m.p. (° C.) | M + 1 |
| 1 | C(═O)NH(t-Bu) | CH₃ | CH₂ | J-1A | * | |
| 2 | C(═O)NH(c-Pr) | CH₃ | CH₂ | J-1A | 121-124 | |
| 3 | C(═O)NH(CH₃) | CH₃ | CH₂ | J-1B | | 314 |
| 4 | C(═O)NH(CH₂CH₃) | CH₃ | CH₂ | J-1B | | 328 |
| 5 | C(═O)NH(CH₂CF₃) | CH₃ | CH₂ | J-1B | 62-64 | |
| 6 | C(═O)NH(CH₂CF₂CF₃) | CH₃ | CH₂ | J-1B | * | |
| 7 | C(═O)NH(CH₂CF₃) | CH₃ | CH₂ | J-1A | * | |
| 8 | C(═O)NH(CH₂CF₂CF₃) | CH₃ | CH₂ | J-1A | * | |
| 9 | C(═O)NH(CH₂CF₃) | CH₃ | CH₂ | J-1C | * | |
| 10 | C(═O)NH(c-Pr) | CH₃ | CH₂ | J-1C | * | 485 |
| 11 | C(═O)NH(c-Pr) | CH₃ | CH₂ | J-1D | * | |
| 12 | C(═O)NH(CH₂CF₃) | CH₃ | CH₂ | J-1D | * | |
| 13 | C(═O)NH(CH₂CF₃) | CH₃ | CH₂ | J-1E | * | |
| 14 | C(═O)NH(c-Pr) | CH₃ | CH₂ | J-1E | * | |
| 15 | C(═O)NH(c-Pr) | CH₂CH₃ | CH₂ | J-1A | | 338 |
| 16 | C(═O)NH(CH₂CH₂CH₃) | CH₂CH₃ | CH₂ | J-1A | | 340 |
| 17 | C(═O)NH(CH₂CH₃) | CH₂CH₃ | CH₂ | J-1A | | 326 |
| 18 | C(═O)NH(CH₂CF₃) | CH₂CH₃ | CH₂ | J-1A | | 380 |
| 19 | C(═O)NH(CH₂CH₃) | CH₂CH₃ | CH₂ | J-1B | | 342 |
| 20 | C(═O)NH(CH₂CF₃) | CH₂CH₃ | CH₂ | J-1B | | 396 |
| 21 | C(═O)NH(c-Pr) | CH₃ | CH₂ | J-1B | | 340 |
| 22 | C(═O)NH(c-Pr) | SCH₃ | CH₂ | J-1A | | 356 |
| 23 | C(═O)NH(CH₂CH₃) | SCH₃ | CH₂ | J-1A | | 344 |
| 24 | C(═O)NH(CH₂CF₃) | SCH₃ | CH₂ | J-1A | | 384 |
| 25 | C(═O)NH(CH₂CH₂CH₃) | SCH₃ | CH₂ | J-1A | | 358 |
| 26 | C(═O)NH(c-Pr) | CH₂CH₃ | CH₂ | J-1B | | 354 |
| 27 | C(═O)NH(CH₂CH₂CH₃) | CH₂CH₃ | CH₂ | J-1B | | 356 |
| 28 | C(═O)NH(CH₂CH₂CH₃) | CH₂CH₂CH₃ | CH₂ | J-1B | | 354 |
| 29 | C(═O)NH(CH₂CH₃) | CH₂CH₂CH₃ | CH₂ | J-1A | | 340 |
| 30 | C(═O)NH(c-Pr) | CH₂CH₂CH₃ | CH₂ | J-1A | | 352 |
| 31 | C(═O)NH(CH₂CF₃) | CH₂CH₂CH₃ | CH₂ | J-1A | | 394 |
| 32 | C(═O)NH(CH₂CF₃) | CH₂CH₂CH₃ | CH₂ | J-1B | | 410 |
| 33 | C(═O)NH(CH₂CH₃) | CH₂CH₂CH₃ | CH₂ | J-1B | 62-63 | |
| 34 | C(═O)NH(c-Pr) | CH₂CH₂CH₃ | CH₂ | J-1B | 69-71 | |
| 35 | C(═O)NH(CH₂CH₂CH₃) | CH₂CH₂CH₃ | CH₂ | J-1B | | 370 |
| 36 | C(═O)NH(CH₂CF₃) | c-Pr | CH₂ | J-1B | | 408 |
| 37 | C(═O)NH(c-Pr) | c-Pr | CH₂ | J-1B | | 366 |
| 38 | C(═O)NH(CH₂CH₂CH₃) | c-Pr | CH₂ | J-1B | | 368 |
| 39 | C(═O)NH(CH₂CH₃) | c-Pr | CH₂ | J-1B | | 354 |
| 40 | C(═O)NH(c-Pr) | CH₂CH₃ | CH₂ | J-1A | | 352 |
| 41 | C(═O)NH(CH₂C≡CH) | CH₂CH₃ | CH₂ | J-1A | | 336 |
| 42 | C(═O)NCH₃(CH₂CH₃) | CH₂CH₃ | CH₂ | J-1A | | 340 |
| 43 | C(═O)NH(CH₂CH₂CH₂CH₃) | CH₂CH₃ | CH₂ | J-1A | | 368 |
| 44 | C(═O)NH(CH₂CH═CH₂) | CH₂CH₃ | CH₂ | J-1A | | 338 |
| 45 | C(═O)NH(CH₃) | CH₂CH₃ | CH₂ | J-1A | | 312 |
| 46 | C(═O)NH(CH₂C(CH₃)₃) | CH₂CH₃ | CH₂ | J-1A | | 368 |
| 47 | C(═O)NH(CH(CH₃)₂) | CH₂CH₃ | CH₂ | J-1A | | 340 |
| 48 | C(═O)NH(CH₂CH₂OCH₃) | CH₂CH₃ | CH₂ | J-1A | | 356 |
| 49 | C(═O)NH(CH₂CH(CH₃)₂) | CH₂CH₃ | CH₂ | J-1A | | 354 |
| 50 | C(═O)NH(C(CH₃)₃) | CH₂CH₃ | CH₂ | J-1A | | 354 |
| 51 | C(═O)NH(CH₂CH₂CF₃) | CH₂CH₃ | CH₂ | J-1A | | 394 |
| 52 | C(═O)NH(CH₂CH₂CH₂CH₃) | CH₂CH₃ | CH₂ | J-1A | | 354 |
| 53 | C(═O)NH(CH₂-c-Pr) | CH₂CH₃ | CH₂ | J-1B | | 368 |
| 54 | C(═O)NH(CH₂C≡CH) | CH₂CH₃ | CH₂ | J-1B | | 352 |
| 55 | C(═O)NH(CH₂CH₂CF₃) | CH₂CH₃ | CH₂ | J-1B | | 410 |
| 56 | C(═O)NCH₃(CH₃) | CH₂CH₃ | CH₂ | J-1B | | 342 |
| 57 | C(═O)NH(CH₂CH₂OCH₃) | CH₂CH₃ | CH₂ | J-1B | | 372 |
| 58 | C(═O)NH(CH₂CH═CH₂) | CH₂CH₃ | CH₂ | J-1B | | 354 |
| 59 | C(═O)NH(C(CH₃)₃) | CH₂CH₃ | CH₂ | J-1B | | 370 |
| 60 | C(═O)NH(CH₂CH(CH₃)₂) | CH₂CH₃ | CH₂ | J-1B | | 370 |
| 61 | C(═O)NH(CH(CH₃)₂) | CH₂CH₃ | CH₂ | J-1B | | 384 |
| 62 | C(═O)NH(CH₂CH₂CH₂CH₃) | CH₂CH₃ | CH₂ | J-1B | | 370 |
| 63 | C(═O)NCH₃(CH₂CH₂CH₂CH₃) | CH₂CH₃ | CH₂ | J-1B | | 384 |
| 64 | C(═O)NH(CH₂C(CH₃)₃) | CH₂CH₃ | CH₂ | J-1B | | 356 |
| 65 | C(═O)NH(CH₃) | CH₂CH₃ | CH₂ | J-1B | | 328 |
| 66 | C(═O)NCH₃(OCH₃) | CH₂CH₃ | CH₂ | J-1A | | 342 |
| 67 | C(═O)NCH₃(OCH₃) | CH₂CH₃ | CH₂ | J-1B | | 358 |
| 68 | C(═O)NCH₃(CH₂CH₃) | CH₂CH₃ | CH₂ | J-1B | | 356 |
| 69 | C(═O)NH(OC(CH₃)₃) | CH₂CH₃ | CH₂ | J-1B | | 387 |
| 70 | C(═O)NH(OCH(CH₃)) | CH₂CH₃ | CH₂ | J-1B | | 372 |
| 71 | C(═O)NH(OC(CH₃)₂CH₂CH₃) | CH₂CH₃ | CH₂ | J-1B | * | |
| 72 | C(═O)NH(OCH₂CH₃) | CH₂CH₃ | CH₂ | J-1B | * | |
| 73 | C(═O)NH(OCH₂CH₃) | CH₃ | CH₂ | J-1B | * | |
| 74 | C(═O)NH(CH₂CF₃) | CH₃ | CH₂ | J-2A | | 367 |
| 75 | C(═O)NH(CH₂CH₂CH₃) | CH₂CH₃ | CH₂ | J-2A | * | |
| 76 | C(═O)NH(c-Pr) | CH₂CH₃ | CH₂ | J-2A | * | |
| 77 | C(═O)NH(OCH₂CH₃) | CH₂CH₃ | CH₂ | J-2A | * | |
| 78 | C(═O)NH(CH₂CF₃) | CH₂CH₃ | CH₂ | J-2A | ** | |
| (Ex. 2) | | | | | | |
| 79 | C(═O)NH(CH₂CF₃) | CH₃ | O | J-2A | 105-107 | |
| 80 | C(═O)NH(CH₂CH₂CH₃) | CH₃ | O | J-2A | | 329 |
| 81 | C(═O)NH(c-Pr) | CH₃ | O | J-2A | | 327 |
| 82 | C(═O)NH(CH₂CH₂CH₃) | CH₂CH₃ | O | J-2A | | 343 |
| 83 | C(═O)NH(c-Pr) | CH₂CH₃ | O | J-2A | ** | |
| (Ex. 1) | | | | | | |
| 84 | C(═O)NH(CH₂CF₃) | CH₂CH₃ | O | J-2A | 75-77 | |
| 85 | C(═O)NH(CH₂CH₂CH₂CH₃) | CH₃ | O | J-2A | | 343 |
| 86 | C(═O)NH(CH₂CH₂CH₂CH₃) | CH₂CH₃ | O | J-2A | | 357 |
| 87 | C(═O)NH(CH₂CF₃) | CH₂CH₃ | O | J-5A | * | |
| 88 | C(═O)NH(CH₂CH₂CH₃) | CH₂CH₃ | CH₂ | J-5A | * | |
| 89 | C(═O)NHPh(4-F) | CH₂CH₃ | O | J-2A | 105-107 | |
| 90 | C(═O)NHPh(4-F) | CH₃ | O | J-2A | 128-130 | |
| 91 | C(═O)NH(c-Pr) | CH₂CH₃ | CH₂ | J-5A | * | |
| 92 | C(═O)NH(CH₂CF₃) | CH₂CH₂CH₃ | O | J-2A | 55-57 | |
| 93 | C(═O)NH(c-Pr) | CH₂CH₂CH₃ | O | J-2A | | 355 |
| 94 | C(═O)NHPh(4-F) | CH₂CH₂CH₃ | O | J-2A | 110-112 | |
| 95 | C(═O)NH(CH₂CH₃) | CH₂CH₂CH₃ | CH₂ | J-2A | * | |
| 96 | C(═O)NH(CH₂CH₂CH₃) | CH₂CH₂CH₃ | CH₂ | J-2A | | 356 |
| 97 | C(═O)NH(CH₂CF₃) | CH₂CH₂CH₃ | CH₂ | J-2B | | 379 |
| 98 | C(═O)NH(c-Pr) | CH₂CH₃ | CH₂ | J-2B | * | |
| 99 | C(═O)NH(OCH₃) | CH₂CH₃ | CH₂ | J-2A | * | |
| 100 | C(═O)NH(CH₂CF₃) | OCH₃ | CH₂ | J-2A | | 384 |
| 101 | C(═O)NH(OCH(CH₃)₂) | CH₂CH₃ | CH₂ | J-2A | * | |
| 102 | C(═O)NH(CH₂CF₂CF₃) | CH₂CH₃ | CH₂ | J-2A | * | |
| 103 | C(═O)NH(CH(CH₃)₂) | CH₂CH₃ | CH₂ | J-2A | * | |
| 104 | C(═O)NH(CH₂CF₃) | OCH₂CH₃ | CH₂ | J-2A | * | |
| 105 | C(═O)NH(CH₂CF₃) | CH₂CH₂CH₃ | CH₂ | J-2A | | 395 |
| 106 | C(═O)NH(CH₂CF₃) | CH₂CH₃ | CH₂ | J-2E | * | |
| 107 | C(═O)NH(CH₂CF₃) | CH₂CH₃ | CH₂ | J-31A | * | |
| 108 | C(═O)NH(c-Pr) | CH₂CH₃ | CH₂ | J-31A | * | |
| 109 | C(═O)NH(CH₂CH₂CH₃) | CH₂CH₃ | CH₂ | J-31A | * | |
| 110 | Ph(4-CF₃) | CH₂CH₃ | CH₂ | J-2A | * | |
| 111 | Ph(4-F) | CH₂CH₃ | CH₂ | J-2A | * | |
| 112 | Ph(4-Br) | CH₂CH₃ | CH₂ | J-2A | * | |
| 113 | Ph(4-CF₃) | CH₂CH₂CH₃ | CH₂ | J-2A | * | |
| 114 | C(═O)NH(CH₂CF₃) | CH₂CH₃ | CH₂ | J-2F | | 426 |
| 115 | Ph(4-CF₃) | CH₂CH₃ | CH₂ | J-31A | | 404 |
| 116 | C(═O)NH(c-Pr) | CH₂CH₂CH₃ | CH₂ | J-2A | | 354 |
| 117 | C(═O)NH(n-Pr) | CH₂CH₃ | CH₂ | J-29C | | 358.0 |
| 118 | C(═O)NH(c-Pr) | CH₂CH₃ | CH₂ | J-29C | | 356 |
| 119 | C(═O)NH(CH₂CF₃) | CH₂CH₃ | CH₂ | J-29C | | 398.0 |
| 120 | C(═O)NH(i-Pr) | CH₂CH₃ | CH₂ | J-29C | | 358.0 |
| 121 | C(═O)NH(OCH₂CH₃) | CH₂CH₃ | CH₂ | J-29C | 97-100 | |
| 122 | C(═O)NH(n-Pr) | CH₂CH₃ | CH₂ | J-34B | 75-77 | |
| 123 | C(═O)NH(CH₂CF₃) | CH₂CH₃ | CH₂ | J-34B | 88-90 | |
| 124 | C(═O)NH(c-Pr) | CH₂CH₃ | CH₂ | J-34B | 87-89 | |
| 125 | C(═O)NH(OCH₂CH₃) | CH₂CH₃ | CH₂ | J-34B | | 333.1 |
| 126 | C(═O)NH(O-i-Pr) | CH₂CH₃ | CH₂ | J-2B | | 355 |
| 127 | C(═O)NH(CH₂CF₃) | CH₂CH₂CH₃ | CH₂ | J-2B | * | |
| 128 | C(═O)NH(CH₂CF₃) | OCH₂CH₃ | CH₂ | J-2B | * | |
| 129 | C(═O)NH(CH₂CH₃) | CH₂CH₂CH₃ | CH₂ | J-29A | | 344 |
| 130 | C(═O)NH(CH₂CH₃) | CH₂CH₃ | CH₂ | J-2B | | 325 |
| 131 | C(═O)NH(OCH₂CH₃) | CH₂CH₃ | CH₂ | J-2B | | 341 |
| 132 | C(═O)NH(CH₂CH₂CH₃) | CH₂CH₃ | CH₂ | J-2B | | 339 |
| 133 | C(═O)NH(c-Pr) | OCH₂CH₃ | CH₂ | J-2B | * | |
| 134 | C(═O)NH(n-Pr) | CH₂CH₃ | CH₂ | J-29D | | 342.1 |
| 135 | C(═O)NH(n-Bu) | CH₂CH₃ | CH₂ | J-29D | | 356.2 |
| 136 | C(═O)NH(n-Pr) | OCH₂CH₃ | CH₂ | J-2B | | 355 |
| 137 | C(═O)NH(i-Pr) | CH₂CH₃ | CH₂ | J-29D | | 342.1 |
| 138 | C(═O)NH(CH₂CF₃) | CH₂CH₂CH₃ | CH₂ | J-29A | * | |
| 139 | C(═O)NH(i-Pr) | CH₂CH₃ | CH₂ | J-2A | * | |
| 140 | C(═O)NH(CH₂CF₃) | OCH₂CH₃ | CH₂ | J-29A | * | |
| 141 | C(═O)NH(n-Bu) | CH₂CH₃ | CH₂ | J-2A | | 355 |
| 142 | C(═O)NH(n-Bu) | OCH₂CH₃ | CH₂ | J-2A | | 374 |
| 143 | C(═O)NH(CH₂CF₃) | CH₂CH₂Cl | CH₂ | J-2A | 107-110 | |
| 144 | C(═O)NH(c-Pr) | CH₂CH₂Cl | CH₂ | J-2A | 114-118 | |
| 145 | C(═O)NH(n-Pr) | CH₂CH₂Cl | CH₂ | J-2A | 72-75 | |
| 146 | C(═O)NH(CH₂CF₃) | CH₂CH₃ | CH₂ | J-35A | 106-109 | |
| 147 | C(═O)NH(CH₂CN) | CH₂CH₃ | CH₂ | J-2A | * | |
| 148 | C(═O)NH(CH₂CF₃) | n-Bu | CH₂ | J-2A | | 409 |
| 149 | C(═O)NH(i-Pr) | CH₂CH₃ | CH₂ | J-2E | | 371 |
| 150 | C(═O)NHPh(4-F) | CH₂CH₂CH₃ | CH₂ | J-29A | | 410 |
| 151 | C(═O)NH(c-Pr) | n-Bu | CH₂ | J-2A | * | |
| 152 | C(═O)NH(OCH₂CH₃) | n-Bu | CH₂ | J-2A | | 371 |
| 153 | C(═O)NH(OCH(CH₃)₂) | OCH₂CH₃ | CH₂ | J-2A | * | |
| 154 | C(═O)NH(CH₂CH₃) | CH₂CH₃ | CH₂ | J-29A | * | |
| 155 | C(═O)NH(CH₂CF₃) | CH₂CH₃ | CH₂ | J-2D | 119-122 | |
| 156 | C(═O)NH(c-Pr) | CF₃ | CH₂ | J-1A | 61-64 | |
| 157 | C(═O)NH(c-Pr) | CH₂CH₃ | CH₂ | J-2D | | 321 |
| 158 | C(═O)NH(n-Pr) | CH₂CH₃ | CH₂ | J-2D | | 323 |
| 159 | C(═O)NH(n-Pr) | Br | CH₂ | J-1A | | 389.7 |
| 160 | C(═O)NH(c-Pr) | Br | CH₂ | J-1A | 122-126 | |
| 161 | C(═O)NH(CH₂CF₃) | Br | CH₂ | J-1A | | 429.5 |
| 162 | C(═O)NH(n-Bu) | Br | CH₂ | J-1A | | 403.6 |
| 163 | C(═O)NH(c-Pr) | CH₂CH₃ | CH₂ | J-36A | 92-94 | |
| 164 | C(═O)NH(n-Pr) | CH₂CH₃ | CH₂ | J-36A | 76-79 | |
| 165 | Ph(4-CF₃) | CH₂CH₃ | C═O | J-2A | | 414 |
| 166 | C(═O)NH(i-Pr) | CH₂OCH₃ | CH₂ | J-2A | | 357 |
| 167 | C(═O)NH(c-Pr) | CH₂OCH₃ | CH₂ | J-2A | | 355 |
| 168 | C(═O)NH(n-Pr) | CH₂OCH₃ | CH₂ | J-2A | | 357.6 |
| 169 | C(═O)NH(OCH₂CH₃) | CH₂CH₃ | CH₂ | J-2D | | 325.1 |
| 170 | C(═O)NH(CH₂CF₃) | Cl | CH₂ | J-1A | 97-100 | |
| 171 | C(═O)NH(n-Bu) | Cl | CH₂ | J-1A | | 360.0 |
| 172 | C(═O)NH(c-Pr) | CH₂CH₃ | CH₂ | J-35A | | 329.8 |
| 173 | C(═O)NH(n-Bu) | CH₂CH₃ | CH₂ | J-35A | | 331.7 |
| 174 | C(═O)NH(c-Pr) | Cl | CH₂ | J-1A | 122-125 | |
| 175 | C(═O)NH(CH₂CF₃) | CH₂CH₂F | CH₂ | J-2A | 77-80 | |
| 176 | C(═O)NH(c-Pr) | CH₂CH₂F | CH₂ | J-2A | 81-84 | |
| 177 | C(═O)NH(n-Pr) | CH₂CH₂F | CH₂ | J-2A | | 359.4 |
| 178 | C(═O)NH(CH₂CH₃) | CH₂CH₂F | CH₂ | J-2A | 92-96 | |
| 179 | C(═O)NH(CH₂CH₃) | CH₂CH₂Cl | CH₂ | J-2A | 107-110 | |
| 180 | C(═O)NH(c-Pr) | I | CH₂ | J-1A | 100-103 | |
| 181 | C(═O)NH(CH₂CF₃) | I | CH₂ | J-1A | 82-85 | |
| 182 | C(═O)NH(n-Bu) | I | CH₂ | J-1A | | 452.3 |
| 183 | C(═O)NH(n-Pr) | I | CH₂ | J-1A | | 438.3 |
| 184 | C(═O)NH(CH₂CF₃) | CH₂CH₃ | CH₂ | J-36A | 70-73 | |
| 185 | C(═O)NH(OCH₂CH₃) | CH₂CH₂CH₃ | CH₂ | J-2A | | 357.6 |
| 186 | C(═O)NH(i-Pr) | CH₂CH₂CH₃ | CH₂ | J-2A | | 355 |
| 187 | C(═O)NH(n-Pr) | OCH₂CH₃ | CH₂ | J-2A | | 357.6 |
| 188 | C(═O)NH(i-Pr) | OCH₂CH₃ | CH₂ | J-2A | | 357.6 |
| 189 | C(═O)NH(c-Pr) | OCH₂CH₃ | CH₂ | J-2A | | 355.6 |
| 190 | C(═O)NH(n-Pr) | n-Bu | CH₂ | J-2A | * | |
| 191 | C(═O)NH(n-Pr) | CH₃ | CH₂ | J-2A | | 327 |
| 192 | C(═O)NHN(CH₃)₂ | CH₂CH₃ | CH₂ | J-2A | | 342.6 |
| 193 | C(═O)NH(CH₂CF₃) | CH₂CH₂Cl | CH₂ | J-2A | 97-100 | |
| 194 | C(═O)NH(c-Pr) | CH₂CH₂Cl | CH₂ | J-2A | 99-101 | |
| 195 | C(═O)NH(OCH₂CH₃) | OCH₂CH₃ | CH₂ | J-2A | | 359 |
| 196 | C(═O)NH(CH₂CH₃) | CH₂CH₃ | CH₂ | J-2A | | 327 |
| 197 | C(═O)NH(i-Pr) | CH₃ | CH₂ | J-2A | | 327 |
| 198 | C(═O)NH(n-Pr) | CH₂CH₂CH₃ | CH₂ | J-29A | * | |
| 199 | C(═O)NH(CH₂c-Pr) | CH₂CH₃ | CH₂ | J-2A | | 353 |
| 200 | C(═O)NH(c-Pr) | CH₂CH₃ | CH₂ | J-2E | | 369 |
| 201 | C(═O)NH(i-Pr) | CH₂CH₂CH₃ | CH₂ | J-29A | | 358 |
| 202 | C(═O)NH(c-Pr) | CH₂CH₂CH₃ | CH₂ | J-29A | * | |
| 203 | C(═O)NH(O-i-Pr) | CH₂CH₂CH₃ | CH₂ | J-29A | * | |
| 204 | C(═O)NH(CH₃) | CH₂CH₃ | CH₂ | J-2A | * | |
| 205 | C(═O)NH(c-Pr) | CH₂CH₂CH₃ | CH₂ | J-2B | | 351 |
| 206 | C(═O)NH(CH₂CF₃) | CH₂CH₃ | CH₂ | J-29D | | 382.2 |
| 207 | C(═O)NH(OCH₂CH₃) | CH₂CH₃ | CH₂ | J-29D | 73-75 | |
| 208 | C(═O)NH(OCH₂CH₃) | CH₂CH₃ | CH₂ | J-29E | 112-113 | |
| 209 | C(═O)NH(n-Pr) | CH₂CH₃ | CH₂ | J-29E | | 372.2 |
| 210 | C(═O)NH(i-Pr) | CH₂CH₃ | CH₂ | J-29E | 85-87 | |
| 211 | C(═O)NH(c-Pr) | CH₂CH₃ | CH₂ | J-29D | | 340.2 |
| 212 | C(═O)NH(c-Pr) | CH₂CH₃ | CH₂ | J-29E | 120-122 | |
| 213 | C(═O)NH(CH₂CF₃) | CH₂CH₃ | CH₂ | J-29E | 123-125 | |
| 214 | C(═O)NH(CH₂CF₃) | CH₂CH₃ | CH₂ | J-2C | * | |
| 215 | C(═O)NH(c-Pr) | CH₂CH₃ | O | J-1A | * | |
| 216 | C(═O)NH(n-Pr) | CH₂CH₃ | O | J-1A | * | |
| 217 | C(═O)NH(CH₂CF₃) | CH₂CH₃ | O | J-1A | * | |
| 218 | C(═O)NH(CH₂CF₃) | CH₂CH₃ | O | J-1A | * | |
| 219 | C(═O)NH(n-Pr) | CH₂CH₃ | O | J-1B | * | |
| 220 | C(═O)NH(c-Pr) | CH₂CH₃ | CH₂ | J-2G | * | |
| 221 | C(═O)NH(CH₂CF₃) | CH₂CH₃ | O | J-1B | * | |
| 222 | C(═O)NH(c-Pr) | CH₂CH₃ | O | J-1B | * | |
| 223 | C(═O)NH(CH₂CH₃) | CH₂CH₃ | O | J-1B | * | |
| 224 | C(═O)NH(CH₂CF₃) | CH₂CH₃ | CH₂ | J-2G | * | |
| 225 | C(═O)NH(CH₂CF₃) | CH₂OCH₃ | CH₂ | J-29B | 88-89 | |
| 226 | C(═O)NH(c-Pr) | CH₂CH₃ | CH₂ | J-29B | 92-95 | |
| 227 | C(═O)NH(CH₂CF₃) | CH₂CH₃ | CH₂ | J-29B | 77-79 | |
| 228 | C(═O)NH(n-Pr) | CH₂OCH₃ | CH₂ | J-29B | * | |
| 229 | C(═O)NH(n-Pr) | CH₂CH₃ | CH₂ | J-29B | * | |
| 230 | C(═O)NH(c-Pr) | CH₂OCH₃ | CH₂ | 0 | 95-96 | |
| 231 | C(═O)NH(OCH₂CH₃) | CH₃ | CH₂ | J-2A | * | |

* See Index Table B for ¹H NMR data.
** See Synthesis Example for ¹H NMR data.

INDEX TABLE B

| Cmpd. | |
|---|---|
| No. | ¹H NMR Data^{a} |
| 1 | 7.56 (m, 1 H), 7.45 (m, 1 H), 7.38 (s, 1 H), 7.17 (m, 1 H), 6.70 (br, 1 H), 6.58 (s, 1 H), 5.31 (s, 2 H), |
| | 2.19 (s, 3 H), 1.46 (s, 9 H). |
| 6 | 7.33-7.45 (m, 1 H), 7.12-7.20 (m, 1 H), 7.09 (br s, 1 H), 6.96-7.04 (m, 1 H), 6.95 (s, 1 H), 6.65 (s, 1 H), |
| | 5.30 (s, 2 H), 2.23 (s, 3 H). |
| 7 | 7.59-7.38 (m, 3 H) 7.24-7.12 (m, 2 H), 6.66 (s, 1 H), 5.34 (s, 2 H), 4.08 (m, 2 H), 2.24 (s, 3 H). |
| 8 | 7.58-7.39 (m, 3 H) 7.24-7.10 (m, 2 H), 6.66 (s, 1 H), 5.34 (s, 2 H), 4.15 (m, 2 H), 2.24 (s, 3 H). |
| 9 | 7.61 (m, 1 H), 7.26 (m, 2 H), 7.09 (br s, 1 H), 6.66 (s, 1 H), 5.34 (s, 2 H), 4.07 (m, 2 H), 2.23 (s, 3 H). |
| 10 | 7.61 (m, 1 H), 7.26 (m, 2 H), 6.84 (br s, 1 H), 6.63 (s, 1 H), 5.31 (s, 2 H), 2.84 (m, 1 H), 2.27 (s, 3 H) |
| | 0.83 (m, 2 H), 0.62 (m, 2 H). |
| 11 | 7.61 (m, 1 H), 7.22 (m, 2 H), 6.86 (br s, 1 H), 6.63 (s, 1 H), 5.25 (s, 2 H), 2.86 (m, 1 H), 2.26 (s, 3 H) |
| | 0.83 (m, 2 H), 0.64 (m, 2 H). |
| 12 | 7.61 (m, 1 H), 7.24 (m, 2 H), 7.11 (br s, 1 H), 6.66 (s, 1 H), 5.29 (s, 2 H), 4.09 (m, 2 H), 2.25 (S, 3 H). |
| 13 | 7.29-7.12 (m, 3 H), 6.92 (br s, 1 H), 6.67 (s, 1 H), 5.33 (s, 2 H), 4-07 (m, 2 H), 2.25 (s, 3 H). |
| 14 | 7.26 (m, 1 H) 7.18 (m, 1 H), 6.89 (m, 2 H), 6.64 (s, 1 H), 5.30 (s, 2 H), 2.84 (m, 1 H), 2.23 (s, 3 H), 0.83 |
| | (m, 2 H), 0.63 (m, 2 H). |
| 71 | 8.81 (s, 1 H), 7.34-7.42 (m, 1 H), 7.11-7.21 (m, 1 H), 6.97-7.00 (m, 1 H), 6.95 (s, 1 H), 6.71 (s, 1 H), |
| | 5.31 (s, 2 H), 2.51-2.57 (m, 2 H), 1.67-1.75 (m, 2 H), 1.32 (s, 6 H), 1.20-1.26 (m, 3 H), 0.96-1.02 (m, |
| | 3 H). |
| 72 | 9.18 (s, 1 H), 7.34-7.38 (m, 1 H), 7.14-7.17 (m, 1 H), 6.94-6.97 (m, 1 H), 6.92 (s, 1 H), 6.70 (s, 1 H), |
| | 5.28 (s, 2 H), 4.06-4.12 (m, 2 H), 2.49-2.55 (m, 2 H), 1.32-1.36 (m, 3 H), 1.19-1.24 (m, 3 H), 0.00 (s, |
| | 2 H). |
| 73 | 9.75 (s, 1 H), 7.36-7.41 (m, 1 H), 7.16-7.20 (m, 1 H), 7.02-7.05 (m, 1 H), 6.95 (s, 1 H), 6.80 (s, 1 H), |
| | 5.37 (s, 2 H), 4.13-4.19 (m, 2 H), 2.27 (s, 3 H), 1.34-1.38 (m, 3 H). |
| 75 | 8.68 (d, 1 H), 7.40 (s, 1 H), 7.06 (d, 1 H), 6.83 (br s, 1 H), 6.71 (s, 1 H), 5.37 (s, 2 H), 3.34-3.44 (m, |
| | 2 H), 2.48-2.58 (m, 2 H), 1.55-1.70 (m, 2 H), 1.18-1.34 (m, 3 H), 0.83-1.03 (m, 3 H). |
| 76 | 8.68 (d, 1 H), 7.37 (s, 1 H), 7.03 (d, 1 H), 6.86 (br s, 1 H), 6.72 (s, 1 H), 5.34 (s, 2 H), 2.86 (m, 1 H), |
| | 2.51 (m, 2 H), 1.21-1.27 (m, 3 H), 0.78-0.87 (m, 2 H), 0.54-0.65 (m, 2 H). |
| 77 | 9.21 (br s, 1 H), 8.65-8.75 (m, 1 H), 7.35-7.44 (m, 1 H), 7.01-7.07 (m, 1 H), 6.75 (s, 1 H), 5.47 (s, 2 H), |
| | 4.04-4.16 (m, 2 H), 2.49-2.57 (m, 2 H), 1.31-1.37 (m, 3 H), 1.25-1.29 (m, 3 H). |
| 87 | 7.78-7.91 (m, 1 H), 7.56-7.70, (m, 1 H), 7.10 (br s, 1 H), 6.91-7.04 (m, 1 H), 6.72 (s, 1 H), 5.48 (s, |
| | 2 H), 3.95-4.14 (m, 2 H), 2.53-2.66 (m, 2 H), 1.17-1.30 (m, 3 H). |
| 88 | 7.79-7.84 (m, 1 H), 7.59-7.62 (m, 1 H), 6.90-6.93 (m, 1 H), 6.84 (br s, 1 H), 6.69 (s, 1 H), 5.47 (s, 2 H), |
| | 3.34-3.43 (m, 2 H), 2.54-2.61 (m, 2 H), 1.62 (s, 2 H), 1.20-1.24 (m, 3 H), 0.96-1.00 (m, 3 H). |
| 91 | 7.71-7.94 (m, 1 H), 7.58-7.67 (m, 1 H), 6.87-6.93 (m, 1 H), 6.69 (s, 1 H), 5.45 (s, 2 H), 2.80-2.94 (m, |
| | 1 H), 2.50-2.70 (m, 2 H), 1.16-1.29 (m, 3 H), 0.76-0.98 (m, 2 H), 0.51-0.72 (m, 2 H). |
| 95 | 8.66-8.73 (m, 1 H), 7.38 (s, 1 H), 7.01-7.07 (m, 1 H), 6.77 (br s, 1 H), 6.70 (s, 1 H), 5.36 (s, 2 H), 3.43- |
| | 3.49 (m, 2 H), 2.42-2.52 (m, 2 H), 1.61-1.66 (m, 2 H), 1.22-1.25 (m, 3 H), 0.94-0.98 (m, 3 H). |
| 98 | 8.12-8.15 (m, 1 H), 7.28-7.64 (m, 1 H), 6.86 (br s, 1 H), 6.71-6.76 (m, 1 H), 6.69 (s, 1 H), 6.43-6.56 |
| | (m, 1 H), 5.25 (s, 2 H), 2.82-2.92 (m, 1 H), 2.47-2.53 (m, 2 H), 1.22-1.26 (m, 3 H), 0.78-0.89 (m, 2 H), |
| | 0.59-0.67 (m, 2 H). |
| 99 | 9.23 (s, 1 H), 8.65-8.73 (m, 1 H), 7.36-7.42 (m, 1 H), 6.97-7.14 (m, 2 H), 6.73-6.77 (m, 1 H), 5.35 (s, |
| | 2 H), 3.89 (s, 3 H), 2.49-2.54 (m, 2 H), 1.25-1.27 (m, 3 H). |
| 101 | 8.67-8.72 (m, 1 H), 7.38-7.42 (m, 1 H), 7.04-7.13 (m, 1 H), 6.70-6.80 (m, 1 H), 5.34-5.53 (m, 2 H), |
| | 4.22-4.35 (m, 1 H), 2.49-2.56 (m, 2 H), 1.29-1.33 (m, 6 H), 1.24-1.28 (m, 3 H). |
| 102 | 8.67-8.73 (m, 1 H), 7.40 (s, 1 H), 6.98-7.14 (m, 2 H), 6.75 (s, 1 H), 5.38 (s, 2 H), 4.05-4.21 (m, 2 H), |
| | 2.50-2.57 (m, 2 H), 1.23-1.30 (m, 3 H). |
| 103 | 8.67-8.71 (m, 1 H), 7.40 (s, 1 H), 7.02-7.07 (m, 1 H), 6.68-6.73 (m, 1 H), 6.57-6.67 (m, 1 H), 5.36 (s, |
| | 2 H), 4.20-4.31 (m, 1 H), 2.47-2.54 (m, 2 H), 1.23-1.27 (m, 9 H). |
| 104 | 8.68-8.73 (m, 1 H), 7.50 (s, 1 H), 7.21-7.24 (m, 1 H), 6.97-7.15 (m, 1 H), 6.16 (s, 1 H), 5.24 (s, 2 H), |
| | 4.15-4.20 (m, 2 H), 4.01-4.08 (m, 2 H), 1.37-1.43 (m, 3 H). |
| 106 | 8.05-8.14 (m, 1 H), 7.09 (br s, 1 H), 6.72 (s, 1 H), 6.60-6.68 (m, 1 H), 6.41-6.54 (m, 1 H), 5.26 (s, 2 H), |
| | 4.70-4.80 (m, 2 H), 4.03-4.13 (m, 2 H), 2.47-2.57 (m, 2 H), 1.23-1.26 (m, 3 H). |
| 107 | 7.00 (br s, 1 H), 6.68-6.70 (m, 1 H), 6.35 (s, 1 H), 5.30 (s, 2 H), 4.00-4.09 (m, 2 H), 3.93 (s, 3 H), 2.58- |
| | 2.64 (m, 2 H), 1.25-1.31 (m, 3 H). |
| 108 | 6.79 (br s, 1 H), 6.64-6.68 (m, 1 H), 6.31 (s, 1 H), 5.27 (s, 2 H), 3.91 (s, 3 H), 2.80-2.87 (m, 1 H), 2.52- |
| | 2.63 (m, 2 H), 1.25-1.31 (m, 3 H), 0.77-0.91 (m, 2 H), 0.56-0.67 (m, 2 H). |
| 109 | 6.74 (br s, 1 H), 6.65 (s, 1 H), 6.33 (s, 1 H), 5.28 (s, 2 H), 3.92 (s, 3 H), 3.30-3.45 (m, 2 H), 2.53-2.64 |
| | (m, 2 H), 1.62 (d, 2 H), 1.26-1.29 (m, 3 H), 0.95-1.01 (m, 3 H). |
| 110 | 8.66-8.71 (m, 1 H), 7.89-7.97 (m, 2 H), 7.63-7.66 (m, 2 H), 7.44 (s, 1 H), 7.12-7.19 (m, 1 H), 6.52 (s, |
| | 1 H), 5.42 (s, 2 H), 2.56 (qd, 2 H), 1.29-1.33 (m, 3 H). |
| 111 | 8.66-8.71 (m, 1 H), 7.89-7.97 (m, 2 H), 7.63-7.66 (m, 2 H), 7.44 (s, 1 H), 7.12-7.19 (m, 1 H), 6.52 (s, |
| | 1 H), 5.42 (s, 2 H), 2.56 (qd, 2 H), 1.29-1.33 (m, 3 H). |
| 112 | 8.66-8.68 (m, 1 H), 7.65-7.69 (m, 2 H), 7.50-7.54 (m, 2 H), 7.43 (s, 1 H), 7.11-7.14 (m, 1 H), 6.45 (s, |
| | 1 H), 5.40 (s, 2 H), 2.51-2.57 (m, 2 H), 1.28-1.32 (m, 3 H). |
| 113 | 8.64-8.69 (m, 1 H), 7.87-7.94 (m, 2 H), 7.63-7.68 (m, 2 H), 7.43 (s, 1 H), 7.08-7.15 (m, 1 H), 6.52 (s, |
| | 1 H), 5.43 (s, 2 H), 2.49-2.54 (m, 2 H), 1.64-1.72 (m, 2 H), 0.94-1.03 (m, 3 H). |
| 127 | (500 MHz) 8.15 (m, 1 H), 7.45 (m, 1 H), 6.74 (m, 1 H), 6.70 (m, 1 H), 6.51 (s, 1 H), 5.29 (s, 2 H), 4.10 |
| | (m, 2 H), 2.45 (m, 2 H), 1.60 (m, 2 H), 0.95 (m, 4 H). |
| 128 | (500 MHz) 8.16 (m, 1 H), 7.52 (m, 1 H), 6.88 (m, 1 H), 6.63 (m, 1 H), 6.14 (s, 1 H), 5.15 (s, 2 H), 4.16 |
| | (m, 2 H), 4.05 (m, 2 H), 1.40 (m, 3 H). |
| 133 | (500 MHz) 8.13 (m, 1 H), 7.55 (m, 1 H), 6.85 (m, 2 H), 6.61 (m, 1 H), 6.11 (s, 1 H), 5.12 (s, 2 H), 4.15 |
| | (m, 2 H), 2.84 (m, 1 H), 1.38 (m, 3 H), 0.82 (m, 2 H), 0.60 (m, 2 H). |
| 138 | (500 MHz) 7.00 (br s, 1 H), 6.67 (s, 1 H), 6.33 (s, 1 H), 5.31 (s, 2 H), 4.02-4.09 (m, 2 H), 3.93 (s, 3 H), |
| | 2.56 (m, 2 H), 1.62 (m, 3 H), 0.98 (m, 3 H). |
| 139 | (500 MHz) 8.69 (m, 1 H), 7.40 (m, 1 H), 7.05 (m, 1 H), 6.86 (br s, 1 H), 6.71 (m, 1 H), 5.37 (s, 2 H), |
| | 3.25 (m, 2 H), 2.51 (m, 2 H), 1.88 (m, 1 H), 1.25 (m, 3 H), 0.97 (m, 6 H). |
| 140 | (500 MHz) 6.48 (s, 1 H), 6.10 (s, 1 H), 5.18 (s, 2 H), 4.18 (m, 2 H), 4.05 (m, 2 H), 3.97 (s, 3 H), 1.44 |
| | (m, 3 H). |
| 147 | (500MHz) 8.71 (m, 1 H), 7.40 (m, 1 H), 7.10 (br s, 1 H), 7.06 (m, 1 H), 6.75 (s, 1 H), 5.37 (s, 2 H), 4.36 |
| | (m, 2 H), 2.54 (m, 2 H), 1.27 (m, 3 H). |
| 151 | (500 MHz) 8.68 (m, 1 H), 7.36 (s, 1 H), 7.03 (m, 1 H), 6.85 (m, 1 H), 6.70 (s, 1 H), 5.34 (s, 2 H), 2.86 |
| | (m, 1 H), 2.48 (m, 2 H), 1.59 (m, 2 H), 1.35 (m, 2 H), 0.85 (m, 5 H), 0.61 (m, 2 H). |
| 153 | (500 MHz) 6.48 (s, 1 H), 6.10 (s, 1 H), 5.18 (s, 2 H), 4.18 (m, 2 H), 4.05 (m, 2 H), 3.97 (s, 3 H), 1.44 |
| | (m, 3 H). |
| 154 | (500 MHz) 6.65 (m, 1 H), 6.33 (s, 1 H), 5.28 (s, 2 H), 3.92 (s, 3 H), 3.45 (m, 2 H), 2.59 (m, 2 H), 1.25 |
| | (m, 6 H). |
| 190 | (500 MHz) 8.69 (m, 1 H), 7.38 (s, 1 H), 7.05 (m, 1 H), 6.80 (m, 1 H), 6.70 (s, 1 H), 5.36 (s, 2 H), 3.40 |
| | (m, 2 H), 2.49 (m, 2 H), 1.61 (m, 4 H), 1.30 (m, 2 H), 0.97 (m, 3 H), 0.88 (m, 3 H). |
| 198 | (500 MHz) 6.74 (br s, 1 H), 6.65 (s, 1 H), 6.33 (s, 1 H), 5.28 (s, 2 H), 3.92 (s, 3 H), 3.40 (m, 2 H), 2.59 |
| | (m, 2 H), 1.63 (m, 2 H), 1.28 (m, 3 H), 0.97 (m, 3 H). |
| 202 | (500 MHz) 6.78 (br s, 1 H), 6.64 (m, 1 H), 6.23-6.38 (m, 1 H), 5.27 (s, 2 H), 3.90 (s, 2 H), 2.84 (m, |
| | 1 H), 2.47-2.59 (m, 2 H), 1.66 (m, 2 H), 0.98 (m, 3 H), 0.85 (m, 2 H), 0.60 (m, 2 H). |
| 203 | (500MHz) 9.15 (br s, 1 H), 8.69 (m, 1 H), 7.37 (s, 1 H), 7.05 (m, 1 H), 6.73 (s, 1 H), 5.36 (s, 2 H), 4.25 |
| | (m, 1 H), 2.47 (m, 2 H), 1.69 ? 1.60 (m, 2 H), 1.30 (m, 6 H), 0.95 (m, 3 H). |
| 204 | (500 MHz) 8.69 (m, 2 H), 7.38 (m, 2 H), 7.05 (m, 2 H), 6.71 (m, 1 H), 5.35 (m, 3 H), 2.98 (s, 3 H), 2.52 |
| | (m, 3 H), 1.30 (m, 5 H). |
| 214 | (400 MHz) 8.57 (d, 1 H, J = 5.1 Hz), 7.21 (s, 1 H), 7.10-7.02 (br s, 1 H), 6.92-6.90 (m, 1 H), 6.74 (s, |
| | 1 H), 5.31 (s, 2 H), 4.13-4.00 (m, 2 H), 2.52 (q, 2 H, J = 7.5 Hz), 1.25 (t, 3 H, J = 7.5 Hz). |
| 215 | (300 MHz) 7.49-7.41 (m, 2 H), 7.06 (s, 1 H), 6.83 (d, 1 H, J = 8.4 Hz ), 6.74 (s, 1 H, br), 6.73 (s, 1 H), |
| | 2.85-2.81 (m, 2 H), 2.59 (q, 2 H), 1.24 (t, 3 H, J = 7.3), 0.85-0.78 (m, 2 H), 062-0.56 (m, 2 H). |
| 216 | (300 MHz) 7.50-7.41 (m, 2 H), 7.07 (s, 1 H), 6.85-6.83 (d, 1 H, J = 7.3 Hz), 6.72 (s, 1 H), 6.69 (s, |
| | 1 H), 3.37 (q, 2 H), 2.59 (q, 2 H), 1.63-1.56 (m, 2 H), 1.24 (t, 3 H, J = 7.3), 0.95 (t, 3 H, J = 7.1). |
| 217 | (300 MHz) 7.52-7.43 (m, 2 H), 7.09 (s, 1 H), 6.98-6.91 (m, 1 H), 6.88-6.83 (m, 1 H), 6.76 (s, 1 H), |
| | 4.10-3.99 (m, 2 H), 2.61 (q, 2 H), 1.25 (t, 3 H, J = 7.2). |
| 218 | (300 MHz) 7.51-7.38 (m, 1 H), 7.08 (S, 1 H), 6.88-6.80 (m, 1 H), 6.72 (s, 1 H), 6.66 (S, 1 H), 3.51- |
| | 3.38 (m, 2 H), 2.66-2.54 (m, 2 H), 1.26-1.16 (m, 6 H). |
| 219 | (300 MHz) 7.36 (t, 1 H, J = 8.2 Hz), 7.06-6.97 (m, 1 H), 6.71 (s, 1 H), 6.69-6.57 (m, 2 H), 3.41-3.29 |
| | (m, 2 H), 2.67-2.52 (m, 2 H), 1.69-1.56 (m, 2 H), 1.26-1.19 (m, 3 H), 1.00-0.88 (m, 3 H). |
| | (400 MHz) 8.32 (d, 1 H, J = 5.1 Hz), 7.12 (s, 1 H), 6.87-6.83 (m, 2 H), 6.70 (s, 1 H), 5.23 (s, 2 H), |
| 220 | 2.90-2.84 (m, 1 H), 2.49 (q, 2 H, J = 7.5 Hz), 1.24 (t, 3 H, J = 7.5 Hz), 0.86-0.81 (m, 2 H), 0.65-0.61 |
| | (m, 2 H). |
| 221 | (300 MHz) 7.41-7.33 (m, 1 H), 7.08-7.00 (m, 1 H), 6.99-6.90 (m, 1 H), 6.75 (s, 1 H), 6.69-6.66 (m, |
| | 1 H), 6.66-6.60 (m, 1 H), 4.14-3.96 (m, 2 H), 2.69-2.54 (m, 2 H), 1.29-1.18 (m, 2 H). |
| | (300 MHz) 7.35 (t, 1 H, J = 8.2 Hz), 7.06-6.98 (m, 1 H), 6.75 (br s, 1 H), 6.72 (s, 1 H), 6.65 (br s, 1 H), |
| 222 | 6.66-6.58 (m, 1 H), 2.93-2.81 (m, 1 H), 2.65-2.51 (m, 2 H), 1.33-1.20 (m, 4 H), 0.88-0.76(m, 2 H), |
| | 0.65-0.54 (m, 2 H). |
| 223 | (300 MHz) 7.36 (t, 1 H, J = 8.2 Hz), 7.04-6.99 (m, 1 H), 6.71 (s, 1 H), 6.70-6.58 (m, 3 H), 3.52-3.36 |
| | (m, 2 H), 2.67 2.55(m, 2 H), 1.29-1.16 (m, 6 H). |
| 224 | (400 MHz) 8.34 (d, 1 H, J = 5.1 Hz), 7.16 (s, 1 H), 7.15-7.07 (br s, 1 H), 6.89-6.86 (m, 1 H), 6.73 (s, |
| | 1 H), 5.26 (s, 2 H), 4.11-4.03 (m, 2 H), 2.52 (q, 2 H, J = 7.5 Hz), 1.26 (t, 3 H, J = 7.5 Hz). |
| 228 | (400 MHz) 6.78 (s, 1 H), 6.75 (br s, 1 H), 6.45 (s, 1 H), 5.42 (s, 2 H), 4.42 (s, 2 H), 3.95 (s, 3 H), 3.38 |
| | (q, 2 H, J = 7.2 Hz), 3.29 (s, 3 H), 1.67-1.59 (m, 2 H), 0.97 (t, 3 H, J = 7.2 Hz). |
| 229 | (300 MHz) 6.73 (br, 1 H), 6.64 (s, 1 H), 6.36 (s, 1 H), 6.72 (s, 1 H), 5.28 (s, 1 H), 3.93 (s, 3 H), 3.37 (q, |
| | 2 H), 2.60-2.55 (m, 2 H), 1.64-1.59 (m, 2 H), 1.25 (t, 3 H, J = 7.4), 0.96(t, 3 H, J = 7.4). |
| 231 | 8.70 (s, 1 H), 7.41 (s, 1 H), 7.07 (s, 1 H), 6.71 (s, 1 H), 5.47 (s, 2 H), 4.02-4.17 (m, 2 H), 2.25 (s, 3 H), |
| | 1.29-1.40 (m, 3 H). |

^{a1}H NMR data are in ppm downfield from tetramethylsilane, in CDCl₃ unless otherwise indicated.
Couplings are designated by (s)-singlet, (d)-doublet, (t)-triplet, (m)-multiplet, (q)-quartet, (dd)-doublet of doublets, (dq)-doublet of quartets, (qd)-quartet of doublets and (br s)-broad singlet.

### Biological Examples of the Invention

### Test A

Seeds of plant species selected from barnyardgrass (Echinochloa crus-galli), kochia (Kochia scoparia), common ragweed (Ambrosia elation), Italian ryegrass (Lolium multiflorum), crabgrass, large (large crabgrass, Digitaria sanguinalis), foxtail, giant (giant foxtail, Setaria faberii), morningglory (Ipomoea spp.), pigweed (redroot pigweed, Amaranthus retroflexus), velvetleaf (Abutilon theophrasti), wheat (Triticum aestivum), and corn (Zea mays) were planted into a blend of loam soil and sand and treated preemergence with a directed soil spray using test chemicals formulated in a non-phytotoxic solvent mixture which included a surfactant.

At the same time, plants selected from these crop and weed species and also blackgrass (Alopecurus myosuroides), and galium (catchweed bedstraw, Galium aparine) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for approximately 10 days, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

Postemergence

1000 g ai/ha
Compounds

| | 1 | 2 |
|---|---|---|
| Barnyardgrass | 30 | 50 |
| Crabgrass, Large | 30 | 50 |
| Morningglory | 20 | 80 |
| Velvetleaf | 30 | 70 |

1000 g ai/ha
Compounds

| | 15 | 16 | 17 | 18 | 19 | 20 | 22 | 23 | 24 | 25 | 26 | 27 | 28 | 29 | 30 | 31 | 32 | 33 | 34 | 35 | 36 | 37 | 38 | 39 | 40 | 41 | 42 | 43 | 44 |
|---|---|---|---|---|---|---|---|---|---|---|---|---|---|---|---|---|---|---|---|---|---|---|---|---|---|---|---|---|---|
| Barnyardgrass | 80 | 60 | 40 | 50 | 60 | 60 | 30 | 10 | 20 | 10 | 70 | 60 | 50 | 80 | 70 | 60 | 40 | 30 | 50 | 30 | 20 | 10 | 10 | 10 | 30 | 10 | 20 | 10 | 10 |
| Corn | 70 | 50 | 40 | 70 | 20 | 30 | 10 | 20 | 10 | 40 | 70 | 60 | 20 | 50 | 40 | 20 | 30 | 20 | 40 | 20 | 20 | 30 | 30 | 10 | 30 | 20 | 30 | 20 | 20 |
| Crabgrass, Large | 90 | 80 | 90 | 90 | 80 | 80 | 50 | 30 | 40 | 30 | 90 | 80 | 80 | 70 | 90 | 60 | 70 | 80 | 90 | 70 | 30 | 20 | 10 | 10 | 50 | 20 | 50 | 10 | 20 |
| Foxtail, Giant | 90 | 80 | 90 | 90 | 70 | 70 | 40 | 30 | 40 | 20 | 100 | 70 | 70 | 90 | 100 | 70 | 50 | 70 | 90 | 40 | 30 | 10 | 10 | 10 | 40 | 20 | 30 | 60 | 50 |
| Morningglory | 100 | 100 | 100 | 100 | 90 | 100 | 100 | 100 | 100 | 100 | 100 | 100 | 70 | 90 | 100 | 100 | 100 | 100 | 100 | 100 | 90 | 80 | 100 | 60 | 100 | 100 | 60 | 40 | 70 |
| Pigweed | 80 | 90 | 90 | 90 | 100 | 100 | 60 | 40 | 50 | 10 | 100 | 70 | 80 | 100 | 100 | 100 | 100 | 80 | 100 | 90 | 90 | 100 | 40 | 40 | 90 | 30 | 60 | 50 | 50 |
| Velvetleaf | 100 | 100 | 100 | 100 | 100 | 90 | 80 | 30 | 20 | 50 | 100 | 100 | 100 | 100 | 100 | 100 | 80 | 80 | 100 | 100 | 60 | 70 | 50 | 10 | 90 | 10 | 40 | 10 | 40 |
| Wheat | 50 | 50 | 30 | 40 | 20 | 40 | 20 | 0 | 0 | 0 | 50 | 40 | 20 | 40 | 40 | 30 | 20 | 20 | 30 | 20 | 0 | 20 | 0 | 0 | 20 | 10 | 0 | 10 | 20 |

| | 1000 g ai/ha | | | | | | | | | | | | | | | | | | | | | | | | |
|---|---|---|---|---|---|---|---|---|---|---|---|---|---|---|---|---|---|---|---|---|---|---|---|---|---|
| | Compounds | | | | | | | | | | | | | | | | | | | | | | | | |
| | 45 | 46 | 47 | 48 | 49 | 50 | 51 | 52 | 53 | 54 | 55 | 56 | 57 | 58 | 59 | 60 | 61 | 62 | 63 | 64 | 65 | 66 | 67 | 68 | 74 |
| Barnyardgrass | 10 | 10 | 60 | 10 | 20 | 10 | 10 | 20 | 40 | 10 | 10 | 0 | 10 | 20 | 10 | 20 | 10 | 10 | 0 | 50 | 50 | 20 | 20 | 30 | 90 |
| Corn | 20 | 20 | 40 | 30 | 30 | 20 | 10 | 30 | 40 | 20 | 20 | 10 | 40 | 20 | 10 | 30 | 30 | 50 | 20 | 50 | 30 | 30 | 30 | 30 | 100 |
| Crabgrass, Large | 40 | 30 | 70 | 10 | 30 | 10 | 30 | 30 | 50 | 10 | 50 | 10 | 10 | 30 | 10 | 30 | 20 | 20 | 10 | 60 | 50 | 20 | 30 | 30 | 100 |
| Foxtail, Giant | 70 | 60 | 80 | 20 | 100 | 30 | 10 | 30 | 50 | 10 | 50 | 20 | 10 | 20 | 20 | 40 | 30 | 30 | 20 | 70 | 50 | 40 | 40 | 20 | 90 |
| Morningglory | 90 | 70 | 100 | 50 | 100 | 10 | 100 | 40 | 90 | 70 | 100 | 70 | 60 | 50 | 50 | 100 | 70 | 80 | 60 | 80 | 90 | 50 | 40 | 100 | 100 |
| Pigweed | 60 | 40 | 70 | 10 | 50 | 30 | 50 | 50 | 80 | 70 | 70 | 60 | 50 | 60 | 50 | 60 | 40 | 70 | 70 | 80 | 70 | 50 | 40 | 60 | 100 |
| Velvetleaf | 30 | 70 | 60 | 10 | 50 | 10 | 100 | 40 | 80 | 60 | 90 | 10 | 30 | 50 | 10 | 60 | 40 | 70 | 30 | 80 | 30 | 40 | 30 | 40 | 100 |
| Wheat | 10 | 20 | 40 | 0 | 30 | 0 | 20 | 30 | 30 | 10 | 10 | 0 | 0 | 0 | 0 | 20 | 20 | 20 | 10 | 50 | 20 | 10 | 20 | 20 | 90 |

500 g ai/ha
Compounds

| | 3 | 4 | 5 | 6 | 7 | 8 | 9 | 10 | 11 | 12 | 13 | 14 | 21 | 69 | 70 | 71 | 72 | 73 | 75 | 76 | 77 | 78 | 79 | 80 | 81 | 82 | 83 | 84 | 85 | 86 | 87 | 88 | 89 |
|---|---|---|---|---|---|---|---|---|---|---|---|---|---|---|---|---|---|---|---|---|---|---|---|---|---|---|---|---|---|---|---|---|---|
| Barnyardgrass | 0 | 40 | 40 | 10 | 30 | 10 | 10 | 0 | 10 | 20 | 20 | 10 | 60 | 90 | 90 | 70 | 90 | 70 | 90 | 90 | 100 | 90 | 30 | 40 | 20 | 50 | 40 | 30 | 10 | 20 | 0 | 0 | 0 |
| Blackgrass | — | — | — | — | — | — | — | — | — | — | — | — | — | — | — | — | — | — | — | — | — | — | — | — | — | — | — | — | — | — | — | — | — |
| Common Ragweed | — | — | — | — | — | — | — | — | — | — | — | — | — | — | — | — | — | — | — | — | — | — | — | — | — | — | — | — | — | — | — | — | — |
| Corn | 10 | 40 | 40 | 0 | 30 | 0 | 0 | 0 | 20 | 20 | 0 | 10 | 50 | 70 | 70 | 50 | 80 | 80 | 90 | 90 | 40 | 100 | 10 | 30 | 20 | 60 | 70 | 40 | 10 | 30 | 0 | 10 | 20 |
| Crabgrass, Large | 10 | 50 | 70 | 30 | 40 | 20 | 0 | 0 | 40 | 50 | 30 | 40 | 80 | 90 | 90 | 80 | 90 | 60 | 100 | 100 | 100 | 100 | 60 | 70 | 40 | 60 | 60 | 50 | 20 | 50 | 10 | 10 | 0 |
| Foxtail, Giant | 0 | 30 | 60 | 20 | 30 | 20 | 0 | 0 | 30 | 30 | 20 | 20 | 50 | 90 | 90 | 70 | 90 | 70 | 100 | 100 | 100 | 100 | 30 | 30 | 30 | 60 | 60 | 50 | 20 | 40 | 0 | 0 | 0 |
| Galium | — | — | — | — | — | — | — | — | — | — | — | — | — | — | — | — | — | — | — | — | — | — | — | — | — | — | — | — | — | — | — | — | — |
| Italian Ryegrass | — | — | — | — | — | — | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — | — | — | — | — | — | — |
| Morningglory | 20 | 90 | 100 | 60 | 100 | 100 | 10 | 30 | 80 | 90 | 100 | 80 | 90 | 100 | 100 | 40 | 90 | 40 | 100 | 100 | 90 | 100 | 70 | 70 | 50 | 60 | 70 | 70 | 40 | 60 | 20 | 10 | 0 |
| Pigweed | 70 | 80 | 90 | 60 | 50 | 40 | 0 | 30 | 100 | 100 | 60 | 70 | 100 | 90 | 100 | 80 | 100 | 90 | 100 | 100 | 100 | 100 | 60 | 60 | 50 | 30 | 20 | 20 | 10 | 0 | 10 | 10 | 0 |
| Velvetleaf | 20 | 30 | 60 | 40 | 40 | 10 | 0 | 0 | 40 | 50 | 20 | 30 | 40 | 100 | 90 | 100 | 90 | 80 | 100 | 90 | 100 | 100 | 50 | 50 | 30 | 20 | 30 | 30 | 10 | 40 | 10 | 10 | 0 |
| Wheat | 0 | 0 | 10 | 0 | 20 | 0 | 0 | 0 | 0 | 0 | 0 | 0 | 10 | 70 | 70 | 50 | 80 | 40 | 90 | 90 | 50 | 80 | 20 | 10 | 0 | 50 | 40 | 40 | 0 | 10 | 0 | 0 | 0 |

| | 500 g ai/ha | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | | |
| | 90 | 91 | 92 | 93 | 94 | 95 | 96 | 97 | 98 | 99 | 100 | 101 | 102 | 103 | 104 | 105 | 106 | 107 | 108 | 109 | 110 | 111 | 112 | 113 | 114 | 115 | 116 | 117 | 118 |
| Barnyardgrass | 0 | 10 | 40 | 40 | 0 | 100 | 100 | 100 | 90 | 90 | 80 | 90 | 60 | 90 | 90 | 100 | 80 | 90 | 90 | 90 | 90 | 80 | 20 | 20 | 80 | 60 | 100 | 80 | 80 |
| Blackgrass | — | — | — | — | — | — | — | — | — | — | — | — | — | — | — | — | — | — | — | — | — | — | — | — | — | — | — | — | — |
| Common Ragweed | — | — | — | — | — | — | — | — | — | — | — | — | — | — | — | — | — | — | — | — | — | — | — | — | — | — | — | — | — |
| Corn | 30 | 10 | 40 | 90 | 0 | 100 | 100 | 100 | 90 | 90 | 70 | 100 | 50 | 90 | 100 | 100 | 60 | 90 | 100 | 90 | 90 | 50 | 30 | 20 | 70 | 40 | 100 | 50 | 50 |
| Crabgrass, Large | 0 | 10 | 30 | 80 | 0 | 100 | 100 | 100 | 90 | 100 | 90 | 90 | 80 | 100 | 90 | 100 | 90 | 90 | 100 | 90 | 100 | 100 | 20 | 40 | 90 | 90 | 100 | 80 | 90 |
| Foxtail, Giant | 0 | 10 | 50 | 80 | 0 | 100 | 100 | 100 | 90 | 100 | 80 | 90 | 90 | 90 | 90 | 100 | 80 | 90 | 100 | 90 | 100 | 100 | 20 | 40 | 90 | 80 | 100 | 90 | 70 |
| Galium | — | — | — | — | — | — | — | — | — | — | — | — | — | — | — | — | — | — | — | — | — | — | — | — | — | — | — | — | — |
| Italian Ryegrass | — | — | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — | — | — |
| Morningglory | 10 | 20 | 40 | 60 | 0 | 100 | 100 | 100 | 90 | 100 | 100 | 100 | 60 | 100 | 100 | 100 | 100 | 100 | 100 | 100 | 100 | 100 | 70 | 100 | 80 | 80 | 100 | 90 | 90 |
| Pigweed | 0 | 40 | 40 | 60 | 0 | 100 | 100 | 100 | 100 | 100 | 100 | 90 | 80 | 90 | 100 | 100 | 100 | 100 | 100 | 90 | 100 | 100 | 90 | 100 | 100 | 100 | 100 | 80 | 100 |
| Velvetleaf | 0 | 10 | 20 | 20 | 0 | 100 | 100 | 100 | 100 | 90 | 50 | 80 | 50 | 80 | 100 | 100 | 70 | 100 | 100 | 90 | 100 | 90 | 40 | 100 | 80 | 100 | 100 | 80 | 80 |
| Wheat | 0 | 0 | 20 | 40 | 0 | 90 | 90 | 90 | 80 | 70 | 10 | 100 | 30 | 90 | 70 | 100 | 40 | 80 | 90 | 80 | 90 | 30 | 40 | 20 | 40 | 40 | 100 | 0 | 40 |

500 g ai/ha
Compounds

| | 119 | 120 | 121 | 122 | 123 | 124 | 125 | 126 | 127 | 128 | 129 | 130 | 131 | 132 | 133 | 134 | 135 | 136 | 137 | 138 | 139 | 140 | 141 | 142 | 143 | 144 | 145 |
|---|---|---|---|---|---|---|---|---|---|---|---|---|---|---|---|---|---|---|---|---|---|---|---|---|---|---|---|
| Barnyardgrass | 60 | 90 | 90 | 70 | 60 | 70 | 90 | 100 | 90 | 100 | 90 | 90 | 90 | 90 | 100 | 30 | 10 | 90 | 60 | 100 | 90 | 90 | 80 | 50 | 90 | 90 | 90 |
| Blackgrass | — | — | — | 80 | 50 | 60 | 90 | 90 | 90 | 100 | — | — | — | — | 100 | 50 | 20 | 100 | 50 | — | — | — | — | — | — | — | — |
| Common Ragweed | — | — | — | 30 | 40 | 30 | 60 | 80 | 80 | 70 | — | — | — | — | 100 | 30 | 30 | 100 | 30 | — | — | — | — | — | — | — | — |
| Corn | 50 | 60 | 70 | 60 | 60 | 40 | 90 | 90 | 90 | 90 | 80 | 90 | 100 | 90 | 80 | 30 | 20 | 100 | 30 | 90 | 90 | 80 | 90 | 40 | 90 | 90 | 90 |
| Crabgrass, Large | 60 | 80 | 90 | — | — | — | — | — | — | — | 90 | 90 | 90 | 90 | — | — | — | — | — | 100 | 90 | 90 | 90 | 90 | 90 | 90 | 90 |
| Foxtail, Giant | 50 | 70 | 80 | 90 | 90 | 90 | 90 | 90 | 90 | 100 | 90 | 90 | 90 | 90 | 100 | 70 | 10 | 100 | 70 | 100 | 90 | 90 | 90 | 70 | 90 | 90 | 90 |
| Galium | — | — | — | 70 | 80 | 80 | 90 | 90 | 90 | 100 | — | — | — | — | 90 | 70 | 50 | 100 | 80 | — | — | — | — | — | — | — | — |
| Italian Ryegrass | — | — | — | 50 | 30 | 30 | 80 | 100 | 90 | 100 | — | — | — | — | 80 | 10 | 0 | 90 | 20 | — | — | — | — | — | — | — | — |
| Kochia | — | — | — | 90 | 90 | 90 | 90 | 100 | 100 | 90 | — | — | — | — | 100 | 90 | 100 | 90 | 90 | — | — | — | — | — | — | — | — |
| Morningglory | 100 | 100 | 100 | — | — | — | — | — | — | — | 100 | 100 | 100 | 100 | — | — | — | — | — | 100 | 100 | 90 | 100 | 70 | 100 | 90 | 100 |
| Pigweed | 90 | 80 | 100 | 40 | 100 | 100 | 100 | 90 | 90 | 100 | 100 | 100 | 100 | 100 | 100 | 90 | 40 | 100 | 80 | 100 | 100 | 100 | 100 | 70 | 100 | 100 | 100 |
| Velvetleaf | 70 | 80 | 100 | — | — | — | — | — | — | — | 100 | 100 | 100 | 100 | — | — | — | — | — | 100 | 90 | 80 | 100 | 40 | 90 | 90 | 100 |
| Wheat | 10 | 30 | 60 | 10 | 10 | 0 | 60 | 90 | 90 | 90 | 70 | 90 | 90 | 90 | 90 | 10 | 0 | 90 | 10 | 90 | 90 | 70 | 80 | 10 | 80 | 90 | 80 |

500 g ai/ha
Compounds

| | 146 | 147 | 148 | 149 | 150 | 151 | 152 | 153 | 154 | 159 | 160 | 161 | 162 | 163 | 164 | 165 | 166 | 167 | 168 | 170 | 171 | 172 | 173 | 174 | 175 | 176 | 177 |
|---|---|---|---|---|---|---|---|---|---|---|---|---|---|---|---|---|---|---|---|---|---|---|---|---|---|---|---|
| Barnyardgrass | 10 | 30 | 40 | 80 | 20 | 50 | 50 | 100 | 100 | 30 | 30 | 10 | 20 | 40 | 40 | 10 | 90 | 80 | 90 | 30 | 20 | 10 | 10 | 10 | 90 | 90 | 100 |
| Blackgrass | — | — | — | — | — | — | — | — | — | — | — | — | — | — | — | — | — | — | — | — | — | — | — | — | — | — | — |
| Common Ragweed | — | — | — | — | — | — | — | — | — | — | — | — | — | — | — | — | — | — | — | — | — | — | — | — | — | — | — |
| Corn | 0 | 10 | 40 | 80 | 10 | 50 | 30 | 100 | 100 | 20 | 20 | 10 | 20 | 30 | 40 | 10 | 80 | 70 | 80 | 10 | 20 | 10 | 10 | 10 | 90 | 80 | 100 |
| Crabgrass, Large | 80 | 40 | 50 | 90 | 10 | 80 | 60 | 100 | 100 | 30 | 40 | 30 | 20 | 70 | 60 | 20 | 80 | 80 | 90 | 30 | 30 | 20 | 80 | 10 | 90 | 90 | 100 |
| Foxtail, Giant | 20 | 30 | 40 | 80 | 10 | 70 | 40 | 100 | 100 | 30 | 30 | 20 | 20 | 80 | 40 | 10 | 70 | 90 | 90 | 10 | 20 | 10 | 40 | 0 | 90 | 90 | 100 |
| Galium | — | — | — | — | — | — | — | — | — | — | — | — | — | — | — | — | — | — | — | — | — | — | — | — | — | — | — |
| Italian Ryegrass | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — |
| Morningglory | 80 | 70 | 90 | 100 | 30 | 90 | 60 | 100 | 100 | 40 | 20 | 50 | 40 | 90 | 70 | 20 | 100 | 100 | 100 | 20 | 30 | 70 | 50 | 10 | 90 | 90 | 100 |
| Pigweed | 90 | 90 | 100 | 100 | 80 | 100 | 80 | 100 | 100 | 60 | 80 | 70 | 60 | 90 | 40 | 80 | 100 | 100 | 100 | 80 | 50 | 80 | 50 | 90 | 100 | 100 | 100 |
| Velvetleaf | 20 | 50 | 60 | 90 | 10 | 60 | 60 | 100 | 100 | 70 | 60 | 50 | 40 | 80 | 60 | 20 | 60 | 60 | 70 | 40 | 30 | 20 | 30 | 10 | 80 | 90 | 100 |
| Wheat | 0 | 0 | 20 | 60 | 0 | 10 | 0 | 100 | 100 | 20 | 0 | 0 | 0 | 0 | 20 | 10 | 50 | 30 | 40 | 0 | 0 | 0 | 0 | 0 | 80 | 60 | 100 |

500 g ai/ha
Compounds

| | 178 | 179 | 180 | 181 | 182 | 183 | 184 | 185 | 186 | 187 | 188 | 189 | 190 | 191 | 192 | 195 | 196 | 197 | 198 | 199 | 200 | 201 | 202 | 203 | 204 | 205 | 206 |
|---|---|---|---|---|---|---|---|---|---|---|---|---|---|---|---|---|---|---|---|---|---|---|---|---|---|---|---|
| Barnyardgrass | 80 | 90 | 30 | 10 | 30 | 20 | 60 | 90 | 90 | 90 | 90 | 90 | 60 | 100 | 30 | 90 | 90 | 100 | 100 | 90 | 100 | 100 | 100 | 80 | 50 | 90 | 60 |
| Blackgrass | — | — | — | — | — | — | — | — | — | — | — | — | — | — | — | — | — | — | — | — | — | — | — | — | — | 90 | 50 |
| Common Ragweed | — | — | — | — | — | — | — | — | — | — | — | — | — | — | — | — | — | — | — | — | — | — | — | — | — | 90 | 30 |
| Corn | 90 | 90 | 0 | 20 | 20 | 10 | 60 | 90 | 100 | 100 | 100 | 100 | 40 | 100 | 50 | 90 | 90 | 90 | 90 | 90 | 100 | 100 | 90 | 100 | 30 | 100 | 60 |
| Crabgrass, Large | 90 | 90 | 30 | 10 | 30 | 20 | 70 | 90 | 90 | 90 | 90 | 90 | 70 | 90 | 20 | 100 | 100 | 100 | 100 | 90 | 100 | 100 | 100 | 90 | 80 | — | — |
| Foxtail, Giant | 90 | 90 | 30 | 10 | 20 | 20 | 40 | 90 | 90 | 90 | 90 | 90 | 60 | 90 | 30 | 100 | 100 | 90 | 100 | 90 | 100 | 100 | 90 | 90 | 70 | 90 | 80 |
| Galium | — | — | — | — | — | — | — | — | — | — | — | — | — | — | — | — | — | — | — | — | — | — | — | — | — | 90 | 80 |
| Italian Ryegrass | — | — | — | — | — | — | — | — | — | — | — | — | — | — | — | — | — | — | — | — | — | — | — | — | — | 100 | 50 |
| Kochia | — | — | — | — | — | — | — | — | — | — | — | — | — | — | — | — | — | — | — | — | — | — | — | — | — | 90 | 90 |
| Morningglory | 100 | 100 | 80 | 80 | 30 | 90 | 90 | 90 | 100 | 100 | 100 | 100 | 90 | 90 | 90 | 100 | 100 | 100 | 100 | 100 | 100 | 100 | 90 | 90 | 90 | — | — |
| Pigweed | 100 | 100 | 100 | 100 | 90 | 80 | 90 | 100 | 100 | 90 | 100 | 100 | 90 | 100 | 90 | 100 | 100 | 100 | 100 | 100 | 100 | 100 | 100 | 100 | 90 | 100 | 100 |
| Velvetleaf | 60 | 100 | 30 | 20 | 70 | 40 | 60 | 100 | 100 | 100 | 100 | 100 | 80 | 100 | 80 | 100 | 100 | 80 | 100 | 90 | 90 | 100 | 90 | 90 | 50 | — | — |
| Wheat | 30 | 70 | 0 | 0 | 0 | 0 | 20 | 80 | 90 | 80 | 90 | 90 | 10 | 60 | 10 | 90 | 90 | 60 | 90 | 80 | 100 | 100 | 80 | 80 | 10 | 90 | 20 |

| | 500 g ai/ha | | | | | | | | | | | | | | | | | | | | | | | | |
|---|---|---|---|---|---|---|---|---|---|---|---|---|---|---|---|---|---|---|---|---|---|---|---|---|---|
| | Compounds | | | | | | | | | | | | | | | | | | | | | | | | |
| | 207 | 208 | 209 | 210 | 211 | 212 | 213 | 214 | 215 | 216 | 217 | 218 | 219 | 220 | 221 | 222 | 223 | 224 | 225 | 226 | 227 | 228 | 229 | 230 | 231 |
| Barnyardgrass | 80 | 10 | 0 | 0 | 50 | 10 | 0 | 100 | 40 | 70 | 20 | 20 | 40 | 100 | 10 | 80 | 50 | 100 | 30 | 90 | 80 | 50 | 90 | 70 | 0 |
| Blackgrass | 60 | 10 | 0 | 0 | 50 | 10 | 0 | — | — | — | — | — | — | — | — | — | — | — | — | — | — | — | — | — | — |
| Common Ragweed | 30 | 20 | 0 | 0 | 20 | 10 | 0 | — | — | — | — | — | — | — | — | — | — | — | — | — | — | — | — | — | — |
| Corn | 60 | 10 | 0 | 0 | 40 | 10 | 0 | 90 | 20 | 50 | 20 | 20 | 20 | 90 | 20 | 60 | 30 | 90 | 30 | 80 | 70 | 50 | 70 | 50 | 10 |
| Crabgrass, Large | — | — | — | — | — | — | — | 100 | 60 | 70 | 70 | 40 | 70 | 100 | 30 | 90 | 70 | 100 | 70 | 90 | 90 | 60 | 90 | 80 | 10 |
| Foxtail, Giant | 60 | 10 | 0 | 0 | 60 | 10 | 0 | 100 | 70 | 40 | 30 | 30 | 60 | 100 | 30 | 70 | 30 | 100 | 60 | 90 | 90 | 60 | 90 | 60 | 0 |
| Galium | 80 | 50 | 0 | 20 | 70 | 40 | 0 | — | — | — | — | — | — | — | — | — | — | — | — | — | — | — | — | — | — |
| Italian Ryegrass | 50 | 0 | 0 | 0 | 30 | 10 | 0 | — | — | — | — | — | — | — | — | — | — | — | — | — | — | — | — | — | — |
| Kochia | 60 | 50 | 0 | 20 | 90 | 70 | 0 | — | — | — | — | — | — | — | — | — | — | — | — | — | — | — | — | — | — |
| Morningglory | — | — | — | — | — | — | — | 100 | 100 | 70 | 70 | 100 | 70 | 100 | 80 | 50 | 30 | 100 | 60 | 100 | 100 | 100 | 100 | 100 | 0 |
| Pigweed | 100 | 80 | 0 | 20 | 100 | 80 | 0 | 100 | 90 | 30 | 70 | 80 | 60 | 100 | 60 | 100 | 60 | 100 | 0 | 100 | 100 | 80 | 100 | 100 | 0 |
| Velvetleaf | — | — | — | — | — | — | — | 100 | 60 | 40 | 30 | 40 | 90 | 80 | 20 | 50 | 30 | 100 | 30 | 90 | 100 | 30 | 80 | 50 | 0 |
| Wheat | 20 | 0 | 0 | 0 | 20 | 10 | 0 | 90 | 10 | 0 | 0 | 10 | 10 | 90 | 10 | 20 | 10 | 90 | 0 | 90 | 70 | 10 | 70 | 10 | 0 |

| | 125 g ai/ha | | | | | | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | | | | | | | | | | | |
| | 3 | 4 | 5 | 6 | 7 | 8 | 9 | 10 | 11 | 12 | 13 | 14 | 21 | 69 | 70 | 71 | 72 | 73 | 75 | 76 | 77 | 78 | 79 | 80 | 81 | 82 | 83 | 84 | 85 | 86 | 87 | 88 | 89 | 90 | 91 | 92 | 93 | 94 |
| Barnyardgrass | 0 | 10 | 10 | 0 | 0 | 0 | 0 | 0 | 0 | 10 | 0 | 0 | 0 | 80 | 80 | 30 | 90 | 40 | 90 | 80 | 50 | 50 | 0 | 10 | 0 | 20 | 10 | 10 | 0 | 0 | 0 | 0 | 0 | 0 | 0 | 0 | 0 | 0 |
| Blackgrass | — | — | — | — | — | — | — | — | — | — | — | — | — | — | — | — | — | — | — | — | — | — | — | — | — | — | — | — | — | — | — | — | — | — | — | — | — | — |
| Common Ragweed | — | — | — | — | — | — | — | — | — | — | — | — | — | — | — | — | — | — | — | — | — | — | — | — | — | — | — | — | — | — | — | — | — | — | — | — | — | — |
| Corn | 0 | 10 | 10 | 0 | 10 | 0 | 0 | 0 | 0 | 10 | 0 | 0 | 10 | 60 | 60 | 30 | 70 | 40 | 80 | 80 | 30 | 100 | 10 | 10 | 10 | 20 | 10 | 10 | 0 | 10 | 0 | 0 | 0 | 0 | 0 | 0 | 0 | 0 |
| Crabgrass, Large | 0 | 20 | 10 | 10 | 10 | 10 | 0 | 0 | 20 | 30 | 20 | 20 | 30 | 70 | 90 | 40 | 80 | 40 | 100 | 80 | 90 | 90 | 20 | 10 | 10 | 30 | 30 | 20 | 10 | 10 | 0 | 0 | 0 | 0 | 0 | 0 | 20 | 0 |
| Foxtail, Giant | 0 | 10 | 10 | 10 | 0 | 10 | 0 | 0 | 10 | 20 | 0 | 0 | 20 | 80 | 90 | 40 | 80 | 40 | 90 | 80 | 90 | 90 | 10 | 10 | 10 | 10 | 20 | 10 | 0 | 10 | 0 | 0 | 0 | 0 | 0 | 0 | 20 | 0 |
| Galium | — | — | — | — | — | — | — | — | — | — | — | — | — | — | — | — | — | — | — | — | — | — | — | — | — | — | — | — | — | — | — | — | — | — | — | — | — | — |
| Italian Ryegrass | — | — | — | — | — | — | — | — | — | — | — | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — | — | — | — | — | — | — | — | — | — | — | — |
| Morningglory | 10 | 30 | 40 | 30 | 40 | 80 | 0 | 20 | 70 | 50 | 50 | 50 | 80 | 100 | 100 | 40 | 80 | 30 | 100 | 70 | 100 | 100 | 30 | 30 | 30 | 50 | 20 | 20 | 10 | 10 | 10 | 10 | 0 | 0 | 10 | 0 | 0 | 0 |
| Pigweed | 20 | 70 | 50 | 50 | 30 | 10 | 0 | 10 | 60 | 80 | 40 | 50 | 90 | 70 | 90 | 40 | 90 | 70 | 100 | 100 | 100 | 100 | 40 | 40 | 30 | 10 | 10 | 10 | 0 | 0 | 0 | 0 | 0 | 0 | 20 | 0 | 20 | 0 |
| Velvetleaf | 0 | 10 | 20 | 10 | 30 | — | 0 | 0 | 30 | 40 | 0 | 20 | 0 | 100 | 80 | 50 | 80 | 30 | 90 | 30 | 80 | 100 | 40 | 10 | 20 | 10 | 30 | 20 | 0 | 30 | 0 | 0 | 0 | 0 | 10 | 0 | 0 | 0 |
| Wheat | 0 | 0 | 0 | 0 | 0 | 0 | 0 | 0 | 0 | 0 | 0 | 0 | 0 | 30 | 70 | 30 | 70 | 0 | 80 | 80 | 40 | 70 | 0 | 0 | 0 | 0 | 0 | 0 | 0 | 0 | 0 | 0 | 0 | 0 | 0 | 0 | 20 | 0 |

| | 125 g ai/ha | | | | | | | | | | | | | | | | | | | | | | | | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | |
| | 95 | 96 | 97 | 98 | 99 | 100 | 101 | 102 | 103 | 104 | 105 | 106 | 107 | 108 | 109 | 110 | 111 | 112 | 113 | 114 | 115 | 116 | 117 | 118 | 119 | 120 | 121 |
| Barnyardgrass | 90 | 90 | 90 | 90 | 20 | 30 | 80 | 20 | 90 | 90 | 90 | 50 | 80 | 90 | 90 | 80 | 20 | 0 | 10 | 40 | 30 | 100 | 40 | 30 | 10 | 30 | 90 |
| Blackgrass | — | — | — | — | — | — | — | — | — | — | — | — | — | — | — | — | — | — | — | — | — | — | — | — | — | — | — |
| Common Ragweed | — | — | — | — | — | — | — | — | — | — | — | — | — | — | — | — | — | — | — | — | — | — | — | — | — | — | — |
| Corn | 80 | 90 | 100 | 80 | 50 | 20 | 90 | 30 | 80 | 90 | 80 | 30 | 50 | 80 | 80 | 80 | 10 | 0 | 20 | 30 | 20 | 100 | 20 | 20 | 0 | 30 | 40 |
| Crabgrass, Large | 90 | 90 | 100 | 90 | 60 | 50 | 80 | 50 | 90 | 90 | 100 | 50 | 80 | 90 | 90 | 70 | 30 | 10 | 20 | 60 | 50 | 100 | 20 | 40 | 20 | 30 | 70 |
| Foxtail, Giant | 100 | 100 | 100 | 90 | 60 | 30 | 80 | 50 | 90 | 90 | 100 | 50 | 70 | 90 | 90 | 90 | 40 | 10 | 20 | 60 | 30 | 100 | 20 | 10 | 20 | 30 | 50 |
| Galium | — | — | — | — | — | — | — | — | — | — | — | — | — | — | — | — | — | — | — | — | — | — | — | — | — | — | — |
| Italian Ryegrass | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — |
| Morningglory | 90 | 90 | 100 | 90 | 50 | 70 | 100 | 40 | 90 | 100 | 100 | 50 | 90 | 90 | 90 | 100 | 90 | 40 | 40 | 40 | 30 | 100 | 60 | 70 | 90 | 80 | 90 |
| Pigweed | 100 | 100 | 100 | 100 | 70 | 100 | 60 | 50 | 90 | 100 | 100 | 90 | 100 | 90 | 90 | 100 | 80 | 30 | 60 | 100 | 90 | 100 | 50 | 80 | 60 | 50 | 90 |
| Velvetleaf | 90 | 50 | 100 | 70 | 20 | 20 | 50 | 10 | 70 | 90 | 100 | 40 | 80 | 60 | 60 | 90 | 40 | 20 | 40 | 30 | 60 | 90 | 40 | 50 | 50 | 30 | 60 |
| Wheat | 80 | 80 | 80 | 70 | 20 | 0 | 80 | 0 | 80 | 70 | 100 | 10 | 30 | 40 | 50 | 80 | 10 | 10 | 20 | 0 | 0 | 100 | 0 | 0 | 0 | 0 | 0 |

125 g ai/ha
Compounds

| | 122 | 123 | 124 | 125 | 126 | 127 | 128 | 129 | 130 | 131 | 132 | 133 | 134 | 135 | 136 | 137 | 138 | 139 | 140 | 141 | 142 | 143 | 144 | 145 | 146 | 147 | 148 |
|---|---|---|---|---|---|---|---|---|---|---|---|---|---|---|---|---|---|---|---|---|---|---|---|---|---|---|---|
| Barnyardgrass | 0 | 0 | 0 | 30 | 90 | 90 | 100 | 90 | 70 | 80 | 90 | 90 | 10 | 10 | 70 | 0 | 100 | 80 | 70 | 40 | 20 | 60 | 70 | 60 | 0 | 10 | 10 |
| Blackgrass | 10 | 10 | 10 | 30 | 90 | 90 | 90 | — | — | — | — | 90 | 10 | 0 | 80 | 10 | — | — | — | — | — | — | — | — | — | — | — |
| Common Ragweed | 10 | 10 | 20 | 20 | 80 | 30 | 30 | — | — | — | — | 60 | 20 | 10 | 30 | 10 | — | — | — | — | — | — | — | — | — | — | — |
| Corn | 20 | 20 | 10 | 30 | 90 | 90 | 90 | 50 | 70 | 80 | 70 | 60 | 10 | 10 | 60 | 10 | 80 | 50 | 50 | 50 | 10 | 60 | 50 | 60 | 0 | 0 | 10 |
| Crabgrass, Large | — | — | — | — | — | — | — | 80 | 70 | 70 | 90 | — | — | — | — | — | 100 | 90 | 70 | 70 | 50 | 90 | 90 | 90 | 10 | 10 | 20 |
| Foxtail, Giant | 0 | 0 | 0 | 30 | 90 | 90 | 100 | 80 | 60 | 80 | 80 | 100 | 10 | 0 | 90 | 10 | 100 | 90 | 80 | 80 | 20 | 90 | 90 | 90 | 0 | 10 | 10 |
| Galium | 10 | 10 | 10 | 60 | 90 | 90 | 90 | — | — | — | — | 80 | 30 | 20 | 90 | 50 | — | — | — | — | — | — | — | — | — | — | — |
| Italian Ryegrass | 10 | 10 | 10 | 20 | 90 | 80 | 90 | — | — | — | — | 60 | 0 | 0 | 80 | 0 | — | — | — | — | — | — | — | — | — | — | — |
| Kochia | 50 | 50 | 50 | 60 | 100 | 90 | 90 | — | — | — | — | 90 | 60 | 70 | 90 | 50 | — | — | — | — | — | — | — | — | — | — | — |
| Morningglory | — | — | — | — | — | — | — | 90 | 90 | 90 | 90 | — | — | — | — | — | 80 | 60 | 90 | 90 | 30 | 40 | 80 | 80 | 20 | 30 | 80 |
| Pigweed | 30 | 70 | 30 | 70 | 90 | 90 | 100 | 100 | 100 | 100 | 90 | 100 | 40 | 10 | 80 | 50 | 100 | 90 | 100 | 60 | 40 | 100 | 100 | 100 | 60 | 60 | 90 |
| Velvetleaf | — | — | — | — | — | — | — | 90 | 80 | 90 | 100 | — | — | — | — | — | 60 | 40 | 50 | 30 | 30 | 80 | 80 | 70 | 10 | 30 | 20 |
| Wheat | 0 | 0 | 0 | 0 | 90 | 80 | 70 | 50 | 20 | 80 | 80 | 40 | 0 | 0 | 60 | 0 | 80 | 50 | 40 | 30 | 0 | 60 | 50 | 50 | 0 | 0 | 0 |

125 g ai/ha
Compounds

| | 149 | 150 | 151 | 152 | 153 | 154 | 159 | 160 | 161 | 162 | 163 | 164 | 165 | 166 | 167 | 168 | 170 | 171 | 172 | 173 | 174 | 175 | 176 | 177 | 178 | 179 | 180 |
|---|---|---|---|---|---|---|---|---|---|---|---|---|---|---|---|---|---|---|---|---|---|---|---|---|---|---|---|
| Barnyardgrass | 30 | 0 | 10 | 10 | 100 | 100 | 0 | 0 | 10 | 0 | 10 | 10 | 10 | 30 | 20 | 50 | 10 | 0 | 0 | 0 | 0 | 70 | 60 | 100 | 70 | 70 | 0 |
| Blackgrass | — | — | — | — | — | — | — | — | — | — | — | — | — | — | — | — | — | — | — | — | — | — | — | — | — | — | — |
| Common Ragweed | — | — | — | — | — | — | — | — | — | — | — | — | — | — | — | — | — | — | — | — | — | — | — | — | — | — | — |
| Corn | 40 | 0 | 10 | 10 | 90 | 40 | 10 | 0 | 0 | 10 | 0 | 10 | 10 | 30 | 10 | 20 | 0 | 0 | 0 | 0 | 0 | 60 | 40 | 90 | 10 | 50 | 0 |
| Crabgrass, Large | 40 | 10 | 30 | 10 | 100 | 100 | 20 | 20 | 20 | 10 | 20 | 10 | 10 | 40 | 20 | 60 | 20 | 10 | 10 | 30 | 0 | 90 | 80 | 100 | 80 | 90 | 10 |
| Foxtail, Giant | 70 | 0 | 20 | 10 | 100 | 100 | 20 | 20 | 10 | 10 | 10 | 10 | 10 | 50 | 40 | 60 | 0 | 10 | 0 | 0 | 0 | 80 | 80 | 100 | 70 | 90 | 10 |
| Galium | — | — | — | — | — | — | — | — | — | — | — | — | — | — | — | — | — | — | — | — | — | — | — | — | — | — | — |
| Italian Ryegrass | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — |
| Morningglory | 60 | 30 | 50 | 40 | 100 | 100 | 30 | 0 | 30 | 20 | 30 | 50 | 10 | 100 | 80 | 70 | 10 | 0 | 30 | 20 | 0 | 80 | 90 | 100 | 70 | 60 | 0 |
| Pigweed | 90 | 30 | 70 | 60 | 100 | 100 | 50 | 50 | 40 | 40 | 50 | 20 | 30 | 10 | 70 | 80 | 60 | 20 | 30 | 30 | 20 | 100 | 100 | 100 | 100 | 100 | 70 |
| Velvetleaf | 30 | 10 | 30 | 20 | 70 | 80 | 60 | 30 | 30 | 0 | 50 | 20 | 10 | 40 | 30 | 40 | 0 | 20 | 10 | 10 | 0 | 60 | 40 | 40 | 40 | 70 | 0 |
| Wheat | 20 | 0 | 0 | 0 | 90 | 20 | 0 | 0 | 0 | 0 | 0 | 0 | 0 | 0 | 0 | 0 | 0 | 0 | 0 | 0 | 0 | 40 | 10 | 50 | 0 | 30 | 0 |

125 g ai/ha
Compounds

| | 181 | 182 | 183 | 184 | 185 | 186 | 187 | 188 | 189 | 190 | 191 | 192 | 195 | 196 | 197 | 198 | 199 | 200 | 201 | 202 | 203 | 204 | 205 | 206 | 207 | 208 | 209 |
|---|---|---|---|---|---|---|---|---|---|---|---|---|---|---|---|---|---|---|---|---|---|---|---|---|---|---|---|
| Barnyardgrass | 10 | 20 | 10 | 0 | 80 | 90 | 90 | 90 | 90 | 10 | 70 | 10 | 70 | 90 | 80 | 90 | 70 | 90 | 80 | 90 | 40 | 10 | 80 | 0 | 0 | 0 | 0 |
| Blackgrass | — | — | — | — | — | — | — | — | — | — | — | — | — | — | — | — | — | — | — | — | — | — | 70 | 0 | 0 | 0 | 0 |
| Common Ragweed | — | — | — | — | — | — | — | — | — | — | — | — | — | — | — | — | — | — | — | — | — | — | 80 | 0 | 0 | 0 | 0 |
| Corn | 0 | 0 | 0 | 0 | 0 | 100 | 80 | 90 | 80 | 10 | 60 | 20 | 80 | 80 | 30 | 90 | 50 | 80 | 90 | 80 | 50 | 10 | 70 | 30 | 30 | 0 | 0 |
| Crabgrass, Large | 10 | 10 | 10 | 0 | 80 | 90 | 90 | 90 | 90 | 20 | 80 | 10 | 80 | 90 | 80 | 90 | 90 | 100 | 90 | 90 | 70 | 10 | — | — | — | — | — |
| Foxtail, Giant | 0 | 0 | 10 | 0 | 80 | 90 | 90 | 90 | 90 | 20 | 80 | 10 | 80 | 90 | 80 | 90 | 90 | 100 | 90 | 90 | 80 | 10 | 90 | 0 | 0 | 0 | 0 |
| Galium | — | — | — | — | — | — | — | — | — | — | — | — | — | — | — | — | — | — | — | — | — | — | 90 | 20 | 40 | 20 | 0 |
| Italian Ryegrass | — | — | — | — | — | — | — | — | — | — | — | — | — | — | — | — | — | — | — | — | — | — | 70 | 0 | 0 | 0 | 0 |
| Kochia | — | — | — | — | — | — | — | — | — | — | — | — | — | — | — | — | — | — | — | — | — | — | 90 | 80 | 30 | 20 | 0 |
| Morningglory | 10 | 30 | 20 | 0 | 40 | 70 | 90 | 100 | 90 | 30 | 80 | 50 | 70 | 90 | 80 | 80 | 90 | 100 | 90 | 70 | 50 | 80 | — | — | — | — | — |
| Pigweed | 90 | 60 | 60 | 60 | 100 | 100 | 80 | 100 | 100 | 80 | 100 | 50 | 100 | 100 | 100 | 100 | 100 | 100 | 100 | 100 | 90 | 90 | 100 | 80 | 60 | 40 | 0 |
| Velvetleaf | 10 | 50 | 10 | 20 | 90 | 100 | 100 | 100 | 90 | 50 | 70 | 60 | 70 | 80 | 30 | 30 | 80 | 80 | 70 | 70 | 30 | 40 | — | — | — | — | — |
| Wheat | 0 | 0 | 0 | 0 | 50 | 80 | 80 | 80 | 80 | 0 | 0 | 0 | 90 | 40 | 0 | 60 | 70 | 40 | 90 | 60 | 70 | 0 | 70 | 10 | 10 | 0 | 0 |

| | 125 g ai/ha | | | | | | | | | | | | | | | | | | | | | |
|---|---|---|---|---|---|---|---|---|---|---|---|---|---|---|---|---|---|---|---|---|---|---|
| | Compounds | | | | | | | | | | | | | | | | | | | | | |
| | 210 | 211 | 212 | 213 | 214 | 215 | 216 | 217 | 218 | 219 | 220 | 221 | 222 | 223 | 224 | 225 | 226 | 227 | 228 | 229 | 230 | 231 |
| Barnyardgrass | 0 | 0 | 0 | 0 | 100 | 10 | 0 | 0 | 0 | 10 | 80 | 10 | 80 | 0 | 90 | 10 | 60 | 40 | 0 | 50 | 10 | 0 |
| Blackgrass | 0 | 0 | 0 | 0 | — | — | — | — | — | — | — | — | — | — | — | — | — | — | — | — | — | — |
| Common Ragweed | 0 | 0 | 0 | 0 | — | — | — | — | — | — | — | — | — | — | — | — | — | — | — | — | — | — |
| Corn | 0 | 30 | 0 | 0 | 40 | 10 | 0 | 0 | 10 | 10 | 40 | 10 | 0 | 0 | 60 | 10 | 50 | 30 | 10 | 40 | 10 | 0 |
| Crabgrass, Large | — | — | — | — | 100 | 10 | 20 | 20 | 20 | 20 | 90 | 10 | 40 | 20 | 90 | 40 | 70 | 60 | 20 | 50 | 30 | 0 |
| Foxtail, Giant | 0 | 0 | 0 | 0 | 100 | 10 | 0 | 0 | 20 | 20 | 90 | 10 | 20 | 0 | 90 | 10 | 80 | 70 | 10 | 60 | 10 | 0 |
| Galium | 0 | 20 | 20 | 0 | — | — | — | — | — | — | — | — | — | — | — | — | — | — | — | — | — | — |
| Italian Ryegrass | 0 | 0 | 0 | 0 | — | — | — | — | — | — | — | — | — | — | — | — | — | — | — | — | — | — |
| Kochia | 10 | 80 | 10 | 0 | — | — | — | — | — | — | — | — | — | — | — | — | — | — | — | — | — | — |
| Morningglory | — | — | — | — | 80 | 30 | 20 | 20 | 50 | 10 | 50 | 30 | 30 | 0 | 30 | 10 | 70 | 70 | 50 | 70 | 60 | 0 |
| Pigweed | 0 | 70 | 40 | 0 | 100 | 40 | 20 | 20 | 50 | 20 | 90 | 20 | 30 | 20 | 100 | 0 | 100 | 100 | 30 | 90 | 50 | 0 |
| Velvetleaf | — | — | — | — | 80 | 10 | 0 | 0 | 0 | 0 | 70 | 0 | 30 | 0 | 60 | 20 | 50 | 40 | 10 | 50 | 10 | 0 |
| Wheat | 0 | 0 | 0 | 0 | 80 | 0 | 0 | 0 | 0 | 0 | 0 | 0 | 0 | 0 | 30 | 0 | 60 | 20 | 0 | 40 | 0 | 0 |

Preemergence

2000 g ai/ha
Compounds

| | 1 | 2 |
|---|---|---|
| Barnyardgrass | 10 | 90 |
| Crabgrass, Large | 10 | 100 |
| Morningglory | 0 | 100 |
| Velvetleaf | 0 | 100 |

| | 1000 g ai/ha | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | | | | |
| | 15 | 16 | 17 | 18 | 19 | 20 | 22 | 23 | 24 | 25 | 26 | 27 | 28 | 29 | 30 | 31 | 32 | 33 | 34 | 35 | 36 | 37 | 38 | 39 | 40 | 41 | 42 | 43 | 44 | 45 | 46 |
| Barnyardgrass | 90 | 90 | 80 | 80 | 90 | 80 | 0 | 0 | 0 | 0 | 80 | 50 | 50 | 50 | 90 | 50 | 70 | 40 | 60 | 70 | 0 | 0 | 0 | 0 | 30 | 10 | 0 | 0 | 10 | 10 | 10 |
| Corn | 20 | 50 | 10 | 30 | 30 | 20 | 0 | 0 | 0 | 0 | 40 | 30 | 0 | 0 | 20 | 10 | 0 | 0 | 10 | 0 | 0 | 0 | 0 | 0 | 0 | 0 | 0 | 0 | 20 | 0 | 0 |
| Crabgrass, Large | 100 | 100 | 100 | 100 | 100 | 90 | 10 | 0 | 20 | 20 | 100 | 100 | 100 | 100 | 100 | 100 | 100 | 100 | 100 | 100 | 20 | 40 | 20 | 10 | 50 | 30 | 40 | 0 | 50 | 20 | 10 |
| Foxtail, Giant | 100 | 100 | 100 | 100 | 100 | 100 | 20 | 0 | 0 | 10 | 100 | 90 | 100 | 80 | 100 | 100 | 100 | 90 | 100 | 100 | 20 | 30 | 0 | 0 | 80 | 50 | 30 | 10 | 50 | 20 | 20 |
| Morningglory | 90 | 100 | 80 | 70 | 90 | 70 | 0 | 0 | 0 | 0 | 90 | 90 | 60 | 60 | 60 | 40 | 50 | 40 | 60 | 50 | 0 | 0 | 0 | 0 | 40 | 20 | 30 | 0 | 20 | 10 | 20 |
| Pigweed | 90 | 100 | 100 | 90 | 100 | 100 | 0 | 0 | 40 | 0 | 100 | 90 | 40 | 90 | 30 | 90 | 100 | 90 | 80 | 90 | 30 | 20 | 0 | 0 | 30 | 10 | 10 | 0 | 10 | 10 | 0 |
| Velvetleaf | 100 | 30 | 100 | 100 | 80 | 70 | 0 | 0 | 0 | 0 | 100 | 30 | 70 | 40 | 30 | 50 | 30 | 40 | 30 | 50 | 0 | 0 | 0 | 0 | 0 | 0 | 0 | 0 | 0 | 0 | 0 |
| Wheat | 20 | 50 | 20 | 40 | 10 | 20 | 0 | 0 | 0 | 0 | 40 | 20 | 0 | 10 | 10 | 10 | 0 | 10 | 20 | 0 | 0 | 0 | 0 | 0 | 20 | 0 | 0 | 0 | 0 | 0 | 0 |

| | 1000 g ai/ha | | | | | | | | | | | | | | | | | | | | | | |
|---|---|---|---|---|---|---|---|---|---|---|---|---|---|---|---|---|---|---|---|---|---|---|---|
| | Compounds | | | | | | | | | | | | | | | | | | | | | | |
| | 47 | 48 | 49 | 50 | 51 | 52 | 53 | 54 | 55 | 56 | 57 | 58 | 59 | 60 | 61 | 62 | 63 | 64 | 65 | 66 | 67 | 68 | 74 |
| Barnyardgrass | 80 | 10 | 30 | 10 | 10 | 10 | 40 | 10 | 10 | 0 | 10 | 10 | 0 | 30 | 10 | 10 | 0 | 60 | 60 | 0 | 0 | 0 | 100 |
| Corn | 40 | 0 | 0 | 0 | — | 0 | 10 | 0 | 0 | 0 | 0 | 0 | 0 | 0 | 0 | 0 | 0 | 30 | 0 | 0 | 0 | 0 | 90 |
| Crabgrass, Large | 100 | 10 | 90 | 10 | 20 | 50 | 100 | 40 | 100 | 0 | 20 | 70 | 10 | 90 | 60 | 40 | 0 | 100 | 80 | 50 | 50 | 40 | 100 |
| Foxtail, Giant | 100 | 10 | 100 | 30 | 80 | 70 | 90 | 20 | 90 | 10 | 90 | 20 | 10 | 100 | 70 | 60 | 0 | 100 | 70 | 100 | 90 | 60 | 100 |
| Morningglory | 90 | 20 | 30 | 0 | 10 | 30 | 40 | 0 | 20 | 0 | 10 | 20 | 0 | 20 | 10 | 20 | 0 | 30 | 70 | 30 | 0 | 0 | 100 |
| Pigweed | 80 | 0 | 50 | 0 | 10 | 30 | 100 | 90 | 80 | 0 | 10 | 70 | 0 | 90 | 10 | 20 | 0 | 100 | 90 | 50 | 0 | 70 | 100 |
| Velvetleaf | 60 | 0 | 0 | 0 | 0 | 10 | 30 | 0 | 10 | 0 | 0 | 0 | 0 | 0 | 0 | 10 | 0 | 50 | 20 | 20 | 10 | 0 | 100 |
| Wheat | 50 | 0 | 0 | 0 | 0 | 0 | 0 | 0 | 0 | 0 | 0 | 0 | 0 | 0 | 0 | 0 | 0 | 20 | 0 | 0 | 0 | 0 | 100 |

500 g ai/ha
Compounds

| | 3 | 4 | 5 | 6 | 7 | 8 | 9 | 10 | 11 | 12 | 13 | 14 | 21 | 69 | 70 | 71 | 72 | 73 | 75 | 76 | 77 | 78 | 79 | 80 | 81 | 82 | 83 | 84 | 85 |
|---|---|---|---|---|---|---|---|---|---|---|---|---|---|---|---|---|---|---|---|---|---|---|---|---|---|---|---|---|---|
| Barnyardgrass | 0 | 50 | 60 | 0 | 40 | 0 | 10 | 0 | 50 | 30 | 0 | 10 | 90 | 80 | 100 | 80 | 100 | 60 | 100 | 100 | 100 | 100 | 70 | 80 | 70 | 80 | 90 | 70 | 30 |
| Common Ragweed | — | — | — | — | — | — | — | — | — | — | — | — | — | — | — | — | — | — | — | — | — | — | — | — | — | — | — | — | — |
| Corn | 0 | 0 | 0 | 0 | 10 | 0 | 0 | 0 | 0 | 0 | 0 | 0 | — | 50 | 80 | 10 | 70 | 50 | 90 | 80 | 30 | 70 | 0 | 0 | 0 | 60 | 60 | 40 | 0 |
| Crabgrass, Large | 30 | 90 | 100 | 20 | 90 | 0 | 20 | 70 | 90 | 60 | 70 | 90 | — | 100 | 100 | 100 | 100 | 50 | 100 | 100 | 100 | 100 | 90 | 100 | 100 | 100 | 100 | 100 | 80 |
| Foxtail, Giant | 20 | 60 | 70 | 20 | 70 | 0 | 20 | 60 | 60 | 60 | 50 | 90 | 100 | 100 | 100 | 100 | 100 | 60 | 100 | 100 | 100 | 100 | 90 | 90 | 100 | 100 | 100 | 80 | 50 |
| Italian Ryegrass | — | — | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — | — | — |
| Morningglory | 0 | 20 | 40 | 0 | 40 | 0 | 0 | 0 | 30 | 10 | — | 20 | 40 | 90 | 100 | 50 | 90 | 30 | 90 | 100 | 50 | 100 | 50 | 60 | 60 | 50 | 50 | 30 | 10 |
| Pigweed | 100 | 100 | 100 | 50 | 90 | 10 | 30 | 100 | 100 | 100 | 90 | 100 | 100 | 90 | 100 | 100 | 100 | 100 | 100 | 100 | 100 | 100 | 100 | 100 | 90 | 60 | 100 | 100 | 0 |
| Velvetleaf | 0 | 10 | 20 | 0 | 10 | 0 | 0 | 0 | 20 | 0 | 0 | 0 | 50 | 90 | 80 | 40 | 100 | 60 | 100 | 60 | 80 | 100 | 60 | 50 | 60 | 20 | 10 | 50 | 0 |
| Wheat | 0 | 0 | 10 | 0 | 0 | 0 | 0 | 0 | 0 | 0 | 0 | 0 | — | 30 | 70 | 10 | 80 | 30 | 100 | 90 | 40 | 100 | 20 | 10 | 10 | 60 | 60 | 50 | 0 |

| | 500 g ai/ha | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | | |
| | 86 | 87 | 88 | 89 | 90 | 91 | 92 | 93 | 94 | 95 | 96 | 97 | 98 | 99 | 100 | 101 | 102 | 103 | 104 | 105 | 106 | 107 | 108 | 109 | 110 | 111 | 112 | 113 | 114 |
| Barnyardgrass | 50 | 0 | 0 | 0 | 0 | 0 | 90 | 90 | 0 | 100 | 100 | 100 | 100 | 100 | 100 | 100 | 90 | 100 | 100 | 100 | 100 | 100 | 100 | 100 | 100 | 90 | 10 | 60 | 90 |
| Common Ragweed | — | — | — | — | — | — | — | — | — | — | — | — | — | — | — | — | — | — | — | — | — | — | — | — | — | — | — | — | — |
| Corn | 20 | 0 | 0 | 0 | 40 | 0 | 40 | 80 | 0 | 90 | 90 | 90 | 80 | 90 | 70 | 100 | 60 | 90 | 80 | 70 | 30 | 80 | 90 | 80 | 90 | 0 | 0 | 10 | 40 |
| Crabgrass, Large | 80 | 80 | 30 | 10 | 20 | 20 | 100 | 100 | 0 | 100 | 100 | 100 | 100 | 100 | 100 | 100 | 100 | 100 | 100 | 100 | 100 | 100 | 100 | 100 | 100 | 100 | 90 | 100 | 100 |
| Foxtail, Giant | 80 | 0 | 0 | 10 | 10 | 0 | 100 | 100 | 0 | 100 | 100 | 100 | 100 | 100 | 100 | 100 | 100 | 100 | 100 | 100 | 100 | 100 | 100 | 100 | 100 | 100 | 80 | 100 | 90 |
| Italian Ryegrass | — | — | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — | — | — |
| Morningglory | 20 | 10 | 10 | 0 | 10 | 0 | 20 | 50 | 0 | 100 | 100 | 100 | 100 | 100 | 100 | 100 | 50 | 100 | 100 | 100 | 100 | 100 | 100 | 100 | 100 | 30 | 0 | 70 | 40 |
| Pigweed | 0 | 40 | 0 | 0 | 10 | 40 | 100 | 100 | 0 | 100 | 100 | 100 | 100 | 100 | 100 | 100 | 90 | 100 | 100 | 100 | 100 | 100 | 100 | 100 | 100 | 100 | 50 | 100 | 100 |
| Velvetleaf | 0 | 0 | 0 | 0 | 0 | 0 | 0 | 0 | 0 | 100 | 100 | 100 | 100 | 80 | 70 | 100 | 100 | 100 | 100 | 100 | 100 | 100 | 100 | 100 | 100 | 30 | 10 | 40 | 20 |
| Wheat | 0 | 0 | 0 | 0 | 10 | 0 | 20 | 30 | 0 | 100 | 100 | 100 | 90 | 60 | 20 | 100 | 40 | 100 | 100 | 100 | 30 | 90 | 100 | 90 | 100 | 0 | 0 | 30 | 40 |

500 g ai/ha
Compounds

| | 115 | 116 | 117 | 118 | 119 | 120 | 121 | 122 | 123 | 124 | 125 | 126 | 127 | 128 | 129 | 130 | 131 | 132 | 133 | 134 | 135 | 136 | 137 | 138 | 139 | 140 | 141 |
|---|---|---|---|---|---|---|---|---|---|---|---|---|---|---|---|---|---|---|---|---|---|---|---|---|---|---|---|
| Barnyardgrass | 80 | 100 | 80 | 100 | 70 | 100 | 100 | 100 | 100 | 100 | 100 | 100 | 90 | 100 | 100 | 100 | 100 | 100 | 100 | 90 | 0 | 100 | 70 | 100 | 100 | 100 | 100 |
| Common Ragweed | — | — | — | — | — | — | — | — | 50 | 40 | 90 | 100 | 90 | 90 | — | — | — | — | 90 | 80 | 0 | 80 | 100 | — | — | — | — |
| Corn | 20 | 90 | 10 | 50 | 30 | 50 | 90 | — | — | — | — | — | — | — | 30 | 90 | 90 | 80 | — | — | — | — | — | 90 | 90 | 80 | 80 |
| Crabgrass, Large | 100 | 100 | 100 | 100 | 100 | 100 | 100 | — | — | — | — | — | — | — | 100 | 100 | 100 | 100 | — | — | — | — | — | 100 | 100 | 100 | 100 |
| Foxtail, Giant | 90 | 100 | 100 | 90 | 80 | 90 | 80 | 100 | 100 | 100 | 100 | 100 | 100 | 100 | 100 | 100 | 100 | 100 | 100 | 100 | 50 | 100 | 80 | 100 | 100 | 100 | 100 |
| Italian Ryegrass | — | — | — | — | — | — | — | 30 | 30 | 20 | 100 | 100 | 90 | 100 | — | — | — | — | 90 | 10 | 0 | 100 | 40 | — | — | — | — |
| Kochia | — | — | — | — | — | — | — | 90 | 100 | 90 | 100 | 100 | 100 | 100 | — | — | — | — | 100 | 100 | 100 | 100 | 100 | — | — | — | — |
| Morningglory | 40 | 100 | 100 | 100 | 60 | 100 | 100 | — | — | — | — | — | — | — | 100 | 100 | 100 | 100 | — | — | — | — | — | 100 | 100 | 100 | 100 |
| Pigweed | 100 | 100 | 100 | 100 | 100 | 90 | 100 | 100 | 100 | 100 | 100 | 100 | 100 | 100 | 100 | 100 | 100 | 100 | 100 | 100 | 50 | 100 | 100 | 100 | 100 | 100 | 100 |
| Velvetleaf | 20 | 100 | 0 | 80 | 30 | 70 | 100 | — | — | — | — | — | — | — | 100 | 100 | 100 | 100 | — | — | — | — | — | 100 | 100 | 100 | 50 |
| Wheat | 20 | 100 | 0 | 30 | 20 | 40 | 80 | — | — | — | — | — | — | — | 100 | 90 | 100 | 100 | — | — | — | — | — | 90 | 100 | 100 | 90 |

500 g ai/ha
Compounds

| | 142 | 143 | 144 | 145 | 146 | 147 | 148 | 149 | 150 | 151 | 152 | 153 | 154 | 159 | 160 | 161 | 162 | 163 | 164 | 165 | 166 | 167 | 168 | 170 | 171 | 172 | 173 |
|---|---|---|---|---|---|---|---|---|---|---|---|---|---|---|---|---|---|---|---|---|---|---|---|---|---|---|---|
| Barnyardgrass | 90 | 100 | 100 | 100 | 90 | 30 | 40 | 60 | 0 | 60 | 60 | 100 | 100 | 20 | 10 | 0 | 0 | 60 | 80 | 0 | 100 | 100 | 100 | 0 | 0 | 50 | 70 |
| Common Ragweed | — | — | — | — | — | — | — | — | — | — | — | — | — | — | — | — | — | — | — | — | — | — | — | — | — | — | — |
| Corn | 0 | 80 | 80 | 60 | — | 0 | 0 | 30 | 0 | 0 | 0 | 100 | 30 | 0 | 0 | 0 | 0 | 0 | 50 | 0 | 90 | 40 | 20 | 0 | 0 | 10 | 10 |
| Crabgrass, Large | 100 | 100 | 100 | 100 | 100 | 100 | 100 | 100 | 20 | 100 | 80 | 100 | 100 | 90 | 90 | 70 | 30 | 100 | 90 | 0 | 100 | 100 | 100 | 70 | 0 | 90 | 100 |
| Foxtail, Giant | 100 | 100 | 100 | 100 | 90 | 80 | 90 | 100 | 20 | 100 | 60 | 100 | 100 | 30 | 60 | 20 | 60 | 70 | 70 | 0 | 100 | 100 | 100 | 50 | 0 | 50 | 50 |
| Italian Ryegrass | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — |
| Morningglory | 10 | 100 | 100 | 100 | 100 | 70 | 30 | 100 | 0 | 30 | 20 | 100 | 100 | 0 | 0 | 0 | 0 | 70 | 80 | 0 | 100 | 100 | 100 | 0 | 0 | 40 | 90 |
| Pigweed | 100 | 100 | 100 | 100 | 100 | 100 | 100 | 100 | 100 | 100 | 100 | 100 | 100 | 60 | 90 | 80 | 0 | 100 | 80 | 20 | 100 | 100 | 100 | 60 | 0 | 100 | 70 |
| Velvetleaf | 10 | 100 | 100 | 80 | 60 | 30 | 20 | 90 | 0 | 70 | 20 | 100 | 100 | 0 | 10 | 0 | 0 | 50 | 10 | 0 | 100 | 100 | 100 | 0 | 0 | 20 | 20 |
| Wheat | 10 | 100 | 100 | 100 | 10 | 0 | 0 | 50 | 0 | 10 | 0 | 100 | 100 | 0 | 0 | 0 | 0 | 10 | 20 | 0 | 90 | 50 | 70 | 0 | 0 | 0 | 0 |

500 g ai/ha
Compounds

| | 174 | 175 | 176 | 177 | 178 | 179 | 180 | 181 | 182 | 183 | 184 | 185 | 186 | 187 | 188 | 189 | 190 | 191 | 192 | 195 | 196 | 197 | 198 | 199 | 200 | 201 | 202 |
|---|---|---|---|---|---|---|---|---|---|---|---|---|---|---|---|---|---|---|---|---|---|---|---|---|---|---|---|
| Barnyardgrass | 0 | 100 | 100 | 100 | 100 | 100 | 40 | 10 | 0 | 10 | 100 | 100 | 100 | 100 | 100 | 100 | 60 | 100 | 0 | 100 | 100 | 100 | 100 | 100 | 100 | 100 | 100 |
| Common Ragweed | — | — | — | — | — | — | — | — | — | — | — | — | — | — | — | — | — | — | — | — | — | — | — | — | — | — | — |
| Corn | 0 | 80 | 90 | 90 | 50 | 50 | 0 | 0 | 0 | 0 | 30 | 30 | 100 | 90 | 100 | 100 | 0 | 60 | 0 | 90 | 90 | 90 | 90 | 90 | 90 | 90 | 40 |
| Crabgrass, Large | 40 | 100 | 100 | 100 | 100 | 100 | 100 | 60 | 60 | 80 | 100 | 100 | 100 | 100 | 100 | 100 | 100 | 100 | 10 | 100 | 100 | 100 | 100 | 100 | 100 | 100 | 100 |
| Foxtail, Giant | 20 | 100 | 100 | 100 | 100 | 100 | 90 | 20 | 50 | 50 | 90 | 100 | 100 | 100 | 100 | 100 | 90 | 100 | 0 | 90 | 90 | 100 | 90 | 100 | 100 | 100 | 100 |
| Italian Ryegrass | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — |
| Morningglory | 0 | 100 | 100 | 100 | 100 | 100 | 0 | 0 | 0 | 0 | 100 | 90 | 100 | 100 | 100 | 100 | 30 | 100 | 10 | 100 | 100 | 100 | 100 | 100 | 100 | 100 | 100 |
| Pigweed | 90 | 100 | 100 | 100 | 100 | 100 | 100 | 50 | 30 | 50 | 100 | 0 | 0 | 0 | 100 | 0 | 100 | 100 | 0 | 100 | 100 | 100 | 100 | 100 | 100 | 100 | 100 |
| Velvetleaf | 0 | 100 | 90 | 100 | 90 | 100 | 0 | 0 | 0 | 0 | 50 | 100 | 100 | 100 | 100 | 100 | 20 | 100 | 0 | 100 | 100 | 100 | 100 | 100 | 100 | 100 | 100 |
| Wheat | 0 | 100 | 90 | 100 | 30 | 100 | 0 | 0 | 0 | 0 | 20 | 90 | 100 | 100 | 100 | 100 | 0 | 70 | 0 | 100 | 100 | 50 | 100 | 100 | 100 | 100 | 100 |

500 g ai/ha
Compounds

| | 203 | 204 | 205 | 206 | 207 | 208 | 209 | 210 | 211 | 212 | 213 | 214 | 215 | 216 | 217 | 218 | 219 | 220 | 221 | 222 | 223 | 224 | 225 | 226 | 227 | 228 | 229 |
|---|---|---|---|---|---|---|---|---|---|---|---|---|---|---|---|---|---|---|---|---|---|---|---|---|---|---|---|
| Barnyardgrass | 100 | 80 | 100 | 90 | 90 | 10 | 0 | 0 | 70 | 0 | 0 | 100 | 60 | 80 | 20 | 40 | 20 | 100 | 10 | 90 | 40 | 100 | 80 | 100 | 100 | 90 | 100 |
| Common Ragweed | — | — | 100 | 40 | 40 | 10 | 0 | 0 | 40 | 0 | 0 | — | — | — | — | — | — | — | — | — | — | — | — | — | — | — | — |
| Corn | 80 | 10 | — | — | — | — | — | — | — | — | — | 90 | 0 | 10 | 0 | 0 | 0 | 90 | 0 | 0 | 0 | 90 | 20 | 50 | 50 | 30 | 50 |
| Crabgrass, Large | 100 | 100 | — | — | — | — | — | — | — | — | — | 100 | 100 | 90 | 90 | 80 | 90 | 100 | 90 | 100 | 90 | 100 | 90 | 100 | 100 | 100 | 100 |
| Foxtail, Giant | 100 | 100 | 100 | 100 | 20 | 30 | 0 | 0 | 80 | 0 | 0 | 100 | 70 | 40 | 40 | 40 | 20 | 100 | 40 | 80 | 20 | 100 | 70 | 100 | 100 | 80 | 100 |
| Italian Ryegrass | — | — | 100 | 20 | 20 | 10 | 0 | 0 | 20 | 0 | 0 | — | — | — | — | — | — | — | — | — | — | — | — | — | — | — | — |
| Kochia | — | — | 100 | 100 | 100 | 20 | 0 | 0 | 100 | 20 | 0 | — | — | — | — | — | — | — | — | — | — | — | — | — | — | — | — |
| Morningglory | 100 | 80 | — | — | — | — | — | — | — | — | — | 100 | 10 | 20 | 0 | 10 | 20 | 40 | 10 | 0 | 20 | 100 | 100 | 100 | 100 | 90 | 100 |
| Pigweed | 100 | 100 | 100 | 100 | 100 | 80 | 0 | 0 | 90 | 70 | 0 | 100 | 80 | 70 | 100 | 70 | 100 | 100 | 20 | 100 | 90 | 100 | 90 | 100 | 100 | 100 | 100 |
| Velvetleaf | 100 | 60 | — | — | — | — | — | — | — | — | — | 100 | 10 | 40 | 0 | 0 | 20 | 100 | 10 | 20 | — | 100 | 90 | 100 | 90 | 30 | 100 |
| Wheat | 100 | 0 | — | — | — | — | — | — | — | — | — | 90 | 0 | 0 | 0 | 0 | 0 | 90 | 0 | 0 | 0 | 90 | 20 | 90 | 70 | 0 | 70 |

| | | | | | | | | | | | | | 500 g ai/ha | | | | | | | | | | | | | | | | | | |
|---|---|---|---|---|---|---|---|---|---|---|---|---|---|---|---|---|---|---|---|---|---|---|---|---|---|---|---|---|---|---|---|
| | | | | | | | | | | | | | Compounds | | | | | | | | | | | | | | | | | | |
| | | | | | | | | | | | | | 230 | | 231 | | | | | | | | | | | | | | | | |
| | | | | Barnyardgrass | | | | | | | | | 90 | | 0 | | | | | | | | | | | | | | | | |
| | | | | Common Ragweed | | | | | | | | | — | | — | | | | | | | | | | | | | | | | |
| | | | | Corn | | | | | | | | | 20 | | 0 | | | | | | | | | | | | | | | | |
| | | | | Crabgrass, Large | | | | | | | | | 100 | | 0 | | | | | | | | | | | | | | | | |
| | | | | Foxtail, Giant | | | | | | | | | 90 | | 0 | | | | | | | | | | | | | | | | |
| | | | | Italian Ryegrass | | | | | | | | | — | | — | | | | | | | | | | | | | | | | |
| | | | | Kochia | | | | | | | | | — | | — | | | | | | | | | | | | | | | | |
| | | | | Morningglory | | | | | | | | | 100 | | 0 | | | | | | | | | | | | | | | | |
| | | | | Pigweed | | | | | | | | | 100 | | 0 | | | | | | | | | | | | | | | | |
| | | | | Velvetleaf | | | | | | | | | 80 | | 0 | | | | | | | | | | | | | | | | |
| | | | | Wheat | | | | | | | | | 20 | | 0 | | | | | | | | | | | | | | | | |
| | 125 g ai/ha | | | | | | | | | | | | | | | | | | | | | | | | | | | | | | |
| | Compounds | | | | | | | | | | | | | | | | | | | | | | | | | | | | | | |
| | 3 | 4 | 5 | 6 | 7 | 8 | 9 | 10 | 11 | 12 | 13 | 14 | 21 | 69 | 70 | 71 | 72 | 73 | 75 | 76 | 77 | 78 | 79 | 80 | 81 | 82 | 83 | 84 | 85 | 86 | 87 |
| Barnyardgrass | 0 | 0 | 0 | 0 | 0 | 0 | 0 | 0 | 10 | 0 | 0 | 0 | 0 | 50 | 80 | 60 | 90 | 50 | 100 | 80 | 90 | 100 | 10 | 10 | 20 | 30 | 10 | 10 | 0 | 0 | 0 |
| Common Ragweed | — | — | — | — | — | — | — | — | — | — | — | — | — | — | — | — | — | — | — | — | — | — | — | — | — | — | — | — | — | — | — |
| Corn | 0 | 0 | 0 | 0 | 0 | 0 | 0 | 0 | 0 | 0 | 0 | 0 | 0 | 30 | 50 | 0 | 50 | 30 | 80 | 50 | 10 | 60 | 0 | 0 | 0 | 20 | 10 | 10 | 0 | 0 | 0 |
| Crabgrass, Large | 0 | 40 | 20 | 0 | — | 0 | 0 | 20 | 60 | 40 | 10 | 10 | — | 90 | 100 | 90 | 90 | 30 | 100 | 90 | 100 | 100 | 60 | 50 | 60 | 70 | 100 | 90 | 20 | 50 | 0 |
| Foxtail, Giant | 0 | 0 | 10 | 0 | 10 | 0 | 0 | 10 | 20 | 10 | 0 | 10 | — | 80 | 90 | 80 | 70 | 30 | 100 | 80 | 100 | 100 | 10 | 40 | 30 | 40 | 70 | 40 | 0 | 0 | 0 |
| Italian Ryegrass | — | — | — | — | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — | — | — | — | — |
| Morningglory | 0 | 0 | 0 | 0 | 0 | 0 | 0 | — | 10 | 0 | 0 | 0 | 0 | 70 | 80 | 20 | 60 | 10 | 90 | 70 | 20 | 100 | 0 | 20 | 0 | 10 | 10 | 0 | 0 | 0 | 0 |
| Pigweed | 0 | 50 | 10 | 30 | 0 | 0 | — | 60 | 60 | 70 | — | 100 | — | 60 | 100 | 80 | 100 | 90 | 100 | 100 | 100 | 100 | 30 | 60 | 40 | 0 | 90 | 50 | 0 | 0 | 0 |
| Velvetleaf | 0 | 0 | 0 | 0 | 0 | 0 | 0 | 0 | 0 | 0 | 0 | 0 | — | 70 | 40 | 30 | 70 | 10 | 80 | 20 | 20 | 100 | 0 | 0 | 0 | 0 | 0 | 0 | 0 | 0 | 0 |
| Wheat | 0 | 0 | 0 | 0 | 0 | 0 | 0 | 0 | 0 | 0 | 0 | 0 | 0 | 0 | 50 | 0 | 50 | 0 | 80 | 30 | 20 | 50 | 0 | 0 | 0 | 10 | 0 | 0 | 0 | 0 | 0 |

| | 125 g ai/ha | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | | |
| | 88 | 89 | 90 | 91 | 92 | 93 | 94 | 95 | 96 | 97 | 98 | 99 | 100 | 101 | 102 | 103 | 104 | 105 | 106 | 107 | 108 | 109 | 110 | 111 | 112 | 113 | 114 | 115 | 116 |
| Barnyardgrass | 0 | 0 | 0 | 0 | 0 | 0 | 0 | 100 | 100 | 100 | 90 | 40 | 60 | 100 | 20 | 100 | 100 | 100 | 50 | 100 | 90 | 100 | 100 | 10 | 0 | 20 | 30 | 20 | 100 |
| Common Ragweed | — | — | — | — | — | — | — | — | — | — | — | — | — | — | — | — | — | — | — | — | — | — | — | — | — | — | — | — | — |
| Corn | 0 | 0 | 0 | 0 | 0 | 0 | 0 | 50 | 80 | 90 | 70 | 50 | 10 | 70 | 10 | 70 | 60 | 60 | 20 | 30 | 30 | 60 | 80 | 0 | 0 | 10 | 0 | 0 | 90 |
| Crabgrass, Large | 0 | 0 | 0 | 0 | 20 | 70 | 0 | 100 | 100 | 100 | 100 | 100 | 100 | 100 | 60 | 100 | 100 | 100 | 100 | 100 | 100 | 100 | 100 | 90 | 70 | 80 | 90 | 90 | 100 |
| Foxtail, Giant | 0 | 0 | 0 | 0 | 20 | 30 | 0 | 100 | 100 | 100 | 100 | 90 | 90 | 100 | 90 | 100 | 100 | 100 | 100 | 100 | 100 | 100 | 100 | 90 | 70 | 80 | 70 | 90 | 100 |
| Italian Ryegrass | — | — | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — | — | — |
| Morningglory | 0 | 0 | 0 | 0 | 0 | 0 | 0 | 100 | 100 | 100 | 100 | 60 | 20 | 80 | 10 | 100 | 100 | 100 | 30 | 90 | 100 | 100 | 90 | 0 | 0 | 40 | 0 | 0 | 100 |
| Pigweed | 0 | 0 | 0 | 0 | 40 | 30 | 0 | 100 | 100 | 100 | 100 | 90 | 100 | 100 | 90 | 100 | 100 | 100 | 100 | 100 | 100 | 100 | 100 | 20 | 0 | 100 | 100 | 90 | 100 |
| Velvetleaf | 0 | 0 | 0 | 0 | 0 | 0 | 0 | 90 | 50 | 90 | 100 | 10 | 10 | 40 | 0 | 100 | 100 | 100 | 10 | 90 | 60 | 70 | 90 | 10 | 0 | 30 | 20 | 20 | 100 |
| Wheat | 0 | 0 | 0 | 0 | 0 | 0 | 0 | 70 | 60 | 90 | 30 | 0 | 0 | 70 | 0 | 40 | 90 | 60 | 10 | 10 | 10 | 30 | 80 | 0 | 0 | 10 | 0 | 0 | 100 |

125 g ai/ha
Compounds

| | 117 | 118 | 119 | 120 | 121 | 122 | 123 | 124 | 125 | 126 | 127 | 128 | 129 | 130 | 131 | 132 | 133 | 134 | 135 | 136 | 137 | 138 | 139 | 140 | 141 | 142 | 143 |
|---|---|---|---|---|---|---|---|---|---|---|---|---|---|---|---|---|---|---|---|---|---|---|---|---|---|---|---|
| Barnyardgrass | 30 | 40 | 0 | 20 | 70 | 0 | 0 | 0 | 80 | 90 | 90 | 100 | 90 | 90 | 100 | 100 | 90 | 0 | 0 | 100 | 10 | 100 | 90 | 90 | 70 | 0 | 90 |
| Common Ragweed | — | — | — | — | — | 10 | 10 | 10 | 50 | 90 | 30 | 70 | — | — | — | — | 80 | 10 | 0 | 70 | 10 | — | — | — | — | — | — |
| Corn | 0 | 0 | 0 | 20 | 30 | — | — | — | — | — | — | — | 10 | 0 | 60 | 40 | — | — | — | — | — | 10 | 30 | 10 | 40 | 0 | 50 |
| Crabgrass, Large | 90 | 100 | 70 | 100 | 90 | — | — | — | — | — | — | — | 100 | 100 | 100 | 100 | — | — | — | — | — | 100 | 100 | 100 | 100 | 100 | 100 |
| Foxtail, Giant | 70 | 70 | 20 | 60 | 60 | 20 | 20 | 20 | 80 | 100 | 90 | 100 | 100 | 100 | 90 | 100 | 100 | 20 | 0 | 100 | 20 | 100 | 100 | 100 | 90 | 50 | 100 |
| Italian Ryegrass | — | — | — | — | — | 0 | 0 | 0 | 20 | 90 | 40 | 30 | — | — | — | — | 40 | 0 | 0 | 30 | 0 | — | — | — | — | — | — |
| Kochia | — | — | — | — | — | 10 | 10 | 20 | 40 | 100 | 100 | 100 | — | — | — | — | 100 | 80 | 70 | 100 | 30 | — | — | — | — | — | — |
| Morningglory | 30 | 70 | 20 | 70 | 100 | — | — | — | — | — | — | — | 100 | 100 | 100 | 100 | — | — | — | — | — | 90 | 40 | 80 | 50 | 0 | 100 |
| Pigweed | 90 | 100 | 80 | 70 | 100 | 60 | 70 | 20 | 100 | 100 | 90 | 100 | 100 | 100 | 100 | 100 | 100 | 20 | 0 | 100 | 60 | 100 | 100 | 100 | 100 | 50 | 100 |
| Velvetleaf | 0 | 0 | 0 | 10 | 20 | — | — | — | — | — | — | — | 100 | 70 | 90 | 100 | — | — | — | — | — | 100 | 30 | 60 | 10 | 0 | 50 |
| Wheat | 0 | 0 | 0 | 0 | 10 | — | — | — | — | — | — | — | 50 | 10 | 90 | 80 | — | — | — | — | — | 80 | 50 | 70 | 30 | 0 | 70 |

125 g ai/ha
Compounds

| | 144 | 145 | 146 | 147 | 148 | 149 | 150 | 151 | 152 | 153 | 154 | 159 | 160 | 161 | 162 | 163 | 164 | 165 | 166 | 167 | 168 | 170 | 171 | 172 | 173 | 174 | 175 |
|---|---|---|---|---|---|---|---|---|---|---|---|---|---|---|---|---|---|---|---|---|---|---|---|---|---|---|---|
| Barnyardgrass | 100 | 90 | 0 | 0 | 10 | 10 | 0 | 0 | 0 | 90 | 90 | 0 | 0 | 0 | 0 | 10 | 10 | 0 | 50 | 30 | 20 | 0 | 0 | 0 | 0 | 0 | 100 |
| Common Ragweed | — | — | — | — | — | — | — | — | — | — | — | — | — | — | — | — | — | — | — | — | — | — | — | — | — | — | — |
| Corn | 10 | 30 | 0 | 0 | 0 | 0 | 0 | 0 | 0 | 90 | 0 | 0 | 0 | 0 | 0 | 0 | 0 | 0 | 10 | 0 | 0 | 0 | 0 | 0 | 0 | 0 | 50 |
| Crabgrass, Large | 100 | 100 | 80 | 50 | 50 | 100 | 0 | 80 | 40 | 100 | 100 | 10 | 50 | 10 | 0 | 50 | 80 | 0 | 100 | 80 | 100 | 10 | 0 | 50 | 60 | 10 | 100 |
| Foxtail, Giant | 100 | 100 | 10 | 10 | 10 | 70 | 0 | 50 | 10 | 100 | 100 | 0 | 0 | 0 | 0 | 20 | 10 | 0 | 100 | 90 | 90 | 0 | 0 | 20 | 0 | 0 | 100 |
| Italian Ryegrass | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — |
| Morningglory | 80 | 100 | 20 | 20 | 0 | 10 | 0 | 0 | 0 | 100 | 100 | 0 | 0 | 0 | 0 | 0 | 0 | 0 | 100 | 50 | 80 | 0 | 0 | 30 | 10 | 0 | 100 |
| Pigweed | 100 | 100 | 100 | 90 | 100 | 80 | 50 | 70 | 100 | 100 | 100 | 10 | 50 | 10 | 0 | 30 | 20 | 10 | 100 | 100 | 100 | 0 | 0 | 80 | 10 | 20 | 100 |
| Velvetleaf | 50 | 50 | 10 | 0 | 0 | 20 | 0 | 0 | 0 | 90 | 90 | 0 | 0 | 0 | 0 | 10 | 0 | 0 | 60 | 30 | 20 | 0 | 0 | 10 | 0 | 0 | 40 |
| Wheat | 50 | 40 | 0 | 0 | 0 | 0 | 0 | 0 | 0 | 100 | 0 | 0 | 0 | 0 | 0 | 0 | 0 | 0 | 10 | 0 | 10 | 0 | 0 | 0 | 0 | 0 | 20 |

125 g ai/ha
Compounds

| | 176 | 177 | 178 | 179 | 180 | 181 | 182 | 183 | 184 | 185 | 186 | 187 | 188 | 189 | 190 | 191 | 192 | 195 | 196 | 197 | 198 | 199 | 200 | 201 | 202 | 203 | 204 |
|---|---|---|---|---|---|---|---|---|---|---|---|---|---|---|---|---|---|---|---|---|---|---|---|---|---|---|---|
| Barnyardgrass | 80 | 100 | 80 | 100 | 0 | 0 | 0 | 0 | 0 | 80 | 100 | 90 | 100 | 90 | 0 | 20 | 0 | 90 | 90 | 90 | 100 | 100 | 90 | 100 | 90 | 80 | 0 |
| Common Ragweed | — | — | — | — | — | — | — | — | — | — | — | — | — | — | — | — | — | — | — | — | — | — | — | — | — | — | — |
| Corn | 10 | 80 | 0 | 10 | 0 | 0 | 0 | 0 | 0 | 10 | 80 | 40 | 100 | 80 | 0 | 10 | 0 | 0 | 10 | 0 | 0 | 70 | 60 | 10 | 10 | 20 | 0 |
| Crabgrass, Large | 100 | 100 | 100 | 100 | 30 | 10 | 0 | 10 | 50 | 100 | 100 | 100 | 100 | 100 | 80 | 100 | 0 | 100 | 100 | 100 | 100 | 100 | 100 | 100 | 100 | 100 | 100 |
| Foxtail, Giant | 90 | 100 | 90 | 100 | 10 | 0 | 0 | 10 | 60 | 100 | 100 | 100 | 100 | 100 | 20 | 100 | 0 | 90 | 100 | 100 | 100 | 100 | 100 | 100 | 100 | 80 | 50 |
| Italian Ryegrass | — | — | — | — | — | — | — | — | — | — | — | — | — | — | — | — | — | — | — | — | — | — | — | — | — | — | — |
| Kochia | — | — | — | — | — | — | — | — | — | — | — | — | — | — | — | — | — | — | — | — | — | — | — | — | — | — | — |
| Morningglory | 80 | 100 | 80 | 80 | 0 | 0 | 0 | 0 | 20 | 40 | 100 | 100 | 100 | 100 | 0 | 90 | 0 | 90 | 100 | 100 | 100 | 90 | 40 | 100 | 100 | 10 | 30 |
| Pigweed | 100 | 100 | 100 | 100 | 10 | 10 | 0 | 10 | 60 | 0 | 0 | 0 | 100 | 0 | 100 | 100 | 0 | 100 | 100 | 100 | 100 | 100 | 100 | 100 | 100 | 100 | 100 |
| Velvetleaf | 50 | 40 | 30 | 50 | 0 | 0 | 0 | 0 | 0 | 70 | 100 | 100 | 100 | 100 | 0 | 10 | 0 | 80 | 80 | 70 | 30 | 100 | 20 | 100 | 30 | 30 | 10 |
| Wheat | 20 | 10 | 0 | 10 | 0 | 0 | 0 | 0 | 0 | 70 | 90 | 80 | 100 | 90 | 0 | 0 | 0 | 80 | 20 | 0 | 60 | 90 | 0 | 80 | 30 | 80 | 0 |

125 g ai/ha
Compounds

| | 205 | 206 | 207 | 208 | 209 | 210 | 211 | 212 | 213 | 214 | 215 | 216 | 217 | 218 | 219 | 220 | 221 | 222 | 223 | 224 | 225 | 226 | 227 | 228 | 229 | 230 | 231 |
|---|---|---|---|---|---|---|---|---|---|---|---|---|---|---|---|---|---|---|---|---|---|---|---|---|---|---|---|
| Barnyardgrass | 90 | 0 | 0 | 0 | 0 | 0 | 0 | 0 | 0 | 100 | 0 | 0 | 0 | 0 | 0 | 80 | 0 | 0 | 0 | 90 | 10 | 80 | 40 | 10 | 50 | 10 | 0 |
| Common Ragweed | 70 | 0 | 0 | 0 | 0 | 0 | 0 | 0 | 0 | — | — | — | — | — | — | — | — | — | — | — | — | — | — | — | — | — | — |
| Corn | — | — | — | — | — | — | — | — | — | 10 | 0 | 0 | 0 | 0 | 0 | 0 | 0 | 0 | 0 | 30 | 0 | 30 | 0 | 0 | 10 | 0 | 0 |
| Crabgrass, Large | — | — | — | — | — | — | — | — | — | 100 | 50 | 60 | 50 | 10 | 40 | 100 | 10 | 80 | 20 | 100 | 20 | 100 | 100 | 90 | 100 | 90 | 0 |
| Foxtail, Giant | 100 | 0 | 0 | 0 | 0 | 0 | 0 | 0 | 0 | 100 | 10 | 10 | 0 | 0 | 0 | 100 | 0 | 0 | 0 | 100 | 20 | 100 | 70 | 30 | 90 | 60 | 0 |
| Italian Ryegrass | 50 | 0 | 0 | 0 | 0 | 0 | 0 | 0 | 0 | — | — | — | — | — | — | — | — | — | — | — | — | — | — | — | — | — | — |
| Kochia | 100 | 50 | 30 | 10 | 0 | 0 | 50 | 0 | 0 | — | — | — | — | — | — | — | — | — | — | — | — | — | — | — | — | — | — |
| Morningglory | — | — | — | — | — | — | — | — | — | 80 | 0 | 0 | 0 | 0 | 0 | 20 | 0 | 0 | 0 | 30 | 30 | 100 | 10 | 30 | 50 | 50 | 0 |
| Pigweed | 100 | 90 | 50 | 20 | 0 | 0 | 50 | 20 | 0 | 100 | 10 | 0 | 20 | 0 | 0 | 100 | 0 | 20 | 0 | 100 | 20 | 100 | 90 | 90 | 100 | 90 | 0 |
| Velvetleaf | — | — | — | — | — | — | — | — | — | 100 | 0 | 0 | 0 | 0 | 0 | — | 0 | 0 | 0 | 100 | 0 | 60 | 30 | 0 | 40 | 10 | 0 |
| Wheat | — | — | — | — | — | — | — | — | — | 80 | 0 | 0 | 0 | 0 | 0 | 0 | 0 | 0 | 0 | 30 | 0 | 40 | 10 | 0 | 20 | 0 | 0 |

### Test B

Seeds selected from rice (Oryza sativa), sedge, umbrella (small-flower umbrella sedge Cyperus difformis), ducksalad (Heteranthera limosa), and barnyardgrass (Echinochloa crus-galli) were grown to the 2-leaf stage for testing. At time of treatment, test pots were flooded to 3 cm above the soil surface, treated by application of test compounds directly to the paddy water, and then maintained at that water depth for the duration of the test.

Treated plants and controls were maintained in a greenhouse for 13 to 15 days, after which time all species were compared to controls and visually evaluated. Plant response ratings, summarized in Table B, are based on a scale of 0 to 100 where 0 is no effect and 100 is complete control. A dash (-) response means no test result.

TABLE B
Flood

| | 250 g ai/ha | | | | | | | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | | | | | | | | | |
| | 74 | 75 | 76 | 77 | 78 | 79 | 80 | 81 | 82 | 83 | 84 | 85 | 86 | 87 | 88 | 89 | 90 | 91 | 92 | 93 | 94 | 95 | 96 | 97 | 98 | 99 | 100 | 101 | 102 | 103 | 104 | 105 | 106 | 107 | 108 | 109 |
| Barnyardgrass | — | 40 | 20 | 0 | 50 | 0 | 0 | 0 | 0 | 0 | 0 | 0 | 0 | 0 | 0 | 0 | 0 | 0 | 0 | 0 | 0 | 40 | 40 | 45 | 0 | 0 | 0 | 0 | 0 | 40 | 75 | 90 | 30 | 40 | 40 | 0 |
| Ducksalad | 75 | 85 | 85 | 80 | 85 | 0 | 0 | 0 | 30 | 60 | 20 | 0 | 0 | 0 | 0 | 0 | 0 | 0 | 50 | 50 | 0 | 85 | 75 | 70 | 70 | 50 | 30 | 70 | 50 | 90 | 0 | 95 | 90 | 70 | 60 | 0 |
| Rice | 0 | 35 | 20 | 20 | 50 | 0 | 0 | 0 | 0 | 10 | 0 | 0 | 0 | 0 | 0 | 0 | 0 | 0 | 0 | 0 | 0 | 20 | 20 | 45 | 0 | 0 | 10 | 0 | 0 | 50 | 40 | 90 | 20 | 30 | 15 | 0 |
| Sedge, Umbrella | 65 | 85 | 80 | 80 | 80 | 0 | 0 | 0 | 75 | 70 | 60 | 0 | 40 | 0 | 0 | 0 | 0 | 0 | 50 | 50 | 0 | 75 | 75 | 70 | 80 | 75 | 30 | 60 | 50 | 90 | 0 | 95 | 90 | 80 | 70 | 0 |

250 g ai/ha
Compounds

| | 110 | 111 | 112 | 113 | 114 | 115 | 116 | 117 | 118 | 119 | 120 | 121 | 122 | 123 | 124 | 125 | 126 | 127 | 128 | 129 | 130 | 131 | 132 | 133 | 134 | 135 | 136 |
|---|---|---|---|---|---|---|---|---|---|---|---|---|---|---|---|---|---|---|---|---|---|---|---|---|---|---|---|
| Barnyardgrass | 30 | 15 | 20 | 40 | 20 | 0 | 65 | 0 | 0 | 0 | 0 | 15 | 20 | 20 | 10 | 20 | 40 | 70 | 70 | 40 | 40 | 50 | 60 | 40 | 0 | 0 | 50 |
| Ducksalad | 80 | 0 | 0 | 60 | 40 | 0 | 100 | 50 | 30 | 40 | 40 | 75 | 50 | 60 | 30 | 60 | 95 | 95 | 95 | 95 | 90 | 90 | 100 | 90 | 0 | 0 | 80 |
| Rice | 35 | 20 | 20 | 30 | 15 | 20 | 55 | 20 | 0 | 0 | 0 | 20 | 30 | 10 | 20 | 10 | 20 | 20 | 30 | 40 | 15 | 40 | 35 | 20 | 0 | 0 | 35 |
| Sedge, Umbrella | 80 | 90 | 0 | 70 | 70 | 80 | 100 | 75 | 85 | 75 | 80 | 75 | 50 | 60 | 20 | 50 | 95 | 85 | 90 | 95 | 90 | 90 | 90 | 90 | 0 | 0 | 80 |

250 g ai/ha
Compounds

| | 137 | 138 | 139 | 140 | 141 | 143 | 144 | 145 | 146 | 147 | 148 | 149 | 150 | 151 | 152 | 153 | 154 | 159 | 160 | 161 | 162 | 163 | 164 | 165 | 166 | 167 | 168 |
|---|---|---|---|---|---|---|---|---|---|---|---|---|---|---|---|---|---|---|---|---|---|---|---|---|---|---|---|
| Barnyardgrass | 0 | 50 | 60 | 55 | 45 | 45 | 30 | 45 | 0 | 0 | 0 | 30 | 0 | 0 | 0 | 45 | 45 | 0 | 0 | 0 | 0 | 0 | 0 | 0 | 10 | 20 | 20 |
| Ducksalad | 30 | 80 | 100 | 95 | 100 | 100 | 95 | 100 | 0 | 0 | 30 | 95 | 0 | 0 | 0 | 90 | 90 | 0 | 0 | 0 | 0 | 0 | 0 | 0 | 85 | 80 | 90 |
| Rice | 0 | 50 | 50 | 40 | 20 | 25 | 0 | 25 | 0 | 0 | 15 | 10 | 15 | 0 | 0 | 40 | 0 | 0 | 0 | 0 | 0 | 0 | 0 | 0 | 10 | 20 | 20 |
| Sedge, Umbrella | 30 | 90 | 100 | 95 | 95 | 95 | 95 | 95 | 0 | 0 | 40 | 95 | 65 | 60 | 0 | 95 | 95 | 0 | 0 | 0 | 0 | 0 | 0 | 0 | 80 | 70 | 90 |

250 g ai/ha
Compounds

| | 170 | 171 | 172 | 173 | 174 | 175 | 176 | 177 | 178 | 179 | 180 | 181 | 182 | 183 | 184 | 185 | 186 | 187 | 188 | 189 | 190 | 191 | 192 | 195 | 196 | 197 | 198 |
|---|---|---|---|---|---|---|---|---|---|---|---|---|---|---|---|---|---|---|---|---|---|---|---|---|---|---|---|
| Barnyardgrass | 0 | 0 | 0 | 0 | 0 | 35 | 35 | 60 | 20 | 25 | 0 | 0 | 0 | 0 | 0 | 20 | 75 | 55 | 75 | 60 | 0 | 0 | 0 | 10 | 30 | 10 | 60 |
| Ducksalad | 0 | 0 | 0 | 0 | 0 | 100 | 100 | 95 | 95 | 100 | 0 | 0 | 0 | 0 | 0 | 50 | 80 | 90 | 90 | 85 | 0 | 80 | 0 | 95 | 80 | 95 | 100 |
| Rice | 0 | 0 | 0 | 0 | 0 | 25 | 0 | 15 | 0 | 0 | 0 | 20 | 0 | 0 | 0 | 10 | 75 | 45 | 60 | 45 | 0 | 0 | 0 | 10 | 20 | 10 | 60 |
| Sedge, Umbrella | 0 | 0 | 0 | 0 | 0 | 85 | 80 | 95 | 85 | 80 | 0 | 40 | 0 | 0 | 0 | 80 | 80 | 90 | 90 | 90 | 45 | 55 | 0 | 95 | 70 | 85 | 100 |

250 g ai/ha
Compounds

| | 199 | 200 | 201 | 202 | 203 | 204 | 205 | 206 | 207 | 208 | 209 | 210 | 211 | 212 | 213 | 214 | 215 | 216 | 217 | 218 | 219 | 220 | 221 | 222 | 223 | 224 | 225 |
|---|---|---|---|---|---|---|---|---|---|---|---|---|---|---|---|---|---|---|---|---|---|---|---|---|---|---|---|
| Barnyardgrass | 55 | 25 | 70 | 30 | 25 | 0 | 50 | 0 | 0 | 0 | 0 | 0 | 0 | 0 | 0 | 50 | 0 | 0 | 0 | 0 | 0 | 0 | 0 | 0 | 0 | 20 | 0 |
| Ducksalad | 90 | 80 | 95 | 95 | 95 | 95 | 100 | 0 | 0 | 0 | 0 | 0 | 0 | 0 | 0 | 90 | 30 | 20 | 0 | 0 | 30 | 65 | 0 | 55 | 25 | 75 | 55 |
| Rice | 45 | 0 | 45 | 30 | 0 | 0 | 0 | 0 | 0 | 0 | 0 | 0 | 0 | 0 | 0 | 60 | 10 | 0 | 0 | 0 | 0 | 15 | 0 | 15 | 25 | 20 | 0 |
| Sedge, Umbrella | 90 | 90 | 95 | 80 | 95 | 90 | 100 | 0 | 0 | 0 | 0 | 0 | 0 | 0 | 0 | 95 | 20 | 30 | 0 | 0 | 75 | 70 | 0 | 65 | 65 | 90 | 50 |

250 g ai/ha
Compounds

| | 226 | 227 | 228 | 229 | 230 | 231 |
|---|---|---|---|---|---|---|
| Barnyardgrass | 50 | 80 | 0 | 60 | 10 | 0 |
| Ducksalad | 90 | 100 | 30 | 100 | 70 | 0 |
| Rice | 30 | 0 | 0 | 40 | 10 | 0 |
| Sedge, Umbrella | 90 | 95 | 30 | 95 | 50 | 0 |

### Test C

Seeds of plant species selected from blackgrass (Alopecurus myosuroides), ryegrass, Italian (Italian ryegrass, Lolium multiflorum), wheat (winter wheat, Triticum aestivum), galium (catchweed bedstraw, Galium aparine), corn (Zea mays), crabgrass, large (large crabgrass, Digitaria sanguinalis), foxtail, giant (giant foxtail, Setaria faberii), johnsongrass (Sorghum halepense), lambsquarters (Chenopodium album), morningglory (Ipomoea coccinea), nutsedge, yellow (yellow nutsedge, Cyperus esculentus), pigweed (redroot pigweed, Amaranthus retroflexus), ragweed (common ragweed, Ambrosia elation), soybean (Glycine max), barnyardgrass (Echinochloa crus-galli), oilseed rape (Brassica napus), waterhemp (common waterhemp, Amaranthus rudis), oat, wild (wild oat, Avena fatua), kochia (Kochia scoparia), Surinam grass (Brachiaria decumbens), foxtail, green (green foxtail, Setaria viridis), goosegrass (Eleusine indica), bromegrass, downy (downy bromegrass, Bromus tectorum), nightshade (eastern black nightshade, Solanum ptycanthum), cocklebur (common cocklebur, Xanthium strumarium), cupgrass, woolly (woolly cupgrass, Eriochloa villosa), bermudagrass (Cynodon dactylon), sunflower, (common oilseed sunflower, Helianthus annuus), Russian thistle (Salsola kali), and velvetleaf (Abutilon theophrasti) were planted into a blend of loam soil and sand and treated preemergence with test chemicals formulated in a non-phytotoxic solvent mixture which included a surfactant.

At the same time, plants selected from these crop and weed species and also chickweed (common chickweed, Stellaria media), canarygrass (littleseed canarygrass, Phalaris minor), and deadnettle (henbit deadnettle, Lamium amplexicaule), were planted in pots containing Redi-Earth® planting medium (Scotts Company, 14111 Scottslawn Road, Marysville, Ohio 43041) comprising spaghnum peat moss, vermiculite, wetting agent and starter nutrients and treated with postemergence applications of test chemicals formulated in the same manner. Plants ranged in height from 2 to 18 cm (1- to 4-leaf stage) for postemergence treatments. Treated plants and controls were maintained in a greenhouse for 13 to 15 days, after which time all species were compared to controls and visually evaluated. Plant response ratings, summarized in Table C, are based on a scale of 0 to 100 where 0 is no effect and 100 is complete control. A dash (-) response means no test result.

Plant species in the flooded paddy test consisted of rice (Oryza sativa), sedge, umbrella (small-flower umbrella sedge, Cyperus difformis), ducksalad (Heteranthera limosa), and barnyardgrass (Echinochloa crus-galli) grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 to 15 days, after which time all species were compared to controls and visually evaluated. Plant response ratings, summarized in Table C, are based on a scale of 0 to 100 where 0 is no effect and 100 is complete control. A dash (-) response means no test result.

TABLE C
Postemergence

| | 250 g ai/ha | | | | | | | | | | | | | | | | | | | | | | | | | | | |
|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | |
| | 20 | 27 | 69 | 70 | 72 | 74 | 76 | 78 | 95 | 96 | 97 | 98 | 103 | 104 | 105 | 107 | 108 | 109 | 110 | 116 | 126 | 127 | 128 | 129 | 130 | 131 | 132 | 133 |
| Barley | 30 | 30 | 25 | 25 | 10 | 5 | — | — | — | — | — | — | — | — | — | — | — | — | — | — | — | — | — | — | — | — | — | — |
| Barnyardgrass | — | — | — | — | — | — | 60 | 80 | 30 | 30 | 45 | 35 | 95 | 90 | 85 | 30 | 35 | 20 | 65 | 20 | 40 | 15 | 60 | 55 | 10 | 45 | 50 | 15 |
| Bermudagrass | 45 | 25 | 25 | 10 | 20 | 5 | — | — | — | — | — | — | — | — | — | — | — | — | — | — | — | — | — | — | — | — | — | — |
| Blackgrass | 20 | 20 | 60 | 25 | 40 | 5 | 10 | 50 | 35 | 20 | 50 | 40 | 60 | 80 | 60 | 35 | 30 | 15 | 60 | 10 | 25 | 40 | 80 | 20 | 10 | 20 | 25 | 10 |
| Bromegrass, Downy | 10 | 10 | 35 | 5 | 15 | 0 | — | — | — | — | — | — | — | — | — | — | — | — | — | — | — | — | — | — | — | — | — | — |
| Canarygrass | 15 | 10 | 45 | 60 | 30 | 5 | — | — | — | — | — | — | — | — | — | — | — | — | — | — | — | — | — | — | — | — | — | — |
| Chickweed | 75 | 75 | 95 | 65 | 80 | 55 | 85 | 85 | 100 | 100 | 100 | 100 | 85 | 90 | 95 | 100 | 100 | 65 | 95 | 95 | 85 | 80 | 95 | 100 | 75 | 80 | 85 | 85 |
| Cocklebur | 40 | 40 | 20 | 45 | 15 | 55 | — | — | — | — | — | — | — | — | — | — | — | — | — | — | — | — | — | — | — | — | — | — |
| Corn | 25 | 15 | 60 | 50 | 60 | 55 | — | 80 | 10 | 45 | 60 | 10 | 95 | 95 | 85 | 40 | 35 | 15 | 25 | 30 | 55 | 25 | 30 | 15 | 25 | 50 | 20 | 35 |
| Crabgrass, Large | 45 | 45 | 65 | 45 | 25 | 75 | 70 | 90 | 45 | 55 | 85 | 50 | 85 | 95 | 95 | 85 | 50 | 45 | 60 | 75 | 60 | 55 | 85 | 20 | 25 | 40 | 55 | 35 |
| Cupgrass, Woolly | 15 | 20 | 50 | 20 | 15 | 20 | — | — | — | — | — | — | — | — | — | — | — | — | — | — | — | — | — | — | — | — | — | — |
| Deadnettle | — | — | 90 | 90 | 70 | 80 | — | — | — | — | — | — | — | — | — | — | — | — | — | — | — | — | — | — | — | — | — | — |
| Foxtail, Giant | 15 | 20 | 65 | 25 | 15 | 25 | 85 | 95 | 60 | 85 | 80 | 85 | 90 | 98 | 85 | 60 | 45 | 50 | 70 | 60 | 60 | 35 | 85 | 45 | 55 | 50 | 55 | 80 |
| Foxtail, Green | 60 | 50 | 85 | 80 | 40 | 5 | — | — | — | — | — | — | — | — | — | — | — | — | — | — | — | — | — | — | — | — | — | — |
| Galium | — | — | 98 | 90 | 80 | 85 | 90 | 80 | 90 | 90 | 90 | 90 | 80 | 85 | 90 | 90 | 90 | 80 | — | 90 | 70 | 80 | 70 | 80 | 75 | 70 | 75 | 80 |
| Goosegrass | 15 | 10 | 20 | 15 | 5 | 10 | — | — | — | — | — | — | — | — | — | — | — | — | — | — | — | — | — | — | — | — | — | — |
| Johnsongrass | 10 | 5 | 80 | 75 | 0 | 15 | 25 | 75 | 30 | 40 | 35 | 20 | 95 | 85 | 75 | 70 | 5 | 10 | 90 | 30 | 25 | 25 | 10 | 10 | 10 | 10 | 75 | 5 |
| Kochia | 98 | 95 | 85 | 70 | 55 | 90 | 90 | 90 | 85 | 85 | 90 | 85 | 90 | 90 | 95 | 90 | 90 | 90 | 90 | 90 | 65 | 90 | 90 | 90 | 90 | 75 | 90 | 90 |
| Lambsquarters | 75 | 65 | 100 | 98 | 98 | 90 | 85 | 98 | 98 | 95 | 90 | 95 | 90 | 95 | 98 | 90 | 85 | 85 | 98 | 85 | 90 | 85 | 98 | 98 | 75 | 85 | 85 | 90 |
| Morningglory | 70 | 75 | 65 | 85 | 65 | 80 | 98 | 100 | 70 | 60 | 90 | 90 | 100 | 98 | 95 | 80 | 90 | 85 | — | 90 | 80 | 85 | 80 | 100 | 98 | 70 | 98 | 100 |
| Nutsedge, Yellow | 0 | 0 | 45 | 20 | 0 | 15 | 5 | 15 | 10 | 15 | 30 | 5 | 20 | 60 | 40 | 40 | 25 | 5 | 10 | 10 | 15 | 25 | 25 | 5 | 10 | 20 | 10 | 15 |
| Oat, Wild | 20 | 15 | 40 | 35 | 35 | 5 | 25 | 30 | 50 | 50 | 40 | 25 | 50 | 85 | 60 | 40 | 20 | 5 | 35 | 30 | 50 | 35 | 20 | 35 | 15 | 10 | 25 | 35 |
| Oilseed Rape | — | — | — | — | — | — | 60 | 55 | 50 | 20 | 60 | 30 | 70 | 85 | 90 | 80 | 55 | 50 | 90 | 40 | 70 | 85 | 90 | 70 | 60 | 60 | 65 | 85 |
| Pigweed | 80 | 50 | 85 | 75 | 65 | 80 | 98 | 98 | 70 | 60 | 90 | 90 | 80 | 98 | 90 | 85 | 90 | 65 | 98 | 85 | 65 | 85 | 90 | 95 | 85 | 98 | 60 | 90 |
| Ragweed | 25 | 10 | 95 | 65 | 35 | 70 | 90 | 95 | 85 | 85 | 90 | 75 | 90 | 95 | 80 | 90 | 85 | 80 | 80 | 85 | 80 | 60 | 85 | 85 | 85 | 85 | 90 | 85 |
| Ryegrass, Italian | 25 | 15 | 60 | 50 | 40 | 0 | 10 | 35 | 30 | 30 | 45 | 10 | 45 | 80 | 45 | 5 | 20 | 10 | 40 | 10 | 15 | 40 | 30 | 5 | 15 | 10 | 10 | 10 |
| Soybean | 75 | 75 | 75 | 65 | 40 | 85 | 98 | 85 | — | 85 | 90 | 85 | 98 | 95 | 95 | 90 | 98 | 75 | 85 | 95 | 65 | 85 | 98 | 98 | 98 | 80 | 98 | 95 |
| Surinam Grass | 10 | 10 | 60 | 30 | 15 | 25 | — | — | — | — | — | — | — | — | — | — | — | — | — | — | — | — | — | — | — | — | — | — |
| Velvetleaf | 60 | 45 | 75 | 55 | 55 | 40 | 80 | 75 | 40 | 30 | 60 | 50 | 55 | 80 | 75 | 50 | 40 | 35 | 75 | 60 | 70 | 70 | 65 | — | 60 | 55 | 50 | 70 |
| Waterhemp | — | — | — | — | — | — | 100 | 95 | 30 | 75 | 90 | 90 | 90 | 98 | 85 | 80 | 80 | 75 | 100 | 65 | 90 | 70 | 95 | 90 | 80 | 80 | 80 | 95 |
| Wheat | 25 | 20 | 25 | 25 | 10 | 5 | 5 | 35 | 20 | 30 | 20 | 20 | 15 | 25 | 45 | 30 | 20 | 0 | 5 | 15 | 5 | 10 | 5 | 5 | 5 | 10 | 15 | 30 |
| Windgrass | 45 | 45 | 85 | 50 | 30 | 5 | — | — | — | — | — | — | — | — | — | — | — | — | — | — | — | — | — | — | — | — | — | — |

| | 250 g ai/ha | | | | | | | | | | | | | | | | | | | | | | | | 125 g ai/ha | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | | Compounds | | |
| | 136 | 138 | 139 | 144 | 153 | 154 | 177 | 185 | 186 | 187 | 188 | 189 | 196 | 197 | 198 | 199 | 200 | 201 | 202 | 205 | 206 | 214 | 220 | 224 | 20 | 27 | 69 |
| Barley | — | — | — | — | — | — | — | — | — | — | — | — | — | — | — | — | — | — | — | — | — | — | — | — | 30 | 20 | 25 |
| Barnyardgrass | 45 | 65 | 20 | 15 | 15 | 10 | 20 | 20 | 55 | 60 | 65 | 55 | 35 | 10 | 35 | 45 | 10 | 60 | 25 | 50 | 5 | 65 | 15 | 10 | — | — | — |
| Bermudagrass | — | — | — | — | — | — | — | — | — | — | — | — | — | — | — | — | — | — | — | — | — | — | — | — | 15 | 5 | 20 |
| Blackgrass | 40 | 50 | 35 | 35 | 5 | 40 | 35 | 5 | 60 | 80 | 70 | 50 | 15 | 15 | 30 | 30 | 15 | 60 | 30 | 30 | 5 | 50 | 20 | 20 | 15 | 15 | 35 |
| Bromegrass, Downy | — | — | — | — | — | — | — | — | — | — | — | — | — | — | — | — | — | — | — | — | — | — | — | — | 5 | 5 | 20 |
| Canarygrass | — | — | — | — | — | — | — | — | — | — | — | — | — | — | — | — | — | — | — | — | — | — | — | — | 15 | 10 | 30 |
| Chickweed | 100 | 90 | 80 | 90 | 80 | 90 | 90 | 85 | 100 | 85 | 98 | 90 | 80 | 80 | 80 | 80 | 90 | 85 | 85 | 100 | 75 | 85 | 80 | 90 | 75 | 40 | — |
| Cocklebur | — | — | — | — | — | — | — | — | — | — | — | — | — | — | — | — | — | — | — | — | — | — | — | — | 35 | 35 | 20 |
| Corn | 25 | 30 | 20 | 25 | 20 | 10 | 15 | 65 | 65 | 65 | 60 | 60 | 15 | 10 | 15 | 65 | 20 | 60 | 25 | 40 | 5 | 10 | 10 | 10 | 20 | 15 | 45 |
| Crabgrass, Large | 40 | 70 | 20 | 55 | 45 | 10 | 10 | 45 | 80 | 95 | 95 | 90 | 25 | 15 | 30 | 60 | 25 | 50 | 70 | 65 | 5 | 55 | 10 | 20 | 35 | 10 | 30 |
| Cupgrass, Woolly | — | — | — | — | — | — | — | — | — | — | — | — | — | — | — | — | — | — | — | — | — | — | — | — | 15 | 10 | 45 |
| Deadnettle | — | — | — | — | — | — | — | — | — | — | — | — | — | — | — | — | — | — | — | — | — | — | — | — | — | — | 85 |
| Foxtail, Giant | 70 | 50 | 65 | 25 | 65 | 10 | 45 | 5 | 75 | 70 | 90 | 75 | 20 | 10 | 55 | 50 | 15 | 85 | 35 | 65 | 5 | 20 | 5 | 5 | 10 | 10 | 40 |
| Foxtail, Green | — | — | — | — | — | — | — | — | — | — | — | — | — | — | — | — | — | — | — | — | — | — | — | — | 55 | 30 | 70 |
| Galium | 80 | 80 | 70 | 85 | 80 | 85 | 90 | 70 | 100 | 90 | 90 | 90 | 90 | 85 | 90 | 90 | 75 | 95 | 95 | 80 | 45 | 90 | 80 | 90 | — | — | 85 |
| Goosegrass | — | — | — | — | — | — | — | — | — | — | — | — | — | — | — | — | — | — | — | — | — | — | — | — | 5 | 0 | 15 |
| Johnsongrass | 10 | 20 | 20 | 15 | 5 | 5 | 5 | 5 | 95 | 25 | 65 | 35 | 15 | 5 | 10 | 55 | 20 | 85 | 25 | 45 | 5 | 5 | 25 | 5 | 5 | 5 | 75 |
| Kochia | 90 | 90 | 85 | 95 | 90 | 95 | 90 | 80 | 95 | 90 | 90 | 90 | 90 | 90 | 90 | 95 | 90 | 90 | 90 | 95 | 75 | 90 | 90 | 90 | 98 | 80 | 75 |
| Lambsquarters | 95 | 95 | 85 | 95 | 85 | 75 | 85 | 65 | 98 | 90 | 95 | 85 | 75 | 85 | 85 | 95 | 95 | 95 | 90 | 85 | 30 | 80 | 25 | 60 | 65 | 50 | 100 |
| Morningglory | 100 | 95 | 80 | 98 | 80 | 95 | 80 | 75 | 75 | 98 | 98 | 98 | 75 | 98 | 98 | 85 | 75 | 98 | 98 | 95 | 65 | 85 | 70 | 65 | 60 | 60 | 65 |
| Nutsedge, Yellow | 5 | 5 | 5 | 35 | 15 | 5 | 15 | 10 | 15 | 10 | 10 | 20 | 10 | 5 | 5 | 20 | 10 | 10 | 20 | 15 | 0 | 5 | 10 | 5 | 0 | 0 | 10 |
| Oat, Wild | 45 | 30 | 40 | 45 | 20 | 25 | 40 | 15 | 80 | 50 | 85 | 60 | 15 | 5 | 45 | 80 | 20 | 40 | 30 | 10 | 5 | 40 | 15 | 10 | 20 | 15 | 15 |
| Oilseed Rape | 85 | 60 | 50 | 65 | 45 | 70 | 70 | 70 | 85 | 80 | 90 | 90 | 70 | 10 | 60 | 65 | 60 | 80 | 80 | 50 | 50 | 70 | 40 | 55 | — | — | — |
| Pigweed | 80 | 85 | 60 | 95 | 80 | 95 | 85 | 85 | 85 | 75 | 85 | 90 | 70 | 70 | 90 | 90 | 80 | 90 | 95 | 100 | 40 | 85 | 60 | 75 | 75 | 40 | 80 |
| Ragweed | 70 | 60 | 25 | 75 | 85 | 35 | 75 | 30 | 70 | 85 | 90 | 90 | 40 | 55 | 80 | 75 | 45 | 85 | 80 | 80 | 20 | 70 | 45 | 35 | 15 | 5 | 65 |
| Ryegrass, Italian | 30 | 10 | 5 | 5 | 5 | 5 | 5 | 0 | 60 | 30 | 35 | 40 | 5 | 5 | 5 | 10 | 15 | 15 | 5 | 5 | 0 | 15 | 5 | 5 | 15 | 10 | 30 |
| Soybean | 95 | 65 | 85 | 95 | 40 | 90 | 95 | 50 | 95 | 98 | 98 | 95 | 95 | 95 | 95 | 95 | 90 | 98 | 98 | 90 | 35 | 95 | 75 | 60 | 60 | 70 | 70 |
| Surinam Grass | — | — | — | — | — | — | — | — | — | — | — | — | — | — | — | — | — | — | — | — | — | — | — | — | 10 | 10 | 35 |
| Velvetleaf | 85 | 50 | 25 | 50 | 65 | 55 | 60 | 35 | 80 | 65 | 80 | 75 | 50 | 40 | 50 | 60 | 45 | 80 | 65 | 40 | 10 | 75 | 40 | 25 | 45 | 20 | 70 |
| Waterhemp | 90 | 95 | 65 | 75 | 65 | 85 | 65 | 85 | 90 | 95 | 95 | 90 | 70 | 55 | 90 | 75 | 85 | 85 | 90 | 85 | 45 | 70 | 45 | 45 | — | — | — |
| Wheat | 15 | 15 | 25 | 10 | 15 | 10 | 20 | 10 | 20 | 15 | 40 | 15 | 5 | 5 | 10 | 5 | 10 | 10 | 5 | 5 | 0 | 10 | 5 | 5 | 20 | 15 | 15 |
| Windgrass | — | — | — | — | — | — | — | — | — | — | — | — | — | — | — | — | — | — | — | — | — | — | — | — | 40 | 40 | 55 |

| | 125 g ai/ha | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | | |
| | 70 | 72 | 74 | 75 | 76 | 77 | 78 | 95 | 96 | 97 | 98 | 101 | 103 | 104 | 105 | 107 | 108 | 109 | 110 | 116 | 126 | 127 | 128 | 129 | 130 | 131 | 132 | 133 | 136 |
| Barley | 10 | 5 | 0 | — | — | — | — | — | — | — | — | — | — | — | — | — | — | — | — | — | — | — | — | — | — | — | — | — | — |
| Barnyardgrass | — | — | — | 35 | 15 | 20 | 15 | 0 | 25 | 0 | 5 | 5 | 15 | 45 | 45 | 5 | 5 | 10 | 55 | 15 | 10 | 10 | 15 | 10 | 5 | 10 | 10 | 5 | 5 |
| Bermudagrass | 10 | 15 | 5 | — | — | — | — | — | — | — | — | — | — | — | — | — | — | — | — | — | — | — | — | — | — | — | — | — | — |
| Blackgrass | — | 40 | 5 | 15 | 5 | 5 | 10 | 15 | 15 | 5 | 25 | 15 | 40 | 40 | 35 | 15 | 5 | 10 | 45 | 5 | 20 | 10 | 10 | 35 | 10 | 10 | 10 | 10 | 15 |
| Bromegrass, Downy | 5 | 15 | 0 | — | — | — | — | — | — | — | — | — | — | — | — | — | — | — | — | — | — | — | — | — | — | — | — | — | — |
| Canarygrass | 15 | 20 | 0 | — | — | — | — | — | — | — | — | — | — | — | — | — | — | — | — | — | — | — | — | — | — | — | — | — | — |
| Chickweed | 50 | 50 | 45 | 85 | 80 | 60 | 80 | 100 | 85 | 100 | 100 | 50 | 75 | 80 | 95 | 100 | 100 | 55 | 90 | 80 | 80 | 80 | 85 | 80 | 75 | 70 | 75 | 70 | 100 |
| Cocklebur | 20 | 5 | 50 | — | — | — | — | — | — | — | — | — | — | — | — | — | — | — | — | — | — | — | — | — | — | — | — | — | — |
| Corn | 45 | 10 | 20 | 40 | 20 | 10 | 25 | 10 | 45 | 20 | 5 | 60 | 35 | 35 | 60 | 5 | 15 | 15 | 25 | 15 | 35 | 10 | 10 | 15 | 5 | 5 | 75 | 10 | 15 |
| Crabgrass, Large | 40 | 5 | 30 | 80 | 70 | 20 | 80 | 40 | 30 | 45 | 45 | 20 | 35 | 75 | 90 | 45 | 10 | 10 | — | 50 | 15 | 20 | 30 | 35 | 10 | 15 | 75 | 10 | 40 |
| Cupgrass, Woolly | 15 | 10 | 15 | — | — | — | — | — | — | — | — | — | — | — | — | — | — | — | — | — | — | — | — | — | — | — | — | — | — |
| Deadnettle | 70 | 40 | 70 | — | — | — | — | — | — | — | — | — | — | — | — | — | — | — | — | — | — | — | — | — | — | — | — | — | — |
| Foxtail, Giant | 15 | 5 | 10 | 50 | 65 | 20 | 50 | 45 | 50 | 50 | 55 | 65 | 60 | 90 | 75 | 55 | 40 | 45 | 60 | 15 | 20 | 15 | 25 | 35 | 20 | 40 | 35 | 50 | 40 |
| Foxtail, Green | 80 | 40 | 0 | — | — | — | — | — | — | — | — | — | — | — | — | — | — | — | — | — | — | — | — | — | — | — | — | — | — |
| Galium | 75 | 70 | 80 | 65 | 90 | 20 | 80 | 90 | 85 | — | 65 | 55 | 75 | 85 | 85 | 90 | 80 | 65 | 90 | 80 | 55 | 70 | 80 | 80 | 65 | 50 | 60 | 70 | 60 |
| Goosegrass | 10 | 0 | 5 | — | — | — | — | — | — | — | — | — | — | — | — | — | — | — | — | — | — | — | — | — | — | — | — | — | — |
| Johnsongrass | 10 | 0 | 5 | 10 | 25 | 5 | 55 | 5 | 10 | 30 | 5 | 5 | 20 | 40 | 45 | 5 | 0 | 5 | 75 | 30 | 20 | 30 | 5 | 10 | 5 | 0 | 25 | 5 | 5 |
| Kochia | 55 | 40 | 80 | 90 | 90 | 70 | 90 | 85 | 80 | 85 | 85 | 65 | 85 | 90 | 90 | 85 | 80 | 90 | 90 | 90 | 65 | 65 | 65 | 90 | 90 | 70 | 80 | 90 | 90 |
| Lambsquarters | 95 | 90 | 80 | 90 | 85 | 70 | 95 | 90 | 80 | 90 | 90 | 75 | 75 | 95 | 95 | 70 | 80 | 65 | 85 | 85 | 80 | 85 | 95 | 98 | 65 | 65 | 75 | 90 | 98 |
| Morningglory | 75 | 60 | 75 | 98 | 85 | 40 | 90 | 50 | 30 | — | 70 | 95 | 85 | 95 | 85 | 80 | 80 | 75 | — | 75 | 80 | 60 | 45 | 100 | 85 | 65 | 80 | 90 | 95 |
| Nutsedge, Yellow | 0 | 0 | 5 | 10 | 5 | 5 | 10 | 5 | 5 | 15 | 5 | 40 | 10 | 10 | 20 | 5 | 0 | 5 | 10 | 5 | 5 | 15 | 10 | 5 | 5 | 10 | 20 | 5 | 5 |
| Oat, Wild | 30 | 20 | 5 | 25 | 5 | 5 | 10 | 15 | 20 | 10 | 10 | 10 | 50 | 35 | 45 | 5 | 15 | 5 | 30 | 25 | 20 | 5 | 25 | 35 | 15 | 15 | 10 | 15 | 10 |
| Oilseed Rape | — | — | — | 60 | 55 | 15 | 50 | 50 | 15 | 10 | 30 | 5 | 50 | 60 | 85 | 60 | 55 | 45 | 90 | 35 | 60 | 80 | 60 | 65 | 55 | 40 | 65 | 75 | 70 |
| Pigweed | 70 | 65 | 80 | 80 | 90 | 60 | 90 | 40 | 40 | 50 | 30 | 75 | 65 | 95 | 85 | 85 | 50 | 65 | 90 | — | 40 | 75 | 65 | 100 | 50 | 90 | 35 | 80 | 70 |
| Ragweed | 65 | 20 | — | 85 | 75 | 10 | 85 | 50 | 85 | 70 | 75 | 75 | 80 | 95 | 65 | 85 | 50 | — | 80 | 65 | 55 | 50 | 85 | 75 | 45 | 60 | 55 | 75 | 40 |
| Ryegrass, Italian | 35 | 15 | 0 | 10 | 5 | 5 | 5 | 5 | 20 | 5 | 10 | 10 | 5 | 35 | 30 | 5 | 5 | 5 | 35 | 10 | 10 | 10 | 15 | 5 | 10 | 10 | 10 | 5 | 5 |
| Soybean | 55 | 35 | 85 | 90 | 95 | 35 | 80 | 80 | 85 | 30 | 85 | 40 | 95 | 95 | 95 | 50 | 90 | 75 | 70 | 75 | 55 | 70 | 95 | 98 | 65 | 35 | 95 | 95 | 95 |
| Surinam Grass | 20 | 10 | 15 | — | — | — | — | — | — | — | — | — | — | — | — | — | — | — | — | — | — | — | — | — | — | — | — | — | — |
| Velvetleaf | 30 | 35 | 30 | 40 | 60 | 20 | 50 | 35 | 30 | 30 | 20 | 30 | 45 | 65 | 70 | 35 | 35 | 25 | 70 | 50 | 35 | 45 | 35 | — | 50 | 35 | 35 | 35 | 60 |
| Waterhemp | — | — | — | 75 | 75 | 65 | 80 | 30 | 20 | — | 85 | 75 | 65 | — | 75 | 40 | 50 | 60 | 100 | — | 70 | 70 | 70 | 80 | 70 | 65 | 65 | 75 | 75 |
| Wheat | 20 | 10 | 0 | 5 | 5 | 5 | 10 | 10 | 10 | — | 5 | 5 | 5 | 10 | 10 | 20 | 10 | 0 | 5 | 5 | 5 | 5 | 10 | 5 | 5 | 10 | 10 | 15 | 10 |
| Windgrass | 50 | 25 | 0 | — | — | — | — | — | — | — | — | — | — | — | — | — | — | — | — | — | — | — | — | — | — | — | — | — | — |

| | 125 g ai/ha | | | | | | | | | | | | | | | | | | | | | | | 62 g ai/ha | | | |
|---|---|---|---|---|---|---|---|---|---|---|---|---|---|---|---|---|---|---|---|---|---|---|---|---|---|---|---|
| | Compounds | | | | | | | | | | | | | | | | | | | | | | | Compounds | | | |
| | 138 | 139 | 144 | 153 | 154 | 177 | 185 | 186 | 187 | 188 | 189 | 196 | 197 | 198 | 199 | 200 | 201 | 202 | 205 | 206 | 214 | 220 | 224 | 20 | 27 | 69 | 70 |
| Barley | — | — | — | — | — | — | — | — | — | — | — | — | — | — | — | — | — | — | — | — | — | — | — | 20 | 20 | 10 | 5 |
| Barnyardgrass | 20 | 10 | 10 | 10 | 5 | 10 | 5 | 10 | 15 | 30 | 20 | 20 | 5 | 15 | 30 | 5 | 35 | 15 | 15 | 5 | 30 | 10 | 10 | — | — | — | — |
| Bermudagrass | — | — | — | — | — | — | — | — | — | — | — | — | — | — | — | — | — | — | — | — | — | — | — | 5 | 5 | 10 | 5 |
| Blackgrass | 20 | 20 | 30 | 5 | 10 | 20 | 5 | 10 | 30 | 40 | 10 | 5 | 5 | 40 | 10 | 15 | 15 | 20 | 5 | 5 | 10 | 5 | 5 | 5 | 15 | 10 | 5 |
| Bromegrass, Downy | — | — | — | — | — | — | — | — | — | — | — | — | — | — | — | — | — | — | — | — | — | — | — | 0 | 5 | 5 | 0 |
| Canarygrass | — | — | — | — | — | — | — | — | — | — | — | — | — | — | — | — | — | — | — | — | — | — | — | 10 | 5 | 15 | 5 |
| Chickweed | 80 | 65 | 80 | 70 | 60 | 70 | 85 | 85 | 80 | 80 | 85 | 80 | 60 | 80 | 80 | 90 | 85 | 80 | 98 | 5 | 70 | 60 | 55 | 45 | 20 | 55 | 45 |
| Cocklebur | — | — | — | — | — | — | — | — | — | — | — | — | — | — | — | — | — | — | — | — | — | — | — | 35 | 25 | 10 | 15 |
| Corn | 15 | 15 | 15 | 30 | 10 | 15 | 5 | 75 | 60 | 20 | 10 | 5 | 5 | 10 | 85 | 10 | 15 | 15 | 20 | 5 | 5 | 5 | 10 | 20 | 10 | 15 | 40 |
| Crabgrass, Large | 30 | 15 | 10 | 15 | 5 | 5 | 5 | 20 | 60 | 70 | 65 | 10 | 10 | 15 | 40 | 10 | 15 | 25 | 45 | 5 | 20 | 5 | 5 | 20 | 5 | 20 | 10 |
| Cupgrass, Woolly | — | — | — | — | — | — | — | — | — | — | — | — | — | — | — | — | — | — | — | — | — | — | — | 10 | 5 | 10 | 0 |
| Deadnettle | — | — | — | — | — | — | — | — | — | — | — | — | — | — | — | — | — | — | — | — | — | — | — | — | — | 70 | 70 |
| Foxtail, Giant | 10 | 10 | 15 | 10 | 5 | 30 | 10 | 60 | 45 | 60 | 25 | 20 | 5 | 15 | 50 | 15 | 30 | 10 | 70 | 5 | 15 | 5 | 5 | 10 | 0 | 30 | 10 |
| Foxtail, Green | — | — | — | — | — | — | — | — | — | — | — | — | — | — | — | — | — | — | — | — | — | — | — | 30 | 30 | 70 | 5 |
| Galium | 75 | 60 | 70 | 70 | 65 | 80 | 45 | 75 | 85 | 90 | 90 | 80 | 50 | 85 | 80 | 75 | 60 | 90 | 80 | 60 | 60 | 60 | 40 | — | — | 85 | 70 |
| Goosegrass | — | — | — | — | — | — | — | — | — | — | — | — | — | — | — | — | — | — | — | — | — | — | — | 5 | 0 | 15 | 5 |
| Johnsongrass | 10 | 10 | 10 | 0 | 5 | 5 | 0 | 5 | 5 | 50 | 5 | 10 | 0 | 5 | 40 | 10 | 30 | 5 | 20 | 5 | 5 | 20 | 5 | 0 | 0 | 10 | 0 |
| Kochia | 90 | 85 | 95 | 85 | 85 | 90 | 75 | 90 | 90 | 90 | 90 | 90 | 85 | 85 | 90 | 90 | 90 | 90 | 95 | 45 | 90 | 90 | 90 | 95 | 70 | 70 | 55 |
| Lambsquarters | 75 | 70 | 70 | 75 | 75 | 75 | 45 | 75 | 75 | 90 | 95 | 75 | 60 | 75 | 70 | 65 | 90 | 85 | 90 | 15 | 75 | 20 | 45 | 60 | 50 | 98 | 95 |
| Morningglory | 85 | 90 | 85 | 70 | 85 | 85 | 85 | 75 | 80 | 98 | 95 | 55 | 65 | 98 | 60 | 75 | 85 | 90 | 80 | 60 | 85 | 40 | 25 | 25 | 60 | 60 | 45 |
| Nutsedge, Yellow | 5 | 5 | 5 | 20 | 5 | 5 | 5 | 5 | 5 | 5 | 5 | 5 | 10 | 5 | 45 | 5 | 5 | 10 | 15 | 0 | 5 | 40 | 5 | 0 | 0 | 0 | 0 |
| Oat, Wild | 10 | 30 | 20 | 5 | 20 | 15 | 10 | 35 | 30 | 60 | 30 | 10 | 5 | 20 | 30 | 10 | 25 | 10 | 5 | 5 | 15 | 10 | 10 | 15 | 10 | 15 | 20 |
| Oilseed Rape | 80 | 50 | 60 | 40 | 60 | 60 | 50 | 80 | 75 | 70 | 65 | 10 | 20 | 70 | 55 | 60 | 50 | 65 | 50 | 5 | 60 | 30 | 50 | — | — | — | — |
| Pigweed | 80 | 40 | 85 | 25 | 85 | 75 | 75 | 85 | 60 | 80 | 85 | 70 | 65 | 80 | 75 | 75 | 80 | 90 | 85 | 40 | 75 | 40 | 50 | 75 | 40 | 75 | 65 |
| Ragweed | 50 | 35 | 35 | 40 | 15 | 70 | 40 | 55 | 50 | 85 | 75 | 15 | 40 | 55 | 20 | 45 | 60 | 65 | 60 | 35 | 35 | 50 | 10 | 15 | 0 | 60 | 60 |
| Ryegrass, Italian | 5 | 10 | 5 | 5 | 0 | 5 | 0 | 10 | 5 | 20 | 5 | 5 | 0 | 5 | 10 | 15 | 5 | 5 | 5 | 0 | 5 | 5 | 5 | 10 | 5 | 15 | 25 |
| Soybean | 90 | 55 | 75 | 35 | 85 | 90 | 40 | 95 | 95 | 95 | 95 | 80 | 90 | 95 | 75 | 90 | 95 | 98 | 70 | 45 | 90 | 30 | 70 | 45 | 65 | 60 | 55 |
| Surinam Grass | — | — | — | — | — | — | — | — | — | — | — | — | — | — | — | — | — | — | — | — | — | — | — | 5 | 5 | 10 | 10 |
| Velvetleaf | 25 | 15 | 35 | 30 | 25 | 45 | 25 | 55 | 55 | 60 | 55 | 55 | 40 | 40 | 35 | 30 | 60 | 40 | 40 | 10 | 20 | 45 | 20 | 45 | 15 | 60 | 20 |
| Waterhemp | 100 | 55 | 75 | 40 | 65 | 75 | 35 | 60 | 70 | 85 | 75 | 60 | 55 | 85 | 50 | 65 | 70 | 85 | 70 | 35 | 60 | 45 | 50 | — | — | — | — |
| Wheat | 15 | 5 | 10 | 0 | 5 | 15 | 5 | 15 | 10 | 15 | 15 | 0 | 5 | 5 | 5 | 10 | 5 | 5 | 5 | 0 | 5 | 0 | 0 | 15 | 15 | 10 | 15 |
| Windgrass | — | — | — | — | — | — | — | — | — | — | — | — | — | — | — | — | — | — | — | — | — | — | — | 10 | 20 | 50 | 25 |

62 g ai/ha
Compounds

| | 72 | 74 | 75 | 76 | 77 | 78 | 95 | 96 | 97 | 98 | 101 | 103 | 104 | 105 | 107 | 108 | 109 | 110 | 116 | 126 | 127 | 128 | 129 | 130 | 131 | 132 | 133 | 136 | 138 |
|---|---|---|---|---|---|---|---|---|---|---|---|---|---|---|---|---|---|---|---|---|---|---|---|---|---|---|---|---|---|
| Barley | 5 | 0 | — | — | — | — | — | — | — | — | — | — | — | — | — | — | — | — | — | — | — | — | — | — | — | — | — | — | — |
| Barnyardgrass | — | — | 10 | 5 | 5 | 15 | 0 | 0 | 0 | 0 | 5 | 15 | 5 | 20 | 0 | 0 | 5 | 35 | 5 | 5 | 5 | 5 | 10 | 5 | 5 | 5 | 5 | 5 | 25 |
| Bermudagrass | 0 | 5 | — | — | — | — | — | — | — | — | — | — | — | — | — | — | — | — | — | — | — | — | — | — | — | — | — | — | — |
| Blackgrass | 10 | 0 | 10 | 5 | 5 | 5 | 5 | 5 | 5 | 5 | 5 | 15 | 10 | 30 | 10 | 5 | 5 | 35 | 5 | 5 | 15 | 5 | 5 | 5 | 5 | 5 | 5 | 10 | 10 |
| Bromegrass, Downy | 5 | 0 | — | — | — | — | — | — | — | — | — | — | — | — | — | — | — | — | — | — | — | — | — | — | — | — | — | — | — |
| Canarygrass | 10 | 0 | — | — | — | — | — | — | — | — | — | — | — | — | — | — | — | — | — | — | — | — | — | — | — | — | — | — | — |
| Chickweed | 40 | 45 | 70 | 80 | 55 | 80 | 100 | 85 | 100 | 100 | 50 | 60 | 70 | 90 | 100 | 100 | 50 | 70 | 80 | 70 | 80 | 75 | 70 | 70 | 70 | 70 | 60 | 100 | 80 |
| Cocklebur | 0 | 20 | — | — | — | — | — | — | — | — | — | — | — | — | — | — | — | — | — | — | — | — | — | — | — | — | — | — | — |
| Corn | 10 | 10 | 20 | 10 | 5 | 15 | 10 | 10 | 5 | 5 | 10 | 15 | 35 | 40 | 5 | 5 | 10 | 15 | 10 | 10 | 5 | 5 | 10 | 5 | 5 | 5 | 10 | 10 | 15 |
| Crabgrass, Large | 5 | 20 | 25 | 10 | 10 | 25 | 10 | 15 | 40 | 15 | 15 | 15 | 70 | 45 | 30 | 10 | 10 | 40 | 20 | 10 | 10 | 5 | 10 | 5 | 5 | 5 | 5 | 5 | 5 |
| Cupgrass, Woolly | 10 | 10 | — | — | — | — | — | — | — | — | — | — | — | — | — | — | — | — | — | — | — | — | — | — | — | — | — | — | — |
| Deadnettle | 40 | 60 | — | — | — | — | — | — | — | — | — | — | — | — | — | — | — | — | — | — | — | — | — | — | — | — | — | — | — |
| Foxtail, Giant | 5 | 10 | 25 | 20 | 10 | 20 | 40 | 45 | 40 | 50 | 30 | 40 | 85 | 40 | 15 | 30 | 10 | 40 | 10 | 20 | 15 | 10 | 20 | 15 | 10 | 10 | 40 | 30 | 5 |
| Foxtail, Green | 40 | 0 | — | — | — | — | — | — | — | — | — | — | — | — | — | — | — | — | — | — | — | — | — | — | — | — | — | — | — |
| Galium | 70 | 60 | 60 | 75 | 15 | — | 80 | 80 | 90 | 55 | 50 | 70 | 60 | 85 | 85 | 60 | 50 | 60 | 50 | 50 | 70 | 65 | 65 | 65 | 55 | 60 | 70 | 60 | 70 |
| Goosegrass | 0 | 5 | — | — | — | — | — | — | — | — | — | — | — | — | — | — | — | — | — | — | — | — | — | — | — | — | — | — | — |
| Johnsongrass | 0 | 0 | 10 | 0 | 5 | 5 | 0 | 0 | 0 | 5 | 5 | 10 | 15 | 35 | 0 | 0 | 5 | — | 5 | 10 | 5 | 5 | 10 | 5 | 0 | 5 | 5 | 5 | 10 |
| Kochia | 25 | 50 | 60 | 90 | 45 | 90 | 70 | 75 | 85 | 75 | 65 | 85 | 90 | 90 | 85 | 60 | 75 | 90 | 90 | 50 | 65 | 65 | 90 | 75 | 70 | 80 | 90 | 90 | 90 |
| Lambsquarters | 70 | 35 | 85 | 75 | 35 | 75 | 80 | 60 | 85 | 70 | 65 | 70 | 95 | 95 | 40 | 60 | 45 | 75 | 75 | 70 | 80 | 80 | 100 | 30 | 40 | 55 | 55 | 85 | 40 |
| Morningglory | 35 | 75 | 80 | 75 | 20 | 80 | 50 | 30 | 80 | 50 | 65 | 75 | 80 | 80 | 80 | 50 | 70 | — | 75 | 60 | 70 | 65 | 100 | 75 | 55 | 75 | 90 | 98 | 85 |
| Nutsedge, Yellow | 0 | 5 | 5 | 5 | 5 | 5 | 0 | 5 | 5 | 5 | 5 | 10 | 5 | 5 | 0 | 0 | 5 | 10 | 5 | 5 | 5 | 5 | 0 | 5 | 5 | 10 | 5 | 5 | 0 |
| Oat, Wild | 5 | 0 | 5 | 5 | 5 | 5 | 5 | 5 | 10 | 5 | 5 | 5 | 15 | 30 | 5 | 10 | 5 | 25 | 20 | 15 | 5 | 10 | 30 | 10 | 10 | 10 | 5 | 5 | 20 |
| Oilseed Rape | — | — | 50 | 35 | 5 | 45 | 30 | 10 | 10 | 25 | 5 | 50 | 50 | 50 | 60 | 50 | 45 | 85 | 35 | 50 | 70 | 60 | 60 | 40 | 35 | 55 | 60 | 60 | 60 |
| Pigweed | 45 | 65 | 60 | 60 | 55 | 75 | 40 | 35 | 40 | 10 | 45 | 50 | 85 | 85 | 85 | 45 | 40 | 85 | 75 | 50 | 70 | 70 | 95 | 65 | 65 | 30 | 60 | 65 | 85 |
| Ragweed | 5 | 45 | 70 | 75 | 10 | 75 | 50 | 10 | 35 | 35 | 20 | 70 | 85 | 40 | 65 | 50 | — | 80 | 45 | 35 | 20 | 40 | 90 | 35 | 30 | 10 | 25 | 35 | 30 |
| Ryegrass, Italian | 10 | 0 | 5 | 5 | 5 | 5 | 5 | 5 | 5 | 10 | 5 | 5 | 5 | 10 | 5 | 5 | 5 | 20 | 5 | 5 | 5 | 10 | 5 | 5 | 5 | 10 | 5 | 5 | 15 |
| Soybean | 20 | 80 | 75 | 75 | 25 | 80 | 50 | 70 | 30 | 70 | 25 | 70 | 95 | 80 | 50 | 90 | 60 | 55 | 75 | 45 | 65 | 95 | 98 | 80 | 25 | 45 | 75 | 70 | 75 |
| Surinam Grass | 10 | 10 | — | — | — | — | — | — | — | — | — | — | — | — | — | — | — | — | — | — | — | — | — | — | — | — | — | — | — |
| Velvetleaf | 5 | 30 | 35 | 40 | 15 | 20 | 15 | 25 | 10 | 20 | 25 | 40 | 40 | 55 | 30 | 10 | 15 | 50 | 50 | 25 | 15 | 15 | 65 | 10 | 25 | 15 | 45 | 55 | 40 |
| Waterhemp | — | — | 45 | 70 | 10 | 70 | 30 | 5 | 50 | 45 | 35 | 65 | 85 | 75 | 40 | 50 | 35 | 100 | 60 | 50 | 65 | 65 | 90 | 55 | 55 | 55 | 60 | 70 | 70 |
| Wheat | 5 | 0 | 5 | 5 | 5 | 10 | 5 | 10 | 15 | 5 | 5 | 5 | 10 | 5 | 5 | 5 | 0 | 5 | 5 | 5 | 5 | 5 | 5 | 0 | 5 | 10 | 5 | 5 | 5 |
| Windgrass | 20 | 0 | — | — | — | — | — | — | — | — | — | — | — | — | — | — | — | — | — | — | — | — | — | — | — | — | — | — | — |

| | |
|---|---|
| 62 g ai/ha | 31 g ai/ha |
| Compounds | Compounds |

| | 139 | 144 | 153 | 154 | 177 | 185 | 186 | 187 | 188 | 189 | 196 | 197 | 198 | 199 | 200 | 201 | 202 | 205 | 206 | 214 | 220 | 224 | 20 | 27 | 69 | 70 | 72 | 74 | 75 |
|---|---|---|---|---|---|---|---|---|---|---|---|---|---|---|---|---|---|---|---|---|---|---|---|---|---|---|---|---|---|
| Barley | — | — | — | — | — | — | — | — | — | — | — | — | — | — | — | — | — | — | — | — | — | — | 15 | 10 | 5 | 0 | 0 | 0 | — |
| Barnyardgrass | 10 | 15 | 10 | 5 | 5 | 0 | 10 | 10 | 15 | 5 | 5 | 0 | 15 | 10 | 5 | 10 | 10 | 10 | 5 | 5 | 0 | 10 | — | — | — | — | — | — | 10 |
| Bermudagrass | — | — | — | — | — | — | — | — | — | — | — | — | — | — | — | — | — | — | — | — | — | — | 5 | 0 | 10 | 5 | 0 | 5 | — |
| Blackgrass | 20 | 5 | 5 | 0 | 15 | 0 | 10 | 5 | 5 | 5 | 15 | 0 | 20 | 10 | 10 | 5 | 10 | 5 | 0 | 5 | 5 | 5 | 5 | 10 | 10 | 0 | 5 | 0 | 5 |
| Bromegrass, Downy | — | — | — | — | — | — | — | — | — | — | — | — | — | — | — | — | — | — | — | — | — | — | 0 | 0 | 5 | 0 | 5 | 0 | — |
| Canarygrass | — | — | — | — | — | — | — | — | — | — | — | — | — | — | — | — | — | — | — | — | — | — | 5 | 5 | 10 | 0 | 5 | 0 | — |
| Chickweed | 60 | 65 | 70 | 70 | 75 | 80 | 80 | 70 | 85 | 80 | 60 | 55 | 70 | 60 | 75 | 80 | 80 | 90 | 5 | 55 | 55 | 55 | 45 | 20 | 10 | 45 | 40 | 20 | 65 |
| Cocklebur | — | — | — | — | — | — | — | — | — | — | — | — | — | — | — | — | — | — | — | — | — | — | 20 | 20 | 5 | 10 | 0 | 20 | — |
| Corn | 10 | 10 | 10 | 5 | 20 | 5 | 40 | 5 | 55 | 10 | 5 | 5 | 10 | 10 | 5 | 10 | 10 | 10 | 5 | 5 | 5 | 5 | 15 | 10 | 10 | 10 | 10 | 10 | 5 |
| Crabgrass, Large | 10 | 10 | 10 | 5 | 5 | 5 | 5 | 20 | 55 | 5 | 5 | 5 | 5 | 10 | 10 | 10 | 10 | 10 | 5 | 5 | 5 | 5 | 10 | 0 | 15 | 5 | 5 | 10 | 20 |
| Cupgrass, Woolly | — | — | — | — | — | — | — | — | — | — | — | — | — | — | — | — | — | — | — | — | — | — | 5 | 0 | 0 | 0 | 5 | 5 | — |
| Deadnettle | — | — | — | — | — | — | — | — | — | — | — | — | — | — | — | — | — | — | — | — | — | — | — | — | 70 | 25 | 40 | 10 | — |
| Foxtail, Giant | 5 | 10 | 5 | 5 | 10 | 5 | 30 | 10 | 40 | 10 | 25 | 0 | 10 | 10 | 10 | 10 | 10 | 35 | 5 | 5 | 5 | 5 | 10 | 0 | 10 | 5 | 5 | 5 | 20 |
| Foxtail, Green | — | — | — | — | — | — | — | — | — | — | — | — | — | — | — | — | — | — | — | — | — | — | 20 | 25 | 40 | — | 20 | 0 | — |
| Galium | 60 | 55 | 60 | 50 | 50 | 40 | 80 | 80 | 80 | 80 | 55 | 50 | 85 | 70 | 50 | 80 | 85 | 80 | 30 | 70 | 40 | 55 | — | — | 65 | 70 | 45 | 60 | 60 |
| Goosegrass | — | — | — | — | — | — | — | — | | — | — | — | — | — | — | — | — | — | — | — | — | — | 0 | 0 | 5 | 5 | 0 | 5 | — |
| Johnsongrass | 5 | 5 | 5 | 0 | 0 | 0 | 5 | 5 | 0 | 15 | 5 | 65 | 5 | 5 | 10 | 5 | 5 | 10 | 0 | 0 | 0 | 5 | 0 | 0 | 0 | 0 | 0 | 0 | 10 |
| Kochia | 75 | 85 | 70 | 85 | 85 | 60 | 85 | 90 | 90 | 90 | 85 | 60 | 80 | 85 | 90 | 85 | 90 | 90 | 35 | 90 | 85 | 85 | 90 | 35 | 65 | 50 | 10 | 20 | 60 |
| Lambsquarters | 55 | 60 | 55 | 50 | 50 | 35 | 65 | 75 | 75 | 70 | 65 | 55 | 50 | 85 | 65 | 80 | 80 | 85 | 10 | 50 | 10 | 40 | 60 | 50 | 80 | 75 | 60 | 20 | 70 |
| Morningglory | 60 | 40 | 85 | 60 | 40 | 45 | 85 | 85 | 90 | 80 | 80 | 75 | 60 | 50 | 65 | 95 | 40 | 70 | 40 | 95 | 55 | 20 | 10 | 5 | 40 | 5 | 0 | 70 | 75 |
| Nutsedge, Yellow | 5 | 5 | 5 | 0 | 5 | 0 | 5 | 5 | 35 | 5 | 0 | 5 | 10 | 10 | 0 | 10 | 5 | 5 | 0 | 10 | 0 | 0 | 0 | 0 | 0 | 0 | 0 | 0 | 5 |
| Oat, Wild | 10 | 10 | 10 | 5 | 5 | 10 | 35 | 15 | 10 | 15 | 5 | 0 | 10 | 10 | 10 | 10 | 5 | 5 | 0 | 15 | 5 | 5 | 15 | 10 | 10 | 20 | 5 | 0 | 5 |
| Oilseed Rape | 45 | 50 | 25 | 45 | 50 | 40 | 60 | 50 | 60 | 60 | 5 | 5 | 50 | 45 | 30 | 60 | 30 | 25 | 5 | 60 | 5 | 50 | — | — | — | — | — | — | 50 |
| Pigweed | 40 | 75 | 25 | 65 | 40 | 50 | 70 | 50 | 75 | 75 | 60 | 60 | 60 | 60 | 60 | 65 | 80 | 85 | 25 | 60 | 25 | 50 | 60 | 40 | 75 | 60 | 45 | 60 | 40 |
| Ragweed | 20 | 20 | 20 | 45 | 40 | 25 | 45 | 65 | 60 | 60 | 10 | 35 | 40 | 25 | 25 | 60 | 40 | 60 | 25 | 10 | 5 | 10 | 5 | 0 | 60 | 60 | 5 | 25 | 55 |
| Ryegrass, Italian | 5 | 5 | 0 | 0 | 5 | 0 | 5 | 5 | 5 | 5 | 5 | 0 | 5 | 5 | 10 | 5 | 5 | 5 | 0 | 5 | 0 | 0 | 5 | 5 | 10 | 15 | 5 | 0 | 5 |
| Soybean | 30 | 65 | 25 | 50 | 95 | 40 | 90 | 95 | 95 | 90 | 85 | 70 | 80 | 30 | — | 95 | 65 | 35 | 35 | 70 | 40 | 45 | 25 | 20 | 35 | 15 | 20 | 65 | 50 |
| Surinam Grass | — | — | — | — | — | — | — | — | — | — | — | — | — | — | — | — | — | — | — | — | — | — | 5 | 5 | 5 | 10 | 5 | 5 | — |
| Velvetleaf | 20 | 25 | 30 | 20 | 25 | 15 | 30 | 40 | 50 | 30 | 55 | 55 | 35 | 35 | 20 | 55 | 40 | 20 | 10 | 20 | 40 | 10 | 25 | 15 | 55 | 20 | 5 | 10 | 30 |
| Waterhemp | 50 | 70 | 10 | 50 | 40 | 50 | 75 | 60 | 85 | 85 | 45 | 50 | 40 | 45 | 50 | 75 | 80 | 65 | 15 | 65 | 30 | 45 | — | — | — | — | — | — | 45 |
| Wheat | 10 | 10 | 0 | 0 | 0 | 0 | 5 | 5 | 5 | 10 | 0 | 0 | 5 | 5 | 10 | 5 | 0 | 5 | 0 | 5 | 0 | 0 | 10 | 15 | 10 | 15 | 5 | 0 | 5 |
| Windgrass | — | — | — | — | — | — | — | — | — | — | — | — | — | — | — | — | — | — | — | — | — | — | 5 | 20 | 45 | 0 | 10 | 0 | — |

| | 31 g ai/ha | | | | | | | | | | | | | | | | | | | | | | | | | | | |
|---|---|---|---|---|---|---|---|---|---|---|---|---|---|---|---|---|---|---|---|---|---|---|---|---|---|---|---|---|
| | Compounds | | | | | | | | | | | | | | | | | | | | | | | | | | | |
| | 76 | 77 | 78 | 95 | 96 | 97 | 98 | 101 | 103 | 104 | 105 | 107 | 108 | 109 | 110 | 116 | 126 | 127 | 128 | 129 | 130 | 131 | 132 | 133 | 136 | 138 | 139 | 144 |
| Barley | — | — | — | — | — | — | — | — | — | — | — | — | — | — | — | — | — | — | — | — | — | — | — | — | — | — | — | — |
| Barnyardgrass | 5 | 0 | 5 | 0 | 0 | 0 | 0 | 5 | 10 | 5 | 10 | 0 | 0 | 5 | 20 | 5 | 5 | 5 | 5 | 5 | 5 | 5 | 5 | 5 | 5 | 10 | 10 | 10 |
| Bermudagrass | — | — | — | — | — | — | — | — | — | — | — | — | — | — | — | — | — | — | — | — | — | — | — | — | — | — | — | — |
| Blackgrass | 5 | 5 | 5 | 5 | 5 | 5 | 5 | 5 | 10 | 10 | 10 | 10 | 0 | 5 | 30 | 5 | 5 | 20 | 10 | 10 | 5 | 5 | 5 | 5 | 5 | 5 | 20 | 20 |
| Bromegrass, Downy | — | — | — | — | — | — | — | — | — | — | — | — | — | — | — | — | — | — | — | — | — | — | — | — | — | — | — | — |
| Canarygrass | — | — | — | — | — | — | — | — | — | — | — | — | — | — | — | — | — | — | — | — | — | — | — | — | — | — | — | — |
| Chickweed | 50 | 50 | 65 | 85 | 80 | 100 | 100 | 50 | 50 | 55 | 90 | 100 | 85 | 40 | 70 | 70 | 60 | 75 | 70 | 50 | 65 | 70 | 75 | 50 | 100 | 70 | 60 | 70 |
| Cocklebur | — | — | — | — | — | — | — | — | — | — | — | — | — | — | — | — | — | — | — | — | — | — | — | — | — | — | — | — |
| Corn | 5 | 0 | 10 | 5 | 5 | 5 | 5 | 10 | 10 | 5 | 15 | 5 | 5 | 5 | — | 5 | 5 | 5 | 5 | 10 | 5 | 5 | 5 | 10 | 10 | 15 | 5 | 5 |
| Crabgrass, Large | 5 | 10 | 15 | 5 | 5 | 15 | 10 | 5 | 10 | 5 | 35 | 5 | 5 | 5 | 35 | 15 | 5 | 10 | 5 | 10 | 5 | 5 | 5 | 5 | 5 | 5 | 10 | 10 |
| Cupgrass, Woolly | — | — | — | — | — | — | — | — | — | — | — | — | — | — | — | — | — | — | — | — | — | — | — | — | — | — | — | — |
| Deadnettle | — | — | — | — | — | — | — | — | — | — | — | — | — | — | — | — | — | — | — | — | — | — | — | — | — | — | — | — |
| Foxtail, Giant | 20 | 5 | 15 | 5 | 40 | 40 | 45 | 10 | 40 | 5 | 15 | 10 | 10 | 10 | 35 | 10 | 10 | 10 | 5 | 15 | 40 | 5 | 5 | 35 | 30 | 5 | 5 | 5 |
| Foxtail, Green | — | — | — | — | — | — | — | — | — | — | — | — | — | — | — | — | — | — | — | — | — | — | — | — | — | — | — | — |
| Galium | 70 | 5 | 80 | 60 | 55 | 70 | 50 | 35 | 50 | 60 | 55 | 50 | 60 | 50 | 60 | 50 | 50 | 70 | 60 | 60 | 60 | 45 | 55 | 70 | 60 | 65 | 50 | 40 |
| Goosegrass | — | — | — | — | — | — | — | — | — | — | — | — | — | — | — | — | — | — | — | — | — | — | — | — | — | — | — | — |
| Johnsongrass | 0 | 5 | 0 | 0 | 0 | 0 | 0 | 5 | 5 | 5 | 5 | 0 | 0 | 5 | 40 | 5 | 5 | 5 | 5 | 5 | 0 | 0 | 5 | 5 | 5 | 10 | 5 | 5 |
| Kochia | 75 | 40 | 85 | 50 | 60 | 85 | 40 | 60 | 85 | 85 | 90 | 50 | 40 | 50 | 90 | 90 | 55 | 60 | 60 | 90 | 65 | 40 | 70 | 85 | 90 | 100 | 60 | 75 |
| Lambsquarters | 75 | 35 | 60 | 40 | 40 | 30 | 60 | 50 | 65 | 80 | 75 | 40 | 60 | 45 | 40 | 65 | 35 | 65 | 60 | 55 | 25 | 15 | 25 | 50 | 80 | — | 40 | 60 |
| Morningglory | 75 | — | 70 | 50 | 5 | 50 | 45 | 60 | 75 | 0 | 80 | 75 | 50 | 65 | — | 75 | 40 | 65 | 75 | 80 | 35 | 35 | 30 | 75 | 90 | 60 | 60 | 35 |
| Nutsedge, Yellow | 5 | 5 | 5 | 0 | 0 | 0 | 0 | 5 | 10 | 5 | 5 | 0 | 0 | 0 | 5 | 5 | 5 | 5 | 5 | 0 | 0 | 5 | 5 | 5 | 5 | 0 | 5 | 10 |
| Oat, Wild | 5 | 5 | 5 | 5 | 5 | 10 | 5 | 0 | 5 | 5 | 25 | 5 | 10 | 0 | 5 | 15 | 35 | 5 | 5 | 30 | 5 | 5 | 10 | 5 | 5 | 10 | 10 | 15 |
| Oilseed Rape | 30 | 5 | 45 | 30 | 10 | 10 | 20 | 5 | 5 | 40 | 45 | 5 | 50 | 40 | 60 | 30 | 45 | 65 | 50 | 60 | 35 | 30 | 25 | 50 | 60 | 55 | 20 | 50 |
| Pigweed | 60 | 50 | 40 | 10 | 30 | 40 | 10 | 35 | 50 | 50 | 75 | 50 | 40 | 10 | 85 | 60 | 25 | 35 | 65 | 85 | 30 | 35 | 10 | 50 | 40 | 75 | 30 | 65 |
| Ragweed | 55 | 5 | 35 | 45 | 5 | 20 | 15 | 15 | 35 | 85 | 30 | 10 | 10 | — | 50 | 15 | 30 | 30 | 50 | 55 | 30 | 5 | 10 | 25 | 15 | 10 | 35 | 10 |
| Ryegrass, Italian | 5 | 5 | 5 | 5 | 5 | 0 | 5 | 5 | 5 | 5 | 5 | 0 | 0 | 5 | 20 | 5 | 5 | 5 | 5 | 0 | 5 | 5 | 10 | 5 | 5 | 5 | 0 | 5 |
| Soybean | 65 | 10 | 50 | 45 | 15 | 30 | 70 | 20 | 45 | 90 | 80 | 20 | 30 | 55 | 35 | 60 | 40 | 40 | 65 | 85 | 70 | 20 | 65 | 85 | 90 | 65 | 15 | 35 |
| Surinam Grass | — | — | — | — | — | — | — | — | — | — | — | — | — | — | — | — | — | — | — | — | — | — | — | — | — | — | — | — |
| Velvetleaf | 15 | 15 | 15 | 10 | 10 | 10 | 15 | 15 | 25 | 25 | 40 | 30 | 5 | 10 | 30 | 35 | 30 | 10 | 10 | 25 | 5 | 5 | 5 | 25 | 30 | 30 | 10 | 15 |
| Waterhemp | 60 | 10 | 50 | 30 | 5 | 50 | 10 | 30 | 55 | 35 | 65 | 5 | 5 | 35 | 100 | 40 | 40 | 50 | 60 | 65 | 30 | 55 | 35 | 60 | 60 | 75 | 30 | 65 |
| Wheat | 0 | 0 | 5 | 5 | 10 | 15 | 5 | 0 | 0 | 5 | 5 | 0 | 0 | 0 | 5 | 0 | 5 | 5 | 5 | 0 | 0 | 5 | 5 | 5 | 5 | 5 | 5 | 5 |
| Windgrass | — | — | — | — | — | — | — | — | — | — | — | — | — | — | — | — | — | — | — | — | — | — | — | — | — | — | — | — |

| | 31 g ai/ha | | | | | | | | | | | | | | | | | | | |
|---|---|---|---|---|---|---|---|---|---|---|---|---|---|---|---|---|---|---|---|---|
| | Compounds | | | | | | | | | | | | | | | | | | | |
| | 153 | 154 | 177 | 185 | 186 | 187 | 188 | 189 | 196 | 197 | 198 | 199 | 200 | 201 | 202 | 205 | 206 | 214 | 220 | 224 |
| Barley | — | — | — | — | — | — | — | — | — | — | — | — | — | — | — | — | — | — | — | — |
| Barnyardgrass | 5 | 5 | 5 | 0 | 5 | 5 | 5 | 5 | 5 | 0 | 5 | 10 | 0 | 5 | 10 | 10 | 5 | 5 | 0 | 0 |
| Bermudagrass | — | — | — | — | — | — | — | — | — | — | — | — | — | — | — | — | — | — | — | — |
| Blackgrass | 0 | 0 | 20 | 0 | 5 | 10 | 5 | 5 | 20 | 0 | 0 | 0 | 5 | 20 | 5 | 5 | 0 | 5 | 5 | 5 |
| Bromegrass, Downy | — | — | — | — | — | — | — | — | — | — | — | — | — | — | — | — | — | — | — | — |
| Canarygrass | — | — | — | — | — | — | — | — | — | — | — | — | — | — | — | — | — | — | — | — |
| Chickweed | 50 | 60 | 60 | 70 | 70 | 98 | 70 | 80 | 50 | 50 | 70 | 70 | 75 | 75 | 75 | 65 | 5 | 50 | 50 | 50 |
| Cocklebur | — | — | — | — | — | — | — | — | — | — | — | — | — | — | — | — | — | — | — | — |
| Corn | 5 | 5 | 5 | 5 | 5 | 5 | 5 | 5 | 5 | 5 | 5 | 5 | 5 | 10 | 5 | 10 | 5 | 0 | 5 | 5 |
| Crabgrass, Large | 5 | 5 | 5 | 5 | 5 | 10 | 5 | 5 | 5 | 5 | 5 | 10 | 10 | 15 | 5 | 5 | 5 | 5 | 5 | 5 |
| Cupgrass, Woolly | — | — | — | — | — | — | — | — | — | — | — | — | — | — | — | — | — | — | — | — |
| Deadnettle | — | — | — | — | — | — | — | — | — | — | — | — | — | — | — | — | — | — | — | — |
| Foxtail, Giant | 5 | 5 | 5 | 5 | 5 | 5 | 5 | 10 | 5 | 0 | 10 | 10 | 5 | 10 | 5 | 20 | 15 | 5 | 0 | 5 |
| Foxtail, Green | — | — | — | — | — | — | — | — | — | — | — | — | — | — | — | — | — | — | — | — |
| Galium | 50 | 50 | 50 | 40 | 70 | 60 | 80 | 80 | 40 | 40 | 50 | 60 | 50 | 60 | 65 | 65 | 30 | 75 | 50 | 50 |
| Goosegrass | — | — | — | — | — | — | — | — | — | — | — | — | — | — | — | — | — | — | — | — |
| Johnsongrass | 0 | 0 | 0 | 0 | 5 | 0 | 0 | 0 | 0 | 0 | 5 | 5 | 10 | 5 | 0 | 5 | 0 | 0 | 0 | 20 |
| Kochia | 50 | 80 | 60 | 50 | 80 | 85 | 90 | 85 | 80 | 40 | 80 | 80 | 90 | 85 | 85 | 85 | 30 | 85 | 80 | 80 |
| Lambsquarters | 65 | 40 | 65 | 70 | 95 | 60 | 60 | 60 | 60 | 55 | 20 | 25 | 55 | 65 | 70 | 85 | 10 | 35 | 30 | 35 |
| Morningglory | 65 | 15 | 60 | 40 | 85 | 70 | 85 | 75 | 40 | 75 | 95 | 30 | 65 | 80 | 35 | 65 | 40 | 75 | 10 | 25 |
| Nutsedge, Yellow | 5 | 0 | 10 | 0 | 5 | 5 | 5 | 5 | 5 | 0 | 5 | 5 | 0 | 10 | 0 | 5 | 0 | 0 | 0 | 0 |
| Oat, Wild | 5 | 5 | 5 | 5 | 10 | 10 | 15 | 10 | 5 | 0 | 10 | 5 | 5 | 5 | 5 | 5 | 0 | 10 | 0 | 5 |
| Oilseed Rape | 20 | 35 | 35 | 5 | 50 | 50 | 50 | 30 | 5 | 5 | 50 | 40 | 30 | 30 | 30 | 20 | 5 | 55 | 10 | 30 |
| Pigweed | 15 | 65 | 20 | 60 | 55 | 45 | 45 | 65 | 50 | 35 | 30 | 50 | 60 | 65 | 70 | 45 | 10 | 55 | 10 | 40 |
| Ragweed | 35 | 25 | 10 | 5 | 15 | 15 | 10 | 40 | 5 | 20 | 10 | 15 | 10 | 10 | 40 | 35 | 10 | 10 | 15 | 10 |
| Ryegrass, Italian | 0 | 0 | 5 | 0 | 5 | 5 | 5 | 5 | 0 | 0 | 0 | 5 | 5 | 5 | 5 | 5 | 0 | 5 | 0 | 0 |
| Soybean | 20 | 50 | 40 | 20 | 50 | 95 | 90 | 85 | 65 | 40 | 75 | 20 | 50 | 80 | 90 | 40 | 30 | 75 | 10 | 25 |
| Surinam Grass | — | — | — | — | — | — | — | — | — | — | — | — | — | — | — | — | — | — | — | — |
| Velvetleaf | 25 | 10 | 20 | 15 | 20 | 35 | 20 | 40 | 30 | 25 | 35 | 20 | 20 | 30 | 25 | 10 | 10 | 10 | 10 | 10 |
| Waterhemp | 20 | 40 | 35 | 20 | 50 | 70 | 55 | 40 | 60 | 15 | 40 | 40 | 45 | 60 | 55 | 65 | 10 | 65 | 40 | 40 |
| Wheat | 0 | 0 | 0 | 0 | 5 | 0 | 5 | 10 | 0 | 0 | 0 | 5 | 5 | 5 | 0 | 5 | 0 | 0 | 0 | 0 |
| Windgrass | — | — | — | — | — | — | — | — | — | — | — | — | — | — | — | — | — | — | — | — |

| | 16 g ai/ha | | |
|---|---|---|---|
| | Compounds | | |
| | 75 | 77 | 101 |
| Barnyardgrass | 5 | 0 | 5 |
| Blackgrass | 5 | 0 | 5 |
| Chickweed | 65 | 50 | 45 |
| Corn | 0 | 0 | 5 |
| Crabgrass, Large | 20 | 5 | 5 |
| Foxtail, Giant | 15 | 5 | 10 |
| Galium | 50 | 5 | 20 |
| Johnsongrass | 10 | 5 | 0 |
| Kochia | 30 | 40 | 40 |
| Lambsquarters | 70 | 15 | 50 |
| Morningglory | 25 | 10 | 60 |
| Nutsedge, Yellow | 0 | 0 | 5 |
| Oat, Wild | 5 | 5 | 0 |
| Oilseed Rape | 20 | 5 | 5 |
| Pigweed | 40 | 25 | 20 |
| Ragweed | 40 | 5 | 15 |
| Ryegrass, Italian | 5 | 0 | 5 |
| Soybean | 40 | 10 | 15 |
| Velvetleaf | 25 | 10 | 10 |
| Waterhemp | — | 10 | 10 |
| Wheat | 0 | 0 | 0 |

Preemergence

| | 250 g ai/ha | | | | | | | | | | | | | | | | | | | | | | | | | |
|---|---|---|---|---|---|---|---|---|---|---|---|---|---|---|---|---|---|---|---|---|---|---|---|---|---|---|
| | Compounds | | | | | | | | | | | | | | | | | | | | | | | | | |
| | 70 | 72 | 74 | 76 | 78 | 95 | 96 | 97 | 98 | 103 | 104 | 105 | 107 | 108 | 109 | 110 | 116 | 125 | 126 | 127 | 128 | 129 | 130 | 131 | 132 | 133 |
| Barnyardgrass | — | — | — | 100 | 100 | 100 | 100 | 100 | 100 | 100 | 100 | 100 | 100 | 100 | 100 | 90 | 100 | 100 | 100 | 95 | 100 | 100 | 100 | 100 | 100 | 100 |
| Bermudagrass | 100 | 100 | 98 | — | — | — | — | — | — | — | — | — | — | — | — | — | — | — | — | — | — | — | — | — | — | — |
| Blackgrass | 85 | 85 | 90 | — | — | 100 | 100 | 100 | 100 | 100 | 90 | 90 | 100 | 100 | 100 | — | 95 | 80 | 90 | 85 | 90 | 90 | 98 | 98 | 98 | 95 |
| Bromegrass, Downy | 80 | 90 | 95 | — | — | — | — | — | — | — | — | — | — | — | — | — | — | — | — | — | — | — | — | — | — | — |
| Cocklebur | 100 | 100 | 100 | — | — | — | — | — | — | — | — | — | — | — | — | — | — | — | — | — | — | — | — | — | — | — |
| Corn | 85 | 75 | 98 | 100 | 98 | 95 | 95 | 90 | 95 | 100 | 98 | 95 | 95 | 95 | 100 | 10 | 95 | 45 | 98 | 45 | 70 | 65 | 85 | 98 | 95 | 95 |
| Crabgrass, Large | 100 | 98 | 100 | 100 | 100 | 100 | 98 | 100 | 100 | 100 | 100 | 100 | 98 | 100 | 100 | 100 | 100 | 95 | 100 | 100 | 100 | 100 | 100 | 100 | 100 | 100 |
| Cupgrass, Woolly | 90 | 65 | 98 | — | — | — | — | — | — | — | — | — | — | — | — | — | — | — | — | — | — | — | — | — | — | — |
| Foxtail, Giant | 98 | 100 | 100 | 100 | 100 | 100 | 100 | 100 | 100 | 100 | 100 | 100 | 100 | 100 | 100 | 100 | 100 | 90 | 100 | 98 | 100 | 100 | 100 | 100 | 100 | 100 |
| Foxtail, Green | 98 | 98 | 95 | — | — | — | — | — | — | — | — | — | — | — | — | — | — | — | — | — | — | — | — | — | — | — |
| Galium | 98 | 100 | 100 | 100 | 100 | 100 | 100 | 100 | 100 | 100 | 100 | 100 | 100 | 100 | 100 | 100 | 100 | 90 | 100 | 100 | 100 | 100 | 100 | 98 | 100 | 100 |
| Goosegrass | 98 | 98 | 100 | — | — | — | — | — | — | — | — | — | — | — | — | — | — | — | — | — | — | — | — | — | — | — |
| Johnsongrass | 95 | 75 | 100 | 100 | 100 | 100 | 100 | 100 | 100 | 100 | 100 | 100 | 100 | 100 | 100 | 95 | 100 | 10 | 98 | 90 | 98 | 100 | 95 | 95 | 100 | 98 |
| Kochia | 98 | 80 | 98 | — | — | — | — | — | — | — | — | — | — | — | — | — | — | — | — | — | — | — | — | — | — | — |
| Lambsquarters | 100 | 100 | 95 | — | — | 100 | 100 | 100 | 100 | 100 | 100 | 100 | 100 | 100 | 100 | — | 100 | 100 | 100 | 100 | 98 | 100 | 98 | 100 | 100 | 100 |
| Morningglory | 95 | 85 | 100 | — | — | 100 | 100 | 100 | 100 | 100 | 100 | 100 | 100 | 100 | 100 | — | 100 | 95 | 100 | 100 | 100 | 100 | 100 | 100 | 100 | 100 |
| Nightshade | 98 | 100 | 98 | — | — | — | — | — | — | — | — | — | — | — | — | — | — | — | — | — | — | — | — | — | — | — |
| Nutsedge, Yellow | 85 | 85 | 98 | 98 | 98 | 90 | 90 | 90 | 95 | 95 | 98 | 95 | 100 | 85 | 60 | 5 | 90 | 55 | 90 | 85 | 90 | 75 | 75 | 90 | 70 | 85 |
| Oat, Wild | 65 | 85 | 95 | — | — | — | — | — | — | — | — | — | — | — | — | — | — | — | — | — | — | — | — | — | — | — |
| Oilseed Rape | — | — | — | — | — | 100 | 100 | 100 | 100 | 100 | 100 | 100 | 100 | 100 | 100 | — | 98 | 95 | 100 | 100 | 100 | 100 | 100 | 100 | 100 | 100 |
| Pigweed | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 |
| Ragweed | 40 | 60 | 90 | 100 | 100 | 100 | 100 | 100 | 100 | — | 100 | 98 | 100 | 100 | 100 | 75 | 100 | 75 | 90 | 90 | 90 | 100 | 100 | 100 | 100 | 100 |
| Russian Thistle | 80 | 95 | — | — | — | — | — | — | — | — | — | — | — | — | — | — | — | — | — | — | — | — | — | — | — | — |
| Ryegrass, Italian | 80 | 95 | 80 | — | — | 100 | 100 | 100 | 100 | 98 | 95 | 95 | 100 | 100 | 100 | — | 95 | 95 | 90 | 80 | 95 | 95 | 98 | 95 | 98 | 95 |
| Soybean | 75 | 75 | 98 | 100 | 98 | 98 | 98 | 95 | 98 | 100 | 98 | 95 | 95 | 98 | 98 | 20 | 95 | 15 | 98 | 85 | 90 | 98 | 98 | 98 | 95 | 95 |
| Sunflower | 5 | 20 | 95 | — | — | — | — | — | — | — | — | — | — | — | — | — | — | — | — | — | — | — | — | — | — | — |
| Surinam Grass | 85 | 75 | 100 | — | — | — | — | — | — | — | — | — | — | — | — | — | — | — | — | — | — | — | — | — | — | — |
| Velvetleaf | 100 | 100 | 100 | 100 | 100 | 100 | 100 | 100 | 100 | 100 | 100 | 100 | 100 | 100 | 100 | 75 | 100 | 40 | 100 | 100 | 100 | 100 | 100 | 100 | 100 | 100 |
| Waterhemp | — | — | — | 100 | 100 | 100 | 100 | 100 | 100 | 100 | 100 | 100 | 100 | 100 | 100 | 100 | 100 | 90 | 100 | 100 | 100 | 100 | 100 | 100 | 100 | 100 |
| Wheat | 75 | 85 | 90 | — | — | 90 | 95 | 90 | 90 | 98 | 90 | 90 | 50 | 70 | 85 | — | 90 | 50 | 90 | 85 | 85 | 90 | 95 | 95 | 95 | 90 |
| | 250 g ai/ha | | | | | | | | | | | | | | | | | | | | | | | | | |
| | Compounds | | | | | | | | | | | | | | | | | | | | | | | | | |
| | 136 | 139 | 143 | 144 | 145 | 153 | 154 | 175 | 176 | 177 | 179 | 185 | 186 | 187 | 188 | 189 | 195 | 196 | 197 | 198 | 199 | 200 | 201 | 202 | 203 | 205 |
| Barnyardgrass | 100 | 100 | 100 | 100 | 100 | 100 | 100 | 100 | 100 | 100 | 100 | 100 | 100 | 100 | 100 | 100 | 100 | 100 | 100 | 100 | 100 | 98 | 100 | 100 | 100 | 100 |
| Bermudagrass | — | — | — | — | — | — | — | — | — | — | — | — | — | — | — | — | — | — | — | — | — | — | — | — | — | — |
| Blackgrass | 90 | 90 | 90 | 90 | 90 | 90 | 90 | 90 | 90 | 90 | 95 | 80 | 90 | 95 | 90 | 90 | 90 | 90 | 90 | 90 | 90 | 90 | 90 | 90 | 90 | 100 |
| Bromegrass, Downy | — | — | — | — | — | — | — | — | — | — | — | — | — | — | — | — | — | — | — | — | — | — | — | — | — | — |
| Cocklebur | — | — | — | — | — | — | — | — | — | — | — | — | — | — | — | — | — | — | — | — | — | — | — | — | — | — |
| Corn | 85 | 80 | 95 | 85 | 90 | 98 | 95 | 90 | 90 | 98 | 95 | 75 | 98 | 98 | 98 | 98 | 95 | 98 | 95 | 98 | 90 | 80 | 95 | 95 | 98 | 98 |
| Crabgrass, Large | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 |
| Cupgrass, Woolly | — | — | — | — | — | — | — | — | — | — | — | — | — | — | — | — | — | — | — | — | — | — | — | — | — | — |
| Foxtail, Giant | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 |
| Foxtail, Green | — | — | — | — | — | — | — | — | — | — | — | — | — | — | — | — | — | — | — | — | — | — | — | — | — | — |
| Galium | 100 | 100 | 100 | 100 | 100 | 100 | 100 | 100 | 100 | 100 | 100 | 100 | 95 | 98 | 100 | 100 | 100 | 100 | 95 | 100 | 100 | 100 | 100 | 100 | 100 | 100 |
| Goosegrass | — | — | — | — | — | — | — | — | — | — | — | — | — | — | — | — | — | — | — | — | — | — | — | — | — | — |
| Johnsongrass | 100 | 98 | 95 | 100 | 98 | 95 | 85 | 100 | 85 | 98 | 98 | 60 | 100 | 100 | 100 | 100 | 85 | 100 | 85 | 100 | 100 | 98 | 100 | 100 | 55 | 100 |
| Kochia | — | — | — | — | — | — | — | — | — | — | — | — | — | — | — | — | — | — | — | — | — | — | — | — | — | — |
| Lambsquarters | 100 | 100 | — | 98 | — | 100 | 98 | 100 | 100 | 98 | 100 | 100 | 100 | 100 | 100 | 100 | — | — | — | — | 98 | 100 | — | — | 100 | 100 |
| Morningglory | 100 | 98 | 100 | 100 | 100 | 100 | 100 | 100 | 100 | 100 | 100 | 98 | 100 | 100 | 100 | 100 | 100 | 100 | 100 | 100 | 100 | 100 | 100 | 100 | 100 | 100 |
| Nightshade | — | — | — | — | — | — | — | — | — | — | — | — | — | — | — | — | — | — | — | — | — | — | — | — | — | — |
| Nutsedge, Yellow | 85 | 50 | 90 | 90 | 90 | 95 | 60 | 98 | 85 | 95 | 85 | 75 | 98 | 98 | 98 | 95 | 95 | 90 | 85 | 65 | 85 | 60 | 80 | 50 | 90 | 90 |
| Oat, Wild | — | — | — | — | — | — | — | — | — | — | — | — | — | — | — | — | — | — | — | — | — | — | — | — | — | — |
| Oilseed Rape | 100 | 100 | 100 | 100 | 98 | 95 | 95 | 100 | 100 | 95 | 100 | 100 | 95 | 100 | 100 | 95 | 98 | 100 | 100 | 100 | 100 | 98 | 95 | 100 | 100 | 100 |
| Pigweed | 100 | 100 | 100 | 100 | 100 | 98 | 100 | 100 | 100 | 100 | 98 | 100 | 100 | 100 | 100 | 100 | 100 | 100 | 100 | 100 | 100 | 100 | 98 | 100 | 100 | 100 |
| Ragweed | 98 | 95 | 100 | 98 | 98 | 98 | 98 | 95 | 95 | 98 | 98 | 100 | 98 | 100 | 98 | 98 | 98 | 98 | 98 | 100 | 95 | 85 | 98 | 100 | 100 | 100 |
| Russian Thistle | — | — | — | — | — | — | — | — | — | — | — | — | — | — | — | — | — | — | — | — | — | — | — | — | — | — |
| Ryegrass, Italian | 95 | 90 | 90 | 90 | 90 | 90 | 90 | 90 | 85 | 90 | 90 | 90 | 90 | 90 | 90 | 90 | 90 | 90 | 90 | 90 | 90 | 95 | 90 | 90 | 90 | 95 |
| Soybean | 100 | 60 | 98 | 98 | 95 | 80 | 98 | 98 | 98 | 98 | 98 | 65 | 98 | 98 | 98 | 98 | 60 | 98 | 98 | 98 | 95 | 85 | 98 | 98 | 55 | 100 |
| Sunflower | — | — | — | — | — | — | — | — | — | — | — | — | — | — | — | — | — | — | — | — | — | — | — | — | — | — |
| Surinam Grass | — | — | — | — | — | — | — | — | — | — | — | — | — | — | — | — | — | — | — | — | — | — | — | — | — | — |
| Velvetleaf | 100 | 100 | 100 | 100 | 100 | 100 | 100 | 100 | 100 | 100 | 100 | 98 | 100 | 100 | 100 | 100 | 100 | 100 | 100 | 100 | 100 | 100 | 100 | 100 | 100 | 100 |
| Waterhemp | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 98 | 100 | 100 | 100 |
| Wheat | 95 | 90 | 85 | 90 | 90 | 90 | 90 | 90 | 85 | 90 | 85 | 90 | 90 | 90 | 90 | 90 | 90 | 80 | 80 | 90 | 90 | 75 | 90 | 90 | 90 | 95 |
| | 250 g ai/ha | | | | 125 g ai/ha | | | | | | | | | | | | | | | | | | | | | |
| | Compounds | | | | Compounds | | | | | | | | | | | | | | | | | | | | | |
| | 214 | 220 | 224 | 226 | 70 | 72 | 74 | 75 | 76 | 77 | 78 | 95 | 96 | 97 | 98 | 101 | 103 | 104 | 105 | 107 | 108 | 109 | 110 | 116 | 125 | 126 |
| Barnyardgrass | 100 | 95 | 100 | 100 | — | — | — | 100 | 100 | 100 | 100 | 100 | 100 | 100 | 100 | 100 | 100 | 100 | 100 | 100 | 100 | 100 | 65 | 100 | 80 | 100 |
| Bermudagrass | — | — | — | — | 100 | 98 | 98 | — | — | — | — | — | — | — | — | — | — | — | — | — | — | — | — | — | — | — |
| Blackgrass | 90 | 70 | 90 | 90 | 65 | 85 | 90 | — | — | — | — | 100 | 100 | 100 | 100 | 95 | 100 | 90 | 90 | 100 | 100 | 100 | — | 95 | 60 | 90 |
| Bromegrass, Downy | — | — | — | — | 40 | 80 | 50 | — | — | — | — | — | — | — | — | — | — | — | — | — | — | — | — | — | — | — |
| Cocklebur | — | — | — | — | 45 | 10 | 100 | — | — | — | — | — | — | — | — | — | — | — | — | — | — | — | — | — | — | — |
| Corn | 40 | 40 | 60 | 70 | 70 | 60 | 75 | 100 | 100 | 55 | 95 | 80 | 85 | 90 | 90 | 100 | 100 | 98 | 80 | 90 | 95 | 95 | 5 | 75 | 10 | 90 |
| Crabgrass, Large | 100 | 100 | 100 | 100 | 98 | 98 | 100 | 100 | 100 | 100 | 100 | 98 | 98 | 98 | 100 | 100 | 100 | 100 | 100 | 98 | 100 | 100 | 100 | 100 | 75 | 100 |
| Cupgrass, Woolly | — | — | — | — | 85 | 45 | 98 | — | — | — | — | — | — | — | — | — | — | — | — | — | — | — | — | — | — | — |
| Foxtail, Giant | 100 | 100 | 100 | 100 | 90 | 85 | 100 | 100 | 100 | 100 | 98 | 100 | 100 | 100 | 100 | 100 | 100 | 100 | 100 | 100 | 100 | 100 | 100 | 100 | 90 | 98 |
| Foxtail, Green | — | — | — | — | 95 | 90 | 95 | — | — | — | — | — | — | — | — | — | — | — | — | — | — | — | — | — | — | — |
| Galium | 80 | 98 | 90 | 90 | 98 | 100 | 100 | 100 | 100 | 10 | 100 | 100 | 100 | 100 | 100 | 80 | 100 | 100 | 100 | 95 | 100 | 100 | — | 100 | 100 | 100 |
| Goosegrass | — | — | — | — | 95 | 98 | 100 | — | — | — | — | — | — | — | — | — | — | — | — | — | — | — | — | — | — | — |
| Johnsongrass | 90 | 80 | 98 | 85 | 80 | 65 | 98 | 100 | 100 | 70 | 100 | 95 | 100 | 98 | 100 | 75 | 100 | 100 | 100 | 90 | 100 | 100 | 80 | 100 | 5 | 95 |
| Kochia | — | — | — | — | 98 | 50 | 98 | — | — | — | — | — | — | — | — | — | — | — | — | — | — | — | — | — | — | — |
| Lambsquarters | 100 | 100 | 100 | 100 | 100 | 100 | 90 | — | — | — | — | 100 | 100 | 100 | 100 | 95 | 100 | 100 | 100 | 100 | 100 | 100 | — | 100 | 75 | 100 |
| Morningglory | 100 | 75 | 100 | 100 | 75 | 85 | 100 | — | — | — | — | 100 | 100 | 100 | 100 | 100 | 100 | 100 | 100 | 100 | 100 | 100 | — | 100 | 75 | 98 |
| Nightshade | — | — | — | — | 98 | 98 | 95 | — | — | — | — | — | — | — | — | — | — | — | — | — | — | — | — | — | — | — |
| Nutsedge, Yellow | 65 | 5 | 90 | 20 | 70 | 60 | 80 | 85 | 90 | 60 | 98 | 85 | 80 | 90 | — | 75 | 85 | 98 | 95 | 85 | — | 40 | 5 | 80 | 15 | 75 |
| Oat, Wild | — | — | — | — | 50 | 80 | 85 | — | — | — | — | — | — | — | — | — | — | — | — | — | — | — | — | — | — | — |
| Oilseed Rape | 95 | 95 | 95 | 95 | — | — | — | — | — | — | — | 100 | 100 | 100 | 100 | 100 | 100 | 100 | 95 | 100 | 100 | 100 | — | 98 | 95 | 100 |
| Pigweed | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 90 | 100 |
| Ragweed | 98 | 95 | 100 | 98 | 30 | 55 | 80 | 100 | 100 | 85 | 98 | 100 | 98 | 100 | 100 | — | — | 100 | 98 | 100 | 95 | 100 | 60 | 100 | 55 | 100 |
| Russian Thistle | — | — | — | — | 75 | 95 | — | — | — | — | — | — | — | — | — | — | — | — | — | — | — | — | — | — | — | — |
| Ryegrass, Italian | 80 | 5 | 30 | 80 | 60 | 85 | 50 | — | — | — | — | 95 | 90 | 90 | 95 | 100 | 95 | 95 | 95 | 60 | 90 | 100 | — | 95 | 20 | 90 |
| Soybean | 95 | 60 | 90 | 100 | 70 | 75 | 85 | 98 | 98 | 50 | 98 | 95 | 95 | 95 | 98 | 50 | 100 | 98 | 95 | 95 | 95 | 98 | 15 | 95 | 10 | 75 |
| Sunflower | — | — | — | — | 5 | 5 | 75 | — | — | — | — | — | — | — | — | — | — | — | — | — | — | — | — | — | — | — |
| Surinam Grass | — | — | — | — | 65 | 65 | 85 | — | — | — | — | — | — | — | — | — | — | — | — | — | — | — | — | — | — | — |
| Velvetleaf | 100 | 100 | 100 | 100 | 75 | 100 | 100 | 100 | 100 | 100 | 100 | 100 | 100 | 100 | 100 | 100 | 100 | 100 | 100 | 100 | 100 | 100 | 70 | 100 | 30 | 100 |
| Waterhemp | 100 | 100 | 100 | 100 | — | — | — | 100 | 100 | 100 | 100 | 100 | 100 | 98 | 100 | 100 | 100 | 100 | 100 | 100 | 100 | 100 | 100 | 100 | 85 | 100 |
| Wheat | 40 | 0 | 60 | 10 | 65 | 50 | 15 | — | — | — | — | 60 | 85 | 65 | 70 | 85 | 95 | 90 | 90 | 50 | 60 | 85 | — | 90 | 5 | 90 |
| | 125 g ai/ha | | | | | | | | | | | | | | | | | | | | | | | | | |
| | Compounds | | | | | | | | | | | | | | | | | | | | | | | | | |
| | 127 | 128 | 129 | 130 | 131 | 132 | 133 | 136 | 139 | 143 | 144 | 145 | 153 | 154 | 175 | 176 | 177 | 178 | 179 | 185 | 186 | 187 | 188 | 189 | 195 | 196 |
| Barnyardgrass | 95 | 98 | 100 | 100 | 100 | 100 | 100 | 100 | 100 | 100 | 100 | 100 | 100 | 100 | 100 | 100 | 100 | 100 | 100 | 98 | 100 | 100 | 100 | 100 | 100 | 100 |
| Bermudagrass | — | — | — | — | — | — | — | — | — | — | — | — | — | — | — | — | — | — | — | — | — | — | — | — | — | — |
| Blackgrass | 60 | 90 | 90 | 98 | 95 | 98 | 90 | 90 | 90 | 90 | 90 | 90 | 90 | 90 | 90 | 90 | 90 | 85 | 90 | 90 | 90 | 90 | 90 | 90 | 90 | 90 |
| Bromegrass, Downy | — | — | — | — | — | — | — | — | — | — | — | — | — | — | — | — | — | — | — | — | — | — | — | — | — | — |
| Cocklebur | — | — | — | — | — | — | — | — | — | — | — | — | — | — | — | — | — | — | — | — | — | — | — | — | — | — |
| Corn | 65 | 65 | 55 | 45 | 95 | 95 | 45 | 60 | 85 | 90 | 60 | 80 | 95 | 40 | 85 | 90 | 90 | 25 | 30 | 45 | 95 | 95 | 98 | 85 | 70 | 65 |
| Crabgrass, Large | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 | 100 |
| Cupgrass, Woolly | — | — | — | — | — | — | — | — | — | — | — | — | — | — | — | — | — | — | — | — | — | — | — | — | — | — |
| Foxtail, Giant | 100 | 100 | 100 | 100 | 100 | 100 | 100 | 100 | 100 | 100 | 100 | 100 | 100 | 100 | 100 | 100 | 100 | 100 | 100 | 98 | 100 | 100 | 100 | 100 | 100 | 100 |
| Foxtail, Green | — | — | — | — | — | — | — | — | — | — | — | — | — | — | — | — | — | — | — | — | — | — | — | — | — | — |
| Galium | 100 | 100 | 95 | 98 | 75 | 100 | 98 | 100 | 100 | 95 | 100 | 95 | 100 | 100 | 100 | 98 | 100 | 95 | 100 | 95 | 100 | 95 | 100 | 100 | 95 | 100 |
| Goosegrass | — | — | — | — | — | — | — | — | — | — | — | — | — | — | — | — | — | — | — | — | — | — | — | — | — | — |
| Johnsongrass | 80 | 95 | 98 | 75 | 85 | 100 | 90 | 98 | 85 | 95 | 75 | 98 | 75 | 55 | 70 | 65 | 100 | 25 | 75 | 20 | 100 | 100 | 100 | 100 | 75 | 100 |
| Kochia | — | — | — | — | — | — | — | — | — | — | — | — | — | — | — | — | — | — | — | — | — | — | — | — | — | — |
| Lambsquarters | 90 | 98 | 100 | 100 | 100 | 100 | 100 | 98 | 100 | — | 98 | — | 98 | 100 | 100 | 100 | 98 | 100 | 100 | 100 | 100 | 100 | 100 | 100 | — | — |
| Morningglory | 100 | 98 | 100 | 100 | 100 | 100 | 100 | 100 | 98 | 100 | 100 | 100 | 100 | 100 | 100 | 98 | 100 | 100 | 100 | 85 | 100 | 100 | 98 | 98 | 100 | 100 |
| Nightshade | — | — | — | — | — | — | — | — | — | — | — | — | — | — | — | — | — | — | — | — | — | — | — | — | — | — |
| Nutsedge, Yellow | 70 | 80 | 60 | 75 | 95 | 80 | 70 | 50 | 35 | 80 | 65 | 60 | 85 | 35 | 70 | 75 | 80 | 35 | 75 | 75 | 85 | 85 | 95 | 85 | 85 | 80 |
| Oat, Wild | — | — | — | — | — | — | — | — | — | — | — | — | — | — | — | — | — | — | — | — | — | — | — | — | — | — |
| Oilseed Rape | 100 | 100 | 100 | 98 | 98 | 100 | 100 | 100 | 100 | 95 | 100 | 100 | 95 | 95 | 100 | 95 | 90 | 98 | 100 | 85 | 100 | 95 | 100 | 95 | 95 | 100 |
| Pigweed | 100 | 100 | 100 | 100 | 100 | 100 | 100 | 60 | 90 | 100 | 100 | 100 | 100 | 100 | 100 | 100 | 100 | 100 | 100 | 100 | 100 | 100 | 100 | 100 | 100 | 100 |
| Ragweed | 60 | 85 | 98 | 100 | 100 | 100 | 100 | 75 | 90 | 95 | 95 | 95 | 98 | 98 | 98 | 100 | 98 | 90 | 98 | 95 | 95 | 98 | 100 | 100 | 90 | 98 |
| Russian Thistle | — | — | — | — | — | — | — | — | — | — | — | — | — | — | — | — | — | — | — | — | — | — | — | — | — | — |
| Ryegrass, Italian | 80 | 90 | 95 | 65 | 85 | 98 | 90 | 95 | 90 | 90 | 85 | 85 | 90 | 50 | 90 | 70 | 85 | 15 | 90 | 90 | 90 | 90 | 90 | 90 | 90 | 90 |
| Soybean | 65 | 80 | 98 | 90 | 85 | 90 | 85 | 95 | 30 | 98 | 80 | 85 | 45 | 95 | 98 | 98 | 95 | 75 | 85 | 25 | 98 | 98 | 95 | 98 | 25 | 90 |
| Sunflower | — | — | — | — | — | — | — | — | — | — | — | — | — | — | — | — | — | — | — | — | — | — | — | — | — | — |
| Surinam Grass | — | — | — | — | — | — | — | — | — | — | — | — | — | — | — | — | — | — | — | — | — | — | — | — | — | — |
| Velvetleaf | 100 | 100 | 100 | 90 | 100 | 100 | 100 | 100 | 95 | 100 | 100 | 100 | 90 | 90 | 100 | 90 | 50 | 70 | 100 | 100 | 100 | 100 | 100 | 100 | 100 | 100 |
| Waterhemp | 100 | 100 | 100 | 100 | 100 | 100 | 100 | 100 | 100 | 100 | 100 | 100 | 100 | 100 | 100 | 100 | 98 | 100 | 100 | 100 | 100 | 100 | 100 | 100 | 100 | 100 |
| Wheat | 85 | 70 | 85 | 40 | 50 | 70 | 90 | 95 | 85 | 80 | 80 | 50 | 90 | 50 | 85 | 5 | 50 | 0 | 80 | 85 | 90 | 90 | 90 | 90 | 85 | 85 |
| | 125 g ai/ha | | | | | | | | | | | | 62 g ai/ha | | | | | | | | | | | | | |
| | Compounds | | | | | | | | | | | | Compounds | | | | | | | | | | | | | |
| | 197 | 198 | 199 | 200 | 201 | 202 | 203 | 205 | 214 | 220 | 224 | 226 | 70 | 72 | 74 | 75 | 76 | 77 | 78 | 95 | 96 | 97 | 98 | 101 | 103 | 104 |
| Barnyardgrass | 100 | 100 | 100 | 90 | 100 | 100 | 100 | 100 | 100 | 35 | 100 | 95 | — | — | — | 100 | 100 | 75 | 100 | 100 | 95 | 100 | 100 | 100 | 100 | 100 |
| Bermudagrass | — | — | — | — | — | — | — | — | — | — | — | — | 98 | 98 | 90 | — | — | — | — | — | — | — | — | — | — | — |
| Blackgrass | 90 | 90 | 90 | 90 | 90 | 90 | 85 | 95 | 90 | 40 | 80 | 70 | 65 | 80 | 85 | — | — | — | — | 100 | 100 | 100 | 100 | 90 | 100 | 90 |
| Bromegrass, Downy | — | — | — | — | — | — | — | — | — | — | — | — | 35 | 45 | 5 | — | — | — | — | — | — | — | — | — | — | — |
| Cocklebur | — | — | — | — | — | — | — | — | — | — | — | — | 0 | 0 | 100 | — | — | — | — | — | — | — | — | — | — | — |
| Corn | 45 | 85 | 75 | 35 | 70 | 60 | 75 | 80 | 35 | 5 | 15 | 30 | 60 | 45 | 60 | 98 | 80 | 15 | 65 | 50 | 60 | 60 | 45 | 95 | 100 | 65 |
| Crabgrass, Large | 100 | 100 | 100 | 100 | 100 | 100 | 100 | 100 | 100 | 100 | 100 | 100 | 98 | 95 | 100 | 100 | 100 | 100 | 100 | 98 | 98 | 98 | 100 | 100 | 100 | 100 |
| Cupgrass, Woolly | — | — | — | — | — | — | — | — | — | — | — | — | 65 | 45 | — | — | — | — | — | — | — | — | — | — | — | — |
| Foxtail, Giant | 100 | 100 | 100 | 98 | 100 | 100 | 100 | 100 | 100 | 100 | 100 | 100 | 75 | 80 | 100 | 100 | 100 | 85 | 98 | 100 | 100 | 100 | 100 | 98 | 100 | 100 |
| Foxtail, Green | — | — | — | — | — | — | — | — | — | — | — | — | 95 | 85 | 95 | — | — | — | — | — | — | — | — | — | — | — |
| Galium | 98 | 100 | 100 | 100 | 98 | 100 | 100 | 95 | 90 | 80 | 100 | 80 | 95 | 98 | 100 | 100 | 100 | 5 | 100 | 100 | 100 | 100 | 100 | 80 | 100 | 98 |
| Goosegrass | — | — | — | — | — | — | — | — | — | — | — | — | 80 | 98 | 85 | — | — | — | — | — | — | — | — | — | — | — |
| Johnsongrass | 80 | 100 | 98 | 90 | 100 | 100 | 40 | 90 | 75 | 5 | 60 | 100 | 65 | 55 | 25 | 100 | 100 | — | 95 | 85 | 90 | 90 | 70 | 40 | 100 | 100 |
| Kochia | — | — | — | — | — | — | — | — | — | — | — | — | 65 | 45 | 98 | — | — | — | — | — | — | — | — | — | — | — |
| Lambsquarters | — | — | 100 | 100 | — | — | 100 | 100 | 100 | 100 | 100 | 100 | 98 | 100 | 90 | — | — | — | — | 100 | 100 | 100 | 100 | 85 | 100 | 98 |
| Morningglory | 100 | 100 | 98 | 98 | 100 | 100 | 85 | 100 | 100 | 65 | 40 | 100 | 25 | 65 | 100 | — | — | — | — | 100 | 98 | 100 | 95 | 100 | 100 | 100 |
| Nightshade | — | — | — | — | — | — | — | — | — | — | — | — | 98 | 98 | 90 | — | — | — | — | — | — | — | — | — | — | — |
| Nutsedge, Yellow | 75 | 30 | 80 | 40 | 55 | 30 | 85 | 75 | 65 | 5 | 70 | 5 | 65 | 40 | 60 | 75 | 70 | 25 | 85 | 80 | 80 | 85 | 75 | 25 | 80 | 95 |
| Oat, Wild | — | — | — | — | — | — | — | — | — | — | — | — | 10 | 60 | 40 | — | — | — | — | — | — | — | — | — | — | — |
| Oilseed Rape | 95 | 100 | 100 | 98 | 100 | 100 | 100 | 100 | 95 | 85 | 95 | 100 | — | — | — | — | — | — | — | 100 | 100 | 100 | 100 | 98 | 100 | 100 |
| Pigweed | 100 | 100 | 100 | 100 | 98 | 100 | 100 | 100 | 100 | 100 | 100 | 100 | 100 | 100 | 100 | 100 | 100 | 100 | 100 | 100 | 100 | 100 | 100 | 90 | 100 | 100 |
| Ragweed | 95 | 98 | 95 | 70 | 95 | 95 | 98 | 98 | 100 | 75 | 95 | 95 | 25 | 50 | 60 | 90 | 100 | 70 | 98 | 95 | 90 | 80 | 100 | — | — | 100 |
| Russian Thistle | — | — | — | — | — | — | — | — | — | — | — | — | 75 | 90 | — | — | — | — | — | — | — | — | — | — | — | — |
| Ryegrass, Italian | 85 | 90 | 90 | 90 | 90 | 90 | 90 | 90 | 70 | 5 | 50 | 35 | 30 | 85 | 5 | — | — | — | — | 85 | 90 | 65 | 80 | 60 | 95 | 90 |
| Soybean | 98 | 98 | 90 | 70 | 95 | 85 | 35 | 100 | 90 | 35 | 65 | 95 | 15 | 60 | 55 | 98 | 98 | 20 | 98 | 90 | 80 | 95 | 70 | 40 | 100 | 95 |
| Sunflower | — | — | — | — | — | — | — | — | — | — | — | — | 0 | 0 | 40 | — | — | — | — | — | — | — | — | — | — | — |
| Surinam Grass | — | — | — | — | — | — | — | — | — | — | — | — | 25 | 15 | 50 | — | — | — | — | — | — | — | — | — | — | — |
| Velvetleaf | 65 | 100 | 100 | 100 | 100 | 100 | 100 | 100 | 100 | 75 | 100 | 100 | 60 | 100 | 65 | 100 | 85 | 60 | 100 | 100 | 85 | 100 | 100 | 75 | 100 | 100 |
| Waterhemp | 100 | 100 | 100 | 100 | 98 | 100 | 100 | 100 | 100 | 100 | 100 | 100 | — | — | — | 100 | 100 | 98 | 100 | 100 | 100 | 98 | 98 | 100 | 100 | 100 |
| Wheat | 5 | 90 | 90 | 45 | 90 | 85 | 90 | 85 | 10 | 15 | 10 | 0 | 10 | 20 | 5 | — | — | — | — | 55 | 50 | 5 | 45 | 85 | 90 | 60 |
| | 62 g ai/ha | | | | | | | | | | | | | | | | | | | | | | | | | |
| | Compounds | | | | | | | | | | | | | | | | | | | | | | | | | |
| | 105 | 107 | 108 | 109 | 110 | 116 | 125 | 126 | 127 | 128 | 129 | 130 | 131 | 132 | 133 | 136 | 139 | 143 | 144 | 145 | 153 | 154 | 175 | 176 | 177 | 178 |
| Barnyardgrass | 100 | 100 | 100 | 100 | 30 | 100 | 50 | 100 | 60 | 90 | 100 | 85 | 95 | 100 | 60 | 100 | 98 | 100 | 98 | — | 95 | 95 | 80 | 100 | 100 | 60 |
| Bermudagrass | — | — | — | — | — | — | — | — | — | — | — | — | — | — | — | — | — | — | — | — | — | — | — | — | — | — |
| Blackgrass | 90 | 100 | 100 | 100 | — | 90 | 10 | 60 | 60 | 90 | 80 | 98 | 80 | 98 | 90 | 90 | 90 | 85 | 40 | 85 | 90 | 90 | 90 | 5 | 85 | 30 |
| Bromegrass, Downy | — | — | — | — | — | — | — | — | — | — | — | — | — | — | — | — | — | — | — | — | — | — | — | — | — | — |
| Cocklebur | — | — | — | — | — | — | — | — | — | — | — | — | — | — | — | — | — | — | — | — | — | — | — | — | — | — |
| Corn | — | 45 | 30 | 85 | 5 | 65 | 5 | 65 | 25 | 45 | 15 | 30 | 90 | 25 | 25 | 70 | 30 | 75 | 20 | 50 | 50 | 5 | 30 | 5 | 40 | 5 |
| Crabgrass, Large | 100 | 98 | 100 | 100 | 98 | 100 | 25 | 100 | 100 | 95 | 100 | 100 | 100 | 100 | 100 | 100 | 100 | 100 | 100 | — | 98 | 100 | 100 | 95 | 100 | 95 |
| Cupgrass, Woolly | — | — | — | — | — | — | — | — | — | — | — | — | — | — | — | — | — | — | — | — | — | — | — | — | — | — |
| Foxtail, Giant | 100 | 100 | 100 | 100 | 98 | 100 | 35 | 85 | 100 | 100 | 100 | 95 | 100 | 100 | 100 | 100 | 100 | 100 | 100 | — | 85 | 90 | 100 | 90 | 98 | 90 |
| Foxtail, Green | — | — | — | — | — | — | — | — | — | — | — | — | — | — | — | — | — | — | — | — | — | — | — | — | — | — |
| Galium | 100 | 90 | 100 | 100 | 90 | 100 | 85 | 98 | 100 | 80 | 100 | 85 | 75 | 100 | 95 | 100 | 80 | 100 | 80 | 95 | 100 | 100 | 100 | 85 | 100 | 90 |
| Goosegrass | — | — | — | — | — | — | — | — | — | — | — | — | — | — | — | — | — | — | — | — | — | — | — | — | — | — |
| Johnsongrass | 95 | 65 | 90 | 90 | 30 | 95 | 0 | 85 | 55 | 85 | 70 | 65 | 65 | 90 | 85 | 85 | 70 | 85 | 35 | — | 35 | 5 | 35 | 20 | 70 | 5 |
| Kochia | — | — | — | — | — | — | — | — | — | — | — | — | — | — | — | — | — | — | — | — | — | — | — | — | — | — |
| Lambsquarters | 100 | 100 | 100 | 100 | — | 100 | 35 | 98 | 80 | 100 | 100 | 100 | 100 | 100 | 100 | 98 | 85 | — | 98 | — | 98 | 98 | 100 | 98 | 98 | 100 |
| Morningglory | 100 | 100 | 100 | 100 | — | 100 | 50 | 70 | 55 | 70 | 98 | 98 | 100 | 100 | 100 | 100 | 35 | 100 | 100 | 100 | 80 | 90 | 100 | 98 | 95 | 95 |
| Nightshade | — | — | — | — | — | — | — | — | — | — | — | — | — | — | — | — | — | — | — | — | — | — | — | — | — | — |
| Nutsedge, Yellow | 85 | 60 | 40 | 25 | 5 | 55 | 5 | 45 | 50 | 75 | 25 | 65 | 50 | 55 | 25 | 35 | 10 | 40 | 45 | 75 | 75 | 15 | 55 | 5 | 60 | 15 |
| Oat, Wild | — | — | — | — | — | — | — | — | — | — | — | — | — | — | — | — | — | — | — | — | — | — | — | — | — | — |
| Oilseed Rape | 95 | 100 | 98 | 100 | — | 98 | 60 | 100 | 100 | 100 | 95 | 98 | 100 | 98 | 100 | 100 | 100 | 100 | 95 | 98 | 95 | 95 | 100 | 90 | 60 | 80 |
| Pigweed | 100 | 100 | 100 | 100 | 100 | 100 | 75 | 85 | 100 | 100 | 100 | 100 | 100 | 100 | 75 | 70 | 90 | 100 | 100 | 100 | 95 | 98 | 100 | 100 | 100 | 100 |
| Ragweed | 98 | 98 | 95 | 100 | 35 | 98 | 10 | 100 | 25 | 65 | 90 | 100 | 85 | 90 | 60 | 75 | 75 | 98 | 90 | 95 | 95 | 80 | 90 | 100 | 85 | 85 |
| Russian Thistle | — | — | — | — | — | — | — | — | — | — | — | — | — | — | — | — | — | — | — | — | — | — | — | — | — | — |
| Ryegrass, Italian | 95 | 60 | 60 | 50 | — | 90 | 30 | 60 | 60 | 70 | 90 | 25 | 55 | 85 | 85 | 90 | 60 | 80 | 30 | 45 | 50 | 10 | 85 | 10 | 40 | 5 |
| Soybean | 70 | 95 | 90 | 95 | — | 90 | 5 | 40 | 45 | 55 | 55 | 55 | 40 | 75 | 40 | 75 | 5 | 85 | 50 | 70 | 10 | 80 | 98 | 75 | 70 | 40 |
| Sunflower | — | — | — | — | — | — | — | — | — | — | — | — | — | — | — | — | — | — | — | — | — | — | — | — | — | — |
| Surinam Grass | — | — | — | — | — | — | — | — | — | — | — | — | — | — | — | — | — | — | — | — | — | — | — | — | — | — |
| Velvetleaf | 100 | 85 | 100 | 80 | 50 | 100 | 20 | 85 | 60 | 100 | 100 | 100 | 80 | 100 | 55 | 100 | 10 | 98 | 75 | 100 | 60 | 40 | 98 | 75 | 35 | 55 |
| Waterhemp | 100 | 100 | 100 | 100 | 100 | 100 | 65 | 98 | 100 | 100 | 100 | 100 | 100 | 100 | 100 | 100 | 100 | 98 | 98 | 100 | 100 | 100 | 100 | 100 | 98 | 100 |
| Wheat | 85 | 15 | 10 | 15 | — | 45 | 0 | 80 | 40 | 30 | 35 | 35 | 85 | 15 | 45 | 65 | 85 | 60 | 40 | 40 | 85 | 5 | 50 | 0 | 10 | 0 |
| | 62 g ai/ha | | | | | | | | | | | | | | | | | | | | 31 g ai/ha | | | | | |
| | Compounds | | | | | | | | | | | | | | | | | | | | Compounds | | | | | |
| | 179 | 185 | 186 | 187 | 188 | 189 | 195 | 196 | 197 | 198 | 199 | 200 | 201 | 202 | 203 | 205 | 214 | 220 | 224 | 226 | 70 | 72 | 74 | 75 | 76 | 77 |
| Barnyardgrass | 98 | 75 | 100 | 100 | 100 | 100 | 85 | 100 | 98 | 100 | 100 | 70 | 100 | 100 | 98 | 98 | 95 | 15 | 40 | 65 | — | — | — | 100 | 95 | 20 |
| Bermudagrass | — | — | — | — | — | — | — | — | — | — | — | — | — | — | — | — | — | — | — | — | 98 | 85 | 50 | — | — | — |
| Blackgrass | 40 | 10 | 90 | 90 | 90 | 90 | 90 | 90 | 85 | 90 | 90 | 80 | 90 | 85 | 50 | 95 | 40 | 0 | 100 | 85 | 65 | 45 | 10 | — | — | — |
| Bromegrass, Downy | — | — | — | — | — | — | — | — | — | — | — | — | — | — | — | — | — | — | — | — | 20 | 5 | 0 | — | — | — |
| Cocklebur | — | — | — | — | — | — | — | — | — | — | — | — | — | — | — | — | — | — | — | — | 0 | 0 | 100 | — | — | — |
| Corn | 10 | 25 | 85 | 85 | 85 | 40 | 45 | 5 | 5 | 55 | 65 | 15 | 60 | 35 | 50 | 70 | 0 | 0 | 5 | 15 | 40 | 30 | 10 | 60 | 40 | 0 |
| Crabgrass, Large | 100 | 100 | 100 | 100 | 100 | 100 | 95 | 98 | 100 | 100 | 100 | 100 | 100 | 100 | 95 | 100 | 100 | 100 | 100 | 100 | 95 | 55 | 98 | 100 | 100 | 85 |
| Cupgrass, Woolly | — | — | — | — | — | — | — | — | — | — | — | — | — | — | — | — | — | — | — | — | 40 | 10 | 65 | — | — | — |
| Foxtail, Giant | 100 | 90 | 100 | 100 | 100 | 100 | 85 | 100 | 98 | 100 | 100 | 85 | 95 | 100 | 98 | 100 | 98 | 75 | 100 | 90 | 65 | 65 | 80 | 85 | 100 | 80 |
| Foxtail, Green | — | — | — | — | — | — | — | — | — | — | — | — | — | — | — | — | — | — | — | — | 15 | 30 | 85 | — | — | — |
| Galium | 100 | 100 | 100 | 95 | 100 | 95 | 85 | 95 | 100 | 95 | 100 | 100 | 95 | 100 | 100 | 90 | 80 | 80 | 80 | 90 | 65 | 70 | 85 | 100 | 98 | 0 |
| Goosegrass | — | — | — | — | — | — | — | — | — | — | — | — | — | — | — | — | — | — | — | — | 60 | 75 | 75 | — | — | — |
| Johnsongrass | 10 | 10 | 98 | 98 | 98 | 98 | 30 | 85 | 40 | 80 | 70 | 80 | 85 | 85 | 0 | 80 | 40 | 5 | 50 | 55 | 20 | 40 | 10 | 100 | 80 | 45 |
| Kochia | — | — | — | — | — | — | — | — | — | — | — | — | — | — | — | — | — | — | — | — | 40 | 5 | 98 | — | — | — |
| Lambsquarters | 100 | 98 | 100 | 100 | 100 | 100 | — | — | — | — | 100 | 100 | — | — | 100 | 95 | 100 | 98 | 100 | 100 | 98 | 100 | 85 | — | — | — |
| Morningglory | 95 | 75 | 70 | 100 | 98 | 98 | 100 | 98 | 100 | 100 | 100 | 95 | 100 | 100 | 95 | 100 | 98 | 5 | 10 | 85 | 0 | 15 | 75 | — | — | — |
| Nightshade | — | — | — | — | — | — | — | — | — | — | — | — | — | — | — | — | — | — | — | — | 70 | 75 | 90 | — | — | — |
| Nutsedge, Yellow | 15 | 40 | 60 | 60 | 65 | 80 | 85 | 65 | 25 | 15 | 45 | 25 | 20 | 5 | 35 | 65 | 55 | 0 | 80 | 5 | 0 | 0 | 15 | 55 | 40 | 10 |
| Oat, Wild | — | — | — | — | — | — | — | — | — | — | — | — | — | — | — | — | — | — | — | — | 5 | 30 | 0 | — | — | — |
| Oilseed Rape | 85 | 80 | 100 | 90 | 100 | 95 | 85 | 98 | 95 | 100 | 95 | 95 | 100 | 100 | 100 | 100 | 95 | 30 | 90 | 85 | — | — | — | — | — | — |
| Pigweed | 100 | 100 | 100 | 100 | 100 | 100 | 98 | 98 | 100 | 100 | 100 | 100 | 100 | 100 | 100 | 100 | 100 | 100 | 100 | 100 | 100 | 100 | 100 | 100 | 100 | 100 |
| Ragweed | 98 | 75 | 100 | 100 | 95 | 98 | 85 | 90 | 85 | 85 | 85 | 35 | 85 | 95 | 80 | 100 | 98 | 65 | 85 | 100 | 0 | 0 | 30 | 90 | 90 | 35 |
| Russian Thistle | — | — | — | — | — | — | — | — | — | — | — | — | — | — | — | — | — | — | — | — | 65 | 5 | — | — | — | — |
| Ryegrass, Italian | 80 | 30 | 80 | 80 | 90 | 90 | 60 | 85 | 35 | 70 | 85 | 90 | 90 | 50 | 60 | 70 | 30 | 0 | 60 | 5 | 20 | 40 | 5 | — | — | — |
| Soybean | 65 | 5 | 65 | 95 | 90 | 95 | 5 | 55 | 80 | 90 | 55 | 70 | 85 | 70 | 5 | 85 | 85 | 10 | 40 | 70 | 5 | 20 | 25 | 85 | 80 | 10 |
| Sunflower | — | — | — | — | — | — | — | — | — | — | — | — | — | — | — | — | — | — | — | — | 0 | 0 | 10 | — | — | — |
| Surinam Grass | — | — | — | — | — | — | — | — | — | — | — | — | — | — | — | — | — | — | — | — | 5 | 10 | 15 | — | — | — |
| Velvetleaf | 80 | 65 | 100 | 100 | 100 | 100 | 60 | 100 | 90 | 85 | 100 | 70 | 100 | 100 | 98 | 100 | 100 | 20 | 65 | 80 | 20 | 20 | 45 | 75 | 75 | 35 |
| Waterhemp | 100 | 100 | 100 | 100 | 100 | 100 | 100 | 100 | 100 | 98 | 100 | 100 | 100 | 100 | 95 | 100 | 100 | 95 | 100 | 100 | — | — | — | 100 | 98 | 98 |
| Wheat | 0 | 60 | 90 | 80 | 90 | 80 | 45 | 65 | 0 | 35 | 85 | 15 | 85 | 85 | 75 | 15 | 0 | 0 | 0 | 0 | 5 | 5 | 0 | — | — | — |
| | 31 g ai/ha | | | | | | | | | | | | | | | | | | | | | | | | | |
| | Compounds | | | | | | | | | | | | | | | | | | | | | | | | | |
| | 78 | 95 | 96 | 97 | 98 | 101 | 103 | 104 | 105 | 107 | 108 | 109 | 110 | 116 | 125 | 126 | 127 | 128 | 129 | 130 | 131 | 132 | 133 | 136 | 139 | 140 |
| Barnyardgrass | 100 | 100 | 85 | 95 | 30 | 75 | 100 | 100 | 98 | 40 | 50 | 100 | 25 | 98 | 5 | 75 | 45 | 80 | 95 | 50 | 55 | 95 | 65 | 75 | 40 | 80 |
| Bermudagrass | — | — | — | — | — | — | — | — | — | — | — | — | — | — | — | — | — | — | — | — | — | — | — | — | — | — |
| Blackgrass | — | 100 | 100 | 90 | 70 | 75 | 100 | 85 | 90 | 95 | 100 | 100 | — | 90 | 0 | 55 | 20 | 50 | 60 | 25 | 40 | 95 | 85 | 85 | 85 | 50 |
| Bromegrass, Downy | — | — | — | — | — | — | — | — | — | — | — | — | — | — | — | — | — | — | — | — | — | — | — | — | — | — |
| Cocklebur | — | — | — | — | — | — | — | — | — | — | — | — | — | — | — | — | — | — | — | — | — | — | — | — | — | — |
| Corn | 60 | 35 | 45 | 50 | 35 | 0 | 75 | 40 | 65 | 30 | 5 | 25 | 5 | 15 | 0 | 25 | 5 | 15 | 0 | 5 | 40 | 55 | 5 | 35 | 25 | 10 |
| Crabgrass, Large | 100 | 98 | 98 | 98 | 98 | 80 | 100 | 100 | 100 | 98 | 100 | 90 | 95 | 100 | 5 | 90 | 80 | 100 | 90 | 100 | 90 | 98 | 100 | 100 | 100 | 98 |
| Cupgrass, Woolly | — | — | — | — | — | — | — | — | — | — | — | — | — | — | — | — | — | — | — | — | — | — | — | — | — | — |
| Foxtail, Giant | 90 | 100 | 100 | 98 | 100 | 80 | 100 | 100 | 100 | 100 | 100 | 100 | 98 | 100 | 20 | 45 | 98 | 98 | 100 | 70 | 85 | 95 | 85 | 100 | 90 | 70 |
| Foxtail, Green | — | — | — | — | — | — | — | — | — | — | — | — | — | — | — | — | — | — | — | — | — | — | — | — | — | — |
| Galium | 85 | 100 | 90 | 85 | 100 | 80 | 100 | 90 | 100 | 75 | 85 | 10 | 50 | 100 | 80 | 85 | 80 | 80 | 100 | 75 | 55 | 98 | — | 85 | 30 | 100 |
| Goosegrass | — | — | — | — | — | — | — | — | — | — | — | — | — | — | — | — | — | — | — | — | — | — | — | — | — | — |
| Johnsongrass | 85 | 60 | 60 | 60 | 65 | 20 | 85 | 90 | 80 | 30 | 35 | 80 | 15 | 60 | 0 | 40 | 55 | 45 | 10 | 35 | 20 | 25 | 40 | 60 | 10 | 25 |
| Kochia | — | — | — | — | — | — | — | — | — | — | — | — | — | — | — | — | — | — | — | — | — | — | — | — | — | — |
| Lambsquarters | — | 100 | 100 | 100 | 100 | 85 | 100 | 98 | 85 | 100 | 100 | 100 | — | 100 | 15 | 85 | 100 | 98 | 98 | 85 | 98 | 100 | 75 | 98 | 35 | 75 |
| Morningglory | — | 100 | 85 | 95 | 45 | 85 | 100 | 100 | 100 | 90 | 98 | 95 | — | 100 | 45 | 55 | 60 | 75 | 45 | 65 | 40 | 60 | 75 | 100 | 5 | 90 |
| Nightshade | — | — | — | — | — | — | — | — | — | — | — | — | — | — | — | — | — | — | — | — | — | — | — | — | — | — |
| Nutsedge, Yellow | 70 | 50 | 50 | 50 | 30 | 5 | 40 | 70 | 50 | 15 | 5 | 5 | — | 25 | 0 | 25 | 25 | 45 | 5 | 25 | 45 | 35 | 20 | 30 | 15 | 15 |
| Oat, Wild | — | — | — | — | — | — | — | — | — | — | — | — | — | — | — | — | — | — | — | — | — | — | — | — | — | — |
| Oilseed Rape | — | 100 | 85 | 100 | 100 | 90 | 100 | 100 | 95 | 98 | 90 | 40 | — | 90 | 30 | 85 | 85 | 100 | 95 | 75 | 95 | 75 | 60 | 100 | 50 | 95 |
| Pigweed | 100 | 100 | 100 | 100 | 100 | 80 | 100 | 100 | 100 | 100 | 100 | 70 | 95 | 100 | 65 | 100 | 100 | 100 | 100 | 100 | 100 | 100 | 100 | 70 | 55 | 100 |
| Ragweed | 80 | 80 | 80 | 50 | 95 | — | — | 98 | 98 | 50 | 95 | 100 | 35 | 80 | 5 | 70 | 15 | 25 | 85 | 70 | 75 | 70 | 10 | 45 | 15 | 70 |
| Russian Thistle | — | — | — | — | — | — | — | — | — | — | — | — | — | — | — | — | — | — | — | — | — | — | — | — | — | — |
| Ryegrass, Italian | — | 50 | 55 | 30 | 30 | 30 | 75 | 80 | 85 | 10 | 30 | 10 | — | 30 | 0 | 15 | 25 | 5 | 5 | 5 | 15 | 15 | 35 | 55 | 10 | 55 |
| Soybean | 98 | 80 | 20 | 50 | 30 | 5 | 90 | 85 | 70 | 15 | 20 | 70 | 10 | 65 | 5 | 25 | 30 | 35 | 40 | 10 | 30 | 35 | 50 | 45 | 5 | 25 |
| Sunflower | — | — | — | — | — | — | — | — | — | — | — | — | — | — | — | — | — | — | — | — | — | — | — | — | — | — |
| Surinam Grass | — | — | — | — | — | — | — | — | — | — | — | — | — | — | — | — | — | — | — | — | — | — | — | — | — | — |
| Velvetleaf | 80 | 50 | 70 | 100 | 100 | 35 | 100 | 100 | 98 | 60 | 55 | 10 | 50 | 80 | 20 | 65 | 60 | 5 | 55 | 55 | 25 | 55 | 35 | 65 | 0 | 45 |
| Waterhemp | 100 | 100 | 85 | 98 | 98 | 95 | 100 | 100 | 100 | 100 | 98 | 75 | 100 | 100 | 65 | 90 | 100 | 100 | 100 | 100 | 100 | 100 | 85 | 85 | 85 | 95 |
| Wheat | — | 5 | 50 | 0 | 30 | 5 | 10 | 40 | 75 | 5 | 10 | 0 | — | 40 | 0 | 45 | 5 | 20 | 0 | 0 | 45 | 15 | 0 | 10 | 30 | 35 |
| | 31 g ai/ha | | | | | | | | | | | | | | | | | | | | | | | | | |
| | Compounds | | | | | | | | | | | | | | | | | | | | | | | | | |
| | 143 | 144 | 145 | 153 | 154 | 175 | 176 | 177 | 178 | 179 | 185 | 186 | 187 | 188 | 189 | 195 | 196 | 197 | 198 | 199 | 200 | 201 | 202 | 203 | 205 | 214 |
| Barnyardgrass | 75 | 75 | 95 | 15 | 55 | 80 | 25 | 65 | 10 | 60 | 55 | 100 | 95 | 100 | 95 | 60 | 98 | 65 | 100 | 95 | 45 | 98 | 95 | 35 | 85 | 40 |
| Bermudagrass | — | — | — | — | — | — | — | — | — | — | — | — | — | — | — | — | — | — | — | — | — | — | — | — | — | — |
| Blackgrass | 85 | 40 | 70 | 80 | 75 | 30 | 5 | 50 | 5 | 5 | 5 | 85 | 90 | 90 | 90 | 5 | 85 | 0 | 85 | 90 | 50 | 85 | 10 | 30 | 80 | 85 |
| Bromegrass, Downy | — | — | — | — | — | — | — | — | — | — | — | — | — | — | — | — | — | — | — | — | — | — | — | — | — | — |
| Cocklebur | — | — | — | — | — | — | — | — | — | — | — | — | — | — | — | — | — | — | — | — | — | — | — | — | — | — |
| Corn | 40 | 5 | 10 | 10 | 0 | 20 | 5 | 5 | 5 | 5 | 5 | 55 | 20 | 55 | 5 | 25 | 5 | 5 | 5 | 20 | 0 | 40 | 5 | 5 | 70 | 0 |
| Crabgrass, Large | 100 | 100 | 100 | 85 | 100 | 100 | 98 | 100 | 100 | 100 | 80 | 100 | 100 | 100 | 100 | 100 | 98 | 100 | 98 | 98 | 100 | 100 | 100 | 80 | 100 | 100 |
| Cupgrass, Woolly | — | — | — | — | — | — | — | — | — | — | — | — | — | — | — | — | — | — | — | — | — | — | — | — | — | — |
| Foxtail, Giant | 100 | 75 | 100 | 60 | 30 | 75 | 35 | 85 | 65 | 98 | 60 | 100 | 100 | 100 | 100 | 65 | 98 | 65 | 98 | 100 | 75 | 100 | 100 | 60 | 100 | 90 |
| Foxtail, Green | — | — | — | — | — | — | — | — | — | — | — | — | — | — | — | — | — | — | — | — | — | — | — | — | — | — |
| Galium | 90 | 30 | 90 | 30 | 90 | 85 | 85 | 100 | 90 | 98 | 80 | 50 | 90 | 98 | 90 | 0 | 98 | 90 | 98 | 40 | 85 | 95 | 98 | 100 | 100 | 80 |
| Goosegrass | — | — | — | — | — | — | — | — | — | — | — | — | — | — | — | — | — | — | — | — | — | — | — | — | — | — |
| Johnsongrass | 70 | 0 | 55 | 5 | 5 | 0 | 0 | 10 | 0 | 10 | 5 | 75 | 75 | 80 | 65 | 5 | 55 | 10 | 25 | 45 | 50 | 85 | 50 | 0 | 70 | 0 |
| Kochia | — | — | — | — | — | — | — | — | — | — | — | — | — | — | — | — | — | — | — | — | — | — | — | — | — | — |
| Lambsquarters | — | 95 | — | 85 | 65 | 98 | 80 | 85 | 80 | 90 | 98 | 100 | 100 | 100 | 98 | — | — | — | — | 95 | 75 | — | — | 75 | 85 | 100 |
| Morningglory | 100 | 60 | 80 | 30 | 35 | 95 | 65 | 25 | 35 | 85 | 15 | 98 | 85 | 98 | 95 | 35 | 98 | 75 | 95 | 60 | 55 | 85 | 90 | 30 | 100 | 25 |
| Nightshade | — | — | — | — | — | — | — | — | — | — | — | — | — | — | — | — | — | — | — | — | — | — | — | — | — | — |
| Nutsedge, Yellow | 30 | 0 | 10 | 20 | 0 | 10 | 5 | 5 | 5 | 10 | 5 | 10 | 25 | 30 | 25 | 20 | 15 | 0 | 5 | 30 | 20 | 5 | 0 | 5 | 40 | 25 |
| Oat, Wild | — | — | — | — | — | — | — | — | — | — | — | — | — | — | — | — | — | — | — | — | — | — | — | — | — | — |
| Oilseed Rape | 100 | 80 | 60 | 60 | 85 | 98 | 90 | 70 | 70 | 60 | 70 | 100 | 50 | 100 | 98 | 30 | 90 | 80 | 95 | 80 | 60 | 100 | 100 | 50 | 100 | 80 |
| Pigweed | 100 | 98 | 100 | 20 | 85 | 100 | 100 | 100 | 100 | 100 | 100 | 100 | 70 | 100 | 100 | — | 98 | 100 | 100 | 100 | 100 | 100 | 100 | 100 | 95 | 100 |
| Ragweed | 98 | 85 | 75 | 50 | 25 | 80 | 85 | 75 | 70 | 90 | 5 | 75 | 80 | 75 | 85 | — | 85 | 60 | 60 | 30 | 5 | 80 | 75 | 75 | 80 | 85 |
| Russian Thistle | — | — | — | — | — | — | — | — | — | — | — | — | — | — | — | — | — | — | — | — | — | — | — | — | — | — |
| Ryegrass, Italian | 35 | 5 | 5 | 5 | 5 | 30 | 5 | 5 | 5 | 5 | 0 | 5 | 50 | 70 | 40 | 10 | 30 | 15 | 25 | 40 | 0 | 65 | 5 | 5 | 60 | 5 |
| Soybean | 35 | 5 | 20 | 5 | 35 | 30 | 30 | 40 | 5 | 35 | 5 | 85 | 85 | 90 | 20 | 5 | 40 | 45 | 75 | 30 | 50 | 70 | 65 | 5 | 80 | 60 |
| Sunflower | — | — | — | — | — | — | — | — | — | — | — | — | — | — | — | — | — | — | — | — | — | — | — | — | — | — |
| Surinam Grass | — | — | — | — | — | — | — | — | — | — | — | — | — | — | — | — | — | — | — | — | — | — | — | — | — | — |
| Velvetleaf | 100 | 15 | 90 | 10 | 10 | 55 | 30 | 0 | 15 | 35 | 20 | 100 | 95 | 100 | 98 | 15 | 40 | 25 | 35 | 40 | 40 | 90 | 60 | 30 | 55 | 35 |
| Waterhemp | 98 | 98 | 98 | 60 | 85 | 100 | 100 | 98 | 90 | 100 | 100 | 100 | 100 | 100 | 100 | — | 100 | 100 | 98 | 98 | 100 | 100 | 100 | 85 | 100 | 100 |
| Wheat | 40 | 5 | 5 | 15 | 0 | 0 | 0 | 10 | 0 | 0 | 0 | 80 | 20 | 85 | 40 | 0 | 0 | 0 | 5 | 60 | 0 | 80 | 0 | 40 | 5 | 0 |

| | 31 g ai/ha | | |
|---|---|---|---|
| | Compounds | | |
| | 220 | 224 | 226 |
| Barnyardgrass | 0 | 25 | 10 |
| Bermudagrass | — | — | — |
| Blackgrass | 0 | 80 | 60 |
| Bromegrass, Downy | — | — | — |
| Cocklebur | — | — | — |
| Corn | 0 | 0 | 10 |
| Crabgrass, Large | 95 | 100 | 80 |
| Cupgrass, Woolly | — | — | — |
| Foxtail, Giant | 10 | 95 | 80 |
| Foxtail, Green | — | — | — |
| Galium | 5 | 80 | 0 |
| Goosegrass | — | — | — |
| Johnsongrass | 10 | 15 | 10 |
| Kochia | — | — | — |
| Lambsquarters | 60 | 100 | 100 |
| Morningglory | 0 | 5 | 85 |
| Nightshade | — | — | — |
| Nutsedge, Yellow | 0 | 85 | 0 |
| Oat, Wild | — | — | — |
| Oilseed Rape | 80 | 80 | 30 |
| Pigweed | 65 | 100 | 100 |
| Ragweed | 80 | 85 | 90 |
| Russian Thistle | — | — | — |
| Ryegrass, Italian | 0 | 10 | 0 |
| Soybean | 10 | 10 | 10 |
| Sunflower | — | — | — |
| Surinam Grass | — | — | — |
| Velvetleaf | 5 | 40 | 25 |
| Waterhemp | 75 | 100 | 100 |
| Wheat | 10 | 0 | 15 |

| | 16 g ai/ha | | | | | 8 g ai/ha | 4 g ai/ha |
|---|---|---|---|---|---|---|---|
| | Compounds | | | | | Compound | Compound |
| | 75 | 77 | 101 | 140 | 178 | 140 | 140 |
| Barnyardgrass | 60 | 10 | 5 | 30 | 0 | 5 | 0 |
| Blackgrass | — | — | 30 | 5 | 0 | 0 | 0 |
| Corn | 10 | 0 | 0 | 0 | 5 | 0 | 0 |
| Crabgrass, Large | 100 | 60 | 65 | 75 | 30 | 55 | 45 |
| Foxtail, Giant | 80 | 20 | 15 | 75 | 5 | 40 | 20 |
| Galium | 90 | 0 | 80 | 98 | 70 | 60 | 50 |
| Johnsongrass | 65 | 10 | 0 | 5 | 0 | 5 | 20 |
| Lambsquarters | — | — | 85 | 40 | 65 | 55 | 5 |
| Morningglory | — | — | 40 | 95 | 15 | 5 | 5 |
| Nutsedge, Yellow | — | 10 | 5 | 0 | 0 | 15 | 0 |
| Oilseed Rape | — | — | 75 | 95 | 50 | 80 | 5 |
| Pigweed | 85 | 70 | 20 | 100 | 45 | 100 | 80 |
| Ragweed | 70 | 10 | — | 25 | 60 | 5 | 5 |
| Ryegrass, Italian | — | — | 0 | 10 | 0 | 5 | 0 |
| Soybean | 35 | 0 | 0 | 5 | 5 | 5 | 10 |
| Velvetleaf | 60 | 30 | 0 | 10 | 10 | 5 | 0 |
| Waterhemp | 80 | 70 | 85 | 85 | 65 | 70 | 70 |
| Wheat | — | — | 0 | 0 | 0 | 0 | 0 |

Flood

| | 500 g ai/ha | 250 g ai/ha | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|
| | Compound | Compounds | | | | | | | | | | | | | | | | | | | | | | | | | | | | | |
| | 205 | 3 | 4 | 5 | 16 | 28 | 29 | 30 | 31 | 32 | 33 | 34 | 35 | 53 | 64 | 70 | 71 | 72 | 73 | 74 | 77 | 106 | 109 | 127 | 128 | 141 | 145 | 176 | 177 | 178 | 195 |
| Barnyardgrass | 60 | 0 | 0 | 0 | 20 | 20 | 20 | 20 | 20 | 0 | 0 | 20 | 10 | 15 | 25 | 65 | 40 | 20 | 0 | 15 | 20 | 30 | 40 | 30 | — | 35 | 40 | 10 | 0 | 20 | 45 |
| Ducksalad | 100 | 60 | 60 | 60 | 100 | 100 | 85 | 100 | — | 70 | 80 | 80 | 60 | 40 | 50 | 85 | 0 | 75 | 55 | 90 | 75 | 70 | 85 | 80 | 90 | 100 | 100 | 100 | 85 | 90 | 85 |
| Rice | 35 | 0 | 0 | 0 | 25 | 0 | 0 | 0 | 15 | 0 | 0 | 10 | 0 | 30 | 25 | 50 | 45 | 30 | 0 | 15 | 0 | 0 | 35 | 0 | 0 | 20 | 40 | 20 | 0 | 20 | 20 |
| Sedge, Umbrella | 100 | 0 | 60 | 75 | 100 | 90 | 80 | 80 | 85 | 80 | 85 | 80 | 80 | 80 | 75 | 90 | 85 | 65 | 30 | 90 | 75 | 98 | 85 | 95 | 85 | 100 | 100 | 100 | 90 | 80 | 85 |

| | 250 g ai/ha | | | | 125 g ai/ha | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|
| | Compounds | | | | Compounds | | | | | | | | | | | | | | | | | | | | | | | | | | | |
| | 200 | 203 | 204 | 205 | 3 | 4 | 16 | 28 | 29 | 30 | 31 | 32 | 33 | 34 | 35 | 53 | 64 | 70 | 71 | 72 | 73 | 74 | 77 | 106 | 109 | 127 | 128 | 141 | 145 | 149 | 168 | 176 |
| Barnyardgrass | 40 | 0 | 0 | 35 | 0 | 0 | 0 | 0 | 0 | 0 | 0 | 0 | 0 | 0 | 10 | 0 | 0 | 45 | 20 | 20 | 0 | 15 | 20 | 20 | 35 | 15 | 35 | 20 | 30 | 0 | 0 | 20 |
| Ducksalad | 100 | 80 | 90 | 100 | 50 | 50 | 95 | 30 | 85 | 80 | — | 50 | 20 | 30 | 20 | 0 | 30 | 85 | 0 | 75 | 45 | 0 | 70 | 70 | 75 | 60 | 80 | 95 | 100 | 70 | 70 | 95 |
| Rice | 20 | 0 | 0 | 0 | 0 | 0 | 25 | 0 | 0 | 0 | 0 | 0 | 0 | 0 | 0 | 10 | 15 | 25 | 30 | 0 | 0 | 15 | 0 | 0 | 20 | 0 | 0 | 20 | 25 | 0 | 0 | 20 |
| Sedge, Umbrella | 100 | 85 | 80 | 100 | 0 | 0 | 100 | 70 | 75 | 0 | 85 | 50 | 30 | 70 | 50 | 0 | 50 | 85 | 75 | 65 | 0 | 0 | 60 | 95 | 75 | 70 | 75 | 95 | 100 | 80 | 80 | 95 |

| | 125 g ai/ha | | | | | | | | 62 g ai/ha | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|---|
| | Compounds | | | | | | | | Compounds | | | | | | | | | | | | | | | | | | | | | | | |
| | 177 | 178 | 195 | 200 | 203 | 204 | 205 | 226 | 3 | 4 | 5 | 16 | 28 | 29 | 30 | 31 | 32 | 33 | 34 | 35 | 53 | 64 | 70 | 71 | 72 | 73 | 74 | 77 | 106 | 109 | 127 | 128 |
| Barnyardgrass | 0 | 0 | 40 | 20 | 0 | 0 | 25 | 0 | 0 | 0 | 0 | 0 | 0 | 0 | 0 | 0 | 0 | 0 | 0 | 0 | 0 | 0 | 0 | 0 | 20 | 0 | 15 | 0 | 0 | 0 | 0 | 0 |
| Ducksalad | 75 | 60 | 85 | 100 | 20 | 50 | 90 | 75 | 50 | 40 | 0 | 85 | 0 | 0 | — | — | 20 | 10 | 10 | 20 | 0 | 0 | 40 | 0 | 70 | 20 | 0 | 40 | 50 | 40 | 40 | 75 |
| Rice | 0 | 0 | 0 | 15 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 15 | 0 | 0 | 0 | 0 | 0 |
| Sedge, Umbrella | 85 | 40 | 80 | 100 | 70 | 80 | 95 | 85 | 0 | 0 | 50 | 85 | 65 | 0 | 0 | 70 | 30 | 30 | 10 | 30 | 0 | 0 | 75 | 70 | 60 | 0 | 0 | 50 | 85 | 60 | 60 | 50 |

| | 62 g ai/ha | | | | | | | | | | | | | 31 g ai/ha | | | | | | | | | | | | | | | | | | |
|---|---|---|---|---|---|---|---|---|---|---|---|---|---|---|---|---|---|---|---|---|---|---|---|---|---|---|---|---|---|---|---|---|
| | Compounds | | | | | | | | | | | | | Compounds | | | | | | | | | | | | | | | | | | |
| | 141 | 145 | 149 | 168 | 176 | 177 | 178 | 195 | 200 | 203 | 204 | 205 | 226 | 3 | 4 | 16 | 28 | 29 | 30 | 31 | 32 | 33 | 34 | 35 | 53 | 64 | 70 | 72 | 73 | 74 | 77 | 106 |
| Barnyardgrass | 0 | 0 | 0 | 0 | 0 | 0 | 0 | 30 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| Ducksalad | 80 | 100 | 55 | 50 | 70 | 50 | 30 | 20 | 30 | 0 | 0 | 75 | 60 | 40 | 0 | 50 | 0 | 0 | 60 | — | 10 | 0 | 0 | 0 | 0 | 0 | 30 | 0 | 0 | 0 | 20 | 40 |
| Rice | 0 | 20 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| Sedge, Umbrella | 70 | 100 | 70 | 50 | 40 | 75 | 0 | 75 | 80 | 40 | 60 | 75 | 25 | 0 | 0 | 70 | 60 | 0 | 0 | 30 | 20 | 10 | 0 | 20 | 0 | 0 | 75 | 50 | 0 | 0 | 0 | 70 |

| | 31 g ai/ha | | | | | | | | | | | | | | |
|---|---|---|---|---|---|---|---|---|---|---|---|---|---|---|---|
| | Compounds | | | | | | | | | | | | | | |
| | 109 | 127 | 128 | 141 | 145 | 149 | 168 | 176 | 177 | 178 | 195 | 200 | 203 | 204 | 226 |
| Barnyardgrass | 0 | 0 | 0 | 0 | 0 | 0 | 0 | 0 | 0 | 0 | 0 | 0 | 0 | 0 | 0 |
| Ducksalad | 30 | 30 | 40 | 40 | 100 | 0 | 0 | 0 | 0 | 0 | 0 | 30 | 0 | 0 | 30 |
| Rice | 0 | 0 | 0 | 0 | 0 | 0 | 0 | 0 | 0 | 0 | 0 | 0 | 0 | 0 | 0 |
| Sedge, Umbrella | 30 | 50 | 30 | 30 | 100 | 60 | 0 | 0 | 50 | 0 | 0 | 50 | 40 | 0 | 20 |

### Test D

Seeds of plant species selected from bluegrass (annual bluegrass, Poa annua), blackgrass (Alopecurus myosuroides), canarygrass (littleseed canarygrass, Phalaris minor), chickweed (common chickweed, Stellaria media), galium (catchweed bedstraw, Galium aparine), bromegrass, downy (downy bromegrass, Bromus tectorum), field poppy (Papaver rhoeas), field violet (Viola arvensis), foxtail, green (green foxtail, Setaria viridis), deadnettle (henbit deadnettle, Lamium amplexicaule), ryegrass, Italian (Italian ryegrass, Lolium multiflorum), kochia (Kochia scoparia), lambsquarters (Chenopodium album), oilseed rape (Brassica napus), pigweed (redroot pigweed, Amaranthus retroflexus), chamomile (scentless chamomile, Matricaria inodora), speedwell (bird's-eye speedwell, Veronica persica), barley, spring (spring barley, Hordeum vulgare), wheat, spring (spring wheat, Triticum aestivum), buckwheat, wild (wild buckwheat, Polygonum convolvulus), mustard, wild (wild mustard, Sinapis arvensis), oat, wild (wild oat, Avena fatua), radish, wild (wild radish, Raphanus raphanistrum), windgrass (Apera spica-venti), Russian thistle (Salsola kali), barley, winter (winter barley, Hordeum vulgare), and wheat, winter (winter wheat, Triticum aestivum) were planted into a silt loam soil and treated preemergence with test chemicals formulated in a non-phytotoxic solvent mixture which included a surfactant.

At the same time, these species were planted in pots containing Redi-Earth® planting medium (Scotts Company, 14111 Scottslawn Road, Marysville, Ohio 43041) comprising spaghnum peat moss, vermiculite, wetting agent and starter nutrients and treated with postemergence applications of the test chemicals formulated in the same manner. Plants ranged in height from 2 to 18 cm (1- to 4-leaf stage). Treated plants and controls were maintained in a controlled growth environment for 14 to 21 days after which time all species were compared to controls and visually evaluated. Plant response ratings, summarized in Table D, are based on a scale of 0 to 100 where 0 is no effect and 100 is complete control. A dash (-) response means no test result.

TABLE D
Postemergence

| | 250 g ai/ha | | | | | | |
|---|---|---|---|---|---|---|---|
| | Compounds | | | | | | |
| | 70 | 76 | 78 | 97 | 103 | 105 | 138 |
| Barley, Spring | 5 | 15 | 30 | 20 | 15 | 45 | 40 |
| Barley, Winter | 5 | 20 | 25 | 25 | 15 | 30 | 40 |
| Blackgrass | 0 | 10 | 65 | 35 | 35 | 70 | 75 |
| Bluegrass | 0 | 20 | 30 | 70 | 30 | 70 | 60 |
| Bromegrass, Downy | 10 | 15 | 25 | 25 | 15 | 30 | 20 |
| Buckwheat, Wild | 35 | 100 | 70 | 85 | 60 | 80 | 80 |
| Canarygrass | 10 | 15 | 25 | 30 | 10 | 40 | 70 |
| Chamomile | 50 | 55 | 75 | 65 | 60 | 80 | 65 |
| Chickweed | 85 | 70 | 80 | 85 | 70 | 80 | 75 |
| Deadnettle | 60 | 70 | 60 | 40 | 30 | 60 | 85 |
| Field Poppy | 100 | 90 | 85 | 80 | 70 | 95 | 75 |
| Field Violet | 75 | 85 | 65 | 90 | 60 | 100 | 95 |
| Foxtail, Green | 60 | 85 | 80 | 80 | 65 | 80 | 80 |
| Galium | 80 | 95 | 85 | 80 | 80 | 85 | 100 |
| Kochia | 95 | 75 | 75 | 75 | 75 | 80 | 80 |
| Lambsquarters | 98 | 100 | 80 | 80 | 60 | 80 | 80 |
| Mustard, Wild | — | 100 | 90 | 90 | 60 | 90 | 100 |
| Oat, Wild | 10 | 15 | 40 | 35 | 25 | 50 | 60 |
| Oilseed Rape | 80 | 70 | 80 | 75 | 45 | 85 | 100 |
| Pigweed | 90 | 80 | 85 | 80 | 70 | 85 | 90 |
| Radish, Wild | 85 | 65 | 75 | 80 | 60 | 80 | 95 |
| Russian Thistle | 90 | 40 | — | — | — | — | — |
| Ryegrass, Italian | 15 | 10 | 45 | 35 | 35 | 65 | 55 |
| Speedwell | 100 | 100 | 90 | 80 | 70 | 95 | 100 |
| Wheat, Spring | 10 | 10 | 25 | 15 | 5 | 40 | 25 |
| Wheat, Winter | 10 | 20 | 55 | 20 | 30 | 70 | 35 |
| Windgrass | 0 | 15 | 60 | 55 | 35 | 70 | 60 |
| | 250 g ai/ha | | | | | | |
| | Compounds | | | | | | |
| | 188 | 188 | 189 | 201 | 202 | 214 | |
| Barley, Spring | 65 | 65 | 25 | 40 | 30 | 40 | |
| Barley, Winter | 65 | 65 | 20 | 35 | 25 | 35 | |
| Blackgrass | 45 | 45 | 20 | 65 | 35 | 70 | |
| Bluegrass | 35 | 35 | 30 | 40 | 35 | 20 | |
| Bromegrass, Downy | 20 | 20 | 20 | 50 | 35 | 20 | |
| Buckwheat, Wild | 70 | 70 | 80 | 70 | 90 | 80 | |
| Canarygrass | 35 | 35 | 30 | 60 | 20 | 30 | |
| Chamomile | 75 | 75 | 65 | 65 | 65 | 70 | |
| Chickweed | 95 | 95 | 85 | 75 | 70 | 70 | |
| Deadnettle | 55 | 55 | 75 | 65 | 65 | 70 | |
| Field Poppy | 95 | 95 | 75 | 65 | 75 | 80 | |
| Field Violet | 100 | 100 | 100 | 75 | 50 | 100 | |
| Foxtail, Green | 55 | 55 | 50 | 55 | 30 | 60 | |
| Galium | 90 | 90 | 85 | 85 | 75 | 100 | |
| Kochia | 85 | 85 | 85 | 85 | 85 | 85 | |
| Lambsquarters | 90 | 90 | 90 | 90 | 80 | 90 | |
| Mustard, Wild | 85 | 85 | 80 | 90 | 75 | 100 | |
| Oat, Wild | 70 | 70 | 35 | 70 | 40 | 40 | |
| Oilseed Rape | 70 | 70 | 60 | 70 | 60 | 100 | |
| Pigweed | 80 | 80 | 75 | 80 | 85 | 95 | |
| Radish, Wild | 90 | 90 | 85 | 65 | 75 | 100 | |
| Russian Thistle | — | — | — | — | — | — | |
| Ryegrass, Italian | 65 | 65 | 30 | 50 | 15 | 45 | |
| Speedwell | 90 | 90 | 90 | 100 | 100 | 100 | |
| Wheat, Spring | 40 | 40 | 25 | 30 | 20 | 20 | |
| Wheat, Winter | 65 | 65 | 50 | 40 | 25 | 40 | |
| Windgrass | 45 | 45 | 35 | 45 | 35 | 40 | |
| | 125 g ai/ha | | | | | | |
| | Compounds | | | | | | |
| | 70 | 74 | 76 | 78 | 97 | 103 | 105 |
| Barley, Spring | 5 | 0 | 10 | 10 | 15 | 10 | 30 |
| Barley, Winter | 5 | 5 | 15 | 15 | 25 | 5 | 20 |
| Blackgrass | 0 | 0 | 10 | 30 | 20 | 20 | 65 |
| Bluegrass | 0 | 5 | 20 | 15 | 40 | 20 | 40 |
| Bromegrass, Downy | 0 | 0 | 10 | 20 | 20 | 15 | 20 |
| Buckwheat, Wild | 20 | 65 | 60 | 65 | 80 | 55 | 70 |
| Canarygrass | 0 | 0 | 10 | 15 | 10 | 10 | 25 |
| Chamomile | 40 | 50 | 50 | 65 | 40 | 30 | 65 |
| Chickweed | 70 | 65 | 60 | 70 | 70 | 40 | 70 |
| Deadnettle | 30 | 30 | 50 | 35 | 30 | 25 | 55 |
| Field Poppy | 80 | 20 | 85 | 70 | 70 | 60 | 80 |
| Field Violet | 75 | 50 | 70 | 60 | 75 | 60 | 90 |
| Foxtail, Green | 30 | 40 | 55 | 70 | 70 | 35 | 70 |
| Galium | 75 | 70 | 75 | 80 | 75 | 75 | 80 |
| Kochia | 98 | 65 | 60 | 80 | 75 | 75 | 80 |
| Lambsquarters | 98 | 85 | 95 | 80 | 80 | 40 | 70 |
| Mustard, Wild | — | 55 | 100 | 90 | 75 | 60 | 85 |
| Oat, Wild | 0 | 5 | 10 | 25 | 20 | 20 | 25 |
| Oilseed Rape | 60 | 35 | 50 | 60 | 65 | 35 | 70 |
| Pigweed | 75 | 65 | 80 | 80 | 80 | 65 | 80 |
| Radish, Wild | 50 | — | 70 | 60 | 70 | 50 | 70 |
| Russian Thistle | 85 | 60 | 10 | — | — | — | — |
| Ryegrass, Italian | 10 | 10 | 5 | 10 | 15 | 5 | 40 |
| Speedwell | 100 | 75 | 75 | 80 | 70 | 55 | 70 |
| Wheat, Spring | 5 | 5 | 5 | 15 | 10 | 0 | 20 |
| Wheat, Winter | 5 | 0 | 15 | 25 | 20 | 10 | 40 |
| Windgrass | 0 | 5 | 10 | 35 | 25 | 25 | 40 |
| | 125 g ai/ha | | | | | | |
| | Compounds | | | | | | |
| | 138 | 176 | 188 | 189 | 201 | 202 | 214 |
| Barley, Spring | 25 | 15 | 25 | 15 | 25 | 25 | 20 |
| Barley, Winter | 20 | 20 | 40 | 20 | 25 | 15 | 30 |
| Blackgrass | 40 | 20 | 35 | 15 | 50 | 25 | 20 |
| Bluegrass | 30 | 25 | 30 | 15 | 35 | 25 | 15 |
| Bromegrass, Downy | 30 | 30 | 15 | 15 | 35 | 35 | 20 |
| Buckwheat, Wild | 70 | 65 | 70 | 70 | 65 | 70 | 70 |
| Canarygrass | 25 | 20 | 20 | 20 | 20 | 15 | 15 |
| Chamomile | 60 | 35 | 70 | 60 | 55 | 60 | 60 |
| Chickweed | 80 | 65 | 75 | 70 | 70 | 45 | 70 |
| Deadnettle | 75 | 45 | 45 | 55 | 60 | 60 | 60 |
| Field Poppy | 65 | 65 | 75 | 75 | 60 | 50 | 70 |
| Field Violet | 75 | 70 | 100 | 100 | 65 | 45 | 100 |
| Foxtail, Green | 65 | 35 | 25 | 15 | 40 | 20 | 35 |
| Galium | 100 | 65 | 85 | 75 | 70 | 70 | 80 |
| Kochia | 80 | 70 | 80 | 80 | 85 | 75 | 80 |
| Lambsquarters | 80 | 75 | 80 | 80 | 90 | 60 | 80 |
| Mustard, Wild | 100 | 70 | 60 | 40 | 85 | 70 | 100 |
| Oat, Wild | 25 | 20 | 25 | 30 | 50 | 30 | 20 |
| Oilseed Rape | 90 | 55 | 55 | 55 | 50 | 50 | 75 |
| Pigweed | 90 | 70 | 60 | 70 | 75 | 80 | 90 |
| Radish, Wild | 100 | 65 | 70 | 65 | 65 | 65 | 70 |
| Russian Thistle | — | — | — | — | — | — | — |
| Ryegrass, Italian | 15 | 10 | 25 | 20 | 20 | 10 | 20 |
| Speedwell | 100 | 70 | 80 | 85 | 70 | 75 | 95 |
| Wheat, Spring | 15 | 0 | 15 | 5 | 15 | 10 | 15 |
| Wheat, Winter | 20 | 15 | 30 | 25 | 25 | 20 | 20 |
| Windgrass | 35 | 25 | 35 | 20 | 30 | 25 | 30 |
| | 62 g ai/ha | | | | | | |
| | Compounds | | | | | | |
| | 70 | 74 | 76 | 78 | 97 | 103 | 105 |
| Barley, Spring | 0 | 0 | 5 | 0 | 5 | 0 | 10 |
| Barley, Winter | 5 | 0 | 10 | 10 | 10 | 5 | 10 |
| Blackgrass | 0 | 5 | 5 | 10 | 10 | 10 | 15 |
| Bluegrass | 0 | 5 | 10 | 10 | 15 | 25 | 15 |
| Bromegrass, Downy | 0 | 0 | 5 | 15 | 15 | 10 | 15 |
| Buckwheat, Wild | 20 | 45 | 50 | 60 | 70 | 50 | 65 |
| Canarygrass | 0 | 0 | 10 | 10 | 10 | 5 | 15 |
| Chamomile | 30 | 20 | 30 | 50 | 20 | 30 | 50 |
| Chickweed | 70 | 25 | 55 | 50 | 60 | 25 | 55 |
| Deadnettle | 10 | 20 | 40 | 20 | 25 | 25 | 20 |
| Field Poppy | 75 | 30 | 70 | 65 | 65 | 55 | 80 |
| Field Violet | 40 | 60 | 20 | 40 | 75 | 55 | 75 |
| Foxtail, Green | 0 | 15 | 15 | 60 | 60 | 20 | 65 |
| Galium | 70 | 40 | 75 | 70 | 60 | 70 | 75 |
| Kochia | 70 | 35 | 50 | 80 | 70 | 65 | 75 |
| Lambsquarters | 98 | 75 | 75 | 60 | 65 | 30 | 65 |
| Mustard, Wild | — | 30 | 70 | 70 | 70 | 45 | 60 |
| Oat, Wild | 0 | 5 | 10 | 20 | 15 | 15 | 20 |
| Oilseed Rape | 50 | 25 | 20 | 50 | 55 | 35 | 60 |
| Pigweed | 60 | 65 | 65 | 75 | 80 | 60 | 75 |
| Radish, Wild | 10 | — | 20 | 50 | 50 | 35 | 55 |
| Russian Thistle | 80 | 0 | 5 | — | — | — | — |
| Ryegrass, Italian | 0 | 5 | 5 | 5 | 10 | 5 | 5 |
| Speedwell | 100 | 70 | 75 | 60 | 75 | 45 | 80 |
| Wheat, Spring | 5 | 0 | 5 | 5 | 5 | 0 | 10 |
| Wheat, Winter | 5 | 0 | 10 | 15 | 15 | 5 | 15 |
| Windgrass | 0 | 0 | 10 | 20 | 20 | 20 | 25 |
| | 62 g ai/ha | | | | | | |
| | Compounds | | | | | | |
| | 138 | 176 | 188 | 189 | 201 | 202 | 214 |
| Barley, Spring | 15 | 10 | 15 | 10 | 25 | 20 | 15 |
| Barley, Winter | 15 | 10 | 15 | 15 | 20 | 15 | 20 |
| Blackgrass | 25 | 15 | 15 | 10 | 25 | 15 | 15 |
| Bluegrass | 15 | 15 | 20 | 15 | 20 | 20 | 15 |
| Bromegrass, Downy | 10 | 25 | 15 | 15 | 30 | 25 | 15 |
| Buckwheat, Wild | 65 | 55 | 60 | 70 | 50 | 40 | 70 |
| Canarygrass | 5 | 15 | 15 | 15 | 15 | 15 | 15 |
| Chamomile | 50 | 15 | 65 | 55 | 35 | 55 | 40 |
| Chickweed | 70 | 35 | 65 | 75 | 55 | 55 | 65 |
| Deadnettle | 65 | 35 | 35 | 45 | 55 | 55 | 50 |
| Field Poppy | 55 | 50 | 60 | 55 | 55 | 50 | 70 |
| Field Violet | 80 | 50 | 85 | 60 | 55 | 25 | 100 |
| Foxtail, Green | 15 | 20 | 25 | 20 | 30 | 20 | 15 |
| Galium | 80 | 65 | 75 | 75 | 70 | 70 | 80 |
| Kochia | 75 | 65 | 75 | 75 | 75 | 70 | 70 |
| Lambsquarters | 80 | 60 | 70 | 75 | 75 | 50 | 65 |
| Mustard, Wild | 100 | 55 | 50 | 40 | 75 | 70 | 70 |
| Oat, Wild | 20 | 15 | 15 | 20 | 25 | 25 | 15 |
| Oilseed Rape | 60 | 40 | 55 | 40 | 40 | 40 | 65 |
| Pigweed | 90 | 65 | 40 | 70 | 70 | 70 | 90 |
| Radish, Wild | 80 | 65 | 70 | 60 | 65 | 55 | 65 |
| Russian Thistle | — | — | — | — | — | — | — |
| Ryegrass, Italian | 15 | 5 | 20 | 10 | 15 | 10 | 10 |
| Speedwell | 100 | 70 | 75 | 70 | 70 | 70 | 90 |
| Wheat, Spring | 15 | 0 | 10 | 0 | 15 | 10 | 15 |
| Wheat, Winter | 15 | 5 | 25 | 20 | 20 | 15 | 15 |
| Windgrass | 20 | 15 | 20 | 15 | 20 | 15 | 20 |

| | 31 g ai/ha | | | | | | |
|---|---|---|---|---|---|---|---|
| | Compounds | | | | | | |
| | 70 | 74 | 76 | 78 | 97 | 103 | 105 |
| Barley, Spring | 0 | 0 | 0 | 0 | 5 | 0 | 10 |
| Barley, Winter | 0 | 0 | 10 | 5 | 5 | 0 | 10 |
| Blackgrass | 0 | 0 | 0 | 5 | 5 | 5 | 10 |
| Bluegrass | 0 | 5 | 5 | 10 | 10 | 10 | 15 |
| Bromegrass, Downy | 0 | 0 | 0 | 10 | 10 | 5 | 10 |
| Buckwheat, Wild | 20 | 60 | 55 | 50 | 60 | 25 | 55 |
| Canarygrass | 0 | 0 | 5 | 5 | 5 | 5 | 10 |
| Chamomile | 30 | 15 | 25 | 30 | 15 | 10 | 25 |
| Chickweed | 30 | 35 | 50 | 25 | 35 | 20 | 20 |
| Deadnettle | 10 | 15 | 25 | 15 | 15 | 20 | 15 |
| Field Poppy | 70 | 75 | 65 | 55 | 60 | 40 | 65 |
| Field Violet | 40 | 30 | 15 | 30 | 50 | 35 | 40 |
| Foxtail, Green0 | 15 | 10 | 35 | 25 | 15 | 30 | 10 |
| Galium | 60 | 20 | 70 | 60 | 70 | 55 | 70 |
| Kochia | 85 | 25 | 40 | 75 | 75 | 60 | 75 |
| Lambsquarters | 85 | 70 | 70 | 20 | 40 | 35 | 40 |
| Mustard, Wild | — | 30 | 40 | 40 | 65 | 25 | 70 |
| Oat, Wild | 0 | 5 | 5 | 15 | 15 | 10 | 15 |
| Oilseed Rape | 40 | 20 | 15 | 35 | 50 | 25 | 40 |
| Pigweed | 60 | 50 | 55 | 70 | 65 | 70 | 85 |
| Radish, Wild | 10 | — | 10 | 40 | 40 | 25 | 35 |
| Russian Thistle | 80 | 0 | 5 | — | — | — | — |
| Ryegrass, Italian | 0 | 5 | 0 | 5 | 5 | 5 | 5 |
| Speedwell | 100 | 60 | 60 | 45 | 50 | 35 | 60 |
| Wheat, Spring | 5 | 0 | 5 | 0 | 5 | 0 | 5 |
| Wheat, Winter | 0 | 0 | 0 | 10 | 10 | 0 | 10 |
| Windgrass | 0 | 0 | 5 | 10 | 15 | 10 | 15 |

| | 31 g ai/ha | | | | | | |
|---|---|---|---|---|---|---|---|
| | Compounds | | | | | | |
| | 138 | 176 | 188 | 189 | 201 | 202 | 214 |
| Barley, Spring | 15 | 10 | 15 | 10 | 25 | 20 | 15 |
| Barley, Winter | 15 | 10 | 15 | 15 | 20 | 15 | 20 |
| Blackgrass | 25 | 15 | 15 | 10 | 25 | 15 | 15 |
| Bluegrass | 15 | 15 | 20 | 15 | 20 | 20 | 15 |
| Bromegrass, Downy | 10 | 25 | 15 | 15 | 30 | 25 | 15 |
| Buckwheat, Wild | 65 | 55 | 60 | 70 | 50 | 40 | 70 |
| Canarygrass | 5 | 15 | 15 | 15 | 15 | 15 | 15 |
| Chamomile | 50 | 15 | 65 | 55 | 35 | 55 | 40 |
| Chickweed | 70 | 35 | 65 | 75 | 55 | 55 | 65 |
| Deadnettle | 65 | 35 | 35 | 45 | 55 | 55 | 50 |
| Field Poppy | 55 | 50 | 60 | 55 | 55 | 50 | 70 |
| Field Violet | 80 | 50 | 85 | 60 | 55 | 25 | 100 |
| Foxtail, Green | 15 | 20 | 25 | 20 | 30 | 20 | 15 |
| Galium | 80 | 65 | 75 | 75 | 70 | 70 | 80 |
| Kochia | 75 | 65 | 75 | 75 | 75 | 70 | 70 |
| Lambsquarters | 80 | 60 | 70 | 75 | 75 | 50 | 65 |
| Mustard, Wild | 100 | 55 | 50 | 40 | 75 | 70 | 70 |
| Oat, Wild | 20 | 15 | 15 | 20 | 25 | 25 | 15 |
| Oilseed Rape | 60 | 40 | 55 | 40 | 40 | 40 | 65 |
| Pigweed | 90 | 65 | 40 | 70 | 70 | 70 | 90 |
| Radish, Wild | 80 | 65 | 70 | 60 | 65 | 55 | 65 |
| Russian Thistle | — | — | — | — | — | — | — |
| Ryegrass, Italian | 15 | 5 | 20 | 10 | 15 | 10 | 10 |
| Speedwell | 100 | 70 | 75 | 70 | 70 | 70 | 90 |
| Wheat, Spring | 15 | 0 | 10 | 0 | 15 | 10 | 15 |
| Wheat, Winter | 15 | 5 | 25 | 20 | 20 | 15 | 15 |
| Windgrass | 20 | 15 | 20 | 15 | 20 | 15 | 20 |

| | 16 g ai/ha | | | |
|---|---|---|---|---|
| | Compounds | | | |
| | 70 | 74 | 76 | 176 |
| Barley, Spring | 0 | 0 | 0 | 0 |
| Barley, Winter | 0 | 0 | 10 | 0 |
| Blackgrass | 0 | 0 | 5 | 5 |
| Bluegrass | 0 | 5 | 5 | 5 |
| Bromegrass, Downy | 0 | 0 | 0 | 10 |
| Buckwheat, Wild | 10 | 60 | 50 | 30 |
| Canarygrass | 0 | 0 | 5 | 5 |
| Chamomile | 10 | 15 | 10 | 10 |
| Chickweed | 30 | 15 | 35 | 20 |
| Deadnettle | 0 | 10 | 15 | 10 |
| Field Poppy | 20 | 10 | 60 | 45 |
| Field Violet | 20 | 25 | 10 | 35 |
| Foxtail, Green | 0 | 0 | 5 | 10 |
| Galium | 50 | 10 | 55 | 55 |
| Kochia | 40 | 15 | 25 | 30 |
| Lambsquarters | 80 | 70 | 65 | 55 |
| Mustard, Wild | | 20 | 10 | 25 |
| Oat, Wild | 0 | 0 | 5 | 5 |
| Oilseed Rape | 20 | 15 | 10 | 20 |
| Pigweed | 40 | 65 | 50 | 55 |
| Radish, Wild | 10 | — | 5 | — |
| Russian Thistle | 0 | 0 | 5 | — |
| Ryegrass, Italian | 0 | 0 | 0 | 5 |
| Speedwell | 100 | 75 | 65 | 50 |
| Wheat, Spring | 0 | 0 | 0 | 0 |
| Wheat, Winter | 0 | 0 | 0 | 5 |
| Windgrass | 0 | 0 | 5 | 5 |

Preemergence

| | 250 g ai/ha | | | | | |
|---|---|---|---|---|---|---|
| | Compounds | | | | | |
| | 70 | 76 | 78 | 97 | 103 | 105 |
| Barley, Spring | 15 | 75 | 100 | 80 | 100 | 85 |
| Barley, Winter | 10 | 75 | 95 | 85 | 85 | 95 |
| Blackgrass | 30 | 100 | 100 | 100 | 100 | 100 |
| Bluegrass | 10 | 100 | 100 | 100 | 85 | 100 |
| Bromegrass, Downy | 20 | 65 | 75 | 100 | 85 | 95 |
| Buckwheat, Wild | 100 | 100 | 100 | 100 | 100 | 100 |
| Canarygrass | 75 | 100 | 100 | 95 | 100 | 100 |
| Chamomile | 40 | 100 | — | — | — | — |
| Chickweed | 100 | — | 100 | 100 | 100 | 100 |
| Deadnettle | 100 | 100 | 100 | — | 30 | 35 |
| Field Poppy | 100 | 100 | 100 | 100 | 100 | 100 |
| Field Violet | 50 | 100 | 100 | 100 | 100 | 100 |
| Foxtail, Green | 100 | 100 | 100 | 100 | 100 | 100 |
| Galium | 40 | 100 | 100 | 100 | 100 | 100 |
| Kochia | 50 | 100 | 100 | 95 | 100 | 100 |
| Lambsquarters | 100 | 100 | 100 | 100 | 100 | 100 |
| Mustard, Wild | 100 | 100 | 100 | 100 | 100 | 100 |
| Oat, Wild | 30 | 100 | — | — | — | — |
| Oilseed Rape | 40 | 100 | 100 | 100 | 95 | 95 |
| Pigweed | 75 | 100 | 100 | 100 | 100 | 100 |
| Radish, Wild | 100 | 100 | 100 | 100 | 100 | 100 |
| Russian Thistle | 100 | 95 | — | — | — | — |
| Ryegrass, Italian | 75 | 95 | — | — | — | — |
| Speedwell | — | 100 | 100 | 100 | 100 | 100 |
| Wheat, Spring | 20 | 75 | 100 | 75 | 85 | 100 |
| Wheat, Winter | 10 | 60 | 95 | 65 | 70 | 90 |
| Windgrass | 0 | 100 | 100 | 100 | 100 | 100 |

| | 250 g ai/ha | | | | | |
|---|---|---|---|---|---|---|
| | Compounds | | | | | |
| | 138 | 188 | 189 | 201 | 202 | 214 |
| Barley, Spring | 75 | 95 | 85 | 75 | 65 | 80 |
| Barley, Winter | 70 | 100 | 65 | 100 | 45 | 65 |
| Blackgrass | 100 | 100 | 100 | 100 | 80 | 100 |
| Bluegrass | 100 | 100 | 85 | 95 | 80 | 100 |
| Bromegrass, Downy | 65 | 100 | 55 | 100 | 50 | 80 |
| Buckwheat, Wild | 100 | 100 | 100 | 100 | 100 | 100 |
| Canarygrass | 100 | 100 | 100 | 100 | 85 | 100 |
| Chamomile | 100 | 100 | 100 | 100 | 100 | 100 |
| Chickweed | 100 | 100 | 100 | 100 | 100 | 100 |
| Deadnettle | 100 | 100 | 100 | 100 | 100 | 100 |
| Field Poppy | 100 | 100 | 100 | 100 | 100 | 100 |
| Field Violet | 100 | 100 | 100 | 100 | 100 | 100 |
| Foxtail, Green | 100 | 100 | 100 | 100 | 100 | 100 |
| Galium | 100 | 100 | 100 | 100 | 100 | 100 |
| Kochia | 100 | 100 | 100 | 100 | 100 | 100 |
| Lambsquarters | 100 | 100 | 100 | 100 | 100 | 100 |
| Mustard, Wild | 100 | 100 | 100 | 100 | 100 | 100 |
| Oat, Wild | 100 | 100 | 100 | 100 | 100 | 95 |
| Oilseed Rape | 100 | 100 | 100 | 100 | 90 | 100 |
| Pigweed | 100 | 100 | 100 | 100 | 100 | 100 |
| Radish, Wild | 100 | 100 | 100 | 100 | 100 | 100 |
| Russian Thistle | — | — | — | — | — | — |
| Ryegrass, Italian | 90 | 95 | 95 | 100 | 80 | 90 |
| Speedwell | 100 | 100 | 100 | 100 | 100 | 100 |
| Wheat, Spring | 50 | 100 | 80 | 100 | 25 | 75 |
| Wheat, Winter | 60 | 100 | 85 | 95 | 60 | 75 |
| Windgrass | 100 | 100 | 100 | 100 | 100 | 100 |

| | 125 g ai/ha | | | | | | |
|---|---|---|---|---|---|---|---|
| | Compounds | | | | | | |
| | 70 | 74 | 76 | 78 | 97 | 103 | 105 |
| Barley, Spring | 0 | 5 | 65 | 90 | 65 | 80 | 65 |
| Barley, Winter | 0 | 0 | 60 | 65 | 70 | 60 | 70 |
| Blackgrass | 10 | 15 | 100 | 80 | 100 | 85 | 100 |
| Bluegrass | 0 | 5 | 55 | 95 | 95 | 65 | 100 |
| Bromegrass, Downy | 10 | 0 | 5 | 70 | 35 | 40 | 55 |
| Buckwheat, Wild | 0 | 65 | 100 | 100 | 100 | 100 | 95 |
| Canarygrass | 20 | 5 | 100 | 90 | 70 | 80 | 100 |
| Chamomile | 15 | 90 | 100 | — | — | — | — |
| Chickweed | 100 | 100 | — | 100 | 100 | 100 | 100 |
| Deadnettle | 50 | 20 | 100 | 25 | 35 | 30 | 20 |
| Field Poppy | 90 | 100 | 100 | 100 | 100 | 100 | 100 |
| Field Violet | 50 | 95 | 100 | 100 | 100 | 100 | 100 |
| Foxtail, Green | 20 | 30 | 100 | 100 | 100 | 100 | 100 |
| Galium | — | — | 100 | 100 | 100 | 100 | 100 |
| Kochia | 30 | 100 | 100 | 100 | 95 | 85 | 100 |
| Lambsquarters | 100 | 85 | 100 | 100 | 100 | 100 | 100 |
| Mustard, Wild | 100 | 80 | 100 | 100 | 100 | 75 | 100 |
| Oat, Wild | 5 | 30 | 90 | — | — | — | — |
| Oilseed Rape | 10 | 45 | 90 | 100 | 30 | 35 | 70 |
| Pigweed | — | 100 | 100 | 100 | 100 | 100 | 100 |
| Radish, Wild | 100 | 100 | 100 | 100 | 100 | 100 | 100 |
| Russian Thistle | 90 | 95 | 80 | — | — | — | — |
| Ryegrass, Italian | 20 | 0 | 65 | — | — | — | — |
| Speedwell | — | 100 | 100 | 100 | 100 | 100 | 100 |
| Wheat, Spring | 10 | 0 | 60 | 80 | 45 | 25 | 75 |
| Wheat, Winter | 0 | 5 | 40 | 80 | 35 | 65 | 75 |
| Windgrass | 0 | 35 | 80 | 100 | 100 | 100 | 100 |

| | 125 g ai/ha | | | | | | |
|---|---|---|---|---|---|---|---|
| | Compounds | | | | | | |
| | 138 | 176 | 188 | 189 | 201 | 202 | 214 |
| Barley, Spring | 40 | 0 | 85 | 70 | 65 | 55 | 65 |
| Barley, Winter | 60 | 0 | 80 | 60 | 45 | 25 | 55 |
| Blackgrass | 85 | 20 | 100 | 80 | 100 | 75 | 90 |
| Bluegrass | 90 | 20 | 100 | 70 | 80 | 50 | 85 |
| Bromegrass, Downy | 20 | 25 | 85 | 20 | 70 | 35 | 35 |
| Buckwheat, Wild | 70 | 85 | 100 | 100 | — | 100 | 90 |
| Canarygrass | 100 | 35 | 100 | 85 | 100 | 75 | 100 |
| Chamomile | 100 | 50 | 100 | 100 | 100 | 100 | 100 |
| Chickweed | 100 | 100 | 100 | 100 | 100 | 100 | 100 |
| Deadnettle | 100 | 100 | 100 | 100 | 100 | 100 | 100 |
| Field Poppy | 100 | 100 | 100 | 100 | 100 | 100 | 100 |
| Field Violet | 100 | 65 | 100 | 100 | 100 | 100 | 100 |
| Foxtail, Green | 100 | 40 | 100 | 100 | 100 | 80 | 100 |
| Galium | 100 | 85 | 100 | 70 | 100 | 70 | 100 |
| Kochia | 100 | 100 | 100 | 100 | 100 | 100 | 100 |
| Lambsquarters | 100 | 95 | 100 | 100 | 100 | 100 | 100 |
| Mustard, Wild | 100 | 100 | 100 | 100 | 100 | 100 | 100 |
| Oat, Wild | 75 | 40 | 100 | 75 | 100 | 65 | 70 |
| Oilseed Rape | 100 | 65 | 100 | 80 | 100 | 70 | 100 |
| Pigweed | 100 | 100 | 100 | 100 | 100 | 100 | 100 |
| Radish, Wild | 100 | 100 | 100 | 100 | 100 | 100 | 100 |
| Russian Thistle | — | — | — | — | — | — | — |
| Ryegrass, Italian | 70 | 30 | 85 | 60 | 85 | 40 | 70 |
| Speedwell | 100 | 100 | 100 | 100 | 100 | 100 | 100 |
| Wheat, Spring | 35 | 5 | 100 | 40 | 75 | 10 | 40 |
| Wheat, Winter | 40 | 5 | 95 | 65 | 70 | 25 | 55 |
| Windgrass | 100 | 40 | 100 | 100 | 100 | 75 | 90 |

| | 62 g ai/ha | | | | | | |
|---|---|---|---|---|---|---|---|
| | Compounds | | | | | | |
| | 70 | 74 | 76 | 78 | 97 | 103 | 105 |
| Barley, Spring | 0 | 0 | 10 | 65 | 55 | 35 | 40 |
| Barley, Winter | 0 | 0 | 15 | 50 | 35 | 35 | 40 |
| Blackgrass | 5 | 10 | 100 | 70 | 50 | 50 | 80 |
| Bluegrass | 0 | 0 | 5 | 80 | 65 | 35 | 80 |
| Bromegrass, Downy | 0 | 5 | 0 | 20 | 25 | 10 | 15 |
| Buckwheat, Wild | 0 | 45 | 70 | 100 | 85 | 100 | 90 |
| Canarygrass | 0 | 0 | 65 | 30 | 70 | 55 | 60 |
| Chamomile | 0 | 80 | 95 | — | — | — | — |
| Chickweed | 10 | 100 | — | 100 | 100 | 100 | 100 |
| Deadnettle | 30 | 10 | 100 | 10 | — | 15 | — |
| Field Poppy | 100 | 95 | 100 | 100 | 100 | 100 | 100 |
| Field Violet | 0 | 60 | 100 | 100 | 100 | 100 | 100 |
| Foxtail, Green | 0 | 10 | 100 | 100 | 100 | 100 | 100 |
| Galium | 10 | — | 100 | 100 | 100 | 100 | 100 |
| Kochia | 20 | 100 | 100 | 100 | 50 | 65 | 100 |
| Lambsquarters | 0 | 20 | 95 | 100 | 100 | 70 | 100 |
| Mustard, Wild | 100 | 25 | 100 | 100 | 100 | 60 | 100 |
| Oat, Wild | 0 | 10 | 35 | — | — | — | — |
| Oilseed Rape | 0 | 40 | 65 | 80 | 5 | 30 | 10 |
| Pigweed | 0 | 70 | 80 | 100 | 100 | 100 | 100 |
| Radish, Wild | 0 | 75 | 100 | 100 | 100 | 100 | 100 |
| Russian Thistle | 90 | 85 | 70 | — | — | — | — |
| Ryegrass, Italian | 0 | 0 | 15 | — | — | — | — |
| Speedwell | — | 100 | 100 | 100 | 100 | 100 | 100 |
| Wheat, Spring | 0 | 0 | 10 | 55 | 35 | 10 | 40 |
| Wheat, Winter | 0 | 0 | 5 | 50 | 10 | 10 | 35 |
| Windgrass | 0 | 25 | 65 | 100 | 80 | 80 | 100 |

| | 62 g ai/ha | | | | | | |
|---|---|---|---|---|---|---|---|
| | Compounds | | | | | | |
| | 138 | 176 | 188 | 189 | 201 | 202 | 214 |
| Barley, Spring | 5 | 0 | 75 | 40 | 20 | 0 | 40 |
| Barley, Winter | 35 | 0 | 70 | 35 | 35 | 0 | 35 |
| Blackgrass | 30 | 10 | 75 | 60 | 90 | 35 | 75 |
| Bluegrass | 55 | 10 | 70 | 50 | 50 | 5 | 65 |
| Bromegrass, Downy | 15 | 15 | 75 | 15 | 60 | 35 | 25 |
| Buckwheat, Wild | 50 | 95 | 100 | 100 | 100 | 60 | 95 |
| Canarygrass | 70 | 25 | 95 | 50 | 100 | 40 | 65 |
| Chamomile | 95 | 10 | 100 | 100 | 100 | 100 | 100 |
| Chickweed | 100 | 85 | 100 | 100 | 100 | 100 | 75 |
| Deadnettle | 100 | 80 | 100 | 100 | 100 | 100 | 100 |
| Field Poppy | 100 | 55 | 100 | 100 | 100 | 100 | 100 |
| Field Violet | 100 | 50 | 100 | 100 | 100 | 25 | 100 |
| Foxtail, Green | 100 | 15 | 100 | 100 | 100 | 35 | 100 |
| Galium | 100 | 60 | 95 | 75 | 50 | 35 | 85 |
| Kochia | 95 | 45 | 100 | 100 | 100 | 100 | 70 |
| Lambsquarters | 85 | 65 | 100 | 100 | 90 | 75 | 90 |
| Mustard, Wild | 100 | 90 | 100 | 100 | 100 | 100 | 100 |
| Oat, Wild | 35 | 10 | 100 | 45 | 60 | 15 | 20 |
| Oilseed Rape | 40 | 35 | 85 | 55 | 100 | 65 | 65 |
| Pigweed | 100 | 40 | 100 | 95 | 80 | 100 | 100 |
| Radish, Wild | 100 | 75 | 100 | 70 | 100 | 100 | 100 |
| Russian Thistle | — | — | — | — | — | — | — |
| Ryegrass, Italian | 35 | 25 | 75 | 40 | 65 | 0 | 55 |
| Speedwell | 100 | 100 | 100 | 100 | 100 | 100 | 100 |
| Wheat, Spring | 10 | 0 | 70 | 10 | 55 | 0 | 10 |
| Wheat, Winter | 30 | 0 | 65 | 15 | 40 | 5 | 15 |
| Windgrass | 45 | 10 | 100 | 65 | 100 | 55 | 85 |

| | 31 g ai/ha | | | | | | |
|---|---|---|---|---|---|---|---|
| | Compounds | | | | | | |
| | 70 | 74 | 76 | 78 | 97 | 103 | 105 |
| Barley, Spring | 0 | 0 | 0 | 20 | 15 | 15 | 15 |
| Barley, Winter | 0 | 0 | 0 | 25 | 5 | 0 | 5 |
| Blackgrass | 0 | 0 | 50 | 35 | 15 | — | 25 |
| Bluegrass | 0 | 0 | 0 | 50 | 25 | 10 | 55 |
| Bromegrass, Downy | 0 | 0 | 0 | 10 | 10 | 5 | 5 |
| Buckwheat, Wild | 0 | 35 | 20 | 95 | 100 | 75 | 85 |
| Canarygrass | 0 | 0 | 5 | 15 | 10 | 5 | 15 |
| Chamomile | 0 | 60 | 80 | — | — | — | — |
| Chickweed | 0 | 70 | — | 100 | 100 | 95 | 95 |
| Deadnettle | 25 | 5 | 50 | 10 | 10 | 5 | 5 |
| Field Poppy | 100 | 80 | 100 | 100 | 100 | 100 | 100 |
| Field Violet | 0 | 60 | 95 | 100 | 100 | 95 | 100 |
| Foxtail, Green | 0 | 5 | 50 | 50 | 80 | 20 | 45 |
| Galium | 0 | — | 50 | 95 | 15 | 50 | 100 |
| Kochia | 10 | 85 | 100 | 95 | 55 | 10 | 95 |
| Lambsquarters | 0 | 50 | 70 | 95 | 95 | 65 | 100 |
| Mustard, Wild | 0 | 25 | 60 | 100 | 100 | 55 | 100 |
| Oat, Wild | 0 | 10 | 10 | — | — | — | — |
| Oilseed Rape | 0 | 15 | 30 | 35 | 0 | 25 | 0 |
| Pigweed | — | 30 | 60 | 100 | 100 | 100 | 100 |
| Radish, Wild | 0 | 15 | 75 | 100 | 50 | 95 | 35 |
| Russian Thistle | 0 | 70 | 65 | — | — | — | — |
| Ryegrass, Italian | 0 | 0 | 0 | — | — | — | — |
| Speedwell | — | 100 | 100 | 100 | 100 | 70 | 100 |
| Wheat, Spring | 0 | 0 | 0 | 5 | 0 | 0 | 10 |
| Wheat, Winter | 0 | 0 | 0 | 15 | 0 | 0 | 15 |
| Windgrass | 0 | 0 | 15 | 90 | 75 | 15 | 75 |

| | 31 g ai/ha | | | | | | |
|---|---|---|---|---|---|---|---|
| | Compounds | | | | | | |
| | 138 | 176 | 188 | 189 | 201 | 202 | 214 |
| Barley, Spring | 0 | 0 | 65 | 0 | 0 | 0 | 10 |
| Barley, Winter | 5 | 0 | 35 | 10 | 25 | 0 | 15 |
| Blackgrass | 15 | 0 | 55 | 35 | 35 | 5 | 35 |
| Bluegrass | 20 | 0 | 55 | 15 | 20 | 10 | 40 |
| Bromegrass, Downy | 10 | 0 | 20 | 0 | 20 | 25 | 15 |
| Buckwheat, Wild | 45 | 15 | 100 | 100 | 15 | 35 | 35 |
| Canarygrass | 15 | 5 | 55 | 10 | 60 | 10 | 40 |
| Chamomile | 0 | 5 | 100 | 65 | 50 | 5 | 50 |
| Chickweed | 100 | 50 | 100 | 95 | 80 | 100 | 90 |
| Deadnettle | 85 | 20 | 100 | 90 | 100 | 100 | 90 |
| Field Poppy | 100 | 15 | 100 | 95 | 100 | 90 | 100 |
| Field Violet | 100 | 0 | 100 | 100 | 100 | 25 | 100 |
| Foxtail, Green | 65 | 5 | 95 | 55 | 65 | 15 | 45 |
| Galium | 15 | 60 | 55 | 50 | 55 | 0 | 0 |
| Kochia | 75 | 5 | 35 | 100 | 95 | 65 | 60 |
| Lambsquarters | 75 | 20 | 100 | 95 | 95 | 30 | 85 |
| Mustard, Wild | 90 | 80 | 100 | 100 | 100 | 70 | 100 |
| Oat, Wild | 15 | 5 | 85 | 40 | 35 | 15 | 0 |
| Oilseed Rape | 5 | 35 | 70 | 20 | 25 | 10 | 50 |
| Pigweed | 95 | 10 | 95 | 70 | 50 | 55 | 100 |
| Radish, Wild | 100 | 35 | 100 | — | 100 | — | 100 |
| Russian Thistle | — | — | — | — | — | — | — |
| Ryegrass, Italian | 15 | 5 | 40 | 25 | 20 | 0 | 10 |
| Speedwell | 100 | 100 | 100 | 100 | 100 | 100 | 100 |
| Wheat, Spring | 0 | 0 | 25 | 0 | 10 | 0 | 0 |
| Wheat, Winter | 0 | 0 | 30 | 0 | 5 | 0 | 5 |
| Windgrass | 35 | 5 | 70 | 35 | 50 | 5 | 65 |

| | 16 g ai/ha | | | |
|---|---|---|---|---|
| | Compounds | | | |
| | 70 | 74 | 76 | 176 |
| Barley, Spring | 0 | 0 | 0 | 0 |
| Barley, Winter | 0 | 0 | 0 | 0 |
| Blackgrass | 0 | 0 | 20 | 0 |
| Bluegrass | 0 | 0 | 0 | 0 |
| Bromegrass, Downy | 0 | 0 | 0 | 0 |
| Buckwheat, Wild | 0 | 25 | 0 | 0 |
| Canarygrass | 0 | 0 | 0 | 0 |
| Chamomile | 0 | 20 | 50 | 5 |
| Chickweed | 0 | 20 | — | 10 |
| Deadnettle | 0 | 0 | 0 | 0 |
| Field Poppy | 20 | 50 | 95 | 10 |
| Field Violet | 0 | 25 | 60 | 0 |
| Foxtail, Green | 0 | 0 | 25 | 0 |
| Galium | 0 | — | 25 | — |
| Kochia | 0 | 50 | 20 | 5 |
| Lambsquarters | 0 | 20 | 30 | 0 |
| Mustard, Wild | — | 15 | — | 10 |
| Oat, Wild | 0 | 20 | 0 | 0 |
| Oilseed Rape | 0 | 5 | 10 | 0 |
| Pigweed | — | 15 | 10 | 10 |
| Radish, Wild | 0 | 0 | — | 15 |
| Russian Thistle | 0 | — | 50 | — |
| Ryegrass, Italian | 0 | 0 | 0 | 0 |
| Speedwell | — | 100 | 100 | 15 |
| Wheat, Spring | 0 | 0 | 0 | 0 |
| Wheat, Winter | 0 | 0 | 0 | 0 |
| Windgrass | 0 | 0 | 5 | 0 |

### Test E

Seeds of plant species selected from bermudagrass (Cynodon dactylon), Surinam grass (Brachiaria decumbens), crabgrass, large (large crabgrass, Digitaria sanguinalis), crabgrass, naked (naked crabgrass, Digitaria nuda), foxtail, green (green foxtail, Setaria viridis), johnsongrass (Sorghum halepense), kochia (Kochia scoparia), morningglory (pitted morningglory, Ipomoea lacunosa), nutsedge, purple (purple nutsedge, Cyperus rotundus), ragweed (common ragweed, Ambrosia elation), mustard, black (black mustard, Brassica nigra), guineagrass (Panicum maximum), dallisgrass (Paspalum dilatatum), barnyardgrass (Echinochloa crus-galli), sandbur (southern sandbur, Cenchrus echinatus), sowthistle (common sowthistle, Sonchus oleraceous), ryegrass, Italian (Italian ryegrass, Lolium multiflorum), signalgrass (broadleaf signalgrass, Brachiaria platyphylla), dayflower, VA (Virginia (VA) dayflower, Commelina virginica), bluegrass (annual bluegrass, Poa annua), quackgrass (Elytrigia repens), mallow (common mallow, Malva sylvestris), buckwheat, wild (wild buckwheat, Polygonum convolvulus), leafy spurge (Euphorbia esula), common chickweed (Stellaria media), wild poinsettia (Euphorbia heterophylla), pigweed, palmer (palmer pigweed, Amaranthus palmeri), and pigweed (redroot pigweed, Amaranthus retroflexus) were planted into a blend of loam soil and sand and treated preemergence with test chemicals formulated in a non-phytotoxic solvent mixture which included a surfactant.

Treated plants and controls were maintained in a greenhouse for 21 days, after which time all species were compared to controls and visually evaluated. Plant response ratings, summarized in Table E, are based on a scale of 0 to 100 where 0 is no effect and 100 is complete control. A dash (-) response means no test result.

TABLE E
Preemergence

| | 250 g ai/ha | | | | | |
|---|---|---|---|---|---|---|
| | Compounds | | | | | |
| | 78 | 103 | 104 | 105 | 116 | |
| Barnyardgrass | 100 | 100 | 100 | 98 | 100 | |
| Bermudagrass | 100 | 100 | 100 | 100 | 100 | |
| Bluegrass | 100 | 100 | 100 | 100 | 100 | |
| Buckwheat, | 100 | 100 | 100 | 100 | 100 | |
| Wild | | | | | | |
| Common | — | 100 | 100 | 100 | 100 | |
| Chickweed | | | | | | |
| Crabgrass, | 100 | 100 | 100 | 100 | 100 | |
| Large | | | | | | |
| Crabgrass, | 100 | 100 | 100 | 100 | 100 | |
| Naked | | | | | | |
| Dallisgrass | 100 | 100 | 100 | 100 | 100 | |
| Dayflower, VA | 100 | 100 | 100 | 100 | 100 | |
| Foxtail, Green | 100 | 100 | 100 | 100 | 100 | |
| Guineagrass | 100 | 100 | 100 | 100 | 100 | |
| Johnsongrass | 100 | 100 | 100 | 100 | 100 | |
| Kochia | 100 | 100 | 100 | 100 | 100 | |
| Leafy Spurge | 100 | 100 | 100 | 100 | 100 | |
| Mallow | 100 | 100 | 100 | 100 | 100 | |
| Morningglory | 100 | 100 | 100 | 100 | 100 | |
| Mustard, Black | 100 | 100 | 100 | 100 | 100 | |
| Nutsedge, | 100 | 100 | 100 | 95 | 95 | |
| Purple | | | | | | |
| Pigweed | 100 | 100 | 100 | 100 | 100 | |
| Pigweed, | 100 | — | — | — | — | |
| Palmer | | | | | | |
| Quackgrass | 100 | 100 | 100 | 100 | 100 | |
| Ragweed | 100 | 100 | 100 | 100 | 100 | |
| Ryegrass, | 100 | 100 | 100 | 100 | 100 | |
| Italian | | | | | | |
| Sandbur | 100 | 100 | 100 | 98 | 100 | |
| Signalgrass | 100 | 100 | 100 | 98 | 100 | |
| Sowthistle | 100 | — | — | 100 | 100 | |
| Surinam Grass | 100 | 100 | 100 | 100 | 100 | |
| Wild Poinsettia | — | 100 | 100 | 100 | 100 | |
| | 125 g ai/ha | | | | | |
| | Compounds | | | | | |
| | 78 | 103 | 104 | 105 | 116 | |
| Barnyardgrass | 100 | 100 | 100 | 95 | 100 | |
| Bermudagrass | 100 | 98 | 100 | 98 | 98 | |
| Bluegrass | 100 | 98 | 100 | 100 | 98 | |
| Buckwheat, | 100 | 100 | 100 | 100 | 100 | |
| Wild | | | | | | |
| Common | — | 100 | 100 | 100 | 100 | |
| Chickweed | | | | | | |
| Crabgrass, | 100 | 100 | 100 | 100 | 100 | |
| Large | | | | | | |
| Crabgrass, | 100 | 100 | 100 | 100 | 100 | |
| Naked | | | | | | |
| Dallisgrass | 100 | 100 | 100 | 100 | 100 | |
| Dayflower, VA | 100 | 100 | 100 | 95 | 100 | |
| Foxtail, Green | 100 | 100 | 100 | 100 | 100 | |
| Guineagrass | 100 | 100 | 100 | 100 | 100 | |
| Johnsongrass | 100 | 100 | 100 | 98 | 100 | |
| Kochia | 100 | 100 | 100 | 100 | 100 | |
| Leafy Spurge | 100 | 100 | 98 | 100 | 100 | |
| Mallow | 100 | 100 | 98 | 100 | 100 | |
| Morningglory | 100 | 100 | 100 | 100 | 100 | |
| Mustard, Black | 100 | 100 | 100 | 100 | 100 | |
| Nutsedge, | 100 | 98 | 98 | 85 | 80 | |
| Purple | | | | | | |
| Pigweed | 100 | 100 | 100 | 100 | 100 | |
| Pigweed, | 100 | — | — | — | — | |
| Palmer | | | | | | |
| Quackgrass | 100 | 100 | 98 | 100 | 98 | |
| Ragweed | 100 | 100 | 100 | 100 | 100 | |
| Ryegrass, | 100 | 100 | 100 | 100 | 100 | |
| Italian | | | | | | |
| Sandbur | 95 | 100 | 98 | 95 | 100 | |
| Signalgrass | 100 | 100 | 100 | 98 | 100 | |
| Sowthistle | 100 | — | — | 100 | 100 | |
| Surinam Grass | 90 | 100 | 100 | 95 | 100 | |
| Wild Poinsettia | — | 100 | 100 | 100 | 98 | |
| | 62 g ai/ha | | | | | |
| | Compounds | | | | | |
| | 78 | 103 | 104 | 105 | 116 | 189 |
| Barnyardgrass | 100 | 100 | 100 | 85 | 98 | 100 |
| Bermudagrass | 100 | 100 | 98 | 98 | 98 | 98 |
| Bluegrass | 90 | 95 | 100 | 100 | 98 | 80 |
| Buckwheat, | 100 | 100 | 100 | 100 | 100 | 100 |
| Wild | | | | | | |
| Common | — | 100 | 100 | 100 | 100 | — |
| Chickweed | | | | | | |
| Crabgrass, | 100 | 100 | 100 | 100 | 100 | 100 |
| Large | | | | | | |
| Crabgrass, | 95 | 100 | 100 | 100 | 100 | 100 |
| Naked | | | | | | |
| Dallisgrass | 100 | 100 | 100 | 100 | 98 | 100 |
| Dayflower, VA | 100 | 100 | 100 | 95 | 100 | 100 |
| Foxtail, Green | 100 | 100 | 100 | 100 | 100 | 100 |
| Guineagrass | 100 | 100 | 100 | 100 | 100 | 100 |
| Johnsongrass | 85 | 100 | 100 | 98 | 100 | 100 |
| Kochia | 100 | 100 | 100 | 100 | 100 | 100 |
| Leafy Spurge | 100 | 100 | 100 | 100 | 100 | 100 |
| Mallow | 100 | 98 | 100 | 100 | 98 | 100 |
| Morningglory | 90 | 100 | 100 | 100 | 100 | 100 |
| Mustard, Black | 100 | 100 | 100 | 100 | 100 | 100 |
| Nutsedge, | 98 | 90 | 90 | 85 | 80 | 85 |
| Purple | | | | | | |
| Pigweed | 100 | 100 | 100 | 100 | 100 | 100 |
| Pigweed, | 100 | — | — | — | — | — |
| Palmer | | | | | | |
| Quackgrass | 100 | 100 | 95 | 98 | 95 | 100 |
| Ragweed | 100 | 100 | 100 | 100 | 100 | 100 |
| Ryegrass, | 90 | 100 | 100 | 100 | 98 | 100 |
| Italian | | | | | | |
| Sandbur | 90 | 100 | 100 | 70 | 95 | 90 |
| Signalgrass | 100 | 100 | 100 | 98 | 100 | 100 |
| Sowthistle | 100 | — | — | 100 | 100 | 100 |
| Surinam Grass | 90 | 100 | 95 | 80 | 98 | 100 |
| Wild Poinsettia | — | 100 | 100 | 100 | 90 | 100 |
| | 31 g ai/ha | | | | | |
| | Compounds | | | | | |
| | 78 | 103 | 104 | 105 | 116 | 189 |
| Barnyardgrass | 95 | 100 | 100 | 70 | 95 | 100 |
| Bermudagrass | 85 | 98 | 95 | 90 | 95 | 85 |
| Bluegrass | 75 | 90 | 100 | 70 | 65 | 50 |
| Buckwheat, | 100 | 100 | 100 | 100 | 100 | 100 |
| Wild | | | | | | |
| Common | — | 100 | 100 | 100 | 100 | — |
| Chickweed | | | | | | |
| Crabgrass, | 100 | 100 | 100 | 100 | 100 | 100 |
| Large | | | | | | |
| Crabgrass, | 95 | 100 | 100 | 100 | 100 | 100 |
| Naked | | | | | | |
| Dallisgrass | 100 | 100 | 98 | 95 | 100 | 100 |
| Dayflower, VA | 100 | 98 | 98 | 75 | 85 | 100 |
| Foxtail, Green | 100 | 100 | 100 | 98 | 85 | 100 |
| Guineagrass | 100 | 100 | 100 | 90 | 100 | 100 |
| Johnsongrass | 80 | 100 | 100 | 80 | 98 | 100 |
| Kochia | 100 | 100 | 100 | 100 | 100 | 100 |
| Leafy Spurge | 100 | 95 | 98 | 100 | 100 | 100 |
| Mallow | 100 | 98 | 98 | 100 | 100 | 98 |
| Morningglory | 70 | 100 | 98 | 100 | 95 | 100 |
| Mustard, Black | 100 | 100 | 100 | 100 | 100 | 100 |
| Nutsedge, | 75 | 75 | 80 | 70 | 70 | 50 |
| Purple | | | | | | |
| Pigweed | 100 | 100 | 100 | 100 | 100 | 100 |
| Pigweed, | 80 | — | — | — | — | — |
| Palmer | | | | | | |
| Quackgrass | 95 | 98 | 95 | 90 | 80 | 75 |
| Ragweed | 100 | 98 | 100 | 100 | 95 | 100 |
| Ryegrass, | 50 | 98 | 98 | 85 | 90 | 50 |
| Italian | | | | | | |
| Sandbur | 50 | 70 | 90 | 50 | 30 | 20 |
| Signalgrass | 100 | 80 | 75 | 98 | 95 | 95 |
| Sowthistle | — | — | — | 100 | 100 | 100 |
| Surinam Grass | 80 | 100 | 75 | 40 | 65 | 80 |
| Wild Poinsettia | — | 95 | 100 | 80 | 70 | 95 |
| | 16 g ai/ha | | | | | |
| | Compounds | | | | | |
| | 78 | 103 | 104 | 105 | 116 | 189 |
| Barnyardgrass | 35 | 90 | 95 | 35 | 60 | 35 |
| Bermudagrass | 70 | 80 | 90 | 70 | 65 | 65 |
| Bluegrass | 40 | 40 | 85 | 40 | 40 | 50 |
| Buckwheat, | 60 | 95 | 100 | 100 | 98 | 100 |
| Wild | | | | | | |
| Common | — | 98 | 100 | 98 | 98 | — |
| Chickweed | | | | | | |
| Crabgrass, | 90 | 100 | 100 | 98 | 100 | 100 |
| Large | | | | | | |
| Crabgrass, | — | 100 | 95 | 90 | 100 | 100 |
| Naked | | | | | | |
| Dallisgrass | 90 | 90 | 95 | 20 | 40 | 75 |
| Dayflower, VA | 85 | 80 | 98 | 35 | 75 | 90 |
| Foxtail, Green | 50 | 100 | 100 | 95 | 75 | 100 |
| Guineagrass | 90 | 100 | 100 | 75 | 100 | 100 |
| Johnsongrass | 40 | 70 | 95 | 35 | 60 | 25 |
| Kochia | 100 | 95 | 100 | 100 | 100 | 100 |
| Leafy Spurge | 80 | 75 | 100 | 100 | 95 | 100 |
| Mallow | 100 | 95 | 40 | 100 | 95 | 100 |
| Morningglory | 50 | 95 | 98 | 100 | 90 | 90 |
| Mustard, Black | 70 | 100 | 100 | 100 | 100 | 100 |
| Nutsedge, | 35 | 15 | 35 | 40 | — | 50 |
| Purple | | | | | | |
| Pigweed | 100 | 100 | 100 | 100 | 100 | 100 |
| Pigweed, | 40 | — | — | — | — | — |
| Palmer | | | | | | |
| Quackgrass | 60 | 95 | 75 | 65 | 65 | 35 |
| Ragweed | 75 | 5 | 100 | 70 | 25 | 100 |
| Ryegrass, | 10 | 50 | 75 | 65 | 70 | 20 |
| Italian | | | | | | |
| Sandbur | 40 | 40 | 70 | 5 | 15 | 0 |
| Signalgrass | 70 | 65 | 35 | 75 | 95 | 60 |
| Sowthistle | — | — | — | 100 | 100 | 100 |
| Surinam Grass | 20 | 95 | 50 | 35 | 35 | 25 |
| Wild Poinsettia | _{—} | 80 | 95 | 50 | 35 | 65 |

| | 8 g ai/ha | 4 g ai/ha |
|---|---|---|
| | Compound | Compound |
| | 189 | 189 |
| Barnyardgrass | 0 | 0 |
| Bermudagrass | 0 | 0 |
| Bluegrass | 0 | 0 |
| Buckwheat, Wild | 100 | 70 |
| Crabgrass, Large | 95 | 0 |
| Crabgrass, Naked | 95 | 40 |
| Dallisgrass | 5 | 0 |
| Dayflower, VA | 30 | 0 |
| Foxtail, Green | 20 | 0 |
| Guineagrass | 75 | 30 |
| Johnsongrass | 0 | 0 |
| Kochia | 100 | 98 |
| Leafy Spurge | 75 | 35 |
| Mallow | 65 | 90 |
| Morningglory | 30 | 0 |
| Mustard, Black | 98 | 95 |
| Nutsedge, Purple | 10 | 0 |
| Pigweed | 60 | 60 |
| Quackgrass | 0 | 0 |
| Ragweed | 95 | |
| Ryegrass, Italian | 0 | 0 |
| Sandbur | 0 | 0 |
| Signalgrass | 30 | 0 |
| Sowthistle | 98 | 98 |
| Surinam Grass | 0 | 0 |
| Wild Poinsettia | 25 | 10 |

### Test F

Seeds of plant species selected from corn (Zea mays), soybean (Glycine max), velvetleaf (Abutilon theophrasti), lambsquarters (Chenopodium album), wild poinsettia (Euphorbia heterophylla), pigweed, palmer (palmer pigweed, Amaranthus palmeri), common waterhemp (Amaranthus rudis), surinam grass (Brachiaria decumbens), large crabgrass (Digitaria sanguinalis), crabgrass, Brazil (Brazilian crabgrass, Digitaria horizontalis), fall panicum (Panicum dichotomiflorum), giant foxtail (Setaria faberii), green foxtail (Setaria viridis), goosegrass (Eleusine indica), johnsongrass (Sorghum halepense), common ragweed (Ambrosia elation), barnyardgrass (Echinochloa crus-galli), southern sandbur (Cenchrus echinatus), arrowleaf sida (Sida rhombifolia), Italian ryegrass (Lolium multiflorum), dayflower, VA (Virginia (VA) dayflower, Commelina virginica), field bindweed (Convolvulus arvensis), cocklebur (common cocklebur, Xanthium strumarium), morningglory (Ipomoea coccinea), nightshade (eastern black nightshade, Solanum ptycanthum), kochia (Kochia scoparia), yellow nutsedge (Cyperus esculentus), and hairy beggarticks (Bidens pilosa), were planted into a silt loam soil and treated preemergence with test chemicals formulated in a non-phytotoxic solvent mixture which included a surfactant.

At the same time, plants from these crop and weed species and also horseweed (Canada horseweed, Conyza canadensis), smartweed (ladysthumb smartweed, Polygonum persicaria), waterhemp_RES1, (ALS & Triazine resistant common waterhemp, Amaranthus rudis), and waterhemp_RES2, (ALS & HPPD resistant common waterhemp, Amaranthus rudis) were treated with postemergence applications of test chemicals formulated in the same manner. Plants ranged in height from 2 to 18 cm for postemergence treatments (1- to 4-leaf stage). Treated plants and controls were maintained in a greenhouse for 14 to 21 days, after which time all species were compared to controls and visually evaluated. Plant response ratings, summarized in Table F, are based on a scale of 0 to 100 where 0 is no effect and 100 is complete control. A dash (-) response means no test result.

TABLE F

| | 500 g ai/ha | | | |
|---|---|---|---|---|
| | Compounds | | | |
| | 78 | 104 | 105 | 188 |
| Soybean | 100 | 95 | 100 | 98 |
| Arrowleaf Sida | 95 | 95 | 90 | 80 |
| Barnyardgrass | 90 | 95 | 90 | 95 |
| Crabgrass, Brazil | 95 | 95 | 90 | 95 |
| Horseweed | 75 | 45 | 70 | 50 |
| Waterhemp_RES1 | 100 | 85 | 95 | 85 |
| Waterhemp_RES2 | 95 | 85 | 95 | 60 |
| Common Ragweed | 95 | 98 | 95 | 98 |
| Common Waterhemp | 98 | 90 | 80 | 90 |
| Fall Panicum | 95 | 90 | 90 | 90 |
| Field Bindweed | 98 | 95 | 90 | 95 |
| Hairy Beggarticks | 90 | 90 | 85 | 75 |
| Italian Ryegrass | 90 | 85 | 90 | 85 |
| Kochia | 95 | 95 | 95 | 95 |
| Smartweed | 100 | 98 | 100 | 95 |
| Corn | | | | |
| Pigweed, Palmer | 98 | 95 | 80 | 70 |
| Southern Sandbur | 90 | 90 | 90 | 90 |
| Dayflower, VA | 98 | 95 | 90 | 90 |
| Wild Poinsettia | 90 | 100 | 100 | 85 |

| | 250 g ai/ha | | | | | |
|---|---|---|---|---|---|---|
| | Compounds | | | | | |
| | 78 | 104 | 105 | 109 | 188 | |
| Soybean | 95 | 98 | 95 | 95 | 98 | |
| Arrowleaf Sida | 80 | 80 | 80 | 60 | 70 | |
| Barnyardgrass | 70 | 80 | 60 | 50 | 60 | |
| Crabgrass, Brazil | 90 | 90 | 70 | 70 | 90 | |
| Horseweed | 50 | 35 | 50 | — | 40 | |
| Waterhemp_RES1 | 65 | 75 | 90 | — | 80 | |
| Waterhemp_RES2 | 75 | 80 | 85 | 70 | 50 | |
| Common Ragweed | 80 | 98 | 90 | — | 85 | |
| Common Waterhemp | 70 | 85 | 75 | 80 | 80 | |
| Fall Panicum | 90 | 80 | 80 | 30 | 75 | |
| Field Bindweed | 95 | 90 | 80 | 80 | 95 | |
| Hairy Beggarticks | 75 | 85 | 70 | 60 | 60 | |
| Italian Ryegrass | 60 | 60 | 65 | 30 | 60 | |
| Kochia | 90 | 95 | 95 | — | 90 | |
| Smartweed | — | — | — | 80 | — | |
| Corn | 85 | 90 | 80 | 75 | 95 | |
| Pigweed, Palmer | 90 | 75 | 75 | 70 | 60 | |
| Southern Sandbur | 80 | 90 | 70 | 20 | 70 | |
| Dayflower, VA | 90 | 90 | 85 | 70 | 90 | |
| Wild Poinsettia | 80 | 90 | 100 | — | 80 | |
| | 125 g ai/ha | | | | | |
| | Compounds | | | | | |
| | 78 | 104 | 105 | 109 | 110 | 188 |
| Soybean | — | 98 | 90 | 95 | 30 | 95 |
| Arrowleaf Sida | 75 | 70 | 60 | 50 | 85 | 60 |
| Barnyardgrass | 40 | 30 | 15 | 30 | 50 | 50 |
| Crabgrass, Brazil | 70 | 60 | 50 | 40 | 50 | 75 |
| Horseweed | 40 | 35 | 35 | — | — | 30 |
| Waterhemp_RES1 | 60 | 90 | 75 | — | 100 | 50 |
| Waterhemp_RES2 | 70 | 95 | 75 | 60 | 100 | 50 |
| Common Ragweed | 70 | 70 | 90 | — | — | 70 |
| Common Waterhemp | 75 | 85 | 65 | 75 | 100 | 80 |
| Fall Panicum | 50 | 50 | 30 | 20 | 30 | 55 |
| Field Bindweed | 90 | 90 | 70 | 50 | 60 | 90 |
| Hairy Beggarticks | 50 | 60 | 50 | 50 | 70 | 50 |
| Italian Ryegrass | 40 | 35 | 30 | 15 | 30 | 30 |
| Kochia | 90 | 90 | 90 | — | — | 95 |
| Smartweed | — | — | — | 70 | — | — |
| Corn | 50 | 50 | 40 | 20 | 20 | 60 |
| Pigweed, Palmer | 70 | 70 | 60 | 60 | 85 | 50 |
| Southern Sandbur | 30 | 45 | 30 | 10 | 30 | 50 |
| Dayflower, VA | 60 | 70 | 80 | 40 | 60 | 70 |
| Wild Poinsettia | 70 | 95 | 70 | 70 | 95 | 60 |

| | 62 g ai/ha | | | | | |
|---|---|---|---|---|---|---|
| | Compounds | | | | | |
| | 78 | 104 | 105 | 110 | 110 | 188 |
| Soybean | 80 | 95 | 80 | 90 | 35 | 95 |
| Arrowleaf Sida | 60 | 60 | 50 | 40 | 75 | 50 |
| Barnyardgrass | 30 | 10 | 5 | 10 | 30 | 20 |
| Crabgrass, Brazil | 40 | 50 | 35 | 30 | 40 | 30 |
| Horseweed | 30 | 30 | 25 | — | — | 30 |
| Waterhemp_RES1 | 70 | 75 | 70 | — | 90 | 70 |
| Waterhemp_RES2 | 65 | 80 | 70 | 60 | 98 | 70 |
| Common Ragweed | 50 | 50 | 70 | — | — | 60 |
| Common Waterhemp | 60 | 80 | 60 | 60 | 90 | 70 |
| Fall Panicum | 20 | 40 | 20 | 20 | 20 | 20 |
| Field Bindweed | 70 | 70 | 75 | 40 | 50 | 70 |
| Hairy Beggarticks | 40 | 50 | 50 | 50 | 60 | 60 |
| Italian Ryegrass | 10 | 15 | 15 | 0 | 25 | 10 |
| Kochia | 90 | 80 | 90 | — | — | 60 |
| Smartweed | — | — | — | 50 | — | — |
| Corn | 20 | 25 | 30 | 10 | 15 | 20 |
| Pigweed, Palmer | 60 | 80 | 25 | 50 | 85 | 50 |
| Southern Sandbur | 10 | 30 | 10 | 0 | 30 | 20 |
| Dayflower, VA | 50 | 55 | 50 | 30 | 60 | 50 |
| Wild Poinsettia | 50 | 60 | 50 | 60 | 80 | 70 |
| | 31 g ai/ha | | | | | |
| | Compounds | | | | | |
| | 78 | 104 | 105 | 109 | 110 | 188 |
| Soybean | 75 | 95 | 70 | 70 | 30 | 98 |
| Arrowleaf Sida | 50 | 55 | 40 | 30 | 85 | 50 |
| Barnyardgrass | 5 | 0 | 10 | 0 | 20 | 0 |
| Crabgrass, Brazil | 40 | 40 | 30 | 10 | 30 | 20 |
| Horseweed | 25 | 25 | 20 | — | — | 20 |
| Waterhemp_RES1 | 60 | 70 | 70 | — | 98 | 60 |
| Waterhemp_RES2 | 60 | 80 | 60 | 50 | 98 | 65 |
| Common Ragweed | 65 | 40 | 50 | — | — | 50 |
| Common Waterhemp | 70 | 75 | 65 | 50 | 100 | 60 |
| Fall Panicum | 10 | 10 | 10 | 10 | 20 | 20 |
| Field Bindweed | 40 | 70 | 70 | 40 | 60 | 70 |
| Hairy Beggarticks | 30 | 50 | 40 | 45 | 60 | 50 |
| Italian Ryegrass | 0 | 0 | 0 | 0 | 20 | 5 |
| Kochia | 65 | 75 | 60 | — | — | 40 |
| Smartweed | — | — | — | 50 | — | — |
| Corn | 15 | 10 | 10 | 0 | 10 | 10 |
| Pigweed, Palmer | 50 | 60 | 30 | 50 | 90 | 50 |
| Southern Sandbur | 0 | 10 | 0 | 0 | 25 | 0 |
| Dayflower, VA | 20 | 35 | 20 | 20 | 40 | 40 |
| Wild Poinsettia | 25 | 45 | 40 | 50 | 75 | 60 |

| | 16 g ai/ha | | 8 g ai/ha |
|---|---|---|---|
| | Compounds | | Compound |
| | 109 | 110 | 110 |
| Soybean | 50 | 30 | 20 |
| Arrowleaf Sida | 30 | 75 | 60 |
| Barnyardgrass | 0 | 20 | 10 |
| Crabgrass, Brazil | 10 | 30 | 20 |
| Horseweed | | | |
| Waterhemp_RES1 | — | 90 | 85 |
| Waterhemp_RES2 | 30 | 90 | 85 |
| Common Ragweed | | | |
| Common Waterhemp | 50 | 95 | 90 |
| Fall Panicum | 10 | 10 | 10 |
| Field Bindweed | 30 | 40 | 30 |
| Hairy Beggarticks | 35 | 50 | 30 |
| Italian Ryegrass | 0 | 20 | 10 |
| Kochia | | | |
| Smartweed | 20 | — | |
| Corn | 0 | 10 | 10 |
| Pigweed, Palmer | 30 | 85 | 60 |
| Southern Sandbur | 0 | 20 | 20 |
| Dayflower, VA | 15 | 50 | 25 |
| Wild Poinsettia | 40 | 70 | 70 |

Preemergence

| | 250 g ai/ha | | |
|---|---|---|---|
| | Compounds | | |
| | 200 | 201 | 224 |
| Soybean | 75 | 40 | 50 |
| Arrowleaf Sida | 80 | 75 | 35 |
| Barnyardgrass | 50 | 95 | 70 |
| Crabgrass, Brazil | 100 | 100 | 100 |
| Cocklebur | 30 | — | — |
| Common Ragweed | 30 | 65 | 60 |
| Common Waterhemp | 100 | 100 | 100 |
| Morningglory | 75 | 80 | 25 |
| Nightshade | 98 | 98 | 95 |
| Fall Panicum | 90 | 100 | — |
| Field Bindweed | 60 | 100 | 90 |
| Giant Foxtail | 40 | 100 | 98 |
| Goosegrass | 90 | 95 | 95 |
| Green Foxtail | 85 | 80 | 95 |
| | 250 g ai/ha | | |
| | Compounds | | |
| | 200 | 201 | 224 |
| Hairy Beggarticks | 40 | 40 | 10 |
| Italian Ryegrass | 75 | 75 | 60 |
| Johnsongrass | 75 | 95 | 75 |
| Kochia | 100 | 100 | 100 |
| Lambsquarters | 98 | 100 | 100 |
| Large Crabgrass | 100 | 100 | 100 |
| Corn | 35 | 60 | 40 |
| Pigweed, Palmer | 85 | 90 | 100 |
| Southern Sandbur | 60 | 75 | 15 |
| Surinam Grass | 95 | 65 | 65 |
| Velvetleaf | 85 | 100 | 95 |
| Dayflower, VA | 60 | 65 | 85 |
| Wild Poinsettia | 35 | 65 | 85 |
| Yellow Nutsedge | 35 | 25 | 35 |

| | 125 g ai/ha | | | | | |
|---|---|---|---|---|---|---|
| | Compounds | | | | | |
| | 78 | 104 | 105 | 110 | 179 | 188 |
| Soybean | 95 | 90 | 100 | — | 70 | 85 |
| Arrowleaf Sida | 35 | 30 | 15 | 90 | 35 | 20 |
| Barnyardgrass | 100 | 100 | 98 | 35 | 50 | 100 |
| Crabgrass, Brazil | 100 | 100 | 100 | 100 | 100 | 100 |
| Cocklebur | — | — | — | — | — | — |
| Common Ragweed | 100 | 100 | 100 | 90 | 80 | 90 |
| Common Waterhemp | 100 | 100 | 100 | 100 | 75 | 100 |
| Morningglory | 100 | 100 | 95 | 60 | 70 | 100 |
| Nightshade | 98 | 100 | 100 | 95 | 100 | 98 |
| Fall Panicum | 100 | 100 | 100 | 100 | 98 | 100 |
| Field Bindweed | 95 | 95 | 100 | 90 | 75 | 100 |
| Giant Foxtail | 100 | 100 | 100 | 98 | 98 | 100 |
| Goosegrass | 100 | 100 | 98 | 95 | 30 | 100 |
| Green Foxtail | 100 | 100 | 100 | 100 | 70 | 100 |
| Hairy Beggarticks | 80 | 10 | 40 | 50 | 40 | 0 |
| Italian Ryegrass | 90 | 75 | 80 | 60 | 65 | 80 |
| Johnsongrass | 100 | 100 | 98 | 80 | 70 | 95 |
| Kochia | 100 | 100 | 100 | 90 | 90 | 100 |
| Lambsquarters | 100 | 100 | 100 | 100 | 100 | 100 |
| Large Crabgrass | 100 | 100 | 100 | 85 | 90 | 100 |
| Corn | 70 | 60 | 60 | 15 | 30 | 65 |
| Pigweed, Palmer | 100 | 100 | 80 | 100 | 100 | 50 |
| Southern Sandbur | 95 | 90 | 95 | 75 | 35 | 100 |
| Surinam Grass | 100 | 80 | 40 | 10 | 60 | 100 |
| Velvetleaf | 95 | 100 | 40 | 75 | 40 | 100 |
| Dayflower, VA | 100 | 100 | 98 | 65 | 40 | 100 |
| Wild Poinsettia | 98 | 98 | 95 | 80 | 50 | 100 |
| Yellow Nutsedge | 90 | 75 | 65 | 0 | 60 | 70 |

| | 125 g ai/ha | | | | |
|---|---|---|---|---|---|
| | Compounds | | | | |
| | 196 | 197 | 200 | 201 | 224 |
| Soybean | 50 | 65 | 65 | 40 | 20 |
| Arrowleaf Sida | 70 | 10 | 35 | 0 | 30 |
| Barnyardgrass | 65 | 0 | 20 | 85 | 40 |
| Crabgrass, Brazil | 100 | 100 | 100 | 100 | 100 |
| Cocklebur | — | — | — | — | 0 |
| Common Ragweed | 90 | 80 | 5 | 35 | 0 |
| Common Waterhemp | 100 | 85 | 100 | 80 | 100 |
| Morningglory | 80 | 60 | 60 | 65 | 0 |
| Nightshade | 95 | 70 | 75 | 95 | 0 |
| Fall Panicum | 100 | 98 | 80 | 100 | — |
| Field Bindweed | 85 | 80 | 50 | 75 | 90 |
| Giant Foxtail | 100 | 100 | 5 | 75 | 75 |
| Goosegrass | 95 | 70 | 65 | 95 | 60 |
| Green Foxtail | 100 | 65 | 60 | 75 | 70 |
| Hairy Beggarticks | 35 | 20 | 0 | 35 | 0 |
| Italian Ryegrass | 50 | 30 | 60 | 75 | 0 |
| Johnsongrass | 80 | 20 | 70 | 80 | 35 |
| Kochia | 100 | 98 | 90 | 100 | 100 |
| Lambsquarters | 100 | 80 | 70 | 100 | 98 |
| Large Crabgrass | 100 | 100 | 100 | 100 | 100 |
| Corn | 25 | 20 | 0 | 20 | 20 |
| Pigweed, Palmer | 100 | 90 | 95 | 65 | 100 |
| Southern Sandbur | 40 | 15 | 15 | 30 | 35 |
| Surinam Grass | 70 | 20 | 65 | 35 | 50 |
| Velvetleaf | 35 | 25 | 80 | 30 | 80 |
| Dayflower, VA | 75 | 40 | 10 | 35 | 40 |
| Wild Poinsettia | 65 | 60 | 20 | 25 | 65 |
| Yellow Nutsedge | 50 | 25 | 25 | 0 | 35 |

| | 62 g ai/ha | | | | | |
|---|---|---|---|---|---|---|
| | Compounds | | | | | |
| | 78 | 104 | 105 | 110 | 179 | 188 |
| Soybean | 80 | 70 | 80 | 0 | 65 | 30 |
| Arrowleaf Sida | 20 | 20 | 0 | 80 | 0 | 10 |
| Barnyardgrass | 98 | 100 | 98 | 10 | 20 | 80 |
| Crabgrass, Brazil | 100 | 100 | 100 | 100 | 100 | 100 |
| Cocklebur | — | — | — | — | — | — |
| Common Ragweed | 98 | 100 | 35 | 80 | 65 | 90 |
| Common Waterhemp | 100 | 100 | 100 | 100 | 25 | 100 |
| Morningglory | 90 | 80 | 90 | 10 | 60 | 70 |
| Nightshade | 98 | 100 | 100 | 85 | 85 | 95 |
| Fall Panicum | 100 | 100 | 100 | 98 | 70 | 100 |
| Field Bindweed | 90 | 50 | 40 | 50 | 50 | 40 |
| Giant Foxtail | 100 | 100 | 100 | 95 | 40 | 100 |
| Goosegrass | 90 | 75 | 90 | 60 | 30 | 98 |
| Green Foxtail | 100 | 100 | 100 | 100 | 20 | 100 |
| Hairy Beggarticks | 35 | 0 | 20 | 30 | 40 | 0 |
| Italian Ryegrass | 60 | 60 | 60 | 20 | 40 | 70 |
| Johnsongrass | 95 | 75 | 98 | 25 | 20 | 80 |
| Kochia | 100 | 100 | 100 | 80 | 35 | 98 |
| Lambsquarters | 100 | 100 | 100 | 50 | 30 | 100 |
| Large Crabgrass | 100 | 100 | 100 | 75 | 95 | 100 |
| Corn | 65 | 50 | 40 | 0 | 30 | 15 |
| Pigweed, Palmer | 100 | 50 | 50 | 100 | 35 | — |
| Southern Sandbur | 80 | 80 | 75 | 35 | 0 | 65 |
| Surinam Grass | 75 | 75 | 35 | 5 | 20 | 100 |
| Velvetleaf | 75 | 95 | 20 | 40 | 20 | 100 |
| Dayflower, VA | 95 | 98 | 80 | 10 | 10 | 90 |
| Wild Poinsettia | 65 | 80 | 60 | 50 | 40 | 100 |
| Yellow Nutsedge | 70 | 60 | 25 | 0 | 10 | 15 |

| | 62 g ai/ha | | | | |
|---|---|---|---|---|---|
| | Compounds | | | | |
| | 196 | 197 | 200 | 201 | 224 |
| Soybean | 50 | 20 | 0 | — | 25 |
| Arrowleaf Sida | 5 | 5 | 5 | 0 | 35 |
| Barnyardgrass | 10 | 0 | 25 | 10 | 0 |
| Crabgrass, Brazil | 100 | 100 | 95 | 95 | 98 |
| Cocklebur | — | — | 30 | — | 0 |
| Common Ragweed | 70 | 40 | 0 | 0 | 0 |
| Common Waterhemp | 75 | 65 | 20 | 75 | 100 |
| Morningglory | 70 | 30 | 10 | 25 | 0 |
| Nightshade | 75 | 30 | 20 | 80 | — |
| Fall Panicum | 98 | 70 | 35 | 90 | — |
| Field Bindweed | 75 | 50 | 0 | 20 | 65 |
| Giant Foxtail | 70 | 20 | 0 | 75 | 40 |
| Goosegrass | 70 | 5 | 35 | 50 | 35 |
| Green Foxtail | 75 | 35 | 5 | 40 | 35 |
| Hairy Beggarticks | 15 | 25 | 0 | 20 | 0 |
| Italian Ryegrass | 20 | 30 | 0 | 30 | 20 |
| Johnsongrass | 35 | 0 | 20 | 25 | 0 |
| Kochia | 85 | 75 | 70 | 90 | 90 |
| Lambsquarters | 98 | 35 | 65 | 70 | 98 |
| Large Crabgrass | 100 | 35 | 20 | 70 | 98 |
| Corn | 0 | 0 | 0 | 10 | 0 |
| Pigweed, Palmer | 100 | 95 | 0 | 40 | 100 |
| Southern Sandbur | 10 | 0 | 0 | 0 | 5 |
| Surinam Grass | 25 | 20 | 5 | 0 | 70 |
| Velvetleaf | 20 | 15 | 20 | 0 | 50 |
| Dayflower, VA | 35 | 0 | 0 | 0 | 0 |
| Wild Poinsettia | 35 | 35 | 15 | 5 | 60 |
| Yellow Nutsedge | 20 | 5 | 0 | 0 | 0 |
| | 31 g ai/ha | | | | |
| | Compounds | | | | |
| | 78 | 104 | 105 | 110 | 179 |
| Soybean | 95 | 25 | 100 | 15 | 35 |
| Arrowleaf Sida | 0 | 10 | 0 | 65 | 0 |
| Barnyardgrass | 35 | 50 | 30 | 0 | 15 |
| Crabgrass, Brazil | 100 | 95 | 98 | 100 | 75 |
| Cocklebur | — | — | — | — | — |
| Common Ragweed | 90 | 95 | — | 25 | 40 |
| Common Waterhemp | 100 | 100 | 100 | 85 | 0 |
| Morningglory | 100 | 70 | 25 | 10 | 40 |
| Nightshade | 95 | 100 | 70 | 80 | 65 |
| Fall Panicum | 90 | 90 | 100 | 75 | 35 |
| Field Bindweed | 100 | 30 | 75 | 30 | 5 |
| Giant Foxtail | 100 | 100 | 100 | 70 | 25 |
| Goosegrass | 90 | 30 | 40 | 35 | 30 |
| Green Foxtail | 60 | 100 | 75 | 75 | 20 |
| Hairy Beggarticks | 25 | 0 | 0 | 35 | 40 |
| Italian Ryegrass | 5 | 40 | 50 | 20 | 10 |
| Johnsongrass | 30 | 60 | 75 | 15 | 20 |
| Kochia | 100 | 25 | 100 | 70 | 30 |
| Lambsquarters | 100 | 100 | 100 | 65 | — |
| Large Crabgrass | 100 | 100 | 98 | 10 | 40 |
| Corn | 20 | 30 | 40 | 0 | 20 |
| Pigweed, Palmer | 35 | 50 | — | 95 | 0 |
| Southern Sandbur | 5 | 20 | 10 | 20 | 0 |
| Surinam Grass | 50 | 35 | 10 | 5 | 20 |
| Velvetleaf | 10 | 50 | 0 | 30 | 0 |
| Dayflower, VA | 50 | 50 | 50 | 5 | 0 |
| Wild Poinsettia | 50 | 15 | 5 | 35 | 5 |
| Yellow Nutsedge | 20 | 25 | 25 | 0 | 0 |
| | 16 g ai/ha | | | | |
| | Compounds | | | | |
| | 188 | 196 | 197 | 200 | 201 |
| Soybean | — | 20 | 10 | 0 | 0 |
| Arrowleaf Sida | 0 | 0 | 5 | 0 | 0 |
| Barnyardgrass | 25 | 0 | 0 | 15 | 0 |
| Crabgrass, Brazil | 100 | 100 | 85 | 35 | 75 |
| Cocklebur | — | — | — | 20 | — |
| Common Ragweed | 30 | 65 | 35 | 0 | 0 |
| Common Waterhemp | 100 | 50 | 65 | 0 | 0 |
| Morningglory | 30 | 35 | 20 | 0 | 0 |
| Nightshade | 98 | 50 | 20 | 0 | 10 |
| Fall Panicum | 100 | 90 | 0 | 0 | 85 |
| Field Bindweed | 30 | 20 | 35 | 0 | 15 |
| Giant Foxtail | 100 | 40 | 0 | 0 | 0 |
| Goosegrass | 85 | 5 | 5 | 5 | 5 |
| Green Foxtail | 100 | 35 | 0 | 0 | 15 |
| Hairy Beggarticks | 0 | 15 | 0 | 0 | 0 |
| Italian Ryegrass | 35 | 0 | 35 | 0 | 30 |
| Johnsongrass | 70 | 0 | 0 | 10 | 0 |
| Kochia | 90 | 85 | 65 | 30 | 20 |
| Lambsquarters | 100 | 85 | 40 | 0 | 5 |
| Large Crabgrass | 100 | 95 | 35 | 35 | 35 |
| Corn | 20 | 0 | 0 | 0 | 0 |
| Pigweed, Palmer | 35 | 10 | 40 | 0 | 5 |
| Southern Sandbur | 30 | 5 | 0 | 0 | 0 |
| Surinam Grass | 20 | 0 | 0 | 0 | 0 |
| Velvetleaf | 20 | 0 | 0 | 0 | 0 |
| Dayflower, VA | 65 | 15 | 0 | 0 | 0 |
| Wild Poinsettia | 75 | 30 | 15 | 0 | 0 |
| Yellow Nutsedge | 20 | 0 | 0 | 0 | 0 |

| | 16 g ai/ha | | | |
|---|---|---|---|---|
| | Compounds | | | |
| | 78 | 104 | 105 | 110 |
| Soybean | 0 | 0 | 0 | — |
| Arrowleaf Sida | 0 | 0 | 0 | 65 |
| Barnyardgrass | 25 | 20 | 5 | 0 |
| Crabgrass, Brazil | 70 | 10 | 40 | 70 |
| Cocklebur | — | — | — | — |
| Common Ragweed | 30 | 0 | 0 | 0 |
| Common Waterhemp | 100 | 98 | 100 | 65 |
| Morningglory | 40 | 0 | 25 | 20 |
| Nightshade | 60 | 65 | 65 | 65 |
| Fall Panicum | 70 | 85 | 80 | 5 |
| Field Bindweed | 0 | 10 | 10 | 10 |
| Giant Foxtail | 50 | 30 | 65 | 40 |
| Goosegrass | 40 | 25 | 35 | 15 |
| Green Foxtail | 60 | 60 | 25 | 30 |
| Hairy Beggarticks | 20 | 0 | 0 | 35 |
| Italian Ryegrass | 0 | 0 | 0 | 0 |
| Johnsongrass | 20 | 35 | 25 | 0 |
| Kochia | 65 | — | 80 | 25 |
| Lambsquarters | 65 | 60 | 35 | 0 |
| Large Crabgrass | 98 | 90 | 70 | 5 |
| Corn | 10 | 0 | 30 | 0 |
| Pigweed, Palmer | 40 | 0 | 0 | 90 |
| Southern Sandbur | 0 | 0 | 0 | 20 |
| Surinam Grass | 20 | 10 | 10 | 0 |
| Velvetleaf | — | 0 | 0 | 35 |
| Dayflower, VA | 0 | 0 | 40 | 0 |
| Wild Poinsettia | 0 | 10 | 0 | 25 |
| Yellow Nutsedge | 0 | 0 | 0 | 0 |
| | 16 g ai/ha | | | |
| | Compounds | | | |
| | 196 | 197 | 200 | 201 |
| Soybean | 0 | 5 | 0 | 0 |
| Arrowleaf Sida | 0 | 5 | 0 | 0 |
| Barnyardgrass | 0 | 0 | 0 | 0 |
| Crabgrass, Brazil | 75 | 75 | 0 | 0 |
| Cocklebur | — | — | — | — |
| Common Ragweed | 30 | 0 | 0 | 0 |
| Common Waterhemp | 35 | 60 | — | 5 |
| Morningglory | 10 | 10 | 0 | 0 |
| Nightshade | 35 | 10 | — | 0 |
| Fall Panicum | 0 | 0 | 0 | 0 |
| Field Bindweed | 10 | 15 | 0 | 0 |
| Giant Foxtail | 0 | 0 | 0 | 0 |
| Goosegrass | 0 | 0 | 0 | 0 |
| Green Foxtail | 0 | 0 | 0 | 0 |
| Hairy Beggarticks | 0 | 0 | 0 | 0 |
| Italian Ryegrass | 0 | 0 | 0 | 0 |
| Johnsongrass | 0 | 0 | 0 | 0 |
| Kochia | 40 | 0 | 20 | 0 |
| Lambsquarters | 5 | 35 | 0 | 0 |
| Large Crabgrass | 35 | 0 | 0 | 0 |
| Corn | 0 | 0 | 0 | 0 |
| Pigweed, Palmer | 0 | 35 | 0 | 0 |
| Southern Sandbur | 0 | 0 | 0 | 0 |
| Surinam Grass | 0 | 0 | 0 | 0 |
| Velvetleaf | 0 | 0 | 0 | 0 |
| Dayflower, VA | 0 | 0 | 0 | 0 |
| Wild Poinsettia | 5 | 0 | 0 | 0 |
| Yellow Nutsedge | 0 | 0 | 0 | 0 |
| | 8 g ai/ha | | | |
| | Compounds | | | |
| | 78 | 104 | 105 | 110 |
| Soybean | 0 | 0 | 0 | 0 |
| Arrowleaf Sida | 0 | 0 | 0 | 35 |
| Barnyardgrass | 0 | 10 | 0 | 0 |
| Crabgrass, Brazil | 30 | 5 | 5 | 50 |
| Common Ragweed | 0 | 0 | 0 | 0 |
| Common Waterhemp | 65 | 65 | 75 | 65 |
| Morningglory | 0 | 0 | 20 | 0 |
| Nightshade | 0 | 0 | 0 | 50 |
| Fall Panicum | 40 | 15 | 75 | 0 |
| Field Bindweed | 0 | 0 | 0 | 0 |
| Giant Foxtail | 5 | 25 | 10 | 25 |
| Goosegrass | 5 | 5 | 30 | 0 |
| Green Foxtail | 10 | 5 | 0 | 25 |
| Hairy Beggarticks | 0 | 0 | 0 | 50 |
| Italian Ryegrass | 0 | 0 | 0 | 0 |
| Johnsongrass | 20 | 15 | 0 | 0 |
| Kochia | 40 | 0 | 0 | 20 |
| Lambsquarters | 50 | 35 | 35 | 0 |
| Large Crabgrass | 75 | 70 | 65 | 5 |
| Corn | 0 | 0 | 20 | 0 |
| Pigweed, Palmer | 20 | 0 | 0 | 70 |
| Southern Sandbur | 0 | 0 | 0 | 0 |
| Surinam Grass | 20 | 0 | 0 | 0 |
| Velvetleaf | 5 | 0 | 0 | 20 |
| Dayflower, VA | 0 | 0 | 0 | 0 |
| Wild Poinsettia | 0 | 0 | 0 | 25 |
| Yellow Nutsedge | 0 | 0 | 0 | 0 |
| | 8 g ai/ha | | | |
| | Compounds | | | |
| | 179 | 188 | 196 | 197 |
| Soybean | 0 | 0 | 0 | 0 |
| Arrowleaf Sida | 0 | 0 | 0 | 0 |
| Barnyardgrass | 0 | 0 | 0 | 0 |
| Crabgrass, Brazil | 0 | 35 | 30 | 0 |
| Common Ragweed | 0 | 0 | 0 | 0 |
| Common Waterhemp | 0 | 85 | 0 | 20 |
| Morningglory | 0 | 65 | 5 | 0 |
| Nightshade | 0 | 0 | 0 | 0 |
| Fall Panicum | 0 | 75 | 0 | 0 |
| Field Bindweed | 0 | 35 | 5 | 10 |
| Giant Foxtail | 0 | 5 | 0 | 0 |
| Goosegrass | 0 | 5 | 0 | 0 |
| Green Foxtail | 0 | 5 | 0 | 0 |
| Hairy Beggarticks | 35 | 0 | 0 | 0 |
| Italian Ryegrass | 0 | 0 | 0 | 0 |
| Johnsongrass | 0 | 0 | 0 | 0 |
| Kochia | 0 | 0 | 15 | 0 |
| Lambsquarters | — | 50 | 0 | 0 |
| Large Crabgrass | 0 | 90 | 35 | 0 |
| Corn | 0 | 20 | 0 | 0 |
| Pigweed, Palmer | 0 | 0 | 0 | 10 |
| Southern Sandbur | 0 | 0 | 0 | 0 |
| Surinam Grass | 0 | 20 | 0 | 0 |
| Velvetleaf | 0 | 0 | 0 | 0 |
| Dayflower, VA | 0 | 0 | 0 | 0 |
| Wild Poinsettia | 0 | 10 | 0 | 5 |
| Yellow Nutsedge | 0 | 0 | 0 | 0 |

What is claimed is:

1. A method for controlling the growth of undesired vegetation comprising contacting the vegetation or its environment with a herbicidally effective amount of a compound selected from Formula 1, N-oxides and salts thereof, 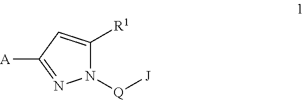
wherein
A is C(═O)N(R^{A})(R^{B}); or
A is a radical selected from the group consisting of 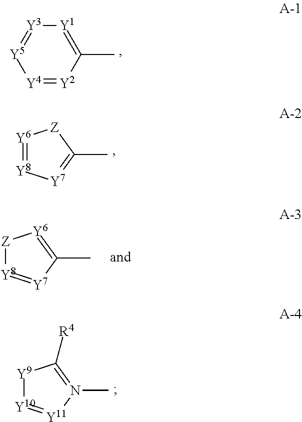
R^{A} is C₁-C₆ alkyl, C₂-C₆ alkenyl, C₂-C₆ alkynyl or C₁-C₄ alkoxy, each optionally substituted with halogen, cyano, C₁-C₄ alkoxy or C₁-C₄ alkylthio; or C₃-C₆ cycloalkyl, C₄-C₈ cycloalkylalkyl, C₃-C₇ oxiranylalkyl, C₃-C₇ oxetanylalkyl or C₃-C₇ thietanylalkyl, each optionally substituted with halogen, cyano, C₁-C₄ alkyl, C₁-C₄ alkoxy or C₁-C₄ alkylthio; or —N(R^{A1})(R^{A2}); or —SOₘ(R^{A3}); or phenyl optionally substituted with halogen, cyano, C₁-C₄ alkyl, C₁-C₃ haloalkyl, C₁-C₄ alkoxy or C₁-C₄ alkylthio;
R^{B} is H, C₁-C₄ alkyl, C₂-C₆ alkylcarbonyl or C₂-C₆ alkoxycarbonyl;
each Y¹, Y², Y³, Y⁴ and Y⁵ is independently N or CR², provided no more than 3 of Y¹, Y², Y³, Y⁴ and Y⁵ are N;
each Y⁶, Y⁷ and Y⁸ is independently N or CR³, provided no more than 2 of Y⁶, Y⁷ and Y⁸ are N;
each Y⁹, Y¹⁰ and Y¹¹ is independently N or CR⁴, provided no more than 2 of Y⁹, Y¹⁰ and Y¹¹ are N;
each Z is independently O or S;
R¹ is halogen, cyano, C₁-C₄ alkoxy, C₁-C₄ haloalkoxy, C₂-C₄ alkoxyalkyl, C₁-C₄ alkyl, C₁-C₄ haloalkyl, C₂-C₄ alkenyl, C₂-C₄ alkynyl, C₃-C₄ alkenyloxy, C₃-C₄ alkynyloxy, C₁-C₄ hydroxyalkyl, SOₙ(R^{S1}), C₂-C₄ alkylthioalkyl, C₂-C₄ alkylsulfonylalkyl, C₁-C₄ alkylamino or C₂-C₄ dialkylamino;
Q is —C(R⁵)(R⁶)— or —O—;
J is phenyl substituted with 1 R⁷ and optionally substituted with up to 2 R⁸; or
J is a 6-membered aromatic heterocyclic ring substituted with 1 R⁷ and optionally substituted with up to 2 R⁸ on carbon ring members; or
J is a 5-membered aromatic heterocyclic ring substituted with 1 R⁹ on carbon ring members and R¹⁰ on nitrogen ring members; and optionally substituted with 1 R¹¹ on carbon ring members;
each R² is independently H, halogen, cyano, nitro, SF₅, C₁-C₄ alkoxy, C₁-C₄ haloalkoxy, C₁-C₄ alkyl, C₁-C₄ haloalkyl, C₂-C₄ alkenyl, C₂-C₄ alkynyl, C₃-C₄ alkenyloxy, C₃-C₄ alkynyloxy or S(O)ₙR^{S2};
each R³ is independently H, halogen, cyano, C₁-C₄ alkyl, C₁-C₄ haloalkyl, C₁-C₄ alkoxy, C₁-C₄ haloalkoxy or S(O)ₙR^{S3};
each R⁴ is independently H, halogen, cyano, C₁-C₄ alkyl, C₁-C₄ haloalkyl, C₁-C₄ alkoxy, C₁-C₄ haloalkoxy or S(O)ₙR^{S4};
R⁵ is H, F, Cl, Br, cyano, C₁-C₄ alkyl, C₁-C₄ haloalkyl, C₂-C₆ alkylcarbonyl or C₂-C₆ alkoxycarbonyl;
R⁶ is H, F, C₁-C₄ alkyl, C₁-C₄ alkoxy or OH; or
R⁵ and R⁶ are taken together with the carbon to which they are attached to form C(═O);
R⁷ is halogen, cyano, SF₅, C₁-C₄ alkyl, C₁-C₄ haloalkyl, C₁-C₄ alkoxy, C₁-C₄ haloalkoxy or S(O)ₙR^{S5};
each R⁸ is independently halogen, cyano, C₁-C₄ alkyl, C₁-C₄ haloalkyl, C₁-C₄ alkoxy, C₁-C₄ haloalkoxy or S(O)ₙR^{S6}; or
R⁷ and R⁸ are taken together to form a 5-membered carbocyclic ring containing ring members selected from up to 2 O atoms and up to 2 S atoms, and optionally substituted on carbon atom ring members with up to 5 halogen atoms;
R⁹ is halogen, cyano, SF₅, C₁-C₄ alkyl, C₁-C₄ haloalkyl, C₁-C₄ alkoxy, C₁-C₄ haloalkoxy or S(O)ₙR^{S7};
R¹⁰ is C₁-C₄ alkyl or C₁-C₄ haloalkyl;
R¹¹ is halogen, cyano, C₁-C₄ alkyl, C₁-C₄ haloalkyl, C₁-C₄ alkoxy, C₁-C₄ haloalkoxy or S(O)ₙR^{S8};
each R^{S1}, R^{S2}, R^{S3}, R^{S4}, R^{S5}, R^{S6}, R^{S7} and R^{S8} is independently C₁-C₄ alkyl or C₁-C₄ haloalkyl; and
each m is independently 0, 1 or 2;
each n is independently 0, 1 or 2;
R^{A1} is H or C₁-C₄ alkyl;
R^{A2} is H or C₁-C₄ alkyl;
R^{A3} is C₁-C₄ alkyl or C₁-C₄ haloalkyl;
provided when
i) A is A-1; and each Y¹, Y², Y³, Y⁴ and Y⁵ is CH, then J is other than phenyl substituted with 1 R⁷ and optionally substituted with up to 2 R⁸;
ii) R⁷ is CF₃, then R⁸ is other than CF₃;
iii) R⁷ is CF₃, then R¹ is other than i-Pr; and
iv) R^{A} is CH₃, then R^{B} is other than CH₃.

2. The method of claim 1 wherein
A is C(═O)N(R^{A})(R^{B}); or A is a radical selected from the group consisting of A-1, A-2 and A-3;
each Y¹ and Y⁵ is independently N or CR²; and each Y², Y³ and Y⁴ is CR²;
each Y⁶ and Y⁷ is independently N or CR³; and Y⁸ is CR³;
R^{A} is C₁-C₆ alkyl or C₁-C₄ alkoxy, each optionally substituted with halogen, cyano or C₁-C₄ alkoxy; or C₃-C₆ cycloalkyl or C₄-C₈ cycloalkylalkyl, each optionally substituted with halogen, cyano, C₁-C₄ alkyl or C₁-C₄ alkoxy; or —N(R^{A1})(R^{A2}); or phenyl optionally substituted with halogen, cyano, C₁-C₄ alkyl, C₁-C₃ haloalkyl or C₁-C₄ alkoxy;
R^{B} is H, C₁-C₄ alkyl or C₂-C₆ alkylcarbonyl;
R¹ is halogen, C₁-C₄ alkoxy, C₁-C₄ alkyl, C₁-C₄ haloalkyl or C₁-C₄ alkylamino;
J is selected from 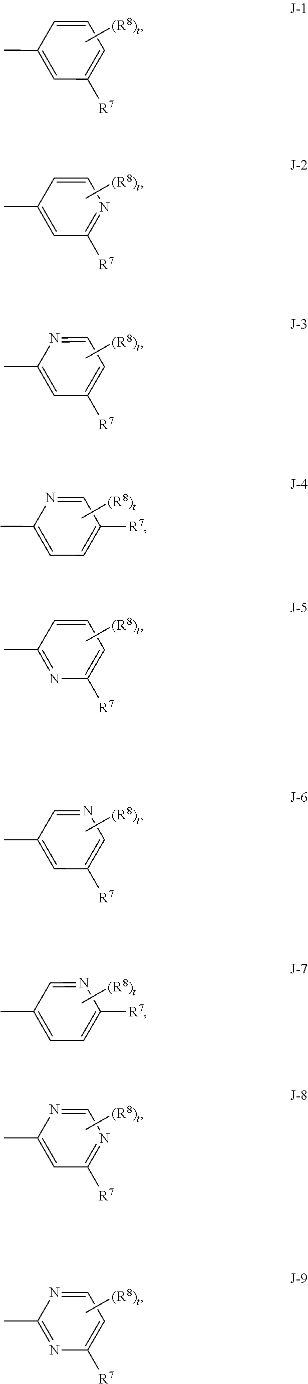 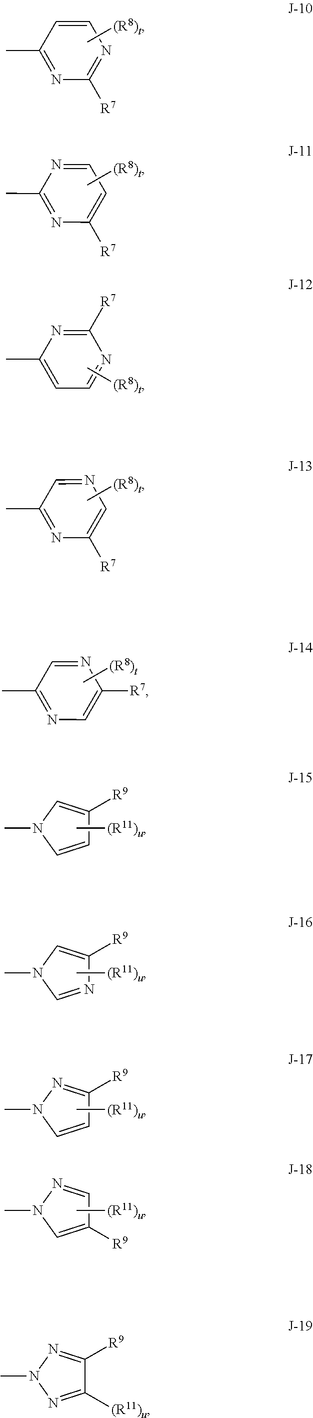 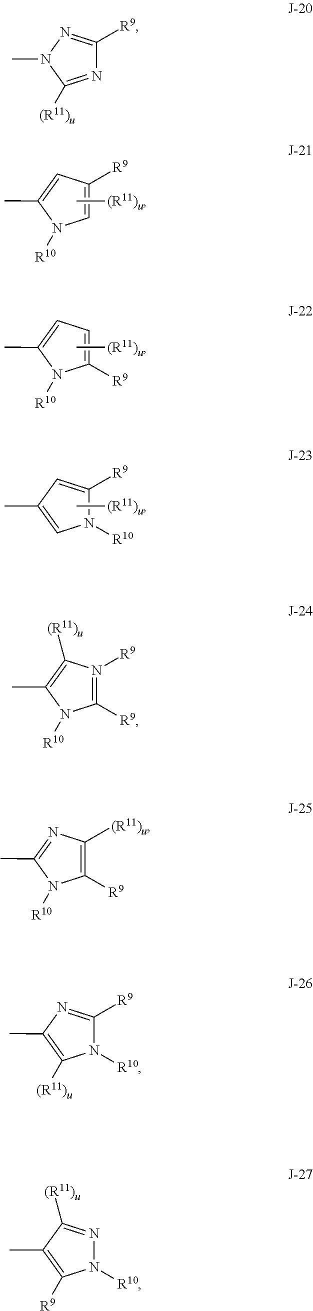 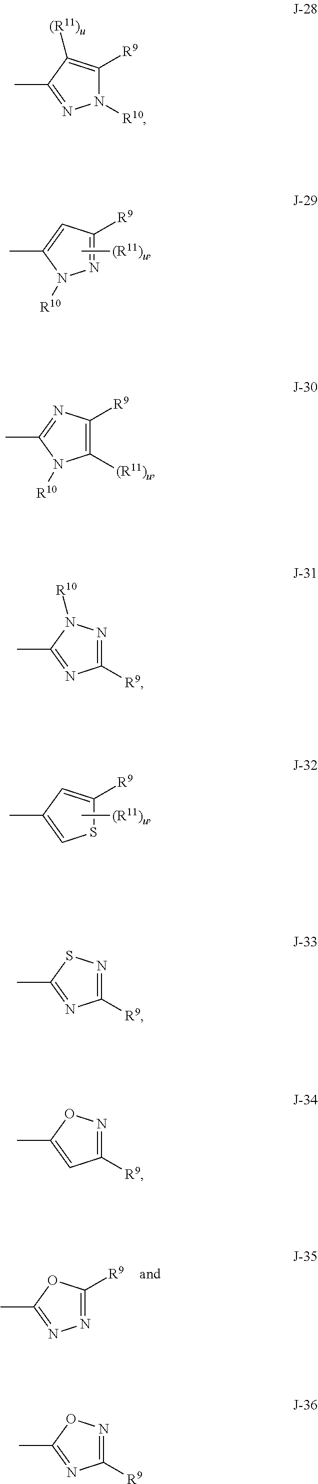
t is 0, 1 or 2; and
u is 0 or 1;
each R² is independently H, halogen, C₁-C₄ alkoxy, C₁-C₄ alkyl or C₁-C₄ haloalkyl;
each R³ is independently H, halogen or C₁-C₄ haloalkyl;
R⁵ is H, F or OH;
R⁶ is H or C₁-C₄ alkyl;
R⁷ is halogen, C₁-C₄ alkyl, C₁-C₄ haloalkyl, C₁-C₄ alkoxy or C₁-C₄ haloalkoxy;
R⁸ is independently halogen or C₁-C₄ haloalkyl; or
R⁷ and R⁸ are taken together with two adjacent carbon atoms to form a 5-membered carbocyclic ring containing ring members selected from up to 2 O atoms, and optionally substituted on carbon atom ring members with up to 5 halogen atoms;
R⁹ is halogen, C₁-C₄ alkyl, C₁-C₄ haloalkyl, C₁-C₄ alkoxy or C₁-C₄ haloalkoxy;
R¹⁰ is CH₃ or CH₂CF₃;
R¹¹ is halogen, C₁-C₄ alkyl, C₁-C₄ haloalkyl, C₁-C₄ alkoxy or C₁-C₄ haloalkoxy;
R^{A1} is H or C₁-C₄ alkyl; and
R^{A2} is H or C₁-C₄ alkyl.

3. The method of claim 2 wherein
A is C(═O)N(R^{A})(R^{B}); or A is A-1;
Y¹ is N or CR²; and each Y², Y³, Y⁴ and Y⁵ is independently CR²;
R^{A} is C₁-C₆ alkyl or C₁-C₄ alkoxy, each optionally substituted with halogen or C₁-C₄ alkoxy; or C₃-C₆ cycloalkyl or C₄-C₈ cycloalkylalkyl, each optionally substituted with halogen, C₁-C₄ alkyl or C₁-C₄ alkoxy; or phenyl optionally substituted with halogen, C₁-C₄ alkyl, C₁-C₃ haloalkyl or C₁-C₄ alkoxy;
R^{B} is H or C₁-C₄ alkyl;
R¹ is C₁-C₄ alkoxy, C₁-C₄ alkyl or C₁-C₄ haloalkyl;
Q is —C(R⁵)(R⁶)—;
J is selected from J-2 through J-14;
t is 0 or 1;
each R² is independently H, halogen, C₁-C₄ alkyl or C₁-C₄ haloalkyl;
R⁵ is H or F;
R⁶ is H;
R⁷ is C₁-C₄ haloalkyl or C₁-C₄ haloalkoxy; and
each R⁸ is independently F, Cl or CF₃.

4. The method of claim 3 wherein
A is C(═O)N(R^{A})(R^{B});
R^{A} is C₁-C₆ alkyl or C₁-C₄ alkoxy, each optionally substituted with halogen; or C₃-C₆ cycloalkyl optionally substituted with halogen or C₁-C₄ alkyl; or phenyl optionally substituted with halogen or C₁-C₃ haloalkyl;
R^{B} is H, CH₃ or CH₂CH₃;
R¹ is C₁-C₄ alkoxy or C₁-C₄ alkyl;
J is selected from J-2 and J-5;
t is 0;
each R² is independently H, F, Cl, CH₃ or CF₃;
R⁵ is H; and
R⁷ is CF₃, OCF₃ or OCHF₂.

5. The method of claim 4 wherein
R^{A} is C₁-C₆ alkyl or C₁-C₄ alkoxy, each optionally substituted with halogen; or C₃-C₆ cycloalkyl optionally substituted with halogen or C₁-C₄ alkyl;
R^{B} is H or CH₃;
R¹ is C₁-C₄ alkyl;
J is J-2; and
R⁷ is CF₃.

6. The method of claim 2 wherein
A is C(═O)N(R^{A})(R^{B});
R^{A} is C₁-C₆ alkyl or C₁-C₄ alkoxy, each optionally substituted with halogen; or cyclopropyl;
R^{B} is H;
R¹ is CH₃, CH₂CH₃ or CH₂CH₂CH₃;
Q is —C(R⁵)(R⁶)—;
J is selected from J-15 through J-33;
u is 0;
R⁵ is H;
R⁶ is H;
R⁹ is C₁-C₄ haloalkyl or C₁-C₄ haloalkoxy; and
R¹⁰ is CH₃.

7. The method of claim 6 wherein
R^{A} is —CH₂CF₃;
R¹ is CH₂CH₃;
J is J-29; and
R⁹ is F, CH₃ or CF₃.

8. The method of claim 1 comprising a compound selected from the group consisting of
5-ethyl-N-(2,2,2-trifluoroethyl)-1-[[(2-trifluoromethyl)-4-pyridinyl]methyl]-1H-pyrazole-3-carboxamide;
N-cyclopropyl-5-ethyl-1-[[2-trifluoromethyl)-4-pyridinyl]methyl]-1H-pyrazole-3-carboxamide; and
5-ethyl-1-[[1-methyl-3-(trifluoromethyl)-1H-pyrazole-5-yl]methyl]-N-(2,2,2-trifluoroethyl)-1H-pyrazole-3-carboxamide.

9. The method of claim 1 wherein the compound of Formula 1 is comprised in a herbicidal composition, said composition further comprising at least one component selected from the group consisting of surfactants, solid diluents and liquid diluents.

10. The method of claim 1 wherein the compound of Formula 1 is comprised in a herbicidal composition, said composition further comprising at least one additional active ingredient selected from the group consisting of other herbicides and herbicide safeners, and at least one component selected from the group consisting of surfactants, solid diluents and liquid diluents.

11. The method of claim 1 wherein the compound of Formula 1 is comprised in a herbicidal mixture, said mixture further (b) at least one additional active ingredient selected from (b1) through (b16) and salts of compounds of (b1) through (b16).

12. A compound selected from Formula 1, N-oxides and salts thereof, 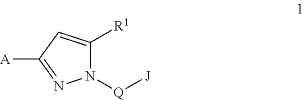
wherein
A is C(═O)N(R^{A})(R^{B}); or
A is a radical selected from the group consisting of 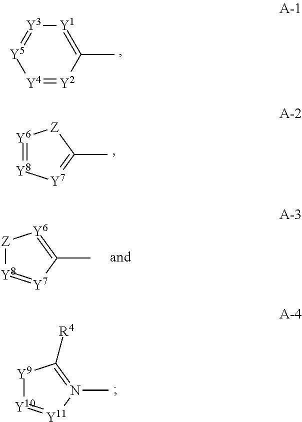
R^{A} is C₁-C₆ alkyl, C₂-C₆ alkenyl, C₂-C₆ alkynyl or C₁-C₄ alkoxy, each optionally substituted with halogen, cyano, C₁-C₄ alkoxy or C₁-C₄ alkylthio; or C₃-C₆ cycloalkyl, C₄-C₈ cycloalkylalkyl, C₃-C₇ oxiranylalkyl, C₃-C₇ oxetanylalkyl or C₃-C₇ thietanylalkyl, each optionally substituted with halogen, cyano, C₁-C₄ alkyl, C₁-C₄ alkoxy or C₁-C₄ alkylthio; or —N(R^{A1})(R^{A2}); or —SOₘ(R^{A3}); or phenyl optionally substituted with halogen, cyano, C₁-C₄ alkyl, C₁-C₃ haloalkyl, C₁-C₄ alkoxy or C₁-C₄ alkylthio;
R^{B} is H, C₁-C₄ alkyl, C₂-C₆ alkylcarbonyl or C₂-C₆ alkoxycarbonyl;
each Y¹, Y², Y³, Y⁴ and Y⁵ is independently N or CR², provided no more than 3 of Y¹, Y², Y³, Y⁴ and Y⁵ are N;
each Y⁶, Y⁷ and Y⁸ is independently N or CR³, provided no more than 2 of Y⁶, Y⁷ and Y⁸ are N;
each Y⁹, Y¹⁰ and Y¹¹ is independently N or CR⁴, provided no more than 2 of Y⁹, Y¹⁰ and Y¹¹ are N;
each Z is independently O or S;
R¹ is halogen, cyano, C₁-C₄ alkoxy, C₁-C₄ haloalkoxy, C₂-C₄ alkoxyalkyl, C₁-C₄ alkyl, C₁-C₄ haloalkyl, C₂-C₄ alkenyl, C₂-C₄ alkynyl, C₃-C₄ alkenyloxy, C₃-C₄ alkynyloxy, C₁-C₄ hydroxyalkyl, SOₙ(R^{S1}), C₂-C₄ alkylthioalkyl, C₂-C₄ alkylsulfonylalkyl, C₁-C₄ alkylamino or C₂-C₄ dialkylamino;
Q is —C(R⁵)(R⁶)— or —O—;
J is phenyl substituted with 1 R⁷ and optionally substituted with up to 2 R⁸; or
J is a 6-membered aromatic heterocyclic ring substituted with 1 R⁷ and optionally substituted with up to 2 R⁸ on carbon ring members; or
J is a 5-membered aromatic heterocyclic ring substituted with 1 R⁹ on carbon ring members and R¹⁰ on nitrogen ring members; and optionally substituted with 1 R¹¹ on carbon ring members;
each R² is independently H, halogen, cyano, nitro, SF₅, C₁-C₄ alkoxy, C₁-C₄ haloalkoxy, C₁-C₄ alkyl, C₁-C₄ haloalkyl, C₂-C₄ alkenyl, C₂-C₄ alkynyl, C₃-C₄ alkenyloxy, C₃-C₄ alkynyloxy or S(O)ₙR^{S2};
each R³ is independently H, halogen, cyano, C₁-C₄ alkyl, C₁-C₄ haloalkyl, C₁-C₄ alkoxy, C₁-C₄ haloalkoxy or S(O)ₙR^{S3};
each R⁴ is independently H, halogen, cyano, C₁-C₄ alkyl, C₁-C₄ haloalkyl, C₁-C₄ alkoxy, C₁-C₄ haloalkoxy or S(O)ₙR^{S4};
R⁵ is F, Cl, Br, cyano, C₁-C₄ alkyl, C₁-C₄ haloalkyl, C₂-C₆ alkylcarbonyl or C₂-C₆ alkoxycarbonyl;
R⁶ is H, F, C₁-C₄ alkyl, C₁-C₄ alkoxy or OH; or
R⁵ and R⁶ are taken together with the carbon to which they are attached to form C(═O);
R⁷ is halogen, cyano, SF₅, C₁-C₄ alkyl, C₁-C₄ haloalkyl, C₁-C₄ alkoxy, C₁-C₄ haloalkoxy or S(O)ₙR^{S5};
each R⁸ is independently halogen, cyano, C₁-C₄ alkyl, C₁-C₄ haloalkyl, C₁-C₄ alkoxy, C₁-C₄ haloalkoxy or S(O)ₙR^{S6}; or
R⁷ and R⁸ are taken together to form a 5-membered carbocyclic ring containing ring members selected from up to 2 O atoms and up to 2 S atoms, and optionally substituted on carbon atom ring members with up to 5 halogen atoms;
R⁹ is halogen, cyano, SF₅, C₁-C₄ alkyl, C₁-C₄ haloalkyl, C₁-C₄ alkoxy, C₁-C₄ haloalkoxy or S(O)ₙR^{S7};
R¹⁰ is C₁-C₄ alkyl or C₁-C₄ haloalkyl;
R¹¹ is halogen, cyano, C₁-C₄ alkyl, C₁-C₄ haloalkyl, C₁-C₄ alkoxy, C₁-C₄ haloalkoxy or S(O)ₙR^{S8};
each R^{S1}, R^{S2}, R^{S3}, R^{S4}, R^{S5}, R^{S6}, R^{S7} and R^{S8} is independently C₁-C₄ alkyl or C₁-C₄ haloalkyl; and
each m is independently 0, 1 or 2;
each n is independently 0, 1 or 2;
R^{A1} is H or C₁-C₄ alkyl;
R^{A2} is H or C₁-C₄ alkyl;
R^{A3} is C₁-C₄ alkyl or C₁-C₄ haloalkyl;
provided when
i) A is A-1; and each Y¹, Y², Y³, Y⁴ and Y⁵ is CH, then J is other than phenyl substituted with 1 R⁷ and optionally substituted with up to 2 R⁸;
ii) R⁷ is CF₃, then R⁸ is other than CF₃;
iii) R⁷ is CF₃, then R¹ is other than i-Pr; and
iv) R^{A} is CH₃, then R^{B} is other than CH₃;
and provided the compound of Formula 1 is other than a) methanone, [3-phenyl-5-(trichloromethyl)-1H-pyrazol-1-yl]-2-thienyl (949912-41-8), b) methanone, 2-furanyl[3-phenyl-5-trichloromethyl-1H-pyrazol-1-yl] (949912-42-9), c) methanone, [3-phenyl-5-(trifluoromethyl)-1H-pyrazol-1-yl]-2-thienyl (860262-65-3), d) methanone, 2-furanyl[3-phenyl-5-(trifluoromethyl)-1H-pyrazol-1-yl] (860262-66-4) and e) methanone, [3-phenyl-5-(trifluoromethyl)-1H-pyrazol-1-yl]-4-pyridinyl (860262-67-5).

13. The compound of claim 12 wherein
A is C(═O)N(R^{A})(R^{B}); or A is a radical selected from the group consisting of A-1, A-2 and A-3;
each Y¹ and Y⁵ is independently N or CR²; and each Y², Y³ and Y⁴ is CR²;
each Y⁶ and Y⁷ is independently N or CR³; and Y⁸ is CR³;
R^{A} is C₁-C₆ alkyl or C₁-C₄ alkoxy, each optionally substituted with halogen, cyano or C₁-C₄ alkoxy; or C₃-C₆ cycloalkyl or C₄-C₈ cycloalkylalkyl, each optionally substituted with halogen, cyano, C₁-C₄ alkyl or C₁-C₄ alkoxy; or —N(R^{A1})(R^{A2}); or phenyl optionally substituted with halogen, cyano, C₁-C₄ alkyl, C₁-C₃ haloalkyl or C₁-C₄ alkoxy;
R^{B} is H, C₁-C₄ alkyl or C₂-C₆ alkylcarbonyl;
R¹ is halogen, C₁-C₄ alkoxy, C₁-C₄ alkyl, C₁-C₄ haloalkyl or C₁-C₄ alkylamino;
J is selected from 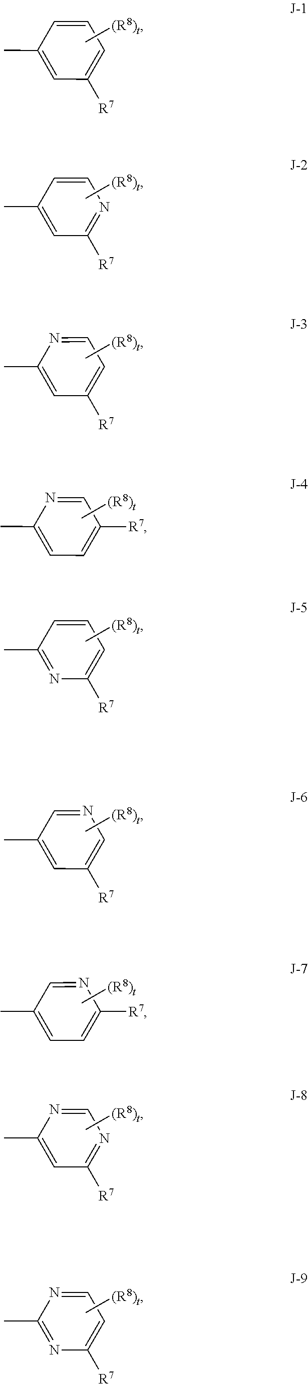 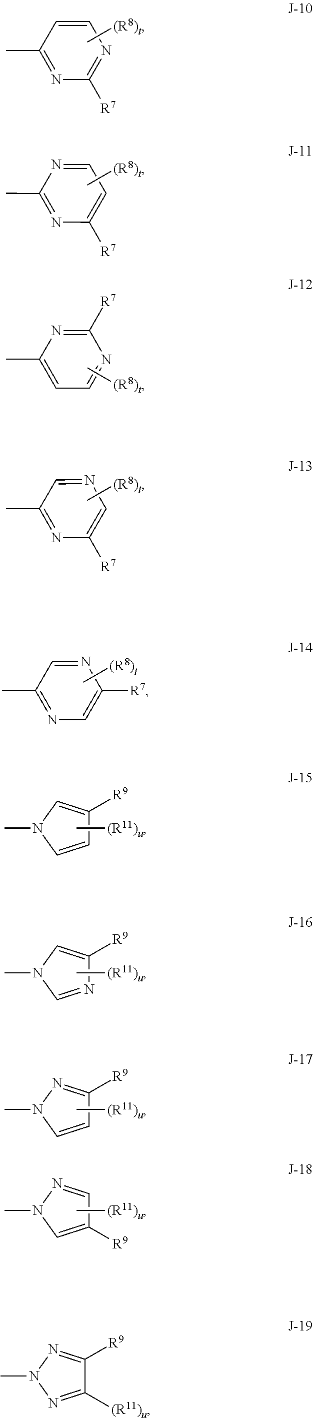 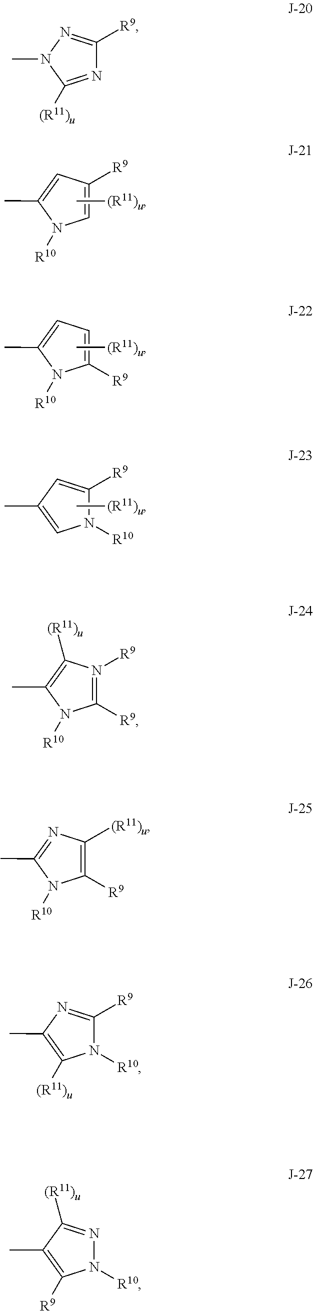 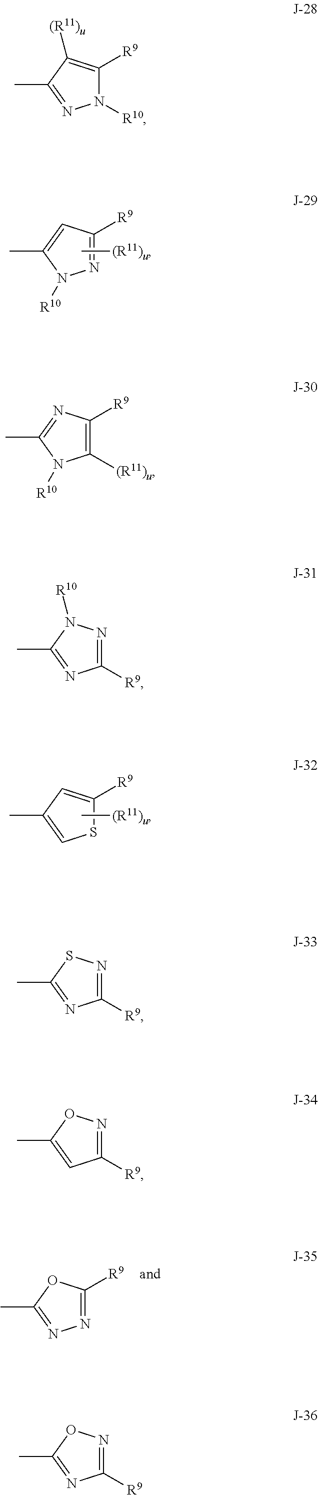
t is 0, 1 or 2; and
u is 0 or 1;
each R² is independently H, halogen, C₁-C₄ alkoxy, C₁-C₄ alkyl or C₁-C₄ haloalkyl;
each R³ is independently H, halogen or C₁-C₄ haloalkyl;
R⁵ is F or OH;
R⁶ is H or C₁-C₄ alkyl;
R⁷ is halogen, C₁-C₄ alkyl, C₁-C₄ haloalkyl, C₁-C₄ alkoxy or C₁-C₄ haloalkoxy;
R⁸ is independently halogen or C₁-C₄ haloalkyl; or
R⁷ and R⁸ are taken together with two adjacent carbon atoms to form a 5-membered carbocyclic ring containing ring members selected from up to 2 O atoms, and optionally substituted on carbon atom ring members with up to 5 halogen atoms;
R⁹ is halogen, C₁-C₄ alkyl, C₁-C₄ haloalkyl, C₁-C₄ alkoxy or C₁-C₄ haloalkoxy;
R¹⁰ is CH₃ or CH₂CF₃;
R¹¹ is halogen, C₁-C₄ alkyl, C₁-C₄ haloalkyl, C₁-C₄ alkoxy or C₁-C₄ haloalkoxy;
R^{A1} is H or C₁-C₄ alkyl; and
R^{A2} is H or C₁-C₄ alkyl.

14. The compound of claim 13 wherein
A is C(═O)N(R^{A})(R^{B}); or A is A-1;
Y¹ is N or CR²; and each Y², Y³, Y⁴ and Y⁵ is independently CR²;
R^{A} is C₁-C₆ alkyl or C₁-C₄ alkoxy, each optionally substituted with halogen or C₁-C₄ alkoxy; or C₃-C₆ cycloalkyl or C₄-C₈ cycloalkylalkyl, each optionally substituted with halogen, C₁-C₄ alkyl or C₁-C₄ alkoxy; or phenyl optionally substituted with halogen, C₁-C₄ alkyl, C₁-C₃ haloalkyl or C₁-C₄ alkoxy;
R^{B} is H or C₁-C₄ alkyl;
R¹ is C₁-C₄ alkoxy, C₁-C₄ alkyl or C₁-C₄ haloalkyl;
Q is —C(R⁵)(R⁶)—;
J is selected from J-2 through J-14;
t is 0 or 1;
each R² is independently H, halogen, C₁-C₄ alkyl or C₁-C₄ haloalkyl;
R⁵ is F;
R⁷ is C₁-C₄ haloalkyl or C₁-C₄ haloalkoxy; and
each R⁸ is independently F, Cl or CF₃.

15. The compound of claim 14 wherein
A is C(═O)N(R^{A})(R^{B});
R^{A} is C₁-C₆ alkyl or C₁-C₄ alkoxy, each optionally substituted with halogen; or C₃-C₆ cycloalkyl optionally substituted with halogen or C₁-C₄ alkyl; or phenyl optionally substituted with halogen or C₁-C₃ haloalkyl;
R^{B} is H, CH₃ or CH₂CH₃;
R¹ is C₁-C₄ alkoxy or C₁-C₄ alkyl;
J is selected from J-2 and J-5;
t is 0;
each R² is independently H, F, Cl, CH₃ or CF₃; and
R⁷ is CF₃, OCF₃ or OCHF₂.

16. The compound of claim 15 wherein
R^{A} is C₁-C₆ alkyl or C₁-C₄ alkoxy, each optionally substituted with halogen; or C₃-C₆ cycloalkyl optionally substituted with halogen or C₁-C₄ alkyl;
R^{B} is H or CH₃;
R¹ is C₁-C₄ alkyl;
J is J-2; and
R⁷ is CF₃.

17. The compound of claim 13 wherein
A is C(═O)N(R^{A})(R^{B});
R^{A} is C₁-C₆ alkyl or C₁-C₄ alkoxy, each optionally substituted with halogen; or cyclopropyl;
R^{B} is H;
R¹ is CH₃, CH₂CH₃ or CH₂CH₂CH₃;
Q is —C(R⁵)(R⁶)—;
J is selected from J-15 through J-33;
u is 0;
R⁹ is C₁-C₄ haloalkyl or C₁-C₄ haloalkoxy; and
R¹⁰ is CH₃.

18. The compound of claim 17 selected from the group consisting of
R^{A} is —CH₂CF₃;
R¹ is CH₂CH₃;
J is J-29; and
R⁹ is F, CH₃ or CF₃.

19. A herbicidal composition comprising a compound of claim 12 and at least one component selected from the group consisting of surfactants, solid diluents and liquid diluents.
